(19) United States
(12) Patent Application Publication
(10) Pub. No.: US 2013/0053365 A1
(43) Pub. Date: Feb. 28, 2013

(54) DIHYDROPYRIDOPHTHALAZINONE INHIBITORS OF POLY(ADP-RIBOSE)POLYMERASE (PARP)

(71) Applicants: Bing Wang, Sane Jose, CA (US); Daniel Chu, Santa Clara, CA (US)

(72) Inventors: Bing Wang, Sane Jose, CA (US); Daniel Chu, Santa Clara, CA (US)

(73) Assignee: BioMarin Pharmaceutical, Inc., Novato, CA (US)

(21) Appl. No.: 13/598,392

(22) Filed: Aug. 29, 2012

Related U.S. Application Data
(60) Provisional application No. 61/528,945, filed on Aug. 30, 2011.

(51) Int. Cl. A61K 31/55; C07D 498/06; A61K 31/551; A61P 35/02; A61P 35/00; A61P 25/00; A61P 29/00; C07D 487/06; A61K 31/553

(52) U.S. Cl. 514/210.21; 540/556; 540/578; 514/215; 540/548; 540/520; 514/212.06; 514/220; 514/211.11

(57) ABSTRACT

Provided are inhibitors of poly(ADP-ribose)polymerase according to Formula (M):

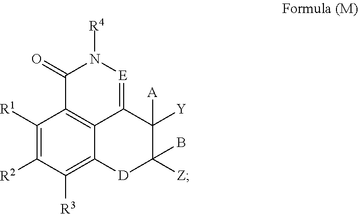

where A, B, Y, Z, D, E, R¹, R², R³, and R⁴ are as defined herein. Also described are pharmaceutical compositions, methods of preparing, and methods of using a compound of Formula M to treat diseases, disorders and conditions that are ameliorated by the inhibition of PARP activity.

### CROSS REFERENCE TO RELATED APPLICATIONS

The present application claims the benefit of priority of U.S. Provisional Application No. 61/528,945, filed Aug. 30, 2011, the content of which is hereby incorporated by reference in its entirety.

### FIELD OF THE INVENTION

Described herein are compounds, methods of making such compounds, pharmaceutical compositions and medicaments containing such compounds, and methods of preparing and using such compounds to treat or prevent diseases or conditions associated with the enzyme poly(ADP-ribose)polymerase (PARP).

### BACKGROUND OF THE INVENTION

The family of poly(ADP-ribose)polymerases (PARP) includes approximately 18 proteins, which all display a certain level of homology in their catalytic domain but differ in their cellular functions (Ame et al., BioEssays., 26(8), 882-893 (2004)). PARP-1 and PARP-2 are unique members of the family, in that their catalytic activities are stimulated by the occurrence of DNA strand breaks.

The mammalian enzyme PARP-1 is a multidomain protein. PARP has been implicated in the signaling of DNA damage through its ability to recognize and rapidly bind to DNA single or double strand breaks (D'Amours, et al., Biochem. J., 342, 249-268 (1999); and Virag, et al., Pharmacological Reviews, vol. 54, no. 3, 375-429 (2002)). It participates in a variety of DNA-related functions including gene amplification, cell division, differentiation, apoptosis, DNA base excision repair as well as effects on telomere length and chromosome stability (d'Adda di Fagagna, et al., Nature Gen., 23(1), 76-80 (1999)).

PARP has an essential role in facilitating DNA repair, controlling RNA transcription, mediating cell death, and regulating immune response. Studies on the mechanism by which PARP-1 modulates DNA repair and other processes identifies its importance in the formation of poly(ADP-ribose) chains within the cellular nucleus. The DNA-bound, activated PARP-1 utilizes NAD+ to synthesize poly(ADP-ribose) on a variety of nuclear target proteins, including topoisomerases, histones and PARP itself. (Althaus, F. R. and Richter, C., ADP-Ribosylation of Proteins: Enzymology and Biological Significance, Springer-Verlag, Berlin (1987); and Rhun, et al., Biochem. Biophys. Res. Commun., 245, 1-10 (1998)).

PARP inhibitors demonstrate efficacy in numerous models of disease particularly in models of ischemia reperfusion injury, inflammatory disease, degenerative diseases, protection from above adverse effects of cytotoxic compounds, and potentiation of cytotoxic cancer therapy. They are efficacious in the prevention of ischemia reperfusion injury in models of myocardial infarction, stoke, other neural trauma, organ transplantation, as well as reperfusion of the eye, kidney, gut and skeletal muscle. Inhibitors are efficacious in inflammatory diseases such as arthritis, gout, inflammatory bowel disease, CNS inflammation such as MS and allergic encephalitis, sepsis, septic shock, hemorrhagic shock, pulmonary fibrosis, and uveitis. PARP inhibitors also show benefit in several models of degenerative disease including diabetes and Parkinson's disease. PARP inhibitors ameliorate the liver toxicity following acetaminophen overdose, cardiac and kidney toxicities from doxorubicin and platinum based antineoplastic agents, as well as skin damage secondary to sulfur mustards. In various cancer models, PARP inhibitors are shown to potentiate radiation and chemotherapy by increasing apoptosis of cancer cells, limiting tumor growth, decreasing metastasis, and prolonging the survival of tumor-bearing animals.

Poly(ADP-ribosyl)ation is also associated with malignant transformation. For example, PARP-1 activity is higher in the isolated nuclei of SV40-transformed fibroblasts, while both leukemic cells and colon cancer cells show higher enzyme activity than the equivalent normal leukocytes and colon mucosa. Furthermore, malignant prostate tumors have increased levels of active PARP as compared to benign prostate cells, which is associated with higher levels of genetic instability. (Miwa, et al., Arch. Biochem. Biophys., 181, 313-321 (1977); Burzio, et al., Proc. Soc. Exp. Biol. Med., 149, 933-938 (1975); Hirai, et al., Cancer Res., 43, 3441-3446 (1983); and Mcnealy, et al., Anticancer Res., 23, M73-1478 (2003)).

In cells treated with alkylating agents, the inhibition of PARP leads to a marked increase in DNA-strand breakage and cell killing. PARP-1 inhibitors also enhance the effects of radiation response by suppressing the repair of potentially lethal damage. PARP inhibitors are also effective in radio-sensitizing hypoxic tumor cells. In certain tumor cell lines, chemical inhibition of PARP activity is also associated with marked sensitization to very low doses of radiation.

Furthermore, PARP-1 knockout (PARP −/−) animals exhibit genomic instability in response to alkylating agents and γ-irradiation. Data indicates that PARP-1 and PARP-2 possess both overlapping and non-redundant functions in the maintenance of genomic stability, making them both interesting targets. (Wang, et al., Genes Dev., 9, 509-520 (1995); Menissier de Murcia, et al., Proc. Natl. Acad. Sci. USA, 94, 7303-7307 (1997); and Menissier de Murcia, et al., EMBO. J., 22(9), 2255-2263 (2003)).

There is also a role for PARP-1 in certain vascular diseases, such as, for example, septic shock, ischemic injury and neurotoxicity. Oxygen radical DNA damage that leads to strand breaks in DNA, which are subsequently recognized by PARP-1, is a major contributing factor to such disease states as shown by PARP-1 inhibitor studies. PARP also plays a role in the pathogenesis of hemorrhagic shock. (Cantoni, et al, Biochim. Biophys. Acta, 1014, 1-7 (1989); Szabo, et al., J. Clin. Invest., 100, 723-735 (1997); Cosi, et al., J. Neurosci. Res., 39, 3846 (1994); Said, et al., Proc. Natl. Acad. Sci. U.S.A., 93, 4688-4692 (1996); and Liaudet, et al., Proc. Natl. Acad. Sci. U.S.A., 97(3), 10203-10208 (2000)).

Furthermore, efficient retroviral infection of mammalian cells is blocked by the inhibition of PARP-1 activity. Such inhibition of recombinant retroviral vector infections occurs in various different cell types. In some embodiments, inhibitors of PARP-1 are used in anti-viral therapies and in cancer treatment. (Gaken, et al., J. Virology, 70(6), 3992-4000 (1996)).

PARP-1 inhibition delays the onset of aging characteristics in human fibroblasts. While not intending to bound by any theory, this may be related to the role that PARP plays in controlling telomere function. (Rattan and Clark, Biochem. Biophys. Res. Comm., 201(2), 665-672 (1994); and d'Adda di Fagagna, et al., Nature Gen., 23(1), 76-80 (1999)).

PARP inhibitors are relevant to the treatment of inflammatory bowel disease, ulcerative colitis and Crohn's disease. (Szabo C., Role of Poly(ADP-Ribose) Polymerase Activation in the Pathogenesis of Shock and Inflammation, In PARP as a Therapeutic Target; Ed J. Zhang, 2002 by CRC Press; 169-204; Zingarelli, B, et al., Immunology, 113(4), 509-517 (2004); and Jijon, H. B., et al., Am. J. Physiol. Gastrointest. Liver Physiol., 279, G641-G651 (2000)).

PARP inhibitors are used for the treatment of cancer which is deficient in Homologous Recombination (HR) dependent DNA double strand break (DSB) repair activity. The HR dependent DNA DSB repair pathway repairs double-strand breaks (DSBs) in DNA via homologous mechanisms to reform a continuous DNA helix. The components of the HR dependent DNA DSB repair pathway include, but are not limited to, ATM (NM_{—}000051), RAD51 (NM_{—}002875), RAD51L1 (NM_{—}002877), RAD51C (NM_{—}002876), RAD51L3 (NM_{—}002878), DMC1 (NM_{—}007068), XRCC2 (NM_{—}005431), XRCC3 (NM_{—}005432), RAD52 (NM_{—}002879), RAD54L (NM_{—}003579), RAD54B (NM_{—}012415), BRCA1 (NM_{—}007295), BRCA2 (NM_{—}000059), RAD50 (NM_{—}005732), MRE11A (NM_{—}005590) and NBS1 (NM_{—}002485). Other proteins involved in the HR dependent DNA DSB repair pathway include regulatory factors such as EMSY. HR components are also described in Wood, et al., Science, 291, 1284-1289 (2001). (K. K. Khanna and S. P. Jackson, Nat. Genet. 27(3): 247-254 (2001); and Hughes-Davies, et al., Cell, 115, pp 523-535).

BRCA1 and BRCA2 are tumor suppressors whose wild-type alleles are frequently lost in tumors of heterozygous carriers. BRCA1 and/or BRCA2 mutations are associated with breast cancer. Amplification of the EMSY gene, which encodes a BRCA2 binding factor, is associated with breast and ovarian cancer. (Jasin M., Oncogene, 21(58), 8981-93 (2002); Tutt, et al., Trends Mol Med., 8(12), 571-6, (2002); and Radice, P. J., Exp Clin Cancer Res., 21(3 Suppl), 9-12 (2002)). Carriers of mutations in BRCA1 and/or BRCA2 are also at elevated risk of cancer of the ovary, prostate and pancreas. The detection of variation in BRCA1 and BRCA2 is described, for example in EP 699 754, EP 705 903, Neuhausen, S. L. and Ostrander, E. A., Genet. Test, 1, 75-83 (1992); Janatova M., et al., Neoplasma, 50(4), 246-50 (2003. Determination of amplification of the BRCA2 binding factor EMSY is described in Hughes-Davies, et al., Cell, 115, 523-535).

The primary function of the DNA mismatch repair (MMR) system is to eliminate single-base mismatches and insertion-deletion loops that may arise during DNA replication. Insertion-deletion loops result from gains or losses of short repeat units within microsatellite sequences, also known as microsatellite instability (MSI). At least six different MMR proteins are required. For mismatch recognition, the MSH2 protein forms a heterodimer with either MSH6 or MSH3 depending on the type of lesion to be repaired (MSH6 is required for the correction of single-base mispairs, whereas both MSH3 and MSH6 may contribute to the correction of insertion-deletion loops). A heterodimer of MLH1 and PMS2 coordinates the interplay between the mismatch recognition complex and other proteins necessary for MMR. These additional proteins may include at least exonuclease 1 (EXO1), possibly helicase(s), proliferating cell nuclear antigen (PCNA), single-stranded DNA-binding protein (RPA), and DNA polymerases δ and ε. In addition to PMS2, MLH1 may heterodimerize with two additional proteins, MLH3 and PMS1. Recent observations indicate that PMS2 is required for the correction of single-base mismatches, and PMS2 and MLH3 both contribute to the correction of insertion-deletion loops. Additional homologs of the human MMR proteins are known that are required for functions other than MMR. These proteins include MSH4 and MSH5 that are necessary for meiotic (and possibly mitotic) recombination but are not presumed to participate in MMR.

Germline mutations of human MMR genes cause susceptibility to hereditary nonpolyposis colon cancer (HNPCC), one of the most common cancer syndromes in humans. An excess of colon cancer and a defined spectrum of extracolonic cancers, diagnosed at an early age and transmitted as an autosomal dominant trait, constitute the clinical definition of the syndrome. MSI, the hallmark of HNPCC, occurs in approximately 15% to 25% of sporadic tumors of the colorectum and other organs as well. According to international criteria, a high degree of MSI (MSI-H) is defined as instability at two or more of five loci or ≧30% to 40% of all microsatellite loci studied, whereas instability at fewer loci is referred to as MSI-low (MSI-L). MSI occurs in a substantial proportion (2% to 50% of tumors) among non-HNPCC cancers (e.g., cancers of the breast, prostate, and lung). On the basis of the proportion of unstable markers, categories MSS, MSI-L, and MSI-H can be distinguished in these cancers in analogy to HNPCC cancers.

### SUMMARY OF THE INVENTION

Provided herein are compounds, compositions and methods for modulating the activity of PARP. Among the compounds that are provided herein, are compounds that are inhibitors of PARP. Also described herein is the use of such compounds, compositions and methods for the treatment of diseases, disorders or conditions associated with the activity of PARP.

The following only summarizes certain aspects of the invention and is not intended to be limiting in nature. These aspects and other aspects and embodiments are described more fully below. All publications, patents, and patent applications cited in this specification are hereby incorporated by reference in their entirety for all purposes. In the event of a discrepancy between the express disclosure of this specification and the references incorporated by reference, the express disclosure of this specification shall control.

One aspect is a Compound according to Formula (M):

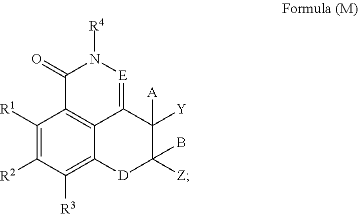

where Y and Z are each independently
a) aryl optionally substituted with 1, 2, or 3 R⁶;
b) heteroaryl optionally substituted with 1, 2, or 3 R⁶; or
c) a substituent independently selected from the group consisting of hydrogen, C₂-C₆alkenyl, C₁-C₆alkoxy, alkoxyalkyl, C₁-C₆alkoxycarbonyl, alkoxycarbonylalkyl, C₁-C₆alkyl, C₂-C₆alkynyl, arylalkyl, C₃-C₈cycloalkyl, cycloalkylalkyl, C₁-C₆haloalkyl, C₁-C₆hydroxyalkyl, oxo, C₃-C₈heterocycloalkyl, heterocycloalkylalkyl, C₁-C₆alkylcarbonyl, arylcarbonyl, heteroarylcarbonyl, C₁-C₆alkylsulfonyl, arylsulfonyl, heteroarylsulfonyl, —C₁₋C₆alkylene-(NR^{A}R^{B}), —C(O)(NR^{A}R^{B}), —C₁₋C₆alkylene-C(O)(NR^{A}R^{B}), —S(O)₂(NR^{A}R^{B}), and —C₁₋C₆alkylene-S(O)₂(NR^{A}R^{B});
E is —CH²≦CH═ and D is —N(R⁵)—; or
E is —N═ and D is —N(R⁵)—CH₂—, —CH₂—N(R⁷)—, —N(R⁵)—N(R⁷)—, —N(R⁵)—O—, —N═CH—, or —CH═N—;
R¹, R², and R³ are each independently selected from the group consisting of hydrogen, halogen, C₂-C₆alkenyl, alkoxy, alkoxycarbonyl, C₁-C₆alkyl, C₃-C₆cycloalkyl, C₂-C₆alkynyl, cyano, C₁-C₆haloalkoxy, C₁-C₆haloalkyl, hydroxyl, C₁-C₆hydroxyalkyl, nitro, —NR^{A}R^{B}, —C₁₋C₆alkylene-(NR^{A}R^{B}), and —C(O)(NR^{A}R^{B});
A and B are each independently selected from hydrogen, Br, Cl, F, I, OH, C₁-C₆alkyl, C₃-C₈cycloalkyl, C₁-C₆alkoxy, alkoxyalkyl where the C₁-C₆alkyl, C₃-C₈cycloalkyl, C₁-C₆alkoxy, and alkoxyalkyl are optionally substituted with 1, 2, 3, or 4 groups independently selected from OH, NO₂, CN, Br, Cl, F, I, C₁-C₆alkyl, and C₃-C₈cycloalkyl, where B is not OH;
R^{A} and R^{B} are independently selected from the group consisting of hydrogen, C₁-C₆alkyl, C₃-C₈cycloalkyl, and C₁-C₆alkylcarbonyl; or
R^{A} and R^{B} taken together with the atom to which they are attached form a 3-10 membered heterocyclyl optionally having one to three heteroatoms or hetero functionalities selected from the group consisting of —O—, —NR^{C}—, —S— and —S(O)_{q}—, where q is 1 or 2 and R^{C} is H, C₁-C₆alkyl, —C(O)(C₁-C₆-alkyl), aryl, aryl-C₁-C₆-alkyl, heteroaryl, or heteroaryl-C₁-C₆-alkyl;
R⁴, R⁵, and R⁷ are each independently selected from the group consisting of hydrogen, C₁-C₆alkyl, C₃-C₈cycloalkyl, alkoxyalkyl, C₁-C₆haloalkyl, C₁-C₆hydroxyalkyl, alkoxycarbonyl, and —C₁-C₆alkylene-(NR^{A}R^{B}); and
R⁶ is selected from OH, NO₂, CN, Br, Cl, F, I, C₁-C₆alkyl, C₃-C₈cycloalkyl, C₃-C₈heterocycloalkyl; C₂-C₆alkenyl, alkoxy, alkoxyalkyl, alkoxycarbonyl, alkoxycarbonylalkyl, C₂-C₆alkynyl, aryl, arylalkyl, C₃-C₈cycloalkylalkyl, C₁-C₆haloalkoxy, C₁-C₆haloalkyl, C₁-C₆hydroxyalkyl, oxo, heteroaryl, heteroarylalkyloxy, heteroaryloxy, heteroarylthio, heteroarylalkylthio, heterocycloalkylalkyloxy, C₃-C₈heterocycloalkylthio, C₃-C₈heterocycloalkyloxy, heterocycloalkylalkylthio, —NR^{A}R^{B}, —C₁₋C₆alkylene-(NR^{A}R^{B}), —C(O)(NR^{A}R^{B}), —C₁₋C₆alkylene-C(O)(NR^{A}R^{B}), —S(O)₂(NR^{A}R^{B}), and —C₁₋C₆alkylene-S(O)₂(NR^{A}R^{B});
optionally as a tautomer, a single stereoisomer or mixture of stereoisomers thereof and additionally optionally as a pharmaceutically acceptable salt thereof.

Another aspect is a pharmaceutical composition comprising a compound of Formula (M) or a pharmaceutically acceptable salt, pharmaceutically acceptable solvate, or pharmaceutically acceptable prodrug and one or more pharmaceutically acceptable carrier(s), excipient(s), binder(s) or diluent(s) thereof.

Another aspect is a method of inhibiting poly(ADP-ribose)polymerase (PARP) in a subject in need of PARP inhibition comprising administering to the subject a therapeutically effective amount of a compound of Formula (M).

Another aspect is a method of treating a disease comprising administering to a subject in need of treatment a therapeutically effective amount of a compound of Formula (M).

Another aspect is a method of treating cancer, comprising administering to a subject in need of treatment a therapeutically effective amount of a compound of Formula (M), optionally as a pharmaceutically acceptable salt, or a therapeutically effective amount of a pharmaceutical composition, in combination with ionizing radiation, one or more therapeutic agents, or a combination thereof.

Another aspect is the use of a compound of Formula (M) in the formulation of a medicament for the treatment of a disease or condition.

Another aspect is an article of manufacture, comprising packaging material, a compound of Formula (M), and a label, where the compound is effective for modulating the activity of the enzyme poly(ADP-ribose)polymerase, or for treatment, prevention or amelioration of one or more symptoms of a poly(ADP-ribose)polymerase-dependent or poly(ADP-ribose)polymerase-mediated disease or condition, where the compound is packaged within the packaging material, and where the label indicates that the compound, or pharmaceutically acceptable salt, pharmaceutically acceptable N-oxide, pharmaceutically active metabolite, pharmaceutically acceptable prodrug, or pharmaceutically acceptable solvate thereof, or a pharmaceutical composition comprising such a compound is used for modulating the activity of poly(ADP-ribose)polymerase, or for treatment, prevention or amelioration of one or more symptoms of a poly(ADP-ribose)polymerase-dependent or poly(ADP-ribose)polymerase-mediated disease or condition.

In another aspect, provided herein is
(1) a method of making of a Compound of Formula IC-1 comprising

(a) treating an intermediate of formula 7a, or a salt thereof:

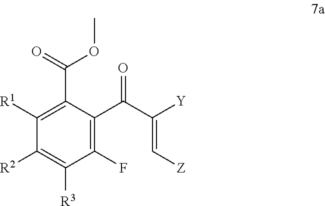

where R¹, R², R³, Y, and Z are as defined in the Summary of the Invention for a Compound of Formula (M); with H₂NNH₂ to yield a compound of Formula IC-1

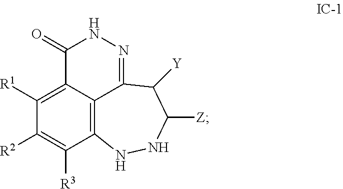

or

(b) alternatively, treating an intermediate of formula 6a, or a salt thereof

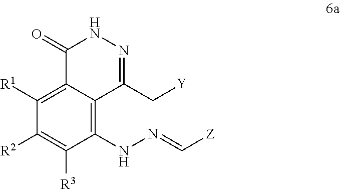

where R¹, R², R³, Y, and Z are as defined in the Summary of the Invention for a Compound of Formula (M); with a base such as CsCO₃ to yield a Compound of Formula IC-1; and

(c) optionally resolving individual isomers to yield compounds of formula IC-1a and IC-1b:

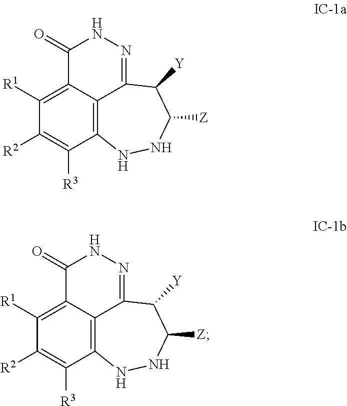

(2) a method of making of a Compound of Formula IB-1 comprising

(a) treating an intermediate of formula 8a, or a salt thereof:

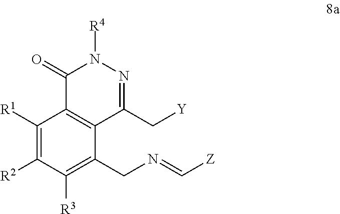

where R¹, R², R³, Y, and Z are as defined in the Summary of the Invention for a Compound of Formula (M); with a base such as CsCO₃ to yield a Compound of Formula IB-1

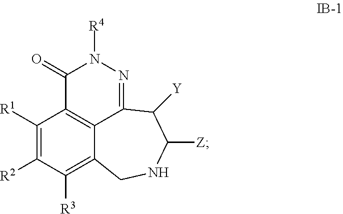

and

(b) optionally resolving individual isomers to yield compounds of formula IB-1a and IB-1b:

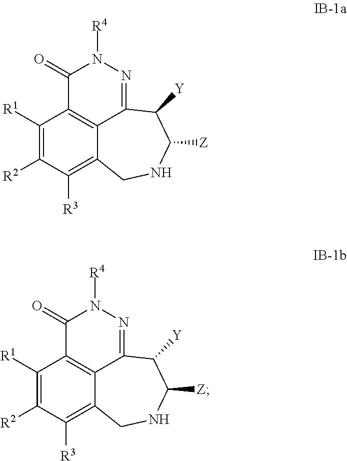

(3) a method of making a Compound of Formula IA-1 comprising

(a) treating an intermediate of formula 10a, or a salt thereof:

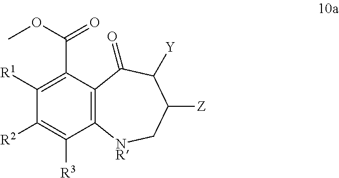

where R′ is hydrogen or a N-protecting group, such as Boc, and R¹, R², R³, Y, and Z are as defined in the Summary of the Invention for a Compound of Formula (M); with H₂NNH₂, followed by removing the R′ protecting group (if present) to yield a Compound of Formula IA-1

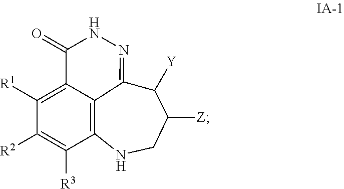

and

(b) optionally resolving individual isomers to yield compounds of formula IA-1a and IA-1b:

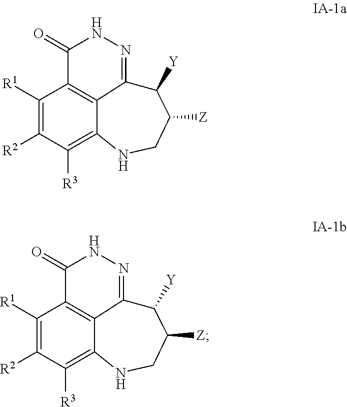

(4) a method of making a Compound of Formula ID-1 comprising

(a) heating an intermediate of formula 11a, or a salt thereof:

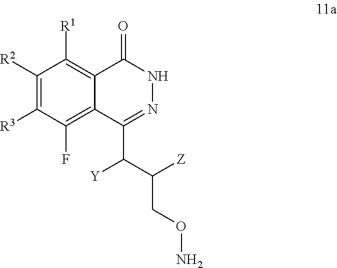

where R¹, R², R³, Y, and Z are as defined in the Summary of the Invention for a Compound of Formula (M); to yield a Compound of Formula ID-1

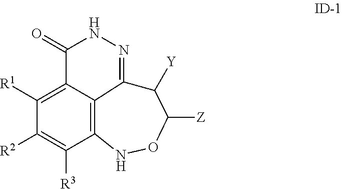

(b) optionally resolving individual isomers to yield compounds of formula ID-1a and ID-1b:

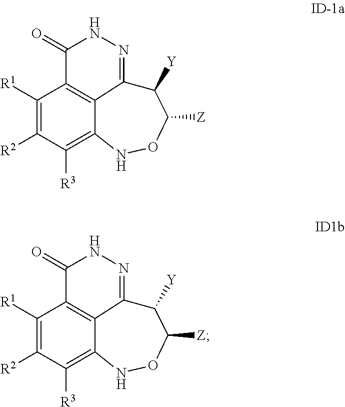

(5) a method of making a Compound of Formula IE-1 comprising

(a) treating an intermediate of formula 12a, or a salt thereof:

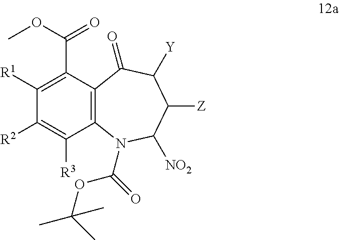

where R¹, R², R³, Y, and Z are as defined in the Summary of the Invention for a Compound of Formula (M); with H₂NNH₂, followed by treating with an acid such as HCl to yield a Compound of Formula IE-1:

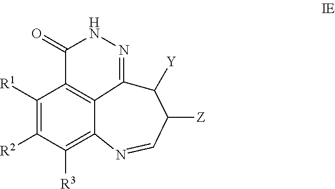

(b) optionally resolving individual isomers to yield compounds of formula IE-1a and IE-1b:

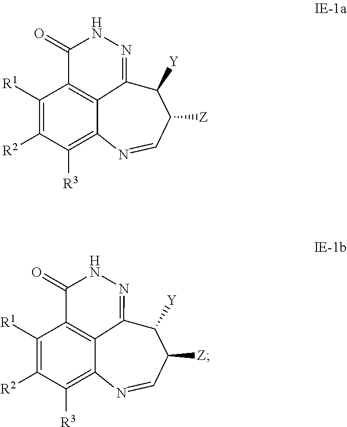

or
(6) a method of making a Compound of Formula IIF-1 comprising

(a) treating an intermediate of formula 13a, or a salt thereof:

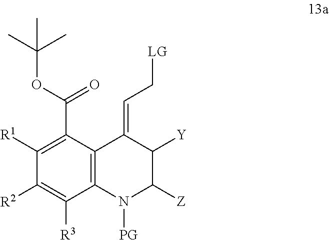

where LG is a leaving group such as mesylate, PG is a N-protecting group, such as Boc, and R¹, R², R³, Y, and Z are as defined in the Summary of the Invention for a Compound of Formula (M); with NH₃, and then deprotecting the nitrogen, such as with an acid where PG is Boc; to yield a Compound of Formula IIF-1

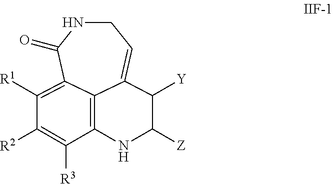

(b) optionally resolving individual isomers to yield compounds of formula IIF-1a and IIF-1b:

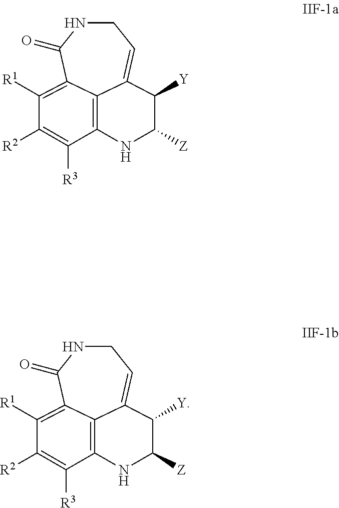

### DETAILED DESCRIPTION OF THE INVENTION

In some embodiments, compounds provided herein have the structure of Formula (M) and pharmaceutically acceptable salts, solvates, esters, acids and prodrugs thereof. Provided herein are compounds having the structure of Formula (M) that are inhibitors of the enzyme poly(ADP-ribose)polymerase (PARP).

In certain embodiments, stereoisomers including enantiomers and diastereoisomers, and chemically protected forms of compounds having a structure represented by Formula (M) are also provided.

The following paragraphs present a number of embodiments of the compounds disclosed herein. In each instance the embodiment includes both the recited compounds as well as a single stereoisomer or mixture of stereoisomers thereof, as well as a pharmaceutically acceptable salt thereof.

In some or any embodiments disclosed is a compound having the structure of Formula (I):

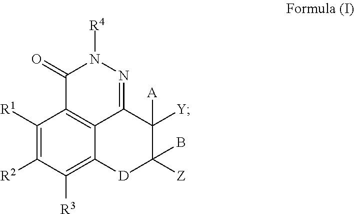

where Y and Z are each independently
a) aryl optionally substituted with 1, 2, or 3 R⁶;
b) heteroaryl optionally substituted with 1, 2, or 3 R⁶; or
c) a substituent independently selected from the group consisting of hydrogen, C₂-C₆alkenyl, C₁-C₆alkoxy, alkoxyalkyl, C₁-C₆alkoxycarbonyl, alkoxycarbonylalkyl, C₁-C₆alkyl, C₂-C₆alkynyl, arylalkyl, C₃-C₈cycloalkyl, cycloalkylalkyl, C₁-C₆haloalkyl, C₁-C₆hydroxyalkyl, oxo, C₃-C₈heterocycloalkyl, heterocycloalkylalkyl, C₁-C₆alkylcarbonyl, arylcarbonyl, heteroarylcarbonyl, alkylsulfonyl, arylsulfonyl, heteroarylsulfonyl, —C₁₋C₆alkylene-(NR^{A}R^{B}), —C(O)(NR^{A}R^{B}), —C₁₋C₆alkylene-C(O)(NR^{A}R^{B}), —S(O)₂(NR^{A}R^{B}), and —C₁₋C₆alkylene-S(O)₂(NR^{A}R^{B});
D is —N(R⁵)—CH₂—, —CH₂—N(R⁷)—, —N(R⁵)—N(R⁷)—, —N(R⁵)—O—, —N═CH—, or —CH═N—;
R¹, R², and R³ are each independently selected from the group consisting of hydrogen, halogen, C₂-C₆alkenyl, C₁-C₆alkoxy, alkoxycarbonyl, C₁-C₆alkyl, C₃-C₈cycloalkyl, C₂-C₆alkynyl, cyano, C₁-C₆haloalkoxy, C₁-C₆haloalkyl, hydroxyl, C₁-C₆hydroxyalkyl, nitro, —NR^{A}R^{B}, —C₁₋C₆alkylene-(NR^{A}R^{B}), and —C(O)(NR^{A}R^{B});
A and B are each independently selected from hydrogen, Br, Cl, F, I, OH, C₁-C₆alkyl, C₃-C₈cycloalkyl, C₁-C₆alkoxy, alkoxyalkyl where the C₁-C₆alkyl, C₃-C₈cycloalkyl, alkoxy, and alkoxyalkyl are optionally substituted with 1, 2, 3, or 4 groups independently selected from OH, NO₂, CN, Br, Cl, F, I, C₁-C₆alkyl, and C₃-C₈cycloalkyl, where B is not OH;
R^{A}, and R^{B} are independently selected from the group consisting of hydrogen, C₁-C₆alkyl, C₃-C₈cycloalkyl, and C₁-C₆alkylcarbonyl; or
R^{A} and R^{B} taken together with the atom to which they are attached form a 3-10 membered heterocyclyl optionally having one to three heteroatoms or hetero functionalities selected from the group consisting of —O—, —S— and —S(O)_{q}—, where q is 1 or 2 and R^{C} is H, C₁-C₆-alkyl, —C(O)(C₁-C₆-alkyl), aryl, aryl-C₁-C₆-alkyl, heteroaryl, or heteroaryl-C₁-C₆-alkyl;
R⁴, R⁵, and R⁷ are each independently selected from the group consisting of hydrogen, C₁-C₆alkyl, C₃-C₈cycloalkyl, alkoxyalkyl, C₁-C₆haloalkyl, C₁-C₆hydroxyalkyl, alkoxycarbonyl, and —C₁₋C₆alkylene-(NR^{A}R^{B}); and
R⁶ is selected from OH, NO₂, CN, Br, Cl, F, I, C₁-C₆alkyl, C₃-C₈cycloalkyl, C₃-C₈heterocycloalkyl; C₂-C₆alkenyl, C₁-C₆alkoxy, alkoxyalkyl, alkoxycarbonyl, alkoxycarbonylalkyl, C₂-C₆alkynyl, aryl, arylalkyl, C₃-C₈cycloalkylalkyl, C₁-C₆haloalkoxy, C₁-C₆haloalkyl, C₁-C₆hydroxyalkyl, oxo, heteroaryl, heteroarylalkyloxy, heteroaryloxy, heteroarylthio, heteroarylalkylthio, heterocycloalkyloxy, C₃-C₈heterocycloalkylthio, heterocycloalkylalkyloxy, heterocycloalkylalkylthio-NR^{A}R^{B}, —C₁₋C₆alkylene-(NR^{A}R^{B}), —C(O)(NR^{A}R^{B}), —C₁₋C₆alkylene-C(O)(NR^{A}R^{B}), —S(O)₂(NR^{A}R^{B}), and —C₁₋C₆alkylene-S(O)₂(NR^{A}R^{B});
optionally as a tautomer, a single stereoisomer or mixture of stereoisomers thereof and additionally optionally as a pharmaceutically acceptable salt thereof.

In some or any embodiments disclosed is a compound having the structure of Formula (II):

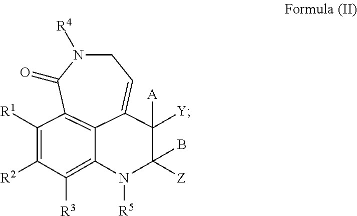

where Y and Z are each independently
a) aryl optionally substituted with 1, 2, or 3 R⁶;
b) heteroaryl optionally substituted with 1, 2, or 3 R⁶; or
c) a substituent independently selected from the group consisting of hydrogen, C₂-C₆alkenyl, C₁-C₆alkoxy, alkoxyalkyl, C₁-C₆alkoxycarbonyl, alkoxycarbonylalkyl, C₁-C₆alkyl, C₂-C₆alkynyl, arylalkyl, C₃-C₈cycloalkyl, cycloalkylalkyl, C₁-C₆haloalkyl, C₁-C₆hydroxyalkyl, oxo, C₃-C₈heterocycloalkyl, heterocycloalkylalkyl, C₁-C₆alkylcarbonyl, arylcarbonyl, heteroarylcarbonyl, C₁-C₆alkylsulfonyl, arylsulfonyl, heteroarylsulfonyl, —C₁₋C₆alkylene-(NR^{A}R^{B}), —C(O)(NR^{A}R^{B}), —C₁₋C₆alkylene-C(O)(NR^{A}R^{B}), —S(O)₂(NR^{A}R^{B}), and —C₁₋C₆alkylene-S(O)₂(NR^{A}R^{B});
R¹, R², and R³ are each independently selected from the group consisting of hydrogen, halogen, C₂-C₆alkenyl, C₁-C₆alkoxy, C₁-C₆alkoxycarbonyl, alkyl, C₃-C₈cycloalkyl, C₂-C₆alkynyl, cyano, C₁-C₆haloalkoxy, C₁-C₆haloalkyl, hydroxyl, C₁-C₆hydroxyalkyl, nitro, —NR^{A}R^{B}, —C₁₋C₆alkylene-(NR^{A}R^{B}), and —C(O)(NR^{A}R^{B});
A and B are each independently selected from hydrogen, Br, Cl, F, I, OH, C₁-C₆alkyl, C₃-C₈cycloalkyl, C₁-C₆alkoxy, alkoxyalkyl where the C₁-C₆alkyl, C₃-C₈cycloalkyl, C₁-C₆alkoxy, and alkoxyalkyl are optionally substituted with at least one substituent selected from OH, NO₂, CN, Br, Cl, F, I, C₁-C₆alkyl, and C₃-C₈cycloalkyl, where B is not OH;
R^{A} and R^{B} are independently selected from the group consisting of hydrogen, C₁-C₆alkyl, C₃-C₈cycloalkyl, and C₁-C₆alkylcarbonyl; or
R^{A} and R^{B} taken together with the atom to which they are attached form a 3-10 membered heterocyclyl optionally having one to three heteroatoms or hetero functionalities selected from the group consisting of —O—, —NR^{C}—, —S— and —S(O)_{q}—, where q is 1 or 2 and R^{C} is H, C₁-C₆-alkyl, —C(O)(C₁-C₆-alkyl), aryl, aryl-C₁-C₆-alkyl, heteroaryl, or heteroaryl-C₁-C₆-alkyl;
R⁴ and R⁵ are each independently selected from the group consisting of hydrogen, C₁-C₆alkyl, C₃-C₈cycloalkyl, alkoxyalkyl, C₁-C₆haloalkyl, C₁-C₆hydroxyalkyl, alkoxycarbonyl, and —C₁₋C₆alkylene-(NR^{A}R^{B}); and
R⁶ is selected from OH, NO₂, CN, Br, Cl, F, I, C₁-C₆alkyl, C₃-C₈cycloalkyl, C₃-C₈heterocycloalkyl; C₂-C₆alkenyl, C₁-C₆alkoxy, alkoxyalkyl, alkoxycarbonyl, alkoxycarbonylalkyl, C₂-C₆alkynyl, aryl, arylalkyl, C₃-C₈cycloalkylalkyl, C₁-C₆haloalkoxy, C₁-C₆haloalkyl, C₁-C₆hydroxyalkyl, oxo, heteroaryl, heteroarylalkyloxy, heteroaryloxy, heteroarylthio, heteroarylalkylthio, heterocycloalkyloxy, C₃-C₈heterocycloalkylthio, heterocycloalkylalkyloxy, heterocycloalkylalkylthio, —NR^{A}R^{B}, —C₁₋C₆alkylene-(NR^{A}R^{B}), —C(O)(NR^{A}R^{B}), —C₁₋C₆alkylene-C(O)(NR^{A}R^{B}), —S(O)₂(NR^{A}R^{B}), and —C₁₋C₆alkylene-S(O)₂(NR^{A}R^{B});
optionally as a tautomer, a single stereoisomer or mixture of stereoisomers thereof and additionally optionally as a pharmaceutically acceptable salt thereof.

In some or any embodiments disclosed is a compound having the structure of Formula (IA):

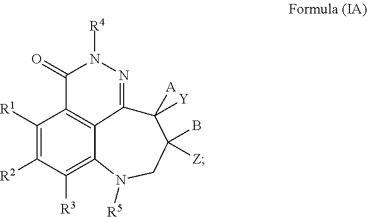

optionally as a tautomer, a single stereoisomer or mixture of stereoisomers thereof and additionally optionally as a pharmaceutically acceptable salt thereof, where A, B, Y, Z, R¹, R², R³, R⁴ and R⁵ are as defined for a Compound of Formula (I).

In some or any embodiments disclosed is a compound having the structure of Formula (IB):

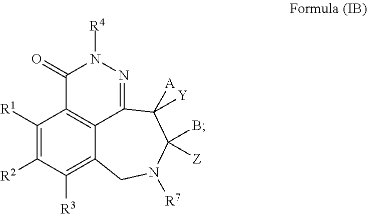

optionally as a tautomer, a single stereoisomer or mixture of stereoisomers thereof and additionally optionally as a pharmaceutically acceptable salt thereof, where A, B, Y, Z, R¹, R², R³, R⁴, R⁵, and R⁷ are as defined for a Compound of Formula (I).

In some or any embodiments disclosed is a compound having the structure of Formula (IC):

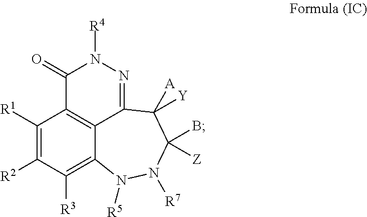

optionally as a tautomer, a single stereoisomer or mixture of stereoisomers thereof and additionally optionally as a pharmaceutically acceptable salt thereof, where A, B, Y, Z, R¹, R², R³, R⁴, R⁵ and R⁷ are as defined for a Compound of Formula (I).

In some or any embodiments disclosed is a compound having the structure of Formula (ID):

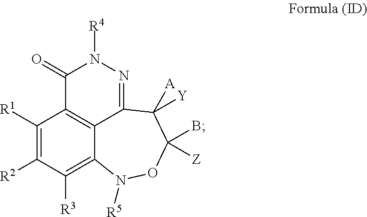

optionally as a tautomer, a single stereoisomer or mixture of stereoisomers thereof and additionally optionally as a pharmaceutically acceptable salt thereof, where A, B, Y, Z, R¹, R², R³, R⁴ and R⁵ are as defined for a Compound of Formula (I).

In some or any embodiments disclosed is a compound having the structure of Formula (IE):

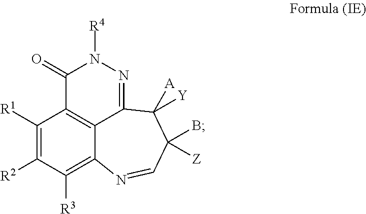

optionally as a tautomer, a single stereoisomer or mixture of stereoisomers thereof and additionally optionally as a pharmaceutically acceptable salt thereof, where A, B, Y, Z, R¹, R², R³, R⁴ and R⁵ are as defined for a Compound of Formula (I).

Embodiment A: In another embodiment, the Compound of Formula (M), (I), (II), (IA), (IB), (IC), (ID), or (IE) is that where R¹ is hydrogen; and all other groups are as defined in the Summary of the Invention for a Compound of Formula (M).

Embodiment B: In another embodiment, the Compound of Formula (M), (I), (II), (IA), (IB), (IC), (ID), or (IE) is that where R² is hydrogen, alkyl, or halo; and all other groups are as defined in the Summary of the Invention for a Compound of Formula (M) or as defined in Embodiment A. In another embodiment, the Compound of Formula (M), (I), (II), (R), (IB), (IC), (ID), or (IE) is that where R² is hydrogen, methyl, or fluoro; and all other groups are as defined in the Summary of the Invention for a Compound of Formula (M) or as defined in Embodiment A. In another embodiment, the Compound of Formula (M), I, II, IA, IB, IC, ID, or IE is that where R² is fluoro; and all other groups are as defined in the Summary of the Invention for a Compound of Formula (M) or as defined in Embodiment A.

Embodiment C: In another embodiment, the Compound of Formula (M), (I), (II), (IA), (IB), (IC), (ID), or (IE) is that where R³ is hydrogen; and all other groups are as defined in the Summary of the Invention for a Compound of Formula (M) and or as defined in any of Embodiments A-B.

Embodiment D: In another embodiment, the Compound of Formula (M), (I), (II), (IA), (IB), (IC), (ID), or (IE) is that where R⁴ is hydrogen; and all other groups are as defined in the Summary of the Invention for a Compound of Formula (M) or as defined in any of Embodiments A-C.

Embodiment E: In another embodiment, the Compound of Formula (M), (I), (II), (IA), (IC), or (ID) is that where R⁵ is hydrogen or alkyl; and all other groups are as defined in the Summary of the Invention for a Compound of Formula (M) and or as defined in any of Embodiments A-D. In another embodiment, the Compound of Formula (M), (I), (II), (IA), (IC), or (ID) is that where R⁵ is hydrogen or methyl; and all other groups are as defined in the Summary of the Invention for a Compound of Formula (M) and or as defined in any of Embodiments A-D. In another embodiment, the Compound of Formula (M), (I), (II), (IA), (IC), or (ID) is that where R³ is hydrogen; and all other groups are as defined in the Summary of the Invention for a Compound of Formula (M) and or as defined in any of Embodiments A-D.

Embodiment F: In another embodiment, the Compound of Formula (M), (I), (IB), or (IC) is that where R⁷ is hydrogen, alkyl, or alkoxycarbonyl; and all other groups are as defined in the Summary of the Invention for a Compound of Formula (M) and or as defined in any of Embodiments A-D. In another embodiment, the Compound of Formula (M), (I), (IB), or (IC) is that where R⁷ is hydrogen, methyl, or tert-butoxycarbonyl; and all other groups are as defined in the Summary of the Invention for a Compound of Formula (M) and or as defined in any of Embodiments A-D. In another embodiment, the Compound of Formula (M), (I), (IB), or (IC) is that where R⁷ is hydrogen or tert-butoxycarbonyl; and all other groups are as defined in the Summary of the Invention for a Compound of Formula (M) and or as defined in any of Embodiments A-D. In another embodiment, the Compound of Formula (M), (I), (IB), or (IC) is that where R⁷ is hydrogen; and all other groups are as defined in the Summary of the Invention for a Compound of Formula (M) and or as defined in any of Embodiments A-D.

Embodiment G: In another embodiment, the Compound of Formula (M), (I), or (IC) is that where R⁵ and R⁷ are hydrogen; and all other groups are as defined in the Summary of the Invention for a Compound of Formula (M) and or as defined in any of Embodiments A-D.

Embodiment G1: In another embodiment, the Compound of Formula (M), (I), or (IC) is that where R⁵ and R⁷ are independently hydrogen or tert-butoxycarbonyl; and all other groups are as defined in the Summary of the Invention for a Compound of Formula (M) and or as defined in any of Embodiments A-D. In another embodiment, the Compound of Formula (M), (I), or (IC) is that where R⁵ is hydrogen and R⁷ is tert-butoxycarbonyl; and all other groups are as defined in the Summary of the Invention for a Compound of Formula (M) and or as defined in any of Embodiments A-D.

Embodiment H: In another embodiment, the Compound of Formula (M), (I), (II), (IA), (IB), (IC), (1D), or (1E) is that where A is hydrogen; and all other groups are as defined in the Summary of the Invention for a Compound of Formula (M) and or as defined in any of Embodiments A-G.

Embodiment J: In another embodiment, the Compound of Formula (M), (I), (II), (IA), (IB), (IC), (1D), or (1E) is that where B is hydrogen; and all other groups are as defined in the Summary of the Invention for a Compound of Formula (M) and or as defined in any of Embodiments A-H.

Embodiment K: In another embodiment, the Compound of Formula (M), (I), (II), (IA), (IB), (IC), (1D), or (1E) is that where A and B are hydrogen; and all other groups are as defined in the Summary of the Invention for a Compound of Formula (M) and or as defined in any of Embodiments A-G.

Embodiment L: In another embodiment, the Compound of Formula (M), (I), (II), (IA), (IB), (IC), (1D), or (1E) is that where Z is phenyl optionally substituted with one R⁶; and all other groups are as defined in the Summary of the Invention for a Compound of Formula (M) and or as defined in any of Embodiments A-H, J, and K.

Embodiment L1: In another embodiment, the Compound of Formula (M), (I), (II), (IA), (IB), (IC), (1D), or (1E) is that where Z is phenyl optionally substituted with one R⁶; where the R⁶ on Z, when present, is halo or —C₁₋C₆alkylene-(NR^{A}R^{B}); and all other groups are as defined in the Summary of the Invention for a Compound of Formula (M) and or as defined in any of Embodiments A-H, J, and K. In another embodiment, the Compound of Formula (M), (I), (II), (IA), (IB), (IC), (1D), or (1E) is that where Z is phenyl optionally substituted with one R⁶; where the R⁶ on Z, when present, is halo or —C₁₋C₆alkylene-(NR^{A}R^{B}); R^{A} and R^{B} are independently hydrogen or C₁-C₆alkyl; and all other groups are as defined in the Summary of the Invention for a Compound of Formula (M) and or as defined in any of Embodiments A-H, J, and K. In another embodiment, the Compound of Formula (M), (I), (II), (IA), (IB), (IC), (1D), or (1E) is that where Z is phenyl optionally substituted with one R⁶; where the R⁶ on Z, when present, is halo or —C₁₋C₆alkylene-(NR^{A}R^{B}); R^{A} and R^{B} taken together with the atom to which they are attached form a 3-10 membered heterocyclyl; and all other groups are as defined in the Summary of the Invention for a Compound of Formula (M) and or as defined in any of Embodiments A-H, J, and K. In another embodiment, the Compound of Formula (M), (I), (II), (IA), (IB), (IC), (1D), or (1E) is that where Z is phenyl optionally substituted with one R⁶; where the R⁶ on Z, when present, is halo, methylaminomethyl, dimethylaminomethyl, azetidinylmethyl, or pyrrolidinylmethyl; and all other groups are as defined in the Summary of the Invention for a Compound of Formula (M) and or as defined in any of Embodiments A-H, J, and K.

Embodiment N: In another embodiment, the Compound of Formula (M), (I), (II), (IA), (IC), (1D), or (1E) is that where A, B, R⁴, and R⁵ are hydrogen; Z is phenyl optionally substituted with one R⁶; and all other groups are as defined in the Summary of the Invention for a Compound of Formula (M) and or as defined in any of Embodiments A-C, F, and L1.

Embodiment O: In another embodiment, the Compound of Formula (M), (I), (IB), or (IC) is that where A, B, R⁴, and R⁷ are hydrogen; Z is phenyl optionally substituted with one R⁶; and all other groups are as defined in the Summary of the Invention for a Compound of Formula (M) and or as defined in any of Embodiments A-C, E, and L1.

Embodiment P: In another embodiment, the Compound of Formula (M), (I), (II), or (IC) is that where A, B, R⁴, R⁵, and R⁷ are hydrogen; Z is phenyl optionally substituted with one R⁶; and all other groups are as defined in the Summary of the Invention for a Compound of Formula (M) and or as defined in any of Embodiments A-C and L1.

Embodiment Q: In another embodiment, the Compound of Formula (M), (I), (II), (IA), (IB), (IC), (1D), or (1E) is that where Y is triazolyl optionally substituted with one R⁶ and all other groups are as defined in the Summary of the Invention for a Compound of Formula (M) or as defined in any of Embodiments A-P.

Embodiment Q1: In another embodiment, the Compound of Formula (M), (I), (II), (IA), (IB), (IC), (1D), or (1E) is that where Y is triazolyl optionally substituted with one R⁶; where the R⁶ on Y, when present, is C₁-C₆alkyl or C₁-C₆hydroxyalkyl; and all other groups are as defined in the Summary of the Invention for a Compound of Formula (M) or as defined in any of Embodiments A-P. In another embodiment, the Compound of Formula (M), (I), (II), (IA), (IB), (IC), (1D), or (1E) is that where Y is triazolyl optionally substituted with one R⁶; where the R⁶ on Y, when present, is methyl, ethyl, propyl, hydroxyethyl, or hydroxypropyl; and all other groups are as defined in the Summary of the Invention for a Compound of Formula (M) or as defined in any of Embodiments A-P. In another embodiment, the Compound of Formula (M), (I), (II), (IA), (IB), (IC), (1D), or (1E) is that where Y is triazolyl optionally substituted with one R⁶; where the R⁶ on Y, when present, is methyl, ethyl, n-propyl, 2-hydroxyethyl, or 3-hydroxypropyl; and all other groups are as defined in the Summary of the Invention for a Compound of Formula (M) or as defined in any of Embodiments A-P.

Embodiment R: In another embodiment, the Compound of Formula (M), (I), (II), (IA), (IB), (IC), (ID), or (1E) is that where A, B, R³, R⁴, R⁵ (when present), and R⁷ (when present) are hydrogen; Z is phenyl optionally substituted with one R⁶; Y is triazolyl optionally substituted with one R⁶; and all other groups are as defined in the Summary of the Invention for a Compound of Formula (M) and or as defined in any of Embodiments A-J.

Embodiment S: In another embodiment, the Compound of Formula (M), (I), (II), or (IC) is that where A, B, R⁴, and R⁵ are hydrogen; R⁷ is hydrogen or alkoxycarbonyl; Z is phenyl optionally substituted with one R⁶; and all other groups are as defined in the Summary of the Invention for a Compound of Formula (M) and or as defined in any of Embodiments A-J. In another embodiment, the Compound of Formula (M), (I), (II), or (IC) is that where A, B, R⁴, and R³ are hydrogen; R⁷ is hydrogen or alkoxycarbonyl; and Y is triazolyl optionally substituted with one R⁶; and all other groups are as defined in the Summary of the Invention for a Compound of Formula (M) and or as defined in any of Embodiments A-J. In another embodiment, the Compound of Formula (M), (I), (II), or (IC) is that where A, B, R⁴, and R⁵ are hydrogen; R⁷ is hydrogen or alkoxycarbonyl; Z is phenyl optionally substituted with one R⁶; and Y is triazolyl optionally substituted with one R⁶; and all other groups are as defined in the Summary of the Invention for a Compound of Formula (M) and or as defined in any of Embodiments A-J.

In another embodiment the Compound of Formula (M) is as follows:

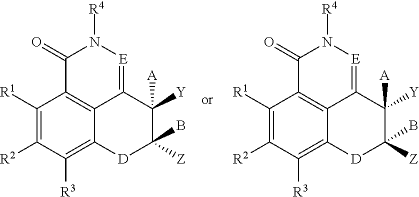

where all groups are as defined in the Summary of the Invention or as defined in any of the above embodiments.

In another embodiment the Compound of Formula (I) is as follows:

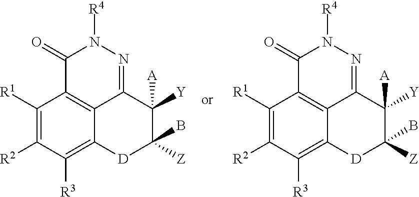

where all groups are as defined in the Summary of the Invention or as defined in any of the above embodiments.

In another embodiment the Compound of Formula (IA) is as follows:

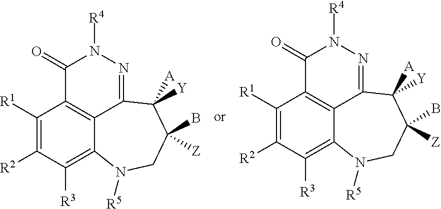

where all groups are as defined in the Summary of the Invention or as defined in any of the above embodiments.

In another embodiment the Compound of Formula (IB) is as follows:

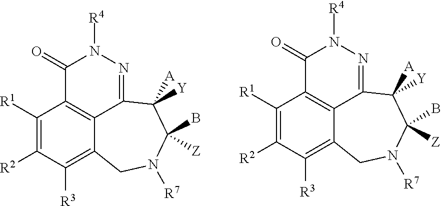

where all groups are as defined in the Summary of the Invention or as defined in any of the above embodiments.

In another embodiment the Compound of Formula (IC) is as follows:

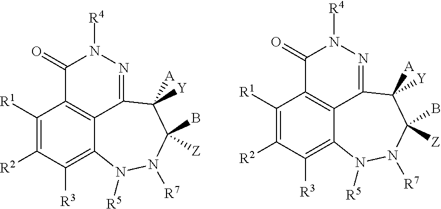

where all groups are as defined in the Summary of the Invention or as defined in any of the above embodiments.

In another embodiment the Compound of Formula (ID) is as follows:

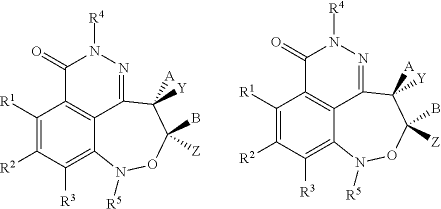

where all groups are as defined in the Summary of the Invention or as defined in any of the above embodiments.

In another embodiment the Compound of Formula (IE) is as follows:

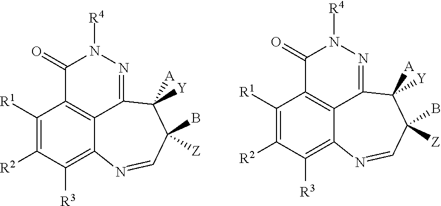

where all groups are as defined in the Summary of the Invention or as defined in any of the above embodiments.

In another embodiment the Compound of Formula (II) is as follows:

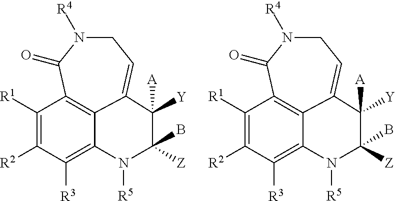

where all groups are as defined in the Summary of the Invention or as defined in any of the above embodiments.

Any combination of the groups described above for the various variables is contemplated herein.

In yet a further embodiment is a compound, optionally as a pharmaceutically acceptable salt, solvate or prodrug thereof, selected from:

| Cmpd | | |
|---|---|---|
| No. | Structure | Name |
| 1 | 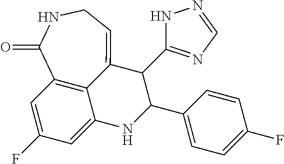 [SMILES: O=C1NCC=C2c3c(cc(F)cc31)NC(c1ccc(F)cc1)C2c1ncn[nH]1] | 9-fluoro-2-(4-fluorophenyl)-3-(1H-1,2,4-triazol-5- yl)-2,3,5,6-tetrahydroazepino[5,4,3-de]quinolin- 7(1H)-one |
| 2 | 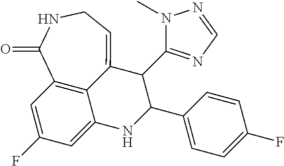 [SMILES: Cn1ncnc1C1C2=CCNC(=O)c3cc(F)cc(c32)NC1c1ccc(F)cc1] | 9-fluoro-2-(4-fluorophenyl)-3-(1-methyl-1H-1,2,4- triazol-5-yl)-2,3,5,6-tetrahydroazepino[5,4,3- de]quinolin-7(1H)-one |
| 3 | 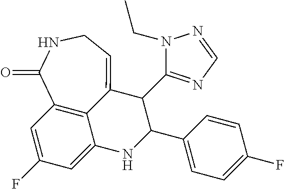 [SMILES: CCn1ncnc1C1C2=CCNC(=O)c3cc(F)cc(c32)NC1c1ccc(F)cc1] | 3-(1-ethyl-1H-1,2,4-triazol-5-yl)-9-fluoro-2-(4- fluorophenyl)-2,3,5,6-tetrahydroazepino[5,4,3- de]quinolin-7(1H)-one |
| 4 | 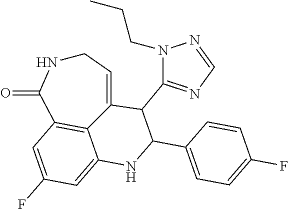 [SMILES: CCCn1ncnc1C1C2=CCNC(=O)c3cc(F)cc(c32)NC1c1ccc(F)cc1] | 9-fluoro-2-(4-fluorophenyl)-3-(1-propyl-1H-1,2,4- triazol-5-yl)-2,3,5,6-tetrahydroazepino[5,4,3- de]quinolin-7(1H)-one |
| 5 | 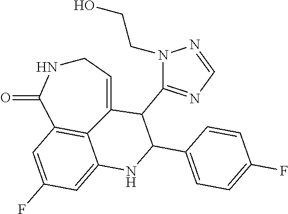 [SMILES: O=C1NCC=C2c3c(cc(F)cc31)NC(c1ccc(F)cc1)C2c1ncnn1CCO] | 9-fluoro-2-(4-fluorophenyl)-3-(1-(2-hydroxyethyl)- 1H-1,2,4-triazol-5-yl)-2,3,5,6- tetrahydroazepino[5,4,3-de]quinolin-7(1H)-one |
| 6 | 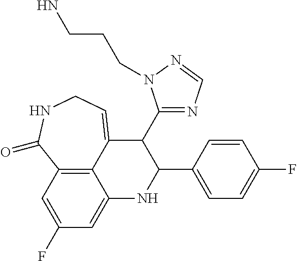 [SMILES: NCCCn1ncnc1C1C2=CCNC(=O)c3cc(F)cc(c32)NC1c1ccc(F)cc1] | 9-fluoro-2-(4-fluorophenyl)-3-(1-(3- hydroxypropyl)-1H-1,2,4-triazol-5-yl)-2,3,5,6- tetrahydroazepino[5,4,3-de]quinolin-7(1H)-one |
| 7 | 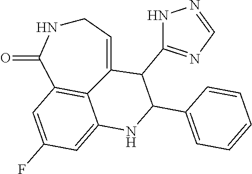 [SMILES: O=C1NCC=C2c3c(cc(F)cc31)NC(c1ccccc1)C2c1ncn[nH]1] | 9-fluoro-2-phenyl-3-(1H-1,2,4-triazol-5-yl)-2,3,5,6- tetrahydroazepino[5,4,3-de]quinolin-7(1H)-one |
| 8 | 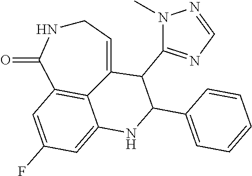 [SMILES: Cn1ncnc1C1C2=CCNC(=O)c3cc(F)cc(c32)NC1c1ccccc1] | 9-fluoro-3-(1-methyl-1H-1,2,4-triazol-5-yl)-2- phenyl-2,3,5,6-tetrahydroazepino[5,4,3- de]quinolin-7(1H)-one |
| 9 | 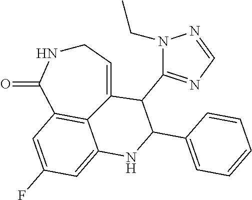 [SMILES: CCn1ncnc1C1C2=CCNC(=O)c3cc(F)cc(c32)NC1c1ccccc1] | 3-(1-ethyl-1H-1,2,4-triazol-5-yl)-9-fluoro-9-phenyl- 2,3,5,6-tetrahydroazepino[5,4,3-de]quinolin- 7(1H)-one |
| 10 | 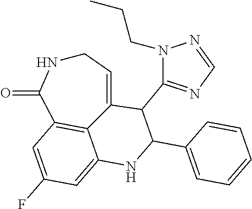 [SMILES: CCCn1ncnc1C1C2=CCNC(=O)c3cc(F)cc(c32)NC1c1ccccc1] | 9-fluoro-2-phenyl-3-(1-propyl-1H-1,2,4-triazol-5- yl)-2,3,5,6-tetrahydroazepino[5,4,3-de]quinolin- 7(1H)-one |
| 11 | 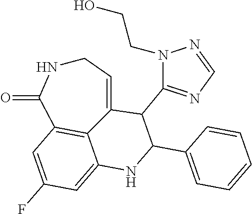 [SMILES: O=C1NCC=C2c3c(cc(F)cc31)NC(c1ccccc1)C2c1ncnn1CCO] | 9-fluoro-3-(1-(2-hydroxyethyl)-1H-1,2,4-triazol-5- yl)-2-phenyl-2,3,5,6-tetrahydroazepino[5,4,3- de]quinolin-7(1H)-one |
| 12 | 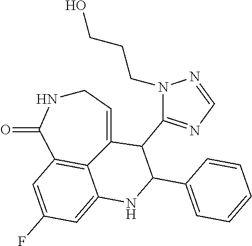 [SMILES: O=C1NCC=C2c3c(cc(F)cc31)NC(c1ccccc1)C2c1ncnn1CCCO] | 9-fluoro-3-(1-(3-hydroxypropyl)-1H-1,2,4-triazol-5- yl)-2-phenyl-2,3,5,6-tetrahydroazepino[5,4,3- de]quinolin-7(1H)-one |
| 13 | 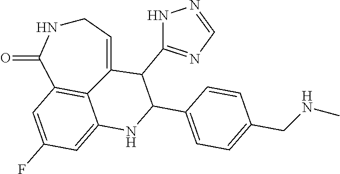 [SMILES: CNCc1ccc(C2Nc3cc(F)cc4c3C(=CCNC4=O)C2c2ncn[nH]2)cc1] | 9-fluoro-2-(4-((methylamino)methyl)phenyl)-3- (1H-1,2,4-triazol-5-yl)-2,3,5,6- tetrahydroazepino[5,4,3-de]quinolin-7(1H)-one |
| 14 | 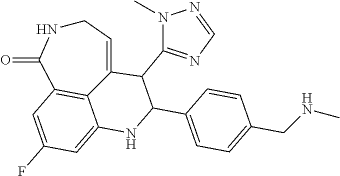 [SMILES: CNCc1ccc(C2Nc3cc(F)cc4c3C(=CCNC4=O)C2c2ncnn2C)cc1] | 9-fluoro-3-(1-methyl-1H-1,2,4-triazol-5-yl)-2-(4- ((methylamino)methyl)phenyl)-2,3,5,6- tetrahydroazepino[5,4,3-de]quinolin-7(1H)-one |
| 15 | 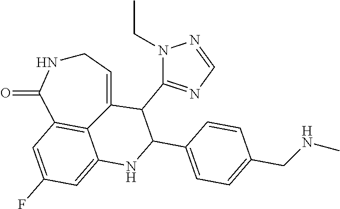 [SMILES: CCn1ncnc1C1C2=CCNC(=O)c3cc(F)cc(c32)NC1c1ccc(CNC)cc1] | 3-(1-ethyl-1H-1,2,4-triazol-5-yl)-9-fluoro-2-(4- ((methylamino)methyl)phenyl)-2,3,5,6- tetrahydroazepino[5,4,3-de]quinolin-7(1H)-one |
| 16 | 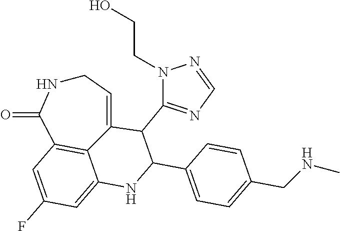 [SMILES: CNCc1ccc(C2Nc3cc(F)cc4c3C(=CCNC4=O)C2c2ncnn2CCO)cc1] | 9-fluoro-3-(1-(2-hydroxyethyl)-1H-1,2,4-triazol-5- yl)-2-(4-((methylamino)methyl)phenyl)-2,3,5,6- tetrahydroazepino[5,4,3-de]quinolin-7(1H)-one |
| 17 | 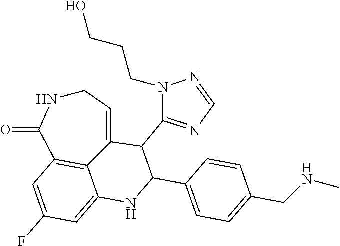 [SMILES: CNCc1ccc(C2Nc3cc(F)cc4c3C(=CCNC4=O)C2c2ncnn2CCCO)cc1] | 9-fluoro-3-(1-(3-hydroxypropyl)-1H-1,2,4-triazol-5- yl)-2-(4-((methylamino)methyl)phenyl)-2,3,5,6- tetrahydroazepino[5,4,3-de]quinolin-7(1H)-one |
| 18 | 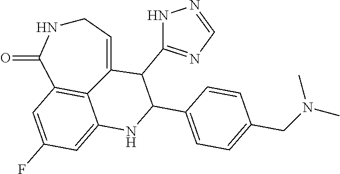 [SMILES: CN(C)Cc1ccc(C2Nc3cc(F)cc4c3C(=CCNC4=O)C2c2ncn[nH]2)cc1] | 2-(4-((dimethylamino)methyl)phenyl)-9-fluoro-3- (1H-1,2,4-triazol-5-yl)-2,3,5,6- tetrahydroazepino[5,4,3-de]quinolin-7(1H)-one |
| 19 | 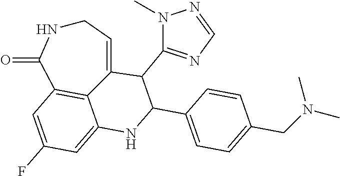 [SMILES: CN(C)Cc1ccc(C2Nc3cc(F)cc4c3C(=CCNC4=O)C2c2ncnn2C)cc1] | 2-(4-((dimethylamino)methyl)phenyl)-9-fluoro-3- (1-methyl-1H-1,2,4-triazol-5-yl)-2,3,5,6- tetrahydroazepino[5,4,3-de]quinolin-7(1H)-one |
| 20 | 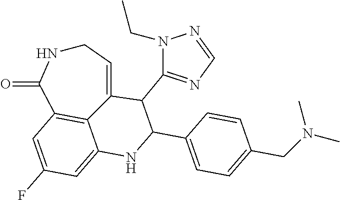 [SMILES: CCn1ncnc1C1C2=CCNC(=O)c3cc(F)cc(c32)NC1c1ccc(CN(C)C)cc1] | 2-(4-((dimethylamino)methyl)phenyl)-3-(1-ethyl- 1H-1,2,4-triazol-5-yl)-9-fluoro-2,3,5,6- tetrahydroazepino[5,4,3-de]quinolin-7(1H)-one |
| 21 | 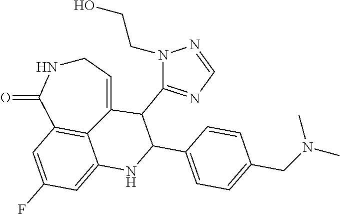 [SMILES: CN(C)Cc1ccc(C2Nc3cc(F)cc4c3C(=CCNC4=O)C2c2ncnn2CCO)cc1] | 2-(4-((dimethylamino)methyl)phenyl)-9-fluoro-3- (1-(2-hydroxyethyl)-1H-1,2,4-triazol-5-yl)-2,3,5,6- tetrahydroazepino[5,4,3-de]quinolin-7(1H)-one |
| 22 | 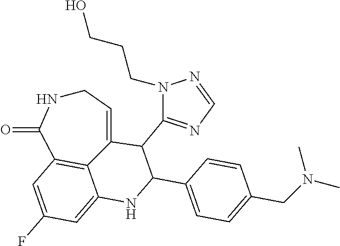 [SMILES: CN(C)Cc1ccc(C2Nc3cc(F)cc4c3C(=CCNC4=O)C2c2ncnn2CCCO)cc1] | 2-(4-((dimethylamino)methyl)phenyl)-9-fluoro-3- (1-(3-hydroxypropyl)-1H-1,2,4-triazol-5-yl)- 2,3,5,6-tetrahydroazepino[5,4,3-de]quinolin- 7(1H)-one |
| 23 | 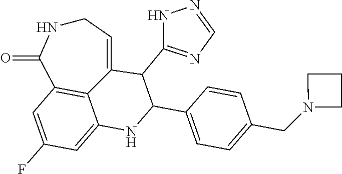 [SMILES: O=C1NCC=C2c3c(cc(F)cc31)NC(c1ccc(CN3CCC3)cc1)C2c1ncn[nH]1] | 2-(4-(azetidin-1-ylmethyl)phenyl)-9-fluoro-3-(1H- 1,2,4-triazol-5-yl)-2,3,5,6-tetrahydroazepino[5,4,3- de]quinolin-7(1H)-one |
| 24 | 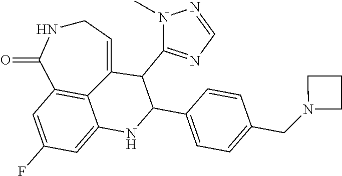 [SMILES: Cn1ncnc1C1C2=CCNC(=O)c3cc(F)cc(c32)NC1c1ccc(CN2CCC2)cc1] | 2-(4-(azetidin-1-ylmethyl)phenyl)-9-fluoro-3-(1- methyl-1H-1,2,4-triazol-5-yl)-2,3,5,6- tetrahydroazepino[5,4,3-de]quinolin-7(1H)-one |
| 25 | 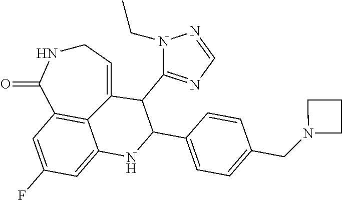 [SMILES: CCn1ncnc1C1C2=CCNC(=O)c3cc(F)cc(c32)NC1c1ccc(CN2CCC2)cc1] | 2-(4-(azetidin-1-ylmethyl)phenyl)-3-(1-ethyl-1H- 1,2,4-triazol-5-yl)-9-fluoro-2,3,5,6- tetrahydroazepino[5,4,3-de]quinolin-7(1H)-one |
| 26 | 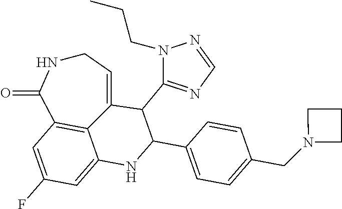 [SMILES: CCCn1ncnc1C1C2=CCNC(=O)c3cc(F)cc(c32)NC1c1ccc(CN2CCC2)cc1] | 2-(4-(azetidin-1-ylmethyl)phenyl)-9-fluoro-3-(1- propyl-1H-1,2,4-triazol-5-yl)-2,3,5,6- tetrahydroazepino[5,4,3-de]quinolin-7(1H)-one |
| 27 | 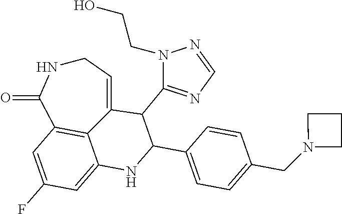 [SMILES: O=C1NCC=C2c3c(cc(F)cc31)NC(c1ccc(CN3CCC3)cc1)C2c1ncnn1CCO] | 2-(4-azetidin-1-ylmethyl)phenyl)-9-fluoro-3-(1-(2- hydroxyethyl)-1H-1,2,4-triazol-5-yl)-2,3,5,6- tetrahydroazepino[5,4,3-de]quinolin-7(1H)-one |
| 28 | 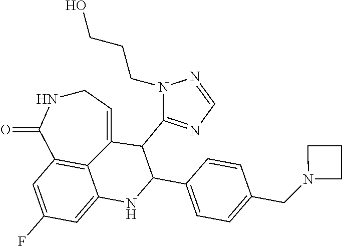 [SMILES: O=C1NCC=C2c3c(cc(F)cc31)NC(c1ccc(CN3CCC3)cc1)C2c1ncnn1CCCO] | 2-(4-(azetidin-1-ylmethyl)phenyl)-9-fluoro-3-(1-(3- hydroxypropyl)-1H-1,2,4-triazol-5-yl)-2,3,5,6- tetrahydroazepino[5,4,3-de]quinolin-7(1H)-one |
| 29 | 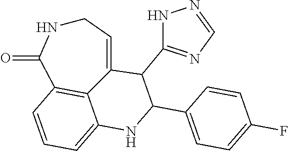 [SMILES: O=C1NCC=C2c3c(cccc31)NC(c1ccc(F)cc1)C2c1ncn[nH]1] | 2-(4-fluorophenyl)-3-(1H-1,2,4-triazol-5-yl)- 2,3,5,6-tetrahydroazepino[5,4,3-de]quinolin- 7(1H)-one |
| 30 | 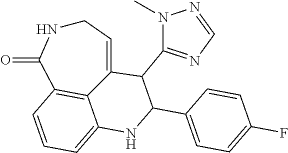 [SMILES: Cn1ncnc1C1C2=CCNC(=O)c3cccc(c32)NC1c1ccc(F)cc1] | 2-(4-fluorophenyl)-3-(1-methyl-1H-1,2,4-triazol-5- yl)-2,3,5,6-tetrahydroazepino[5,4,3-de]quinolin- 7(1H)-one |
| 31 | 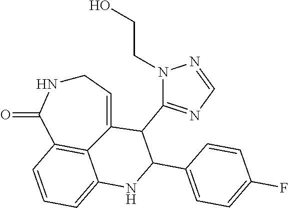 [SMILES: O=C1NCC=C2c3c(cccc31)NC(c1ccc(F)cc1)C2c1ncnn1CCO] | 2-(4-fluorophenyl)-3-(1-(2-hydroxyethyl)-1H-1,2,4- triazol-5-yl)-2,3,5,6-tetrahydroazepino[5,4,3- de]quinolin-7(1H)-one |
| 32 | 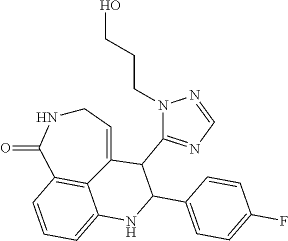 [SMILES: O=C1NCC=C2c3c(cccc31)NC(c1ccc(F)cc1)C2c1ncnn1CCCO] | 2-(4-fluorophenyl)-3-(1-(3-hydroxypropyl)-1H- 1,2,4-triazol-5-yl)-2,3,5,6-tetrahydroazepino[5,4,3- de]quinolin-7(1H)-one |
| 33 | 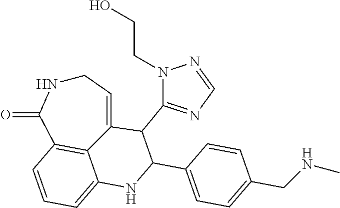 [SMILES: CNCc1ccc(C2Nc3cccc4c3C(=CCNC4=O)C2c2ncnn2CCO)cc1] | 3-(1-(2-hydroxyethyl)-1H-1,2,4-triazol-5-yl)-2-(4- ((methylamino)methyl)phenyl)-2,3,5,6- tetrahydroazepino[5,4,3-de]quinolin-7(1H)-one |
| 34 | 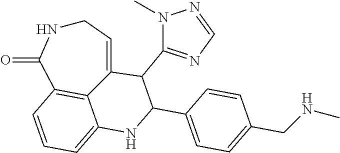 [SMILES: CNCc1ccc(C2Nc3cccc4c3C(=CCNC4=O)C2c2ncnn2C)cc1] | 3-(1-methyl-1H-1,2,4-triazol-5-yl)-2-(4- ((methylamino)methyl)phenyl)-2,3,5,6- tetrahydroazepino[5,4,3-de]quinolin-7(1H)-one |
| 35 | 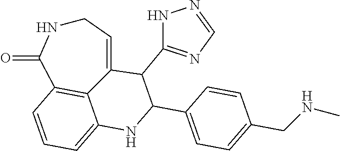 [SMILES: CNCc1ccc(C2Nc3cccc4c3C(=CCNC4=O)C2c2ncn[nH]2)cc1] | 2-(4-((methylamino)methyl)phenyl)-3-(1H-1,2,4- triazol-5-yl)-2,3,5,6-tetrahydroazepino[5,4,3- de]quinolin-7(1H)-one |
| 36 | 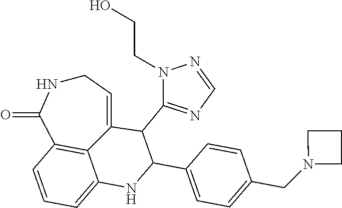 [SMILES: O=C1NCC=C2c3c(cccc31)NC(c1ccc(CN3CCC3)cc1)C2c1ncnn1CCO] | 2-(4-(azetidin-1-ylmethyl)phenyl)-3-(1-(2- hydroxyethyl)-1H-1,2,4-triazol-5-yl)-2,3,5,6- tetrahydroazepino[5,4,3-de]quinolin-7(1H)-one |
| 37 | 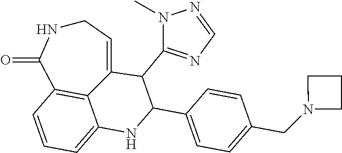 [SMILES: Cn1ncnc1C1C2=CCNC(=O)c3cccc(c32)NC1c1ccc(CN2CCC2)cc1] | 2-(4-(azetidin-1-ylmethyl)phenyl)-3-(1-methyl-1H- 1,2,4-triazol-5-yl)-2,3,5,6-tetrahydroazepino[5,4,3- de]quinolin-7(1H)-one |
| 38 | 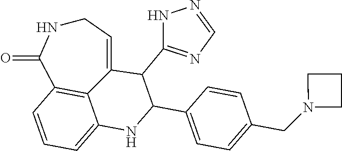 [SMILES: O=C1NCC=C2c3c(cccc31)NC(c1ccc(CN3CCC3)cc1)C2c1ncn[nH]1] | 2-(4-(azetidin-1-ylmethyl)phenyl)-3-(1H-1,2,4- triazol-5-yl)-2,3,5,6-tetrahydroazepino[5,4,3- de]quinolin-7(1H)-one |
| 39 | 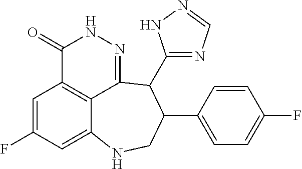 [SMILES: O=c1[nH]nc2c3c(cc(F)cc13)NCC(c1ccc(F)cc1)C2c1ncn[nH]1] | 5-fluoro-9-(4-fluorophenyl)-10-(1H-1,2,4-triazol-5- yl)-7,8,9,10-tetrahydroazepino[4,3,2-de]phthalazin- 3(2H)-one |
| 40 | 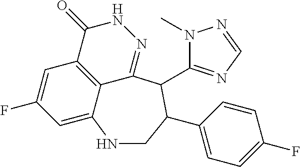 [SMILES: Cn1ncnc1C1c2n[nH]c(=O)c3cc(F)cc(c23)NCC1c1ccc(F)cc1] | 5-fluoro-9-(4-fluorophenyl)-10-(1-methyl-1H-1,2,4- triazol-5-yl)-7,8,9,10-tetrahydroazepino[4,3,2- de]phthalazin-3(2H)-one |
| 41 | 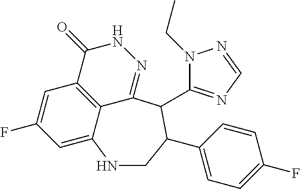 [SMILES: CCn1ncnc1C1c2n[nH]c(=O)c3cc(F)cc(c23)NCC1c1ccc(F)cc1] | 10-(1-ethyl-1H-1,2,4-triazol-5-yl)-5-fluoro-9-(4- fluorophenyl)-7,8,9,10-tetrahydroazepino[4,3,2- de]phthalazin-3(2H)-one |
| 42 | 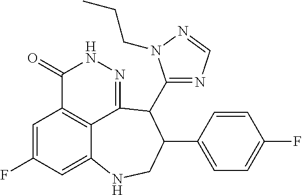 [SMILES: CCCn1ncnc1C1c2n[nH]c(=O)c3cc(F)cc(c23)NCC1c1ccc(F)cc1] | 5-fluoro-9-(4-fluorophenyl)-10-(1-propyl-1H-1,2,4- triazol-5-yl)-7,8,9,10-tetrahydroazepino[4,3,2- de]phthalazin-3(2H)-one |
| 43 | 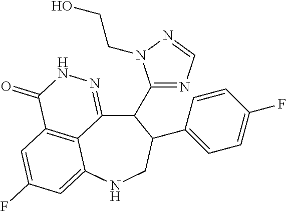 [SMILES: O=c1[nH]nc2c3c(cc(F)cc13)NCC(c1ccc(F)cc1)C2c1ncnn1CCO] | 5-fluoro-9-(4-fluorophenyl)-10-(1-(2- hydroxyethyl)-1H-1,2,4-triazol-5-yl)-7,8,9,10- tetrahydroazepino[4,3,2-de]phthalazin-3(2H)-one |
| 44 | 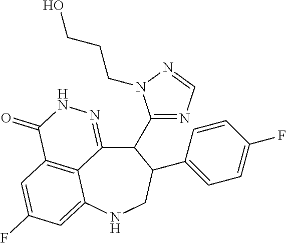 [SMILES: O=c1[nH]nc2c3c(cc(F)cc13)NCC(c1ccc(F)cc1)C2c1ncnn1CCCO] | 5-fluoro-9-(4-fluorophenyl)-10-(1-(3- hydroxypropyl)-1H-1,2,4-triazol-5-yl)-7,8,9,10- tetrahydroazepino[4,3,2-de]phthalazin-3(2H)-one |
| 45 | 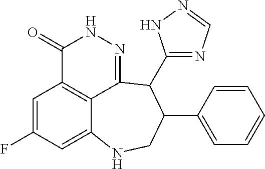 [SMILES: O=c1[nH]nc2c3c(cc(F)cc13)NCC(c1ccccc1)C2c1ncn[nH]1] | 5-fluoro-9-phenyl-10-(1H-1,2,4-triazol-5-yl)- 7,8,9,10-tetrahydroazepino[4,3,2-de]phthalazin- 3(2H)-one |
| 46 | 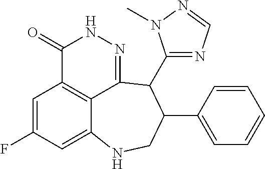 [SMILES: Cn1ncnc1C1c2n[nH]c(=O)c3cc(F)cc(c23)NCC1c1ccccc1] | 5-fluoro-10-(1-methyl-1H-1,2,4-triazol-5-yl)-9- phenyl-7,8,9,10-tetrahydroazepino[4,3,2- de]phthalazin-3(2H)-one |
| 47 | 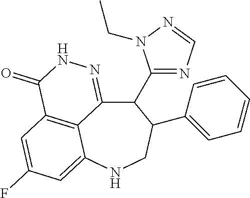 [SMILES: CCn1ncnc1C1c2n[nH]c(=O)c3cc(F)cc(c23)NCC1c1ccccc1] | 10-(1-ethyl-1H-1,2,4-triazol-5-yl)-5-fluoro-9- phenyl-7,8,9,10-tetrahydroazepino[4,3,2- de]phthalazin-3(2H)-one |
| 48 | 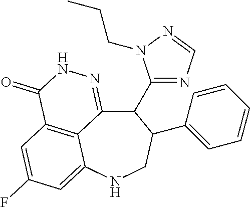 [SMILES: CCCn1ncnc1C1c2n[nH]c(=O)c3cc(F)cc(c23)NCC1c1ccccc1] | 5-fluoro-9-phenyl-10-(1-propyl-1H-1,2,4-triazol-5- yl)-7,8,9,10-tetrahydroazepino[4,3,2-de]phthalazin- 3(2H)-one |
| 49 | 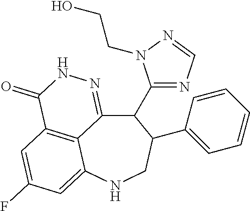 [SMILES: O=c1[nH]nc2c3c(cc(F)cc13)NCC(c1ccccc1)C2c1ncnn1CCO] | 5-fluoro-10-(1-(2-hydroxyethyl)-1H-1,2,4-triazol-5- yl)-9-phenyl-7,8,9,10-tetrahydroazepino[4,3,2- de]phthalazin-3(2H)-one |
| 50 | 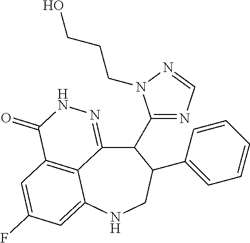 [SMILES: O=c1[nH]nc2c3c(cc(F)cc13)NCC(c1ccccc1)C2c1ncnn1CCCO] | 5-fluoro-10-(1-(3-hydroxypropyl)-1H-1,2,4-triazol- 5-yl)-9-phenyl-7,8,9,10-tetrahydroazepino[4,3,2- de]phthalazin-3(2H)-one |
| 51 | 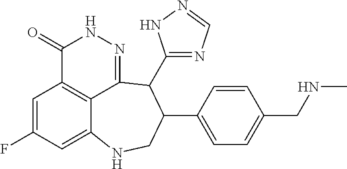 [SMILES: CNCc1ccc(C2CNc3cc(F)cc4c(=O)[nH]nc(c34)C2c2ncn[nH]2)cc1] | 5-fluoro-9-(4-((methylamino)methyl)phenyl)-10- (1H-1,2,4-triazol-5-yl)-7,8,9,10- tetrahydroazepino[4,3,2-de]phthalazin-3(2H)-one |
| 52 | 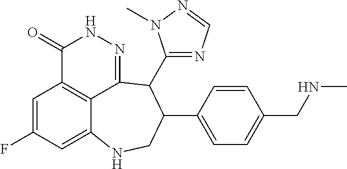 [SMILES: CNCc1ccc(C2CNc3cc(F)cc4c(=O)[nH]nc(c34)C2c2ncnn2C)cc1] | 5-fluoro-10-(1-methyl-1H-1,2,4-triazol-5-yl)-9-(4- ((methylamino)methyl)phenyl)-7,8,9,10- tetrahydroazepino[4,3,2-de]phthalazin-3(2H)-one |
| 53 | 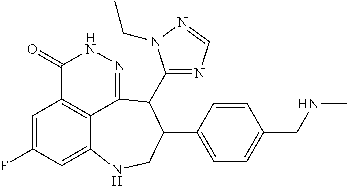 [SMILES: CCn1ncnc1C1c2n[nH]c(=O)c3cc(F)cc(c23)NCC1c1ccc(CNC)cc1] | 10-(1-ethyl-1H-1,2,4-triazol-5-yl)-5-fluoro-9-(4- ((methylamino)methyl)phenyl)-7,8,9,10- tetrahydroazepino[4,3,2-de]phthalazin-3(2H)-one |
| 54 | 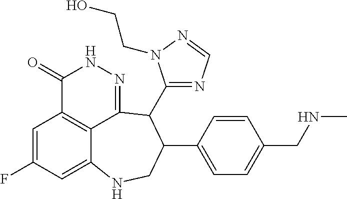 [SMILES: CNCc1ccc(C2CNc3cc(F)cc4c(=O)[nH]nc(c34)C2c2ncnn2CCO)cc1] | 5-fluoro-10-(1-(2-hydroxyethyl)-1H-1,2,4-triazol-5- yl)-9-(4-((methylamino)methyl)phenyl)-7,8,9,10- tetrahydroazepino[4,3,2-de]phthalazin-3(2H)-one |
| 55 | 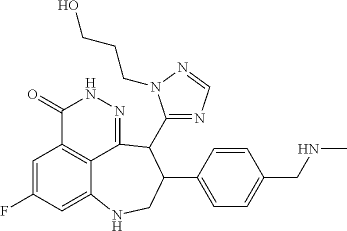 [SMILES: CNCc1ccc(C2CNc3cc(F)cc4c(=O)[nH]nc(c34)C2c2ncnn2CCCO)cc1] | 5-fluoro-10-(1-(3-hydroxypropyl)-1H-1,2,4-triazol- 5-yl)-9-(4-((methylamino)methyl)phenyl)-7,8,9,10- tetrahydroazepino[4,3,2-de]phthalazin-3(2H)-one |
| 56 | 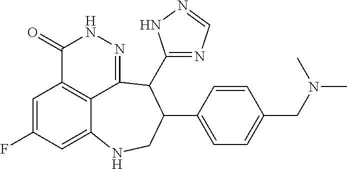 [SMILES: CN(C)Cc1ccc(C2CNc3cc(F)cc4c(=O)[nH]nc(c34)C2c2ncn[nH]2)cc1] | 9-(4-((dimethylamino)methyl)phenyl)-5-fluoro-10- (1H-1,2,4-triazol-5-yl)-7,8,9,10- tetrahydroazepino[4,3,2-de]phthalazin-3(2H)-one |
| 57 | 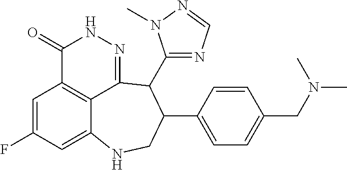 [SMILES: CN(C)Cc1ccc(C2CNc3cc(F)cc4c(=O)[nH]nc(c34)C2c2ncnn2C)cc1] | 9-(4-((dimethylamino)methyl)phenyl)-5-fluoro-10- (1-methyl-1H-1,2,4-triazol-5-yl)-7,8,9,10- tetrahydroazepino[4,3,2-de]phthalazin-3(2H)-one |
| 58 | 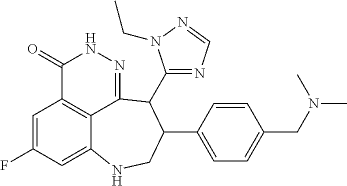 [SMILES: CCn1ncnc1C1c2n[nH]c(=O)c3cc(F)cc(c23)NCC1c1ccc(CN(C)C)cc1] | 9-(4-((dimethylamino)methyl)phenyl)-10-(1-ethyl- 1H-1,2,4-triazol-5-yl)-5-fluoro-7,8,9,10- tetrahydroazepino[4,3,2-de]phthalazin-3(2H)-one |
| 59 | 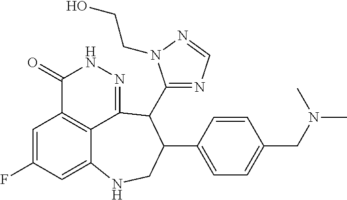 [SMILES: CN(C)Cc1ccc(C2CNc3cc(F)cc4c(=O)[nH]nc(c34)C2c2ncnn2CCO)cc1] | 9-(4-((dimethylamino)methyl)phenyl)-5-fluoro-10- (1-(2-hydroxyethyl)-1H-1,2,4-triazol-5-yl)-7,8,9,10- tetrahydroazepino[4,3,2-de]phthalazin-3(2H)-one |
| 60 | 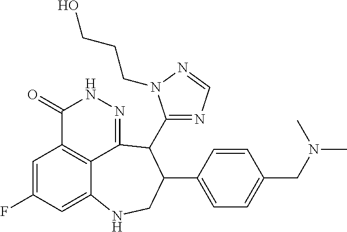 [SMILES: CN(C)Cc1ccc(C2CNc3cc(F)cc4c(=O)[nH]nc(c34)C2c2ncnn2CCCO)cc1] | 9-(4-((dimethylamino)methyl)phenyl)-5-fluoro-10- (1-(3-hydroxypropyl)-1H-1,2,4-triazol-5-yl)- 7,8,9,10-tetrahydroazepino[4,3,2-de]phthalazin- 3(2H)-one |
| 61 | 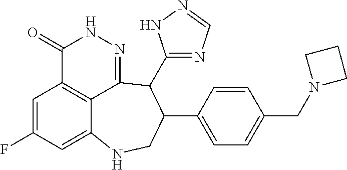 [SMILES: O=c1[nH]nc2c3c(cc(F)cc13)NCC(c1ccc(CN3CCC3)cc1)C2c1ncn[nH]1] | 9-(4-(azetidin-1-ylmethyl)phenyl)-5-fluoro-10-(1H- 1,2,4-triazol-5-yl)-7,8,9,10-tetrahydroazepino[4,3,2- de]phthalazin-3(2H)-one |
| 62 | 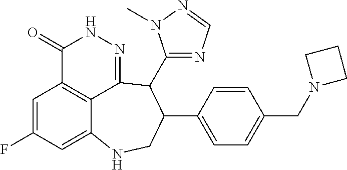 [SMILES: Cn1ncnc1C1c2n[nH]c(=O)c3cc(F)cc(c23)NCC1c1ccc(CN2CCC2)cc1] | 9-(4-(azetidin-1-ylmethyl)phenyl)-5-fluoro-10-(1- methyl-1H-1,2,4-triazol-5-yl)-7,8,9,10- tetrahydroazepino[4,3,2-de]phthalazin-3(2H)-one |
| 63 | 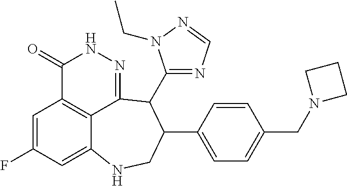 [SMILES: CCn1ncnc1C1c2n[nH]c(=O)c3cc(F)cc(c23)NCC1c1ccc(CN2CCC2)cc1] | 9-(4-azetidin-1-ylmethyl)phenyl)-10-(1-ethyl-1H- 1,2,4-triazol-5-yl)-5-fluoro-7,8,9,10- tetrahydroazepino[4,3,2-de]phthalazin-3(2H)-one |
| 64 | 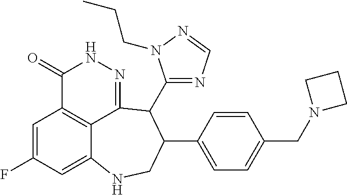 [SMILES: CCCn1ncnc1C1c2n[nH]c(=O)c3cc(F)cc(c23)NCC1c1ccc(CN2CCC2)cc1] | 9-(4-(azetidin-1-ylmethyl)phenyl)-5-fluoro-10-(1- propyl-1H-1,2,4-triazol-5-yl)-7,8,9,10- tetrahydroazepino[4,3,2-de]phthalazin-3(2H)-one |
| 65 | 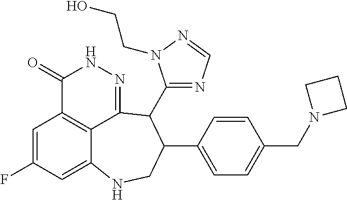 [SMILES: O=c1[nH]nc2c3c(cc(F)cc13)NCC(c1ccc(CN3CCC3)cc1)C2c1ncnn1CCO] | 9-(4-(azetidin-1-ylmethyl)phenyl)-5-fluoro-10-(1- (2-hydroxyethyl)-1H-1,2,4-triazol-5-yl)-7,8,9,10- tetrahydroazepino[4,3,2-de]phthalazin-3(2H)-one |
| 66 | 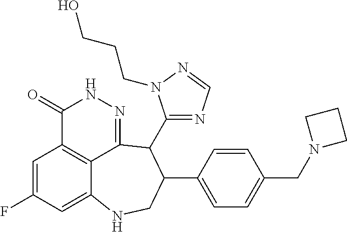 [SMILES: O=c1[nH]nc2c3c(cc(F)cc13)NCC(c1ccc(CN3CCC3)cc1)C2c1ncnn1CCCO] | 9-(4-(azetidin-1-ylmethyl)phenyl)-5-fluoro-10-(1- (3-hydroxypropyl)-1H-1,2,4-triazol-5-yl)-7,8,9,10- tetrahydroazepino[4,3,2-de]phthalazin-3(2H)-one |
| 67 | 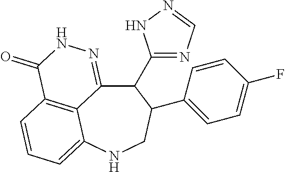 [SMILES: O=c1[nH]nc2c3c(cccc13)NCC(c1ccc(F)cc1)C2c1ncn[nH]1] | 9-(4-fluorophenyl)-10-(1H-1,2,4-triazol-5-yl)- 7,8,9,10-tetrahydroazepino[4,3,2-de]phthalazin- 3(2H)-one |
| 68 | 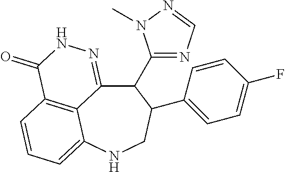 [SMILES: Cn1ncnc1C1c2n[nH]c(=O)c3cccc(c23)NCC1c1ccc(F)cc1] | 9-(4-fluorophenyl)-10-(1-methyl-1H-1,2,4-triazol-5- yl)-7,8,9,10-tetrahydroazepino[4,3,2-de]phthalazin- 3(2H)-one |
| 69 | 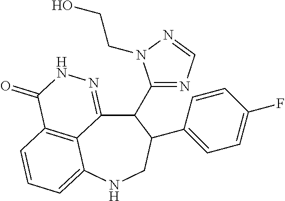 [SMILES: O=c1[nH]nc2c3c(cccc13)NCC(c1ccc(F)cc1)C2c1ncnn1CCO] | 9-(4-fluorophenyl)-10-(1-(2-hydroxyethyl)-1H- 1,2,4-triazol-5-yl)-7,8,9,10-tetrahydroazepino[4,3,2- de]phthalazin-3(2H)-one |
| 70 | 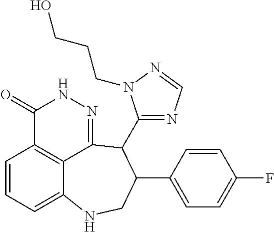 [SMILES: O=c1[nH]nc2c3c(cccc13)NCC(c1ccc(F)cc1)C2c1ncnn1CCCO] | 9-(4-fluorophenyl)-10-(1-(3-hydroxypropyl)-1H- 1,2,4-triazol-5-yl)-7,8,9,10-tetrahydroazepino[4,3,2- de]phthalazin-3(2H)-one |
| 71 | 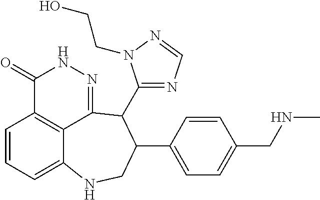 [SMILES: CNCc1ccc(C2CNc3cccc4c(=O)[nH]nc(c34)C2c2ncnn2CCO)cc1] | 10-(1-(2-hydroxyethyl)-1H-1,2,4-triazol-5-yl)-9-(4- ((methylamino)methyl)phenyl)-7,8,9,10- tetrahydroazepino[4,3,2-de]phthalazin-3(2H)-one |
| 72 | 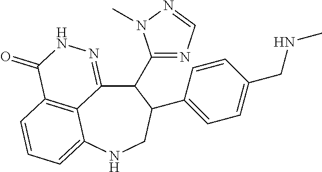 [SMILES: CNCc1ccc(C2CNc3cccc4c(=O)[nH]nc(c34)C2c2ncnn2C)cc1] | 10-(1-methyl-1H-1,2,4-triazol-5-yl)-9-(4- ((methylamino)methyl)phenyl)-7,8,9,10- tetrahydroazepino[4,3,2-de]phthalazin-3(2H)-one |
| 73 | 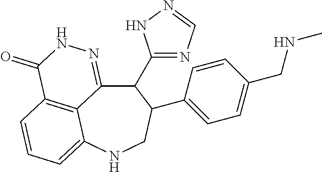 [SMILES: CNCc1ccc(C2CNc3cccc4c(=O)[nH]nc(c34)C2c2ncn[nH]2)cc1] | 9-(4-((methylamino)methyl)phenyl)-10-(1H-1,2,4- triazol-5-yl)-7,8,9,10-tetrahydroazepino[4,3,2-de] phthalazin-3(2H)-one |
| 74 | 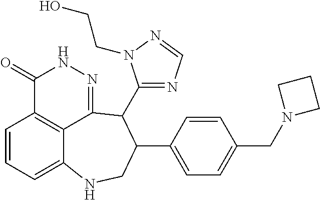 [SMILES: O=c1[nH]nc2c3c(cccc13)NCC(c1ccc(CN3CCC3)cc1)C2c1ncnn1CCO] | 9-(4-(azetidin-1-ylmethyl)phenyl)-10-(1-(2- hydroxyethyl)-1H-1,2,4-triazol-5-yl)-7,8,9,10- tetrahydroazepino[4,3,2-de]phthalazin-3(2H)-one |
| 75 | 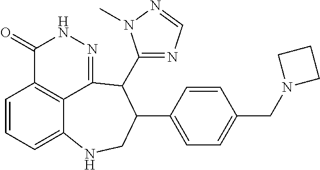 [SMILES: Cn1ncnc1C1c2n[nH]c(=O)c3cccc(c23)NCC1c1ccc(CN2CCC2)cc1] | 9-(4-(azetidin-1-ylmethyl)phenyl)-10-(1-methyl- 1H-1,2,4-triazol-5-yl)-7,8,9,10- tetrahydroazepino[4,3,2-de]phthalazin-3(2H)-one |
| 76 | 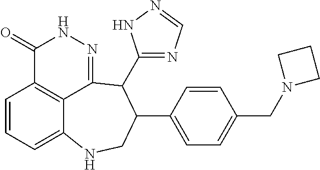 [SMILES: O=c1[nH]nc2c3c(cccc13)NCC(c1ccc(CN3CCC3)cc1)C2c1ncn[nH]1] | 9-(4-(azetidin-1-ylmethyl)phenyl)-10-(1H-1,2,4- triazol-5-yl)-7,8,9,10-tetrahydroazepino[4,3,2- de]phthalazin-3(2H)-one |
| 77 | 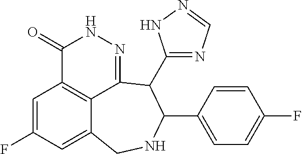 [SMILES: O=c1[nH]nc2c3c(cc(F)cc13)CNC(c1ccc(F)cc1)C2c1ncn[nH]1] | 5-fluoro-9-(4-fluorophenyl)-10-(1H-1,2,4-triazol-5- yl)-7,8,9,10-tetrahydroazepino[5,4,3-de]phthalazin- 3(2H)-one |
| 78 | 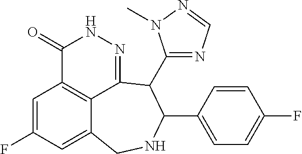 [SMILES: Cn1ncnc1C1c2n[nH]c(=O)c3cc(F)cc(c23)CNC1c1ccc(F)cc1] | 5-fluoro-9-(4-fluorophenyl)-10-(1-methyl-1H-1,2,4- triazol-5-yl)-7,8,9,10-tetrahydroazepino[5,4,3-de] phthalazin-3(2H)-one |
| 79 | 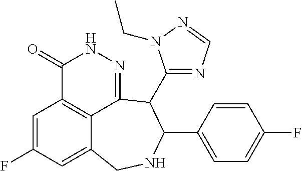 [SMILES: CCn1ncnc1C1c2n[nH]c(=O)c3cc(F)cc(c23)CNC1c1ccc(F)cc1] | 10-(1-ethyl-1H-1,2,4-triazol-5-yl)-5-fluoro-9-(4- fluorophenyl)-7,8,9,10-tetrahydroazepino[5,4,3- de]phthalazin-3(2H)-one |
| 80 | 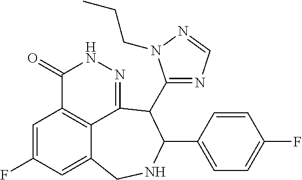 [SMILES: CCCn1ncnc1C1c2n[nH]c(=O)c3cc(F)cc(c23)CNC1c1ccc(F)cc1] | 5-fluoro-9-(4-fluorophenyl)-10-(1-propyl-1H-1,2,4- triazol-5-yl)-7,8,9,10-tetrahydroazepino[5,4,3- de]phthalazin-3(2H)-one |
| 81 | 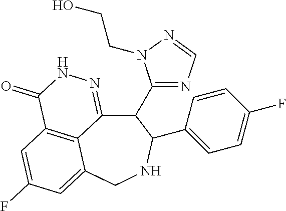 [SMILES: O=c1[nH]nc2c3c(cc(F)cc13)CNC(c1ccc(F)cc1)C2c1ncnn1CCO] | 5-fluoro-9-(4-fluorophenyl)-10-(1-(2- hydroxyethyl)-1H-1,2,4-triazol-5-yl)-7,8,9,10- tetrahydroazepino[5,4,3-de]phthalazin-3(2H)-one |
| 82 | 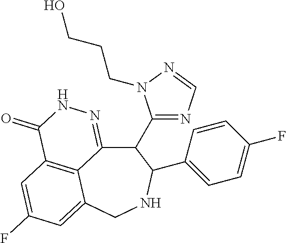 [SMILES: O=c1[nH]nc2c3c(cc(F)cc13)CNC(c1ccc(F)cc1)C2c1ncnn1CCCO] | 5-fluoro-9-(4-fluorophenyl)-10-(1-(3- hydroxypropyl)-1H-1,2,4-triazol-5-yl)-7,8,9,10- tetrahydroazepino[5,4,3-de]phthalazin-3(2H)-one |
| 83 | 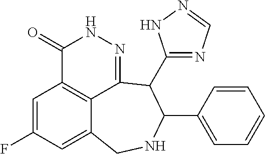 [SMILES: O=c1[nH]nc2c3c(cc(F)cc13)CNC(c1ccccc1)C2c1ncn[nH]1] | 5-fluoro-9-phenyl-10-(1H-1,2,4-triazol-5-yl)- 7,8,9,10-tetrahydroazepino[5,4,3-de]phthalazin- 3(2H)-one |
| 84 | 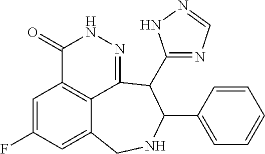 [SMILES: O=c1[nH]nc2c3c(cc(F)cc13)CNC(c1ccccc1)C2c1ncn[nH]1] | 5-fluoro-10-(1-methyl-1H-1,2,4-triazol-5-yl)-9- phenyl-7,8,9,10-tetrahydroazepino[5,4,3- de]phthalazin-3(2H)-one |
| 85 | 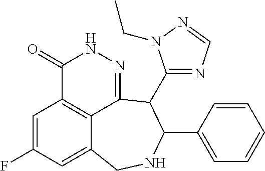 [SMILES: CCn1ncnc1C1c2n[nH]c(=O)c3cc(F)cc(c23)CNC1c1ccccc1] | 10-(1-ethyl-1H-1,2,4-triazol-5-yl)-5-fluoro-9- phenyl-7,8,9,10-tetrahydroazepino[5,4,3- de]phthalazin-3(2H)-one |
| 86 | 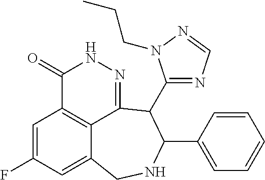 [SMILES: CCCn1ncnc1C1c2n[nH]c(=O)c3cc(F)cc(c23)CNC1c1ccccc1] | 5-fluoro-9-phenyl-10-(1-propyl-1H-1,2,4-triazol-5- yl)-7,8,9,10-tetrahydroazepino[5,4,3-de]phthalazin- 3(2H)-one |
| 87 | 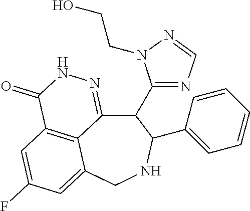 [SMILES: O=c1[nH]nc2c3c(cc(F)cc13)CNC(c1ccccc1)C2c1ncnn1CCO] | 5-fluoro-10-(1-(2-hydroxyethyl)-1H-1,2,4-triazol-5- yl)-9-phenyl-7,8,9,10-tetrahydroazepino[5,4,3- de]phthalazin-3(2H)-one |
| 88 | 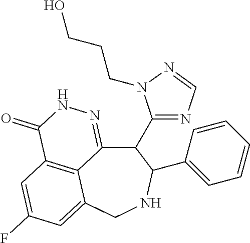 [SMILES: O=c1[nH]nc2c3c(cc(F)cc13)CNC(c1ccccc1)C2c1ncnn1CCCO] | 5-fluoro-10-(1-(3-hydroxypropyl)-1H-1,2,4-triazol- 5-yl)-9-phenyl-7,8,9,10-tetrahydroazepino[5,4,3- de]phthalazin-3(2H)-one |
| 89 | 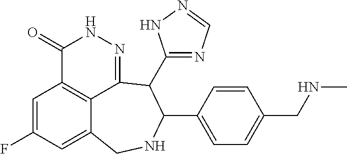 [SMILES: CNCc1ccc(C2NCc3cc(F)cc4c(=O)[nH]nc(c34)C2c2ncn[nH]2)cc1] | 5-fluoro-9-(4-((methylamino)methyl)phenyl)-10- (1H-1,2,4-triazol-5-yl)-7,8,9,10-tetrahydroazepino [5,4,3-de]phthalazin-3(2H)-one |
| 90 | 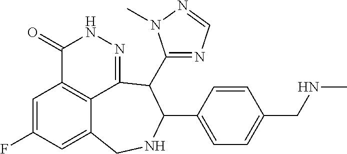 [SMILES: CNCc1ccc(C2NCc3cc(F)cc4c(=O)[nH]nc(c34)C2c2ncnn2C)cc1] | 5-fluoro-10-(1-methyl-1H-1,2,4-triazol-5-yl)-9-(4- ((methylamino)methyl)phenyl)-7,8,9,10- tetrahydroazepino[5,4,3-de]phthalazin-3(2H)-one |
| 91 | 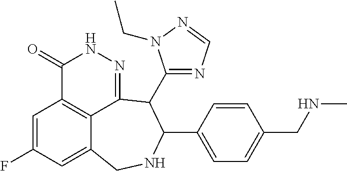 [SMILES: CCn1ncnc1C1c2n[nH]c(=O)c3cc(F)cc(c23)CNC1c1ccc(CNC)cc1] | 10-(1-ethyl-1H-1,2,4-triazol-5-yl)-5-fluoro-9-(4- ((methylamino)methyl)phenyl)-7,8,9,10- tetrahydroazepino[5,4,3-de]phthalazin-3(2H)-one |
| 92 | 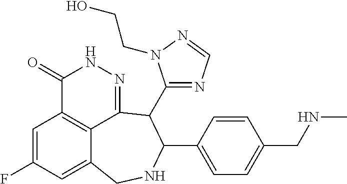 [SMILES: CNCc1ccc(C2NCc3cc(F)cc4c(=O)[nH]nc(c34)C2c2ncnn2CCO)cc1] | 5-fluoro-10-(1-(2-hydroxyethyl)-1H-1,2,4-triazol-5- yl)-9-(4-((methylamino)methyl)phenyl)-7,8,9,10- tetrahydroazepino[5,4,3-de]phthalazin-3(2H)-one |
| 93 | 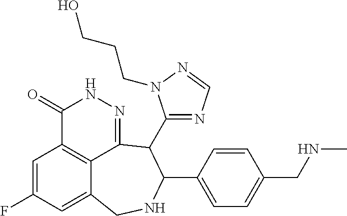 [SMILES: CNCc1ccc(C2NCc3cc(F)cc4c(=O)[nH]nc(c34)C2c2ncnn2CCCO)cc1] | 5-fluoro-10-(1-(3-hydroxypropyl)-1H-1,2,4-triazol- 5-yl)-9-(4-((methylamino)methyl)phenyl)- 7,8,9,10-tetrahydroazepino[5,4,3-de]phthalazin- 3(2H)-one |
| 94 | 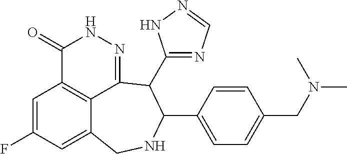 [SMILES: CN(C)Cc1ccc(C2NCc3cc(F)cc4c(=O)[nH]nc(c34)C2c2ncn[nH]2)cc1] | 9-(4-((dimethylamino)methyl)phenyl)-5-fluoro-10- (1H-1,2,4-triazol-5-yl)-7,8,9,10- tetrahydroazepino[5,4,3-de]phthalazin-3(2H)-one |
| 95 | 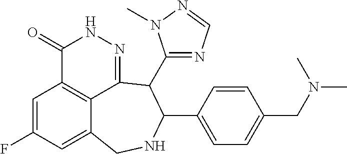 [SMILES: CN(C)Cc1ccc(C2NCc3cc(F)cc4c(=O)[nH]nc(c34)C2c2ncnn2C)cc1] | 9-(4-((dimethylamino)methyl)phenyl)-5-fluoro-10- (1-methyl-1H-1,2,4-triazol-5-yl)-7,8,9,10- tetrahydroazepino[5,4,3-de]phthalazin-3(2H)-one |
| 96 | 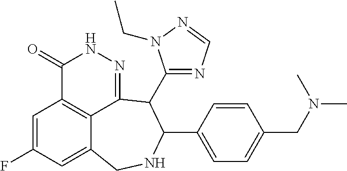 [SMILES: CCn1ncnc1C1c2n[nH]c(=O)c3cc(F)cc(c23)CNC1c1ccc(CN(C)C)cc1] | 9-(4-((dimethylamino)methyl)phenyl)-10-(1-ethyl- 1H-1,2,4-triazol-5-yl)-5-fluoro-7,8,9,10- tetrahydroazepino[5,4,3-de]phthalazin-3(2H)-one |
| 97 | 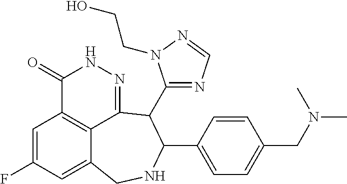 [SMILES: CN(C)Cc1ccc(C2NCc3cc(F)cc4c(=O)[nH]nc(c34)C2c2ncnn2CCO)cc1] | 9-(4-((dimethylamino)methyl)phenyl)-5-fluoro-10- (1-(2-hydroxyethyl)-1H-1,2,4-triazol-5-yl)-7,8,9,10- tetrahydroazepino[5,4,3-de]phthalazin-3(2H)-one |
| 98 | 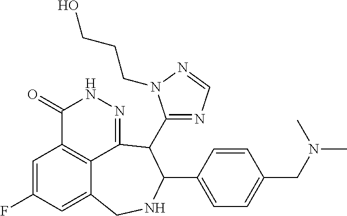 [SMILES: CN(C)Cc1ccc(C2NCc3cc(F)cc4c(=O)[nH]nc(c34)C2c2ncnn2CCCO)cc1] | 9-(4-((dimethylamino)methyl)phenyl)-5-fluoro-10- (1-(3-hydroxypropyl)-1H-1,2,4-triazol-5-yl)- 7,8,9,10-tetrahydroazepino[5,4,3-de]phthalazin- 3(2H)-one |
| 99 | 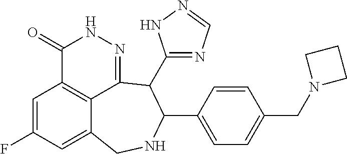 [SMILES: O=c1[nH]nc2c3c(cc(F)cc13)CNC(c1ccc(CN3CCC3)cc1)C2c1ncn[nH]1] | 9-(4-(azetidin-1-ylmethyl)phenyl)-5-fluoro-10-(1H- 1,2,4-triazol-5-yl)-7,8,9,10-tetrahydroazepino [5,4,3-de]phthalazin-3(2H)-one |
| 100 | 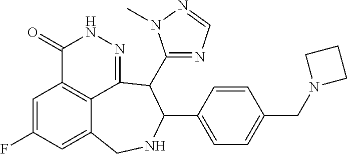 [SMILES: Cn1ncnc1C1c2n[nH]c(=O)c3cc(F)cc(c23)CNC1c1ccc(CN2CCC2)cc1] | 9-(4-(azetidin-1-ylmethyl)phenyl)-5-fluoro-10-(1- methyl-1H-1,2,4-triazol-5-yl)-7,8,9,10- tetrahydroazepino[5,4,3-de]phthalazin-3(2H)-one |
| 101 | 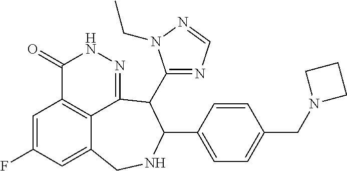 [SMILES: CCn1ncnc1C1c2n[nH]c(=O)c3cc(F)cc(c23)CNC1c1ccc(CN2CCC2)cc1] | 9-(4-(azetidin-1-ylmethyl)phenyl)-10-(1-ethyl-1H- 1,2,4-triazol-5-yl)-5-fluoro-7,8,9,10- tetrahydroazepino[5,4,3-de]phthalazin-3(2H)-one |
| 102 | 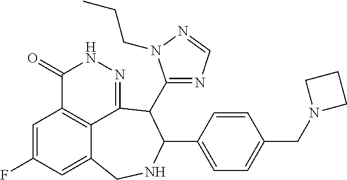 [SMILES: CCCn1ncnc1C1c2n[nH]c(=O)c3cc(F)cc(c23)CNC1c1ccc(CN2CCC2)cc1] | 9-(4-(azetidin-1-ylmethyl)phenyl)-5-fluoro-10-(1- propyl-1H-1,2,4-triazol-5-yl)-7,8,9,10- tetrahydroazepino[5,4,3-de]phthalazin-3(2H)-one |
| 103 | 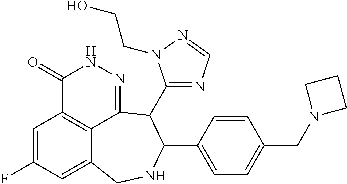 [SMILES: O=c1[nH]nc2c3c(cc(F)cc13)CNC(c1ccc(CN3CCC3)cc1)C2c1ncnn1CCO] | 9-(4-(azetidin-1-ylmethyl)phenyl)-5-fluoro-10-(1- (2-hydroxyethyl)-1H-1,2,4-triazol-5-yl)-7,8,9,10- tetrahydroazepino[5,4,3-de]phthalazin-3(2H)-one |
| 104 | 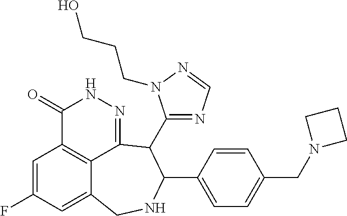 [SMILES: O=c1[nH]nc2c3c(cc(F)cc13)CNC(c1ccc(CN3CCC3)cc1)C2c1ncnn1CCCO] | 9-(4-(azetidin-1-ylmethyl)phenyl)-5-fluoro-10-(1- (3-hydroxyethyl)-1H-1,2,4-triazol-5-yl)-7,8,9,10- tetrahydroazepino[5,4,3-de]phthalazin-3(2H)-one |
| 105 | 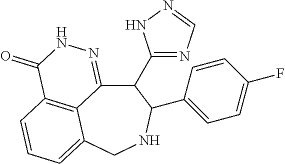 [SMILES: O=c1[nH]nc2c3c(cccc13)CNC(c1ccc(F)cc1)C2c1ncn[nH]1] | 9-(4-fluorophenyl)-10-(1H-1,2,4-triazol-5-yl)- 7,8,9,10-tetrahydroazepino[5,4,3-de]phthalazin- 3(2H)-one |
| 106 | 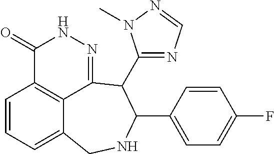 [SMILES: Cn1ncnc1C1c2n[nH]c(=O)c3cccc(c23)CNC1c1ccc(F)cc1] | 9-(4-fluorophenyl)-10-(1-methyl-1H-1,2,4-triazol-5- yl)-7,8,9,10-tetrahydroazepino[5,4,3-de]phthalazin- 3(2H)-one |
| 107 | 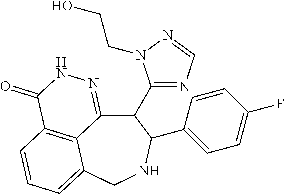 [SMILES: O=c1[nH]nc2c3c(cccc13)CNC(c1ccc(F)cc1)C2c1ncnn1CCO] | 9-(4-fluorophenyl)-10-(1-(2-hydroxyethyl)-1H- 1,2,4-triazol-5-yl)-7,8,9,10-tetrahydroazepino[5,4,3- de]phthalazin-3(2H)-one |
| 108 | 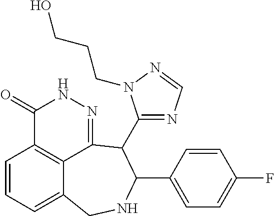 [SMILES: O=c1[nH]nc2c3c(cccc13)CNC(c1ccc(F)cc1)C2c1ncnn1CCCO] | 9-(4-fluorophenyl)-10-(1-(3-hydroxypropyl)-1H- 1,2,4-triazol-5-yl)-7,8,9,10-tetrahydroazepino[5,4,3- de]phthalazin-3(2H)-one |
| 109 | 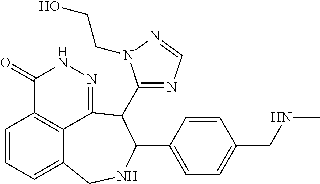 [SMILES: CNCc1ccc(C2NCc3cccc4c(=O)[nH]nc(c34)C2c2ncnn2CCO)cc1] | 10-(1-(2-hydroxyethyl)-1H-1,2,4-triazol-5-yl)-9-(4- ((methylamino)methyl)phenyl)-7,8,9,10- tetrahydroazepino[5,4,3-de]phthalazin-3(2H)-one |
| 110 | 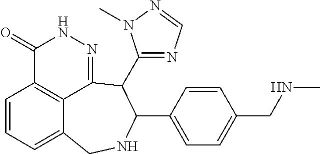 [SMILES: CNCc1ccc(C2NCc3cccc4c(=O)[nH]nc(c34)C2c2ncnn2C)cc1] | 10-(1-methyl-1H-1,2,4-triazol-5-yl)-9-(4- ((methylamino)methyl)phenyl)-7,8,9,10- tetrahydroazepino[5,4,3-de]phthalazin-3(2H)-one |
| 111 | 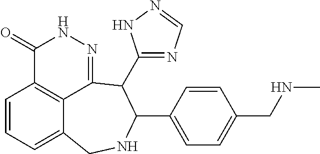 [SMILES: CNCc1ccc(C2NCc3cccc4c(=O)[nH]nc(c34)C2c2ncn[nH]2)cc1] | 9-(4-((methylamino)methyl)phenyl)-10-(1H-1,2,4- triazol-5-yl)-7,8,9,10-tetrahydroazepino[5,4,3- de]phthalazin-3(2H)-one |
| 112 | 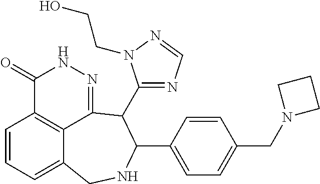 [SMILES: O=c1[nH]nc2c3c(cccc13)CNC(c1ccc(CN3CCC3)cc1)C2c1ncnn1CCO] | 9-(4-(azetidin-1-ylmethyl)phenyl)-10-(1-(2- hydroxyethyl)-1H-1,2,4-triazol-5-yl)-7,8,9,10- tetrahydroazepino[5,4,3-de]phthalazin-3(2H)-one |
| 113 | 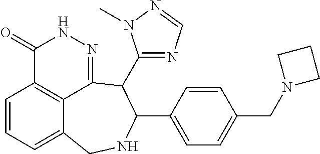 [SMILES: Cn1ncnc1C1c2n[nH]c(=O)c3cccc(c23)CNC1c1ccc(CN2CCC2)cc1] | 9-(4-(azetidin-1-ylmethyl)phenyl)-10-(1-methyl- 1H-1,2,4-triazol-5-yl)-7,8,9,10- tetrahydroazepino[5,4,3-de]phthalazin-3(2H)-one |
| 114 | 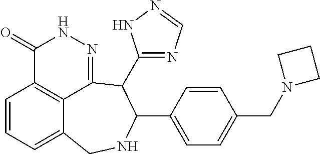 [SMILES: O=c1[nH]nc2c3c(cccc13)CNC(c1ccc(CN3CCC3)cc1)C2c1ncn[nH]1] | 9-(4-(azetidin-1-ylmethyl)phenyl)-10-(1H-1,2,4- triazol-5-yl)-7,8,9,10-tetrahydroazepino[5,4,3- de]phthalazin-3(2H)-one |
| 115 | 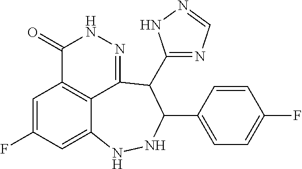 [SMILES: O=c1[nH]nc2c3c(cc(F)cc13)NNC(c1ccc(F)cc1)C2c1ncn[nH]1] | 9-fluoro-3-(4-fluorophenyl)-4-(1H-1,2,4-triazol-5- yl)-1,2,3,4-tetrahydro-[1,2]diazepino[5,4,3- de]phthalazin-7(6H)-one |
| 116 | 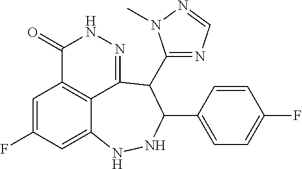 [SMILES: Cn1ncnc1C1c2n[nH]c(=O)c3cc(F)cc(c23)NNC1c1ccc(F)cc1] | 9-fluoro-3-(4-fluorophenyl)-4-(1-methyl-1H-1,2,4- triazol-5-yl)-1,2,3,4-tetrahydro-[1,2]diazepino [5,4,3-de]phthalazin-7(6H)-one |
| 117 | 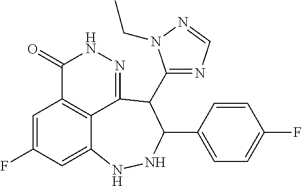 [SMILES: CCn1ncnc1C1c2n[nH]c(=O)c3cc(F)cc(c23)NNC1c1ccc(F)cc1] | 4-(1-ethyl-1H-1,2,4-triazol-5-yl)-9-fluoro-3-(4- fluorophenyl)-1,2,3,4-tetrahydro-[1,2]diazepino [5,4,3-de]phthalazin-7(6H)-one |
| 118 | 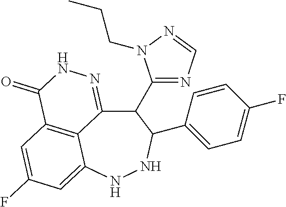 [SMILES: CCCn1ncnc1C1c2n[nH]c(=O)c3cc(F)cc(c23)NNC1c1ccc(F)cc1] | 9-fluoro-3-(4-fluorophenyl)-4-(1-propyl-1H-1,2,4- triazol-5-yl)-1,2,3,4-tetrahydro-[1,2]diazepino [5,4,3-de]phthalazin-7(6H)-one |
| 119 | 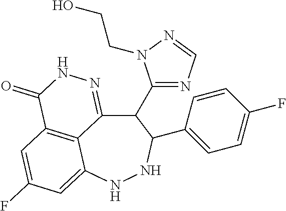 [SMILES: O=c1[nH]nc2c3c(cc(F)cc13)NNC(c1ccc(F)cc1)C2c1ncnn1CCO] | 9-fluoro-3-(4-fluorophenyl)-4-(1-(2-hydroxyethyl)- 1H-1,2,4-triazol-5-yl)-1,2,3,4-tetrahydro- [1,2]diazepino[5,4,3-de]phthalazin-7(6H)-one |
| 120 | 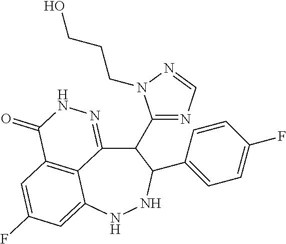 [SMILES: O=c1[nH]nc2c3c(cc(F)cc13)NNC(c1ccc(F)cc1)C2c1ncnn1CCCO] | 9-fluoro-3-(4-fluorophenyl)-4-(1-(3- hydroxypropyl)-1H-1,2,4-triazol-5-yl)- 1,2,3,4-tetrahydro-[1,2]diazepino[5,4,3-de] phthalazin-7(6H)-one |
| 121 | 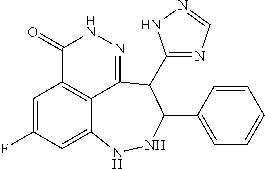 [SMILES: O=c1[nH]nc2c3c(cc(F)cc13)NNC(c1ccccc1)C2c1ncn[nH]1] | 9-fluoro-3-phenyl-4-(1H-1,2,4-triazol-5-yl)-1,2,3,4- tetrahydro-[1,2]diazepino[5,4,3-de]phthalazin- 7(6H)-one |
| 122 | 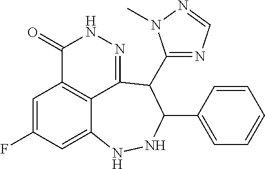 [SMILES: Cn1ncnc1C1c2n[nH]c(=O)c3cc(F)cc(c23)NNC1c1ccccc1] | 9-fluoro-4-(1-methyl-1H-1,2,4-triazol-5-yl)-3- phenyl-1,2,3,4-tetrahydro-[1,2]diazepino[5,4,3-de] phthalazin-7(6H)-one |
| 123 | 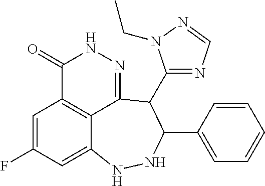 [SMILES: CCn1ncnc1C1c2n[nH]c(=O)c3cc(F)cc(c23)NNC1c1ccccc1] | 4-(1-ethyl-1H-1,2,4-triazol-5-yl)-9-fluoro-3-phenyl- 1,2,3,4-tetrahydro-[1,2]diazepino[5,4,3-de] phthalazin-7(6H)-one |
| 124 | 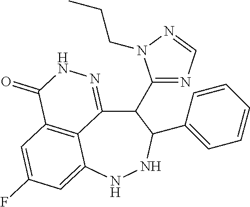 [SMILES: CCCn1ncnc1C1c2n[nH]c(=O)c3cc(F)cc(c23)NNC1c1ccccc1] | 9-fluoro-3-phenyl-4-(1-propyl-1H-1,2,4-triazol-5- yl)-1,2,3,4-tetrahydro-[1,2]diazepino[5,4,3-de] phthalazin-7(6H)-one |
| 125 | 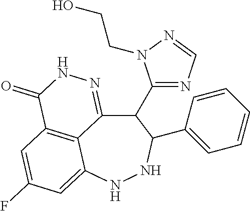 [SMILES: O=c1[nH]nc2c3c(cc(F)cc13)NNC(c1ccccc1)C2c1ncnn1CCO] | 9-fluoro-4-(1-(2-hydroxyethyl)-1H-1,2,4-triazol-5- yl)-3-phenyl-1,2,3,4-tetrahydro-[1,2]diazepino [5,4,3-de]phthalazin-7(6H)-one |
| 126 | 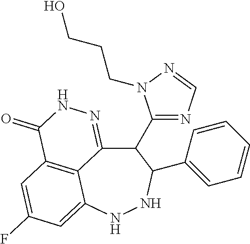 [SMILES: O=c1[nH]nc2c3c(cc(F)cc13)NNC(c1ccccc1)C2c1ncnn1CCCO] | 9-fluoro-4-(1-(2-hydroxyethyl)-1H-1,2,4-triazol-5- yl)-3-phenyl-1,2,3,4-tetrahydro-[1,2]diazepino [5,4,3-de]phthalazin-7(6H)-one |
| 127 | 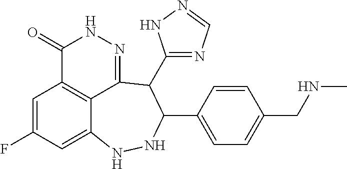 [SMILES: CNCc1ccc(C2NNc3cc(F)cc4c(=O)[nH]nc(c34)C2c2ncn[nH]2)cc1] | 9-fluoro-3-(4-((methylamino)methyl)phenyl)-4- (1H-1,2,4-triazol-5-yl)-1,2,3,4-tetrahydro-[1,2] diazepino[5,4,3-de]phthalazin-7(6H)-one |
| 128 | 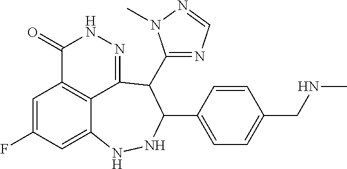 [SMILES: CNCc1ccc(C2NNc3cc(F)cc4c(=O)[nH]nc(c34)C2c2ncnn2C)cc1] | 9-fluoro-4-(1-methyl-1H-1,2,4-triazol-5-yl)-3-(4- ((methylamino)methyl)phenyl)-1,2,3,4-tetrahydro- [1,2]diazepino[5,4,3-de]phthalazin-7(6H)-one |
| 129 | 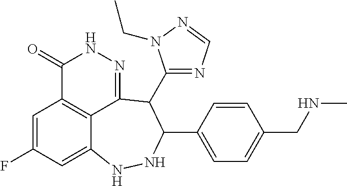 [SMILES: CCn1ncnc1C1c2n[nH]c(=O)c3cc(F)cc(c23)NNC1c1ccc(CNC)cc1] | 4-(1-ethyl-1H-1,2,4-triazol-5-yl)-9-fluoro-3-(4- ((methylamino)methyl)phenyl)-1,2,3,4-tetrahydro- [1,2]diazepino[5,4,3-de]phthalazin-7(6H)-one |
| 130 | 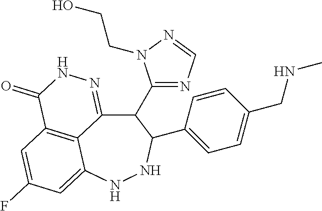 [SMILES: CNCc1ccc(C2NNc3cc(F)cc4c(=O)[nH]nc(c34)C2c2ncnn2CCO)cc1] | 9-fluoro-4-(1-(2-hydroxyethyl)-1H-1,2,4-triazol-5- yl)-3-(4-((methylamino)methyl)phenyl)-1,2,3,4- tetrahydro-[1,2]diazepino[5,4,3-de]phthalazin- 7(6H)-one |
| 131 | 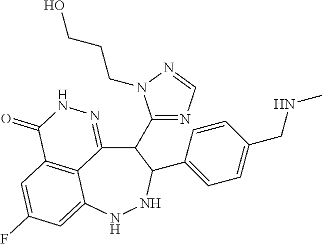 [SMILES: CNCc1ccc(C2NNc3cc(F)cc4c(=O)[nH]nc(c34)C2c2ncnn2CCCO)cc1] | 9-fluoro-4-(1-(3-hydroxypropyl)-1H-1,2,4-triazol-5- yl)-3-(4-((methylamino)methyl)phenyl)-1,2,3,4- tetrahydro-[1,2]diazepino[5,4,3-de]phthalazin- 7(6H)-one |
| 132 | 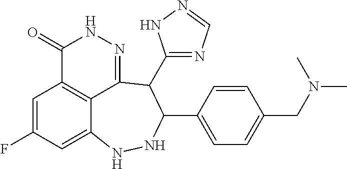 [SMILES: CN(C)Cc1ccc(C2NNc3cc(F)cc4c(=O)[nH]nc(c34)C2c2ncn[nH]2)cc1] | 3-(4-((dimethylamino)methyl)phenyl)-9-fluoro-4- (1H-1,2,4-triazol-5-yl)-1,2,3,4-tetrahydro- [1,2]diazepino[5,4,3-de]phthalazin-7(6H)-one |
| 133 | 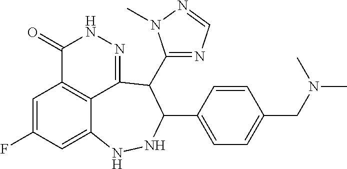 [SMILES: CN(C)Cc1ccc(C2NNc3cc(F)cc4c(=O)[nH]nc(c34)C2c2ncnn2C)cc1] | 3-(4-((dimethylamino)methyl)phenyl)-9-fluoro-4- (1-methyl-1H-1,2,4-triazol-5-yl)-1,2,3,4- tetrahydro-[1,2]diazepino[5,4,3-de]phthalazin- 7(6H)-one |
| 134 | 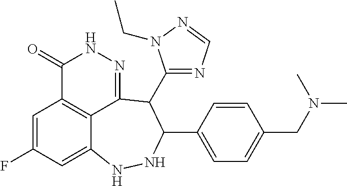 [SMILES: CCn1ncnc1C1c2n[nH]c(=O)c3cc(F)cc(c23)NNC1c1ccc(CN(C)C)cc1] | 3-(4-((dimethylamino)methyl)phenyl)-4-(1-ethyl- 1H-1,2,4-triazol-5-yl)-9-fluoro-1,2,3,4- tetrahydro-[1,2]diazepino[5,4,3-de]phthalazin- 7(6H)-one |
| 135 | 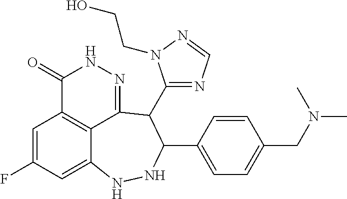 [SMILES: CN(C)Cc1ccc(C2NNc3cc(F)cc4c(=O)[nH]nc(c34)C2c2ncnn2CCO)cc1] | 3-(4-((dimethylamino)methyl)phenyl)-9-fluoro-4- (1-(2-hydroxyethyl)-1H-1,2,4-triazol-5-yl)-1,2,3,4- tetrahydro-[1,2]diazepino[5,4,3-de]phthalazin- 7(6H)-one |
| 136 | 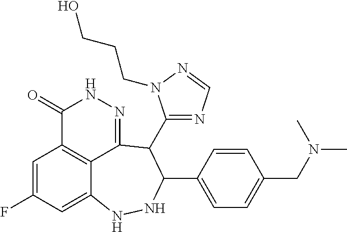 [SMILES: CN(C)Cc1ccc(C2NNc3cc(F)cc4c(=O)[nH]nc(c34)C2c2ncnn2CCCO)cc1] | 3-(4-((dimethylamino)methyl)phenyl)-9-fluoro-4- (1-(3-hydroxypropyl)-1H-1,2,4-triazol-5-yl)- 1,2,3,4-tetrahydro-[1,2]diazepino[5,4,3-de] phthalazin-7(6H)-one |
| 137 | 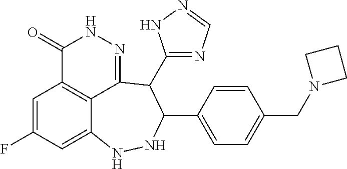 [SMILES: O=c1[nH]nc2c3c(cc(F)cc13)NNC(c1ccc(CN3CCC3)cc1)C2c1ncn[nH]1] | 3-(4-(azetidin-1-ylmethyl)phenyl)-9-fluoro-4-(1H- 1,2,4-triazol-5-yl)-1,2,3,4-tetrahydro-[1,2] diazepino[5,4,3-de]phthalazin-7(6H)-one |
| 138 | 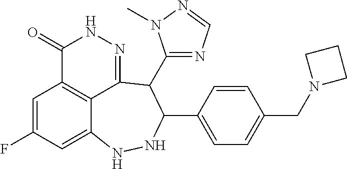 [SMILES: Cn1ncnc1C1c2n[nH]c(=O)c3cc(F)cc(c23)NNC1c1ccc(CN2CCC2)cc1] | 3-(4-(azetidin-1-ylmethyl)phenyl)-9-fluoro-4-(1- methyl-1H-1,2,4-triazol-5-yl)-1,2,3,4-tetrahydro- [1,2]diazepino[5,4,3-de]phthalazin-7(6H)-one |
| 139 | 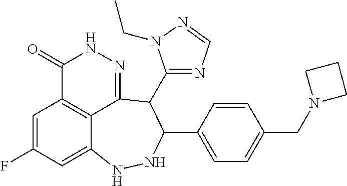 [SMILES: CCn1ncnc1C1c2n[nH]c(=O)c3cc(F)cc(c23)NNC1c1ccc(CN2CCC2)cc1] | 3-(4-(azetidin-1-ylmethyl)phenyl)-4-(1-ethyl-1H- 1,2,4-triazol-5-yl)-9-fluoro-1,2,3,4-tetrahydro- [1,2]diazepino[5,4,3-de]phthalazin-7(6H)-one |
| 140 | 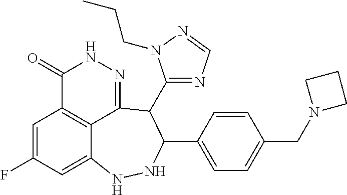 [SMILES: CCCn1ncnc1C1c2n[nH]c(=O)c3cc(F)cc(c23)NNC1c1ccc(CN2CCC2)cc1] | 3-(4-(azetidin-1-ylmethyl)phenyl)-9-fluoro-4-(1- propyl-1H-1,2,4-triazol-5-yl)-1,2,3,4-tetrahydro- [1,2]diazepino[5,4,3-de]phthalazin-7(6H)-one |
| 141 | 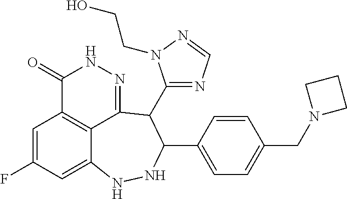 [SMILES: O=c1[nH]nc2c3c(cc(F)cc13)NNC(c1ccc(CN3CCC3)cc1)C2c1ncnn1CCO] | 3-(4-(azetidin-1-ylmethyl)phenyl)-9-fluoro-4-(1-(2- hydroxyethyl)-1H-1,2,4-triazol-5-yl)-1,2,3,4- tetrahydro-[1,2]diazepino[5,4,3-de]phthalazin- 7(6H)-one |
| 142 | 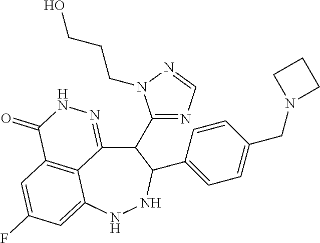 [SMILES: O=c1[nH]nc2c3c(cc(F)cc13)NNC(c1ccc(CN3CCC3)cc1)C2c1ncnn1CCCO] | 3-(4-(azetidin-1-ylmethyl)phenyl)-9-fluoro-4-(1-(3- hydroxypropyl)-1H-1,2,4-triazol-5-yl)-1,2,3,4- tetrahydro-[1,2]diazepino[5,4,3-de]phthalazin- 7(6H)-one |
| 143 | 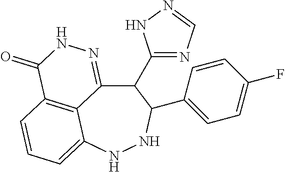 [SMILES: O=c1[nH]nc2c3c(cccc13)NNC(c1ccc(F)cc1)C2c1ncn[nH]1] | 3-(4-fluorophenyl)-4-(1H-1,2,4-triazol-5-yl)- 1,2,3,4-tetrahydro-[1,2]diazepino[5,4,3- de]phthalazin-7(6H)-one |
| 144 | 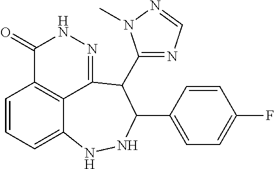 [SMILES: Cn1ncnc1C1c2n[nH]c(=O)c3cccc(c23)NNC1c1ccc(F)cc1] | 3-(4-fluorophenyl)-4-(1-methyl-1H-1,2,4-triazol-5- yl)-1,2,3,4-tetrahydro-[1,2]diazepino[5,4,3- de]phthalazin-7(6H)-one |
| 145 | 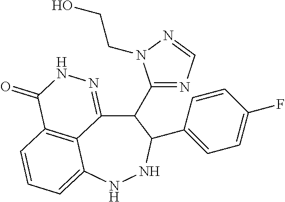 [SMILES: O=c1[nH]nc2c3c(cccc13)NNC(c1ccc(F)cc1)C2c1ncnn1CCO] | 3-(4-fluorophenyl)-4-(1-(2-hydroxyethyl)-1H-1,2,4- triazol-5-yl)-1,2,3,4-tetrahydro- [1,2]diazepino[5,4,3-de]phthalazin-7(6H)-one |
| 146 | 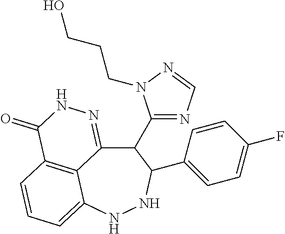 [SMILES: O=c1[nH]nc2c3c(cccc13)NNC(c1ccc(F)cc1)C2c1ncnn1CCCO] | 3-(4-fluorophenyl)-4-(1-(3-hydroxypropyl)-1H- 1,2,4-triazol-5-yl)-1,2,3,4-tetrahydro- [1,2]diazepino[5,4,3-de]phthalazin-7(6H)-one |
| 147 | 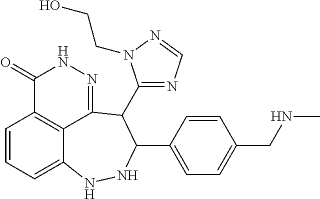 [SMILES: CNCc1ccc(C2NNc3cccc4c(=O)[nH]nc(c34)C2c2ncnn2CCO)cc1] | 4-(1-(2-hydroxyethyl)-1H-1,2,4-triazol-5-yl)-3-(4- ((methylamino)methyl)phenyl)-1,2,3,4-tetrahydro- [1,2]diazepino[5,4,3-de]phthalazin-7(6H)-one |
| 148 | 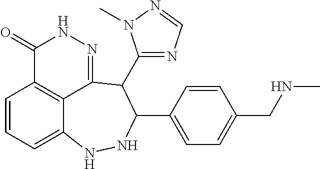 [SMILES: CNCc1ccc(C2NNc3cccc4c(=O)[nH]nc(c34)C2c2ncnn2C)cc1] | 4-(1-methyl-1H-1,2,4-triazol-5-yl)-3-(4- ((methylamino)methyl)phenyl)-1,2,3,4-tetrahydro- [1,2]diazepino[5,4,3-de]phthalazin-7(6H)-one |
| 149 | 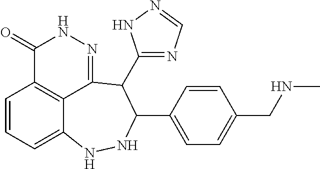 [SMILES: CNCc1ccc(C2NNc3cccc4c(=O)[nH]nc(c34)C2c2ncn[nH]2)cc1] | 3-(4-((methylamino)methyl)phenyl)-4-(1H-1,2,4- triazol-5-yl)-1,2,3,4-tetrahydro- [1,2]diazepino[5,4,3-de]phthalazin-7(6H)-one |
| 150 | 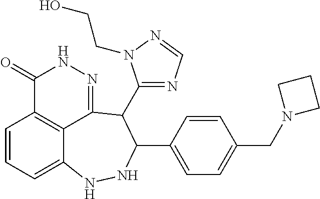 [SMILES: O=c1[nH]nc2c3c(cccc13)NNC(c1ccc(CN3CCC3)cc1)C2c1ncnn1CCO] | 3-(4-(azetidin-1-ylmethyl)phenyl)-4-(1-(2- hydroxyethyl)-1H-1,2,4-triazol-5-yl)-1,2,3,4- tetrahydro-[1,2]diazepino[5,4,3-de]phthalazin- 7(6H)-one |
| 151 | 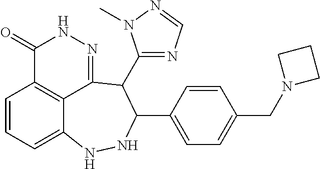 [SMILES: Cn1ncnc1C1c2n[nH]c(=O)c3cccc(c23)NNC1c1ccc(CN2CCC2)cc1] | 3-(4-(azetidin-1-ylmethyl)phenyl)-4-(1-methyl-1H- 1,2,4-triazol-5-yl)-1,2,3,4-tetrahydro- [1,2]diazepino[5,4,3-de]phthalazin-7(6H)-one |
| 152 | 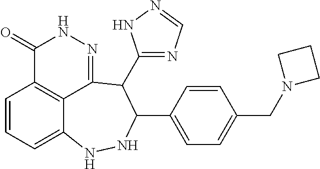 [SMILES: O=c1[nH]nc2c3c(cccc13)NNC(c1ccc(CN3CCC3)cc1)C2c1ncn[nH]1] | 3-(4-(azetidin-1-ylmethyl)phenyl)-4-(1H-1,2,4- triazol-5-yl)-1,2,3,4-tetrahydro- [1,2]diazepino[5,4,3-de]phthalazin-7(6H)-one |
| 153 | 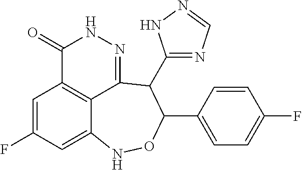 [SMILES: O=c1[nH]nc2c3c(cc(F)cc13)NOC(c1ccc(F)cc1)C2c1ncn[nH]1] | 9-fluoro-3-(4-fluorophenyl)-4-(1H-1,2,4-triazol-5- yl)-3,4-dihydro-1H-[1,2]oxazepino[5,4,3-de] phthalazin-7(6H)-one |
| 154 | 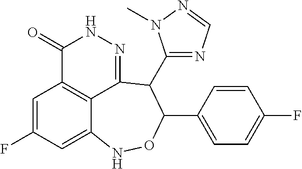 [SMILES: Cn1ncnc1C1c2n[nH]c(=O)c3cc(F)cc(c23)NOC1c1ccc(F)cc1] | 9-fluoro-3-(4-fluorophenyl)-4-(1-methyl-1H-1,2,4- triazol-5-yl)-3,4-dihydro-1H-[1,2]oxazepino[5,4,3- de]phthalazin-7(6H)-one |
| 155 | 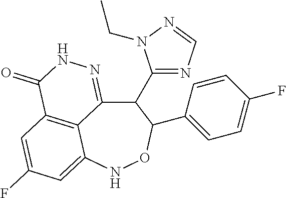 [SMILES: CCn1ncnc1C1c2n[nH]c(=O)c3cc(F)cc(c23)NOC1c1ccc(F)cc1] | 4-(1-ethyl-1H-1,2,4-triazol-5-yl)-9-fluoro-3-(4- fluorophenyl)-3,4-dihydro-1H- [1,2]oxazepino[5,4,3-de]phthalazin-7(6H)-one |
| 156 | 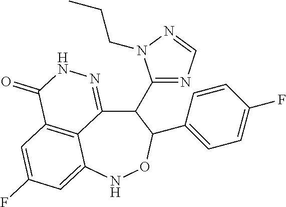 [SMILES: CCCn1ncnc1C1c2n[nH]c(=O)c3cc(F)cc(c23)NOC1c1ccc(F)cc1] | 9-fluoro-3-(4-fluorophenyl)-4-(1-propyl-1H-1,2,4- triazol-5-yl)-3,4-dihydro-1H-[1,2]oxazepino[5,4,3- de]phthalazin-7(6H)-one |
| 157 | 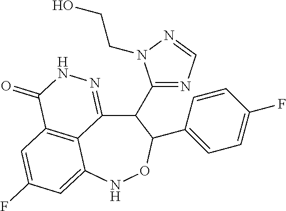 [SMILES: O=c1[nH]nc2c3c(cc(F)cc13)NOC(c1ccc(F)cc1)C2c1ncnn1CCO] | 9-fluoro-3-(4-fluorophenyl)-4-(1-(2-hydroxyethyl)- 1H-1,2,4-triazol-5-yl)-3,4-dihydro-1H- [1,2]oxazepino[5,4,3-de]phthalazin-7(6H)-one |
| 158 | 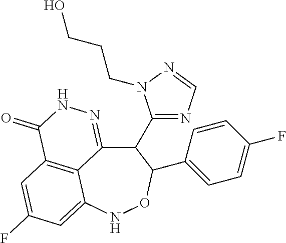 [SMILES: O=c1[nH]nc2c3c(cc(F)cc13)NOC(c1ccc(F)cc1)C2c1ncnn1CCCO] | 9-fluoro-3-(4-fluorophenyl)-4-(1-(3- hydroxypropyl)-1H-1,2,4-triazol-5-yl)-3,4-dihydro- 1H-[1,2]oxazepino[5,4,3-de]phthalazin-7(6H)-one |
| 159 | 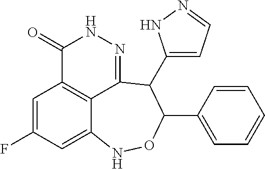 [SMILES: O=c1[nH]nc2c3c(cc(F)cc13)NOC(c1ccccc1)C2c1ccn[nH]1] | 9-fluoro-3-phenyl-4-(1H-1,2,4-triazol-5-yl)-3,4- dihydro-1H-[1,2]oxazepino[5,4,3-de]phthalazin- 7(6H)-one |
| 160 | 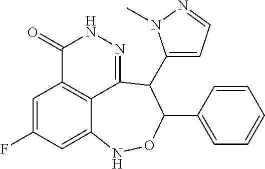 [SMILES: Cn1nccc1C1c2n[nH]c(=O)c3cc(F)cc(c23)NOC1c1ccccc1] | 9-fluoro-4-(1-methyl-1H-1,2,4-triazol-5-yl)-3- phenyl-3,4-dihydro-1H-[1,2]oxazepino[5,4,3- de]phthalazin-7(6H)-one |
| 161 | 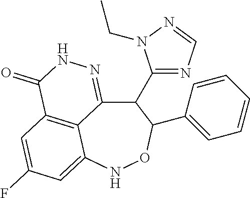 [SMILES: CCn1ncnc1C1c2n[nH]c(=O)c3cc(F)cc(c23)NOC1c1ccccc1] | 4-(1-ethyl-1H-1,2,4-triazol-5-yl)-9-fluoro-3- phenyl-3,4-dihydro-1H-[1,2]oxazepino [5,4,3-de]phthalazin-7(6H)-one |
| 162 | 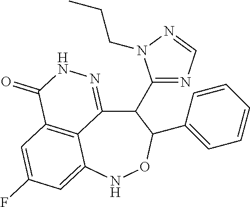 [SMILES: CCCn1ncnc1C1c2n[nH]c(=O)c3cc(F)cc(c23)NOC1c1ccccc1] | 9-fluoro-3-phenyl-4-(1-propyl-1H-1,2,4-triazol-5- yl)-3,4-dihydro-1H-[1,2]oxazepino[5,4,3- de]phthalazin-7(6H)-one |
| 163 | 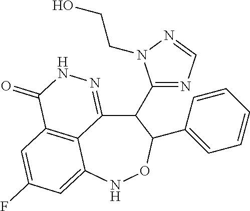 [SMILES: O=c1[nH]nc2c3c(cc(F)cc13)NOC(c1ccccc1)C2c1ncnn1CCO] | 9-fluoro-4-(1-(2-hydroxyethyl)-1H-1,2,4-triazol-5- yl)-3-fluoro-3,4-dihydro-1H-[1,2]oxazepino[5,4,3- de]phthalazin-7(6H)-one |
| 164 | 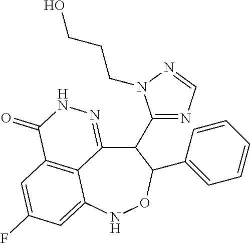 [SMILES: O=c1[nH]nc2c3c(cc(F)cc13)NOC(c1ccccc1)C2c1ncnn1CCCO] | 9-fluoro-4-(1-(3-hydroxypropyl)-1H-1,2,4-triazol-5- yl)-3-phenyl-3,4-dihydro-1H-[1,2]oxazepino[5,4,3- de]phthalazin-7(6H)-one |
| 165 | 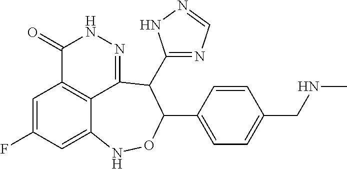 [SMILES: CNCc1ccc(C2ONc3cc(F)cc4c(=O)[nH]nc(c34)C2c2ncn[nH]2)cc1] | 9-fluoro-3-(4-((methylamino)methyl)phenyl)-4- (1H-1,2,4-triazol-5-yl)-3,4-dihydro-1H- [1,2]oxazepino[5,4,3-de]phthalazin-7(6H)-one |
| 166 | 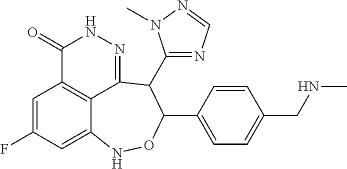 [SMILES: CNCc1ccc(C2ONc3cc(F)cc4c(=O)[nH]nc(c34)C2c2ncnn2C)cc1] | 9-fluoro-4-(1-methyl-1H-1,2,4-triazol-5-yl)-3-(4- ((methylamino)methyl)phenyl)-3,4-dihydro-1H- [1,2]oxazepino[5,4,3-de]phthalazin-7(6H)-one |
| 167 | 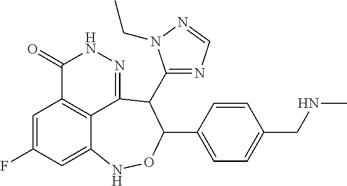 [SMILES: CCn1ncnc1C1c2n[nH]c(=O)c3cc(F)cc(c23)NOC1c1ccc(CNC)cc1] | 4-(1-ethyl-1H-1,2,4-triazol-5-yl)-9-fluoro-3-(4- ((methylamino)methyl)phenyl)-3,4-dihydro-1H- [1,2]oxazepino[5,4,3-de]phthalazin-7(6H)-one |
| 168 | 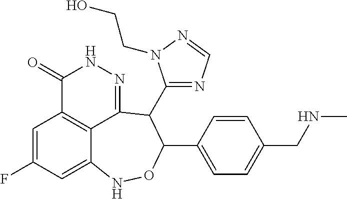 [SMILES: CNCc1ccc(C2ONc3cc(F)cc4c(=O)[nH]nc(c34)C2c2ncnn2CCO)cc1] | 9-fluoro-4-(1-(2-hydroxyethyl)-1H-1,2,4-triazol-5- yl)-3-(4-((methylamino)methyl)phenyl)-3,4- dihydro-1H-[1,2]oxazepino[5,4,3-de]phthalazin- 7(6H)-one |
| 169 | 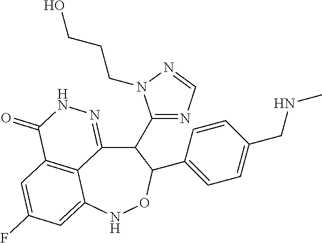 [SMILES: CNCc1ccc(C2ONc3cc(F)cc4c(=O)[nH]nc(c34)C2c2ncnn2CCCO)cc1] | 9-fluoro-4-(1-(3-hydroxypropyl)-1H-1,2,4-triazol-5- yl)-3-(4-((methylamino)methyl)phenyl)-3,4- dihydro-1H-[1,2]oxazepino[5,4,3-de]phthalazin- 7(6H)-one |
| 170 | 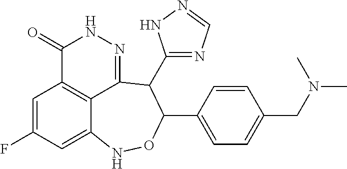 [SMILES: CN(C)Cc1ccc(C2ONc3cc(F)cc4c(=O)[nH]nc(c34)C2c2ncn[nH]2)cc1] | 3-(4-((dimethylamino)methyl)phenyl)-9-fluoro-4- (1H-1,2,4-triazol-5-yl)-3,4-dihydro-1H- [1,2]oxazepino[5,4,3-de]phthalazin-7(6H)-one |
| 171 | 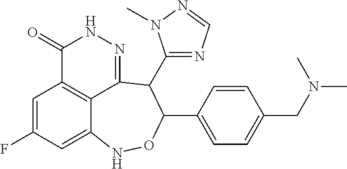 [SMILES: CN(C)Cc1ccc(C2ONc3cc(F)cc4c(=O)[nH]nc(c34)C2c2ncnn2C)cc1] | 3-(4-((dimethylamino)methyl)phenyl)-9-fluoro-4- (1-methyl-1H-1,2,4-triazol-5-yl)-3,4-dihydro-1H- [1,2]oxazepino[5,4,3-de]phthalazin-7(6H)-one |
| 172 | 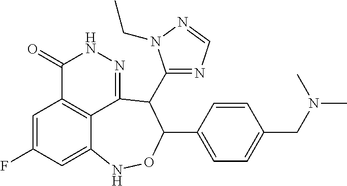 [SMILES: CCn1ncnc1C1c2n[nH]c(=O)c3cc(F)cc(c23)NOC1c1ccc(CN(C)C)cc1] | 3-(4-((dimethylamino)methyl)phenyl)-4-(1-ethyl- 1H-1,2,4-triazol-5-yl)-9-fluoro-3,4-dihydro-1H- [1,2]oxazepino[5,4,3-de]phthalazin-7(6H)-one |
| 173 | 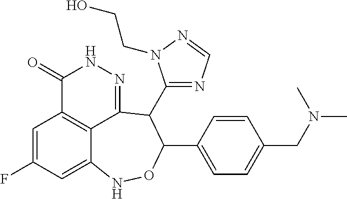 [SMILES: CN(C)Cc1ccc(C2ONc3cc(F)cc4c(=O)[nH]nc(c34)C2c2ncnn2CCO)cc1] | 3-(4-((dimethylamino)methyl)phenyl)-9-fluoro-4- (1-(2-hydroxyethyl)-1H-1,2,4-triazol-5-yl)-3,4- dihydro-1H-[1,2]oxazepino[5,4,3-de]phthalazin- 7(6H)-one |
| 174 | 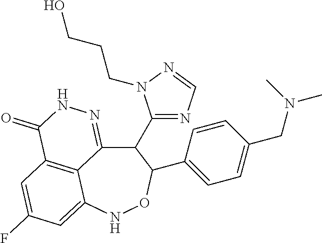 [SMILES: CN(C)Cc1ccc(C2ONc3cc(F)cc4c(=O)[nH]nc(c34)C2c2ncnn2CCCO)cc1] | 3-(4-((dimethylamino)methyl)phenyl)-9-fluoro-4- (1-(3-hydroxypropyl)-1H-1,2,4-triazol-5-yl)-3,4- dihydro-1H-[1,2]oxazepino[5,4,3-de]phthalazin- 7(6H)-one |
| 175 | 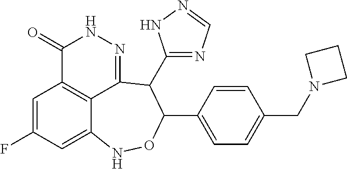 [SMILES: O=c1[nH]nc2c3c(cc(F)cc13)NOC(c1ccc(CN3CCC3)cc1)C2c1ncn[nH]1] | 3-(4-(azetidin-1-ylmethyl)phenyl)-9-fluoro-4-(1H- 1,2,4-triazol-5-yl)-3,4-dihydro-1H- [1,2]oxazepino[5,4,3-de]phthalazin-7(6H)-one |
| 176 | 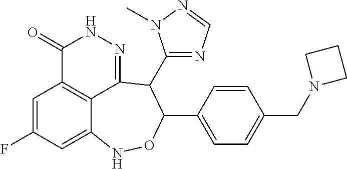 [SMILES: Cn1ncnc1C1c2n[nH]c(=O)c3cc(F)cc(c23)NOC1c1ccc(CN2CCC2)cc1] | 3-(4-(azetidin-1-ylmethyl)phenyl)-9-fluoro-4-(1- methyl-1H-1,2,4-triazol-5-yl)-3,4-dihydro-1H- [1,2]oxazepino[5,4,3-de]phthalazin-7(6H)-one |
| 177 | 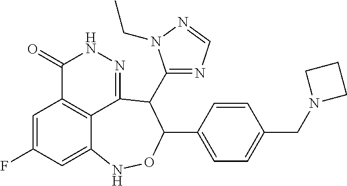 [SMILES: CCn1ncnc1C1c2n[nH]c(=O)c3cc(F)cc(c23)NOC1c1ccc(CN2CCC2)cc1] | 3-(4-(azetidin-1-ylmethyl)phenyl)-4-(1-ethyl-1H- 1,2,4-triazol-5-yl)-9-fluoro-3,4-dihydro-1H- [1,2]oxazepino[5,4,3-de]phthalazin-7(6H)-one |
| 178 | 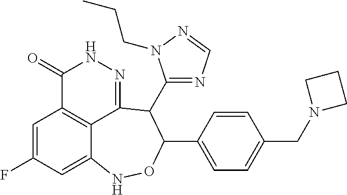 [SMILES: CCCn1ncnc1C1c2n[nH]c(=O)c3cc(F)cc(c23)NOC1c1ccc(CN2CCC2)cc1] | 3-(4-(azetidin-1-ylmethyl)phenyl)-9-fluoro-4-(1- propyl-1H-1,2,4-triazol-5-yl)-3,4-dihydro-1H- [1,2]oxazepino[5,4,3-de]phthalazin-7(6H)-one |
| 179 | 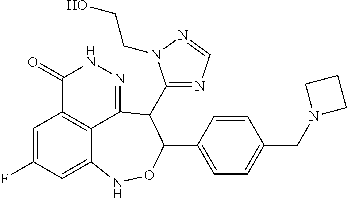 [SMILES: O=c1[nH]nc2c3c(cc(F)cc13)NOC(c1ccc(CN3CCC3)cc1)C2c1ncnn1CCO] | 3-(4-(azetidin-1-ylmethyl)phenyl)-9-fluoro-4-(1-(2- hydroxyethyl)-1H-1,2,4-triazol-5-yl)-3,4-dihydro- 1H-[1,2]oxazepino[5,4,3-de]phthalazin-7(6H)-one |
| 180 | 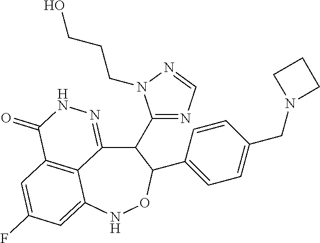 [SMILES: O=c1[nH]nc2c3c(cc(F)cc13)NOC(c1ccc(CN3CCC3)cc1)C2c1ncnn1CCCO] | 3-(4-(azetidin-1-ylmethyl)phenyl)-9-fluoro-4-(1-(3- hydroxypropyl)-1H-1,2,4-triazol-5-yl)-3,4-dihydro- 1H-[1,2]oxazepino[5,4,3-de]phthalazin-7(6H)-one |
| 181 | 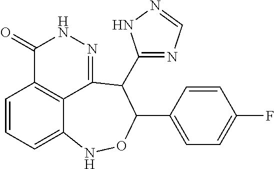 [SMILES: O=c1[nH]nc2c3c(cccc13)NOC(c1ccc(F)cc1)C2c1ncn[nH]1] | 3-(4-fluorophenyl)-4-(1H-1,2,4-triazol-5-yl)-3,4- dihydro-1H-[1,2]oxazepino[5,4,3-de]phthalazin- 7(6H)-one |
| 182 | 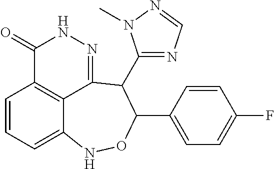 [SMILES: Cn1ncnc1C1c2n[nH]c(=O)c3cccc(c23)NOC1c1ccc(F)cc1] | 3-(4-fluorophenyl)-4-(1-methyl-1H-1,2,4-triazol-5- yl)-3,4-dihydro-1H-[1,2]oxazepino[5,4,3- de]phthalazin-7(6H)-one |
| 183 | 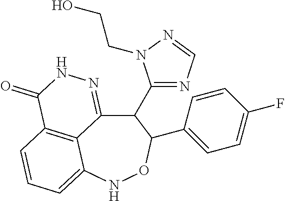 [SMILES: O=c1[nH]nc2c3c(cccc13)NOC(c1ccc(F)cc1)C2c1ncnn1CCO] | 3-(4-fluorophenyl)-4-(1-(2-hydroxyethyl)-1H-1,2,4- triazol-5-yl)-3,4-dihydro-1H-[1,2]oxazepino[5,4,3- de]phthalazin-7(6H)-one |
| 184 | 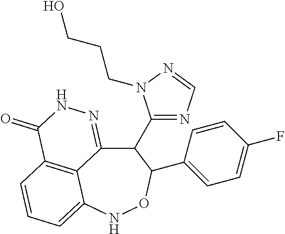 [SMILES: O=c1[nH]nc2c3c(cccc13)NOC(c1ccc(F)cc1)C2c1ncnn1CCCO] | 3-(4-fluorophenyl)-4-(1-(3-hydroxypropyl)-1H- 1,2,4-triazol-5-yl)-3,4-dihydro-1H- [1,2]oxazepino[5,4,3-de]phthalazin-7(6H)-one |
| 185 | 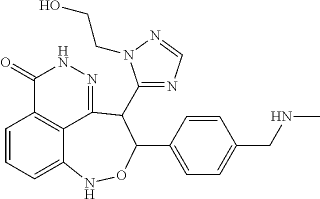 [SMILES: CNCc1ccc(C2ONc3cccc4c(=O)[nH]nc(c34)C2c2ncnn2CCO)cc1] | 4-(1-(2-hydroxyethyl)-1H-1,2,4-triazol-5-yl)-3-(4- ((methylamino)methyl)phenyl)-3,4-dihydro-1H- [1,2]oxazepino[5,4,3-de]phthalazin-7(6H)-one |
| 186 | 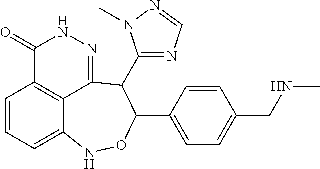 [SMILES: CNCc1ccc(C2ONc3cccc4c(=O)[nH]nc(c34)C2c2ncnn2C)cc1] | 4-(1-methyl-1H-1,2,4-triazol-5-yl)-3-(4- ((methylamino)methyl)phenyl)-3,4-dihydro-1H- [1,2]oxazepino[5,4,3-de]phthalazin-7(6H)-one |
| 187 | 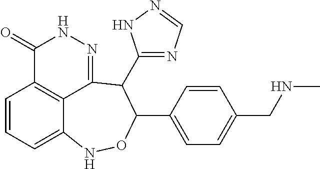 [SMILES: CNCc1ccc(C2ONc3cccc4c(=O)[nH]nc(c34)C2c2ncn[nH]2)cc1] | 3-(4-((methylamino)methyl)phenyl)-4-(1H-1,2,4- triazol-5-yl)-3,4-dihydro-1H-[1,2]oxazepino[5,4,3- de]phthalazin-7(6H)-one |
| 188 | 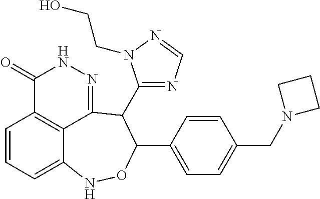 [SMILES: O=c1[nH]nc2c3c(cccc13)NOC(c1ccc(CN3CCC3)cc1)C2c1ncnn1CCO] | 3-(4-(azetidin-1-ylmethyl)phenyl)-4-(1-(2- hydroxyethyl)-1H-1,2,4-triazol-5-yl)-3,4-dihydro- 1H-[1,2]oxazepino[5,4,3-de]phthalazin-7(6H)-one |
| 189 | 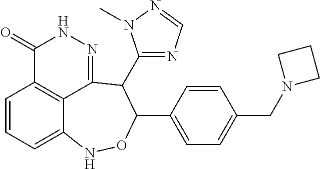 [SMILES: Cn1ncnc1C1c2n[nH]c(=O)c3cccc(c23)NOC1c1ccc(CN2CCC2)cc1] | 3-(4-(azetidin-1-ylmethyl)phenyl)-4-(1-methyl-1H- 1,2,4-triazol-5-yl)-3,4-dihydro-1H- [1,2]oxazepino[5,4,3-de]phthalazin-7(6H)-one |
| 190 | 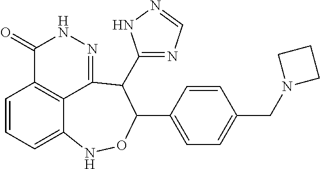 [SMILES: O=c1[nH]nc2c3c(cccc13)NOC(c1ccc(CN3CCC3)cc1)C2c1ncn[nH]1] | 3-(4-(azetidin-1-ylmethyl)phenyl)-4-(1H-1,2,4- triazol-5-yl)-3,4-dihydro-1H-[1,2]oxazepino[5,4,3- de]phthalazin-7(6H)-one |
| 191 | 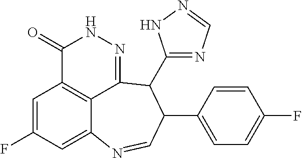 [SMILES: O=c1[nH]nc2c3c(cc(F)cc13)N=CC(c1ccc(F)cc1)C2c1ncn[nH]1] | 5-fluoro-9-(4-fluorophenyl)-10-(1H-1,2,4-triazol-5- yl)-9,10-dihydroazepino[4,3,2-de]phthalazin-3(2H)- one |
| 192 | 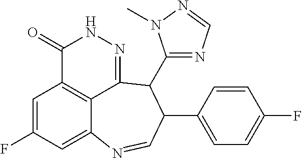 [SMILES: Cn1ncnc1C1c2n[nH]c(=O)c3cc(F)cc(c23)N=CC1c1ccc(F)cc1] | 5-fluoro-9-(4-fluorophenyl)-10-(1-methyl-1H-1,2,4- triazol-5-yl)-9,10-dihydroazepino[4,3,2- de]phthalazin-3(2H)-one |
| 193 | 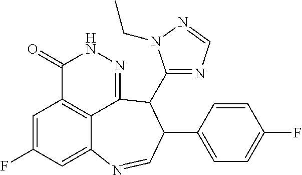 [SMILES: CCn1ncnc1C1c2n[nH]c(=O)c3cc(F)cc(c23)N=CC1c1ccc(F)cc1] | 10-(1-ethyl-1H-1,2,4-triazol-5-yl)-5-fluoro-9-(4- fluorophenyl)-9,10-dihydroazepino[4,3,2- de]phthalazin-3(2H)-one |
| 194 | 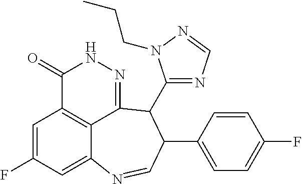 [SMILES: CCCn1ncnc1C1c2n[nH]c(=O)c3cc(F)cc(c23)N=CC1c1ccc(F)cc1] | 5-fluoro-9-(4-fluorophenyl)-10-(1-propyl-1H-1,2,4- triazol-5-yl)-9,10-dihydroazepino[4,3,2- de]phthalazin-3(2H)-one |
| 195 | 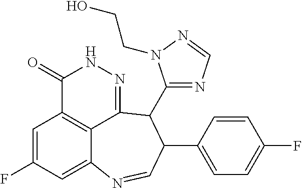 [SMILES: O=c1[nH]nc2c3c(cc(F)cc13)N=CC(c1ccc(F)cc1)C2c1ncnn1CCO] | 5-fluoro-9-(4-fluorophenyl)-10-(1-(2- hydroxyethyl)-1H-1,2,4-triazol-5-yl)-9,10- dihydroazepino[4,3,2-de]phthalazin-3(2H)-one |
| 196 | 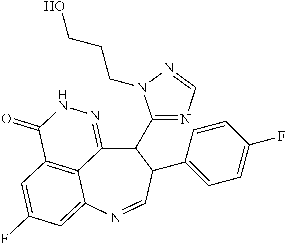 [SMILES: O=c1[nH]nc2c3c(cc(F)cc13)N=CC(c1ccc(F)cc1)C2c1ncnn1CCCO] | 5-fluoro-9-(4-fluorophenyl)-10-(1-(3- hydroxypropyl)-1H-1,2,4-triazol-5-yl)-9,10- dihydroazepino[4,3,2-de]phthalazin-3(2H)-one |
| 197 | 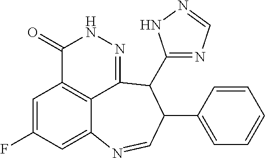 [SMILES: O=c1[nH]nc2c3c(cc(F)cc13)N=CC(c1ccccc1)C2c1ncn[nH]1] | 5-fluoro-9-phenyl-10-(1H-1,2,4-triazol-5-yl)-9,10- dihydroazepino[4,3,2-de]phthalazin-3(2H)-one |
| 198 | 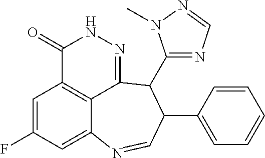 [SMILES: Cn1ncnc1C1c2n[nH]c(=O)c3cc(F)cc(c23)N=CC1c1ccccc1] | 5-fluoro-10-(1-methyl-1H-1,2,4-triazol-5-yl)-9- phenyl-9,10-dihydroazepino[4,3,2-de]phthalazin- 3(2H)-one |
| 199 | 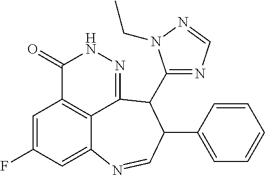 [SMILES: CCn1ncnc1C1c2n[nH]c(=O)c3cc(F)cc(c23)N=CC1c1ccccc1] | 10-(1-ethyl-1H-1,2,4-triazol-5-yl)-5-fluoro-9- phenyl-9,10-dihydroazepino[4,3,2-de]phthalazin- 3(2H)-one |
| 200 | 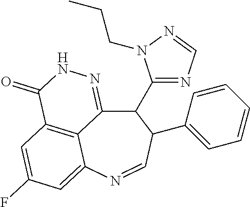 [SMILES: CCCn1ncnc1C1c2n[nH]c(=O)c3cc(F)cc(c23)N=CC1c1ccccc1] | 5-fluoro-9-phenyl-10-(1-propyl-1H-1,2,4-triazol-5- yl)-9,10-dihydroazepino[4,3,2-de]phthalazin-3(2H)- one |
| 201 | 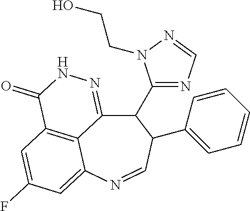 [SMILES: O=c1[nH]nc2c3c(cc(F)cc13)N=CC(c1ccccc1)C2c1ncnn1CCO] | 5-fluoro-10-(1-(2-hydroxyethyl)-1H-1,2,4-triazol-5- yl)-9-phenyl-9,10-dihydroazepino[4,3,2- de]phthalazin-3(2H)-one |
| 202 | 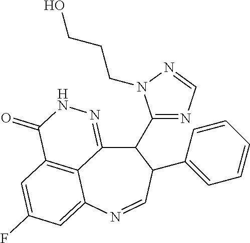 [SMILES: O=c1[nH]nc2c3c(cc(F)cc13)N=CC(c1ccccc1)C2c1ncnn1CCCO] | 5-fluoro-10-(1-(3-hydroxypropyl)-1H-1,2,4-triazol- 5-yl)-9-phenyl-9,10-dihydroazepino[4,3,2- de]phthalazin-3(2H)-one |
| 203 | 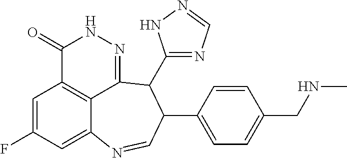 [SMILES: CNCc1ccc(C2C=Nc3cc(F)cc4c(=O)[nH]nc(c34)C2c2ncn[nH]2)cc1] | 5-fluoro-9-(4-((methylamino)methyl)phenyl)-10- (1H-1,2,4-triazol-5-yl)-9,10-dihydroazepino[4,3,2- de]phthalazin-3(2H)-one |
| 204 | 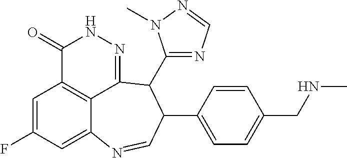 [SMILES: CNCc1ccc(C2C=Nc3cc(F)cc4c(=O)[nH]nc(c34)C2c2ncnn2C)cc1] | 5-fluoro-10-(1-methyl-1H-1,2,4-triazol-5-yl)-9-(4- ((methylamino)methyl)phenyl)-9,10- dihydroazepino[4,3,2-de]phthalazin-3(2H)-one |
| 205 | 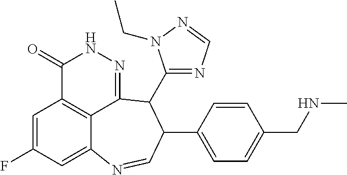 [SMILES: CCn1ncnc1C1c2n[nH]c(=O)c3cc(F)cc(c23)N=CC1c1ccc(CNC)cc1] | 10-(1-ethyl-1H-1,2,4-triazol-5-yl)-5-fluoro-9-(4- ((methylamino)methyl)phenyl)-9,10- dihydroazepino[4,3,2-de]phthalazin-3(2H)-one |
| 206 | 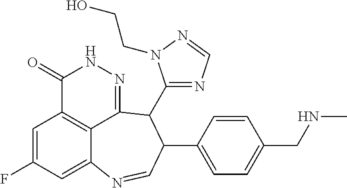 [SMILES: CNCc1ccc(C2C=Nc3cc(F)cc4c(=O)[nH]nc(c34)C2c2ncnn2CCO)cc1] | 5-fluoro-10-(1-(2-hydroxyethyl)-1H-1,2,4-triazol-5- yl)-9-(4-((methylamino)methyl)phenyl)-9,10- dihydroazepino[4,3,2-de]phthalazin-3(2H)-one |
| 207 | 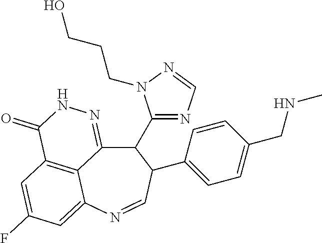 [SMILES: CNCc1ccc(C2C=Nc3cc(F)cc4c(=O)[nH]nc(c34)C2c2ncnn2CCCO)cc1] | 5-fluoro-10-(1-(3-hydroxypropyl)-1H-1,2,4-triazol- 5-yl)-9-(4-((methylamino)methyl)phenyl)-9,10- dihydroazepino[4,3,2-de]phthalazin-3(2H)-one |
| 208 | 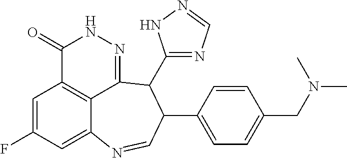 [SMILES: CN(C)Cc1ccc(C2C=Nc3cc(F)cc4c(=O)[nH]nc(c34)C2c2ncn[nH]2)cc1] | 9-(4-((dimethylamino)methyl)phenyl)-5-fluoro-10- (1H-1,2,4-triazol-5-yl)-9,10-dihydroazepino[4,3,2- de]phthalazin-3(2H)-one |
| 209 | 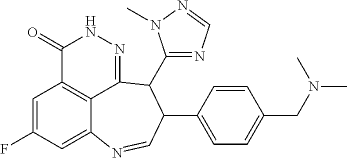 [SMILES: CN(C)Cc1ccc(C2C=Nc3cc(F)cc4c(=O)[nH]nc(c34)C2c2ncnn2C)cc1] | 9-(4-((dimethylamino)methyl)phenyl)-5-fluoro-10- (1-methyl-1H-1,2,4-triazol-5-yl)-9,10- dihydroazepino[4,3,2-de]phthalazin-3(2H)-one |
| 210 | 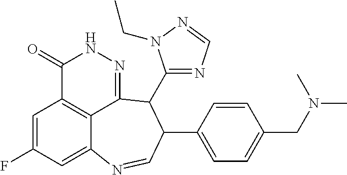 [SMILES: CCn1ncnc1C1c2n[nH]c(=O)c3cc(F)cc(c23)N=CC1c1ccc(CN(C)C)cc1] | 9-(4-((dimethylamino)methyl)phenyl)-10-(1-ethyl- 1H-1,2,4-triazol-5-yl)-5-fluoro-9,10- dihydroazepino[4,3,2-de]phthalazin-3(2H)-one |
| 211 | 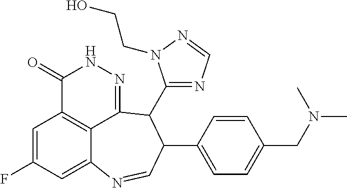 [SMILES: CN(C)Cc1ccc(C2C=Nc3cc(F)cc4c(=O)[nH]nc(c34)C2c2ncnn2CCO)cc1] | 9-(4-((dimethylamino)methyl)phenyl)-5-fluoro-10- (1-(2-hydroxyethyl)-1H-1,2,4-triazol-5-yl)-9,10- dihydroazepino[4,3,2-de]phthalazin-3(2H)-one |
| 212 | 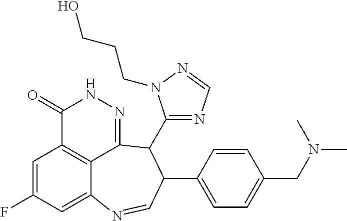 [SMILES: CN(C)Cc1ccc(C2C=Nc3cc(F)cc4c(=O)[nH]nc(c34)C2c2ncnn2CCCO)cc1] | 9-(4-((dimethylamino)methyl)phenyl)-5-fluoro-10- (1-(3-hydroxypropyl)-1H-1,2,4-triazol-5-yl)-9,10- dihydroazepino[4,3,2-de]phthalazin-3(2H)-one |
| 213 | 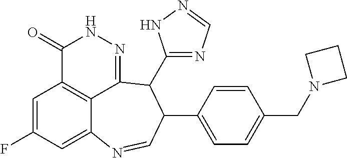 [SMILES: O=c1[nH]nc2c3c(cc(F)cc13)N=CC(c1ccc(CN3CCC3)cc1)C2c1ncn[nH]1] | 9-(4-(azetidin-1-ylmethyl)phenyl)-5-fluoro-10- (1H-1,2,4-triazol-5-yl)-9,10-dihydroazepino[4,3,2- de]phthalazin-3(2H)-one |
| 214 | 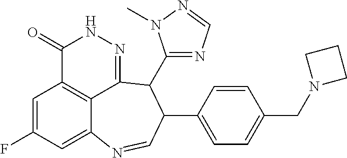 [SMILES: Cn1ncnc1C1c2n[nH]c(=O)c3cc(F)cc(c23)N=CC1c1ccc(CN2CCC2)cc1] | 9-(4-(azetidin-1-ylmethyl)phenyl)-5-fluoro-10-(1- methyl-1H-1,2,4-triazol-5-yl)-9,10- dihydroazepino[4,3,2-de]phthalazin-3(2H)-one |
| 215 | 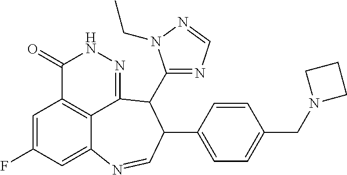 [SMILES: CCn1ncnc1C1c2n[nH]c(=O)c3cc(F)cc(c23)N=CC1c1ccc(CN2CCC2)cc1] | 9-(4-(azetidin-1-ylmethyl)phenyl)-10-(1-ethyl-1H- 1,2,4-triazol-5-yl)-5-fluoro-9,10- dihydroazepino[4,3,2-de]phthalazin-3(2H)-one |
| 216 | 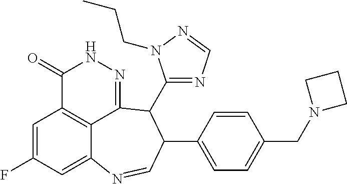 [SMILES: CCCn1ncnc1C1c2n[nH]c(=O)c3cc(F)cc(c23)N=CC1c1ccc(CN2CCC2)cc1] | 9-(4-(azetidin-1-ylmethyl)phenyl)-5-fluoro-10-(1- propyl-1H-1,2,4-triazol-5-yl)-9,10- dihydroazepino[4,3,2-de]phthalazin-3(2H)-one |
| 217 | 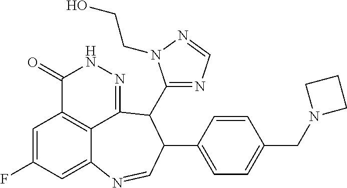 [SMILES: O=c1[nH]nc2c3c(cc(F)cc13)N=CC(c1ccc(CN3CCC3)cc1)C2c1ncnn1CCO] | 9-(4-(azetidin-1-ylmethyl)phenyl)-5-fluoro-10-(1- (2-hydroxyethyl)-1H-1,2,4-triazol-5-yl)-9,10- dihydroazepino[4,3,2-de]phthalazin-3(2H)-one |
| 218 | 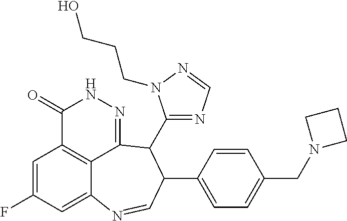 [SMILES: O=c1[nH]nc2c3c(cc(F)cc13)N=CC(c1ccc(CN3CCC3)cc1)C2c1ncnn1CCCO] | 9-(4-(azetidin-1-ylmethyl)phenyl)-5-fluoro-10-(1- (3-hydroxypropyl)-1H-1,2,4-triazol-5-yl)-9,10- dihydroazepino[4,3,2-de]phthalazin-3(2H)-one |
| 219 | 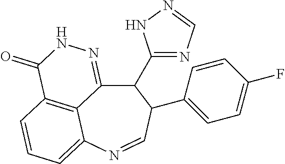 [SMILES: O=c1[nH]nc2c3c(cccc13)N=CC(c1ccc(F)cc1)C2c1ncn[nH]1] | 9-(4-fluorophenyl)-10-(1H-1,2,4-triazol-5-yl)-9,10- dihydroazepino[4,3,2-de]phthalazin-3(2H)-one |
| 220 | 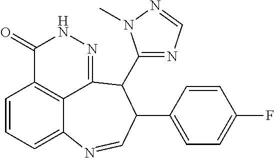 [SMILES: Cn1ncnc1C1c2n[nH]c(=O)c3cccc(c23)N=CC1c1ccc(F)cc1] | 9-(4-fluorophenyl)-10-(1-methyl-1H-1,2,4-triazol-5- yl)-9,10-dihydroazepino[4,3,2-de]phthalazin-3(2H)- one |
| 221 | 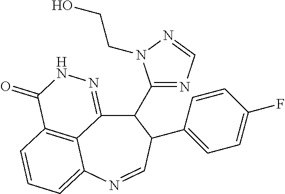 [SMILES: O=c1[nH]nc2c3c(cccc13)N=CC(c1ccc(F)cc1)C2c1ncnn1CCO] | 9-(4-fluorophenyl)-10-(1-(2-hydroxyethyl)-1H- 1,2,4-triazol-5-yl)-9,10-dihydroazepino[4,3,2- de]phthalazin-3(2H)-one |
| 222 | 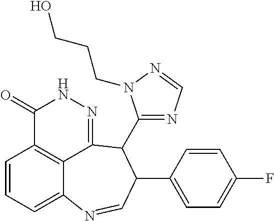 [SMILES: O=c1[nH]nc2c3c(cccc13)N=CC(c1ccc(F)cc1)C2c1ncnn1CCCO] | 9-(4-fluorophenyl)-10-(1-(3-hydroxypropyl)-1H- 1,2,4-triazol-5-yl)-9,10-dihydroazepino[4,3,2- de]phthalazin-3(2H)-one |
| 223 | 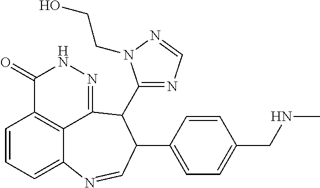 [SMILES: CNCc1ccc(C2C=Nc3cccc4c(=O)[nH]nc(c34)C2c2ncnn2CCO)cc1] | 10-(1-(2-hydroxyethyl)-1H-1,2,4-triazol-5-yl)-9-(4- ((methylamino)methyl)phenyl)-9,10- dihydroazepino[4,3,2-de]phthalazin-3(2H)-one |
| 224 | 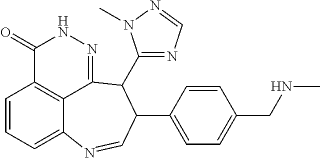 [SMILES: CNCc1ccc(C2C=Nc3cccc4c(=O)[nH]nc(c34)C2c2ncnn2C)cc1] | 10-(1-methyl-1H-1,2,4-triazol-5-yl)-9-(4- ((methylamino)methyl)phenyl)-9,10- dihydroazepino[4,3,2-de]phthalazin-3(2H)-one |
| 225 | 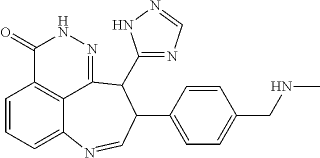 [SMILES: CNCc1ccc(C2C=Nc3cccc4c(=O)[nH]nc(c34)C2c2ncn[nH]2)cc1] | 9-(4-((methylamino)methyl)phenyl)-10-(1H-1,2,4- triazol-5-yl)-9,10-dihydroazepino[4,3,2- de]phthalazin-3(2H)-one |
| 226 | 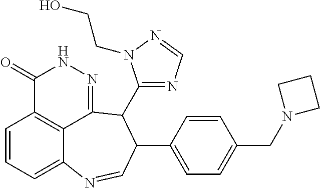 [SMILES: O=c1[nH]nc2c3c(cccc13)N=CC(c1ccc(CN3CCC3)cc1)C2c1ncnn1CCO] | 9-(4-(azetidin-1-ylmethyl)phenyl)-10-(1-(2- hydroxyethyl)-1H-1,2,4-triazol-5-yl)-9,10- dihydroazepino[4,3,2-de]phthalazin-3(2H)-one |
| 227 | 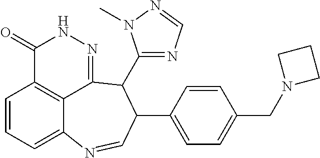 [SMILES: Cn1ncnc1C1c2n[nH]c(=O)c3cccc(c23)N=CC1c1ccc(CN2CCC2)cc1] | 9-(4-(azetidin-1-ylmethyl)phenyl)-10-(1-methyl- 1H-1,2,4-triazol-5-yl)-9,10-dihydroazepino[4,3,2- de]phthalazin-3(2H)-one |
| 228 | 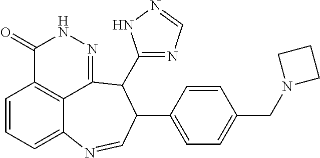 [SMILES: O=c1[nH]nc2c3c(cccc13)N=CC(c1ccc(CN3CCC3)cc1)C2c1ncn[nH]1] | 9-(4-(azetidin-1-ylmethyl)phenyl)-10-(1H-1,2,4- triazol-5-yl)-9,10-dihydroazepino[4,3,2- de]phthalazin-3(2H)-one |

In another embodiment, disclosed herein is a pharmaceutical composition comprising 1) a compound, optionally as a pharmaceutically acceptable salt thereof, of Formula (M), (I), (IA), (IA-1), (IA-1a), (IA-1b), (IB), (IB-1), (IB-1a), (IB-1b), (IC), (IC-1), (IC-1a), (IC-1b), (ID), (ID-1), (ID-1a), (ID-1b), (IE), (IE-1), (IE-1a), (IE-1b), (II), (IIF-1), (IIF-1a), or (IIF-1b) as defined in the Summary of the Invention or in any of the above embodiments and 2) one or more pharmaceutically acceptable carrier(s), excipient(s), binder(s) or diluent(s) thereof.

In one aspect is a method of inhibiting poly(ADP-ribose)polymerase (PARP) in a subject in need of PARP inhibition comprising administering to the subject a therapeutically effective amount of a compound of Formula (M), (I), (IA), (IA-1), (IA-1a), (IA-1b), (IB), (IB-1), (IB-1a), (IB-1b), (IC), (IC-1), (IC-1a), (IC-1b), (ID), (ID-1), (ID-1a), (ID-1b), (IE), (IE-1), (IE-1a), (IE-1b), (II), (IIF-1), (IIF-1a), or (IIF-1b) as defined in the Summary of the Invention or in any of the above embodiments.

In another embodiment, disclosed herein is a method of treating a disease ameliorated by the inhibition of PARP comprising administering to a subject in need of treatment a therapeutically effective amount of a compound of Formula (M), (I), (IA), (IA-1), (IA-1a), (IA-1b), (IB), (IB-1), (IB-1a), (IB-1b), (IC), (IC-1), (IC-1a), (IC-1b), (ID), (ID-1), (ID-1a), (ID-1b), (IE), (IE-1), (IE-1a), (IE-1b), (II), (IIF-1), (IIF-1a), or (IIF-1b) as defined in the Summary of the Invention or in any of the above embodiments.

In another embodiment, disclosed herein is a method of treating a disease which comprises administering to a subject in need of treatment a therapeutically effective amount of a compound of Formula (M), (I), (IA), (IA-1), (IA-1a), (IA-1b), (IB), (IB-1), (IB-1a), (IB-1b), (IC), (IC-1), (IC-1a), (IC-1b), (ID), (ID-1), (ID-1a), (ID-1b), (IE), (IE-1), (IE-1a), (IE-1b), (II), (IIF-1), (IIF-1a), or (IIF-1b) as defined in the Summary of the Invention or in any of the above embodiments where the disease is cancer. In another embodiment the cancer is ameliorated by the inhibition of PARP. In yet another embodiment the cancer is deficient in Homologous Recombination (HR) dependent DNA double strand break (DSB) repair pathway, comprising administering to a subject in need of treatment a therapeutically effective amount of a compound of Formula (M), (I), (IA), (IA-1), (IA-1a), (IA-1b), (IB), (IB-1), (IB-1a), (IB-1b), (IC), (IC-1), (IC-1a), (IC-1b), (ID), (ID-1), (ID-1a), (ID-1b), (IE), (IE-1), (IE-1a), (IE-1b), (II), (IIF-1), (IIF-1a), or (IIF-1b) as defined in the Summary of the Invention or in any of the above embodiments where the cancer comprises one or more cancer cells having a reduced or abrogated ability to repair DNA DSB by HR relative to normal cells. In another embodiment the cancer cells have a BRCA1 or BRCA2 deficient phenotype. In yet another embodiment the cancer cells are deficient in BRCA1 or BRCA2. In a further embodiment the subject is heterozygous for a mutation in a gene encoding a component of the HR dependent DNA DSB repair pathway. In yet a further embodiment the subject is heterozygous for a mutation in BRCA1 and/or BRCA2. In one embodiment the cancer is breast, ovarian, pancreatic or prostate cancer. In another embodiment the treatment further comprises administration of ionizing radiation or a therapeutic agent.

In another embodiment, disclosed herein is a method of treating a disease which comprises administering to a subject in need of treatment a therapeutically effective amount of a compound of Formula (M), (I), (IA), (IA-1), (IA-1a), (IA-1b), (IB), (IB-1), (IB-1a), (IB-1b), (IC), (IC-1), (IC-1a), (IC-1b), (ID), (ID-1), (ID-1a), (ID-1b), (IE), (IE-1), (IE-1a), (IE-1b), (II), (IIF-1), (IIF-1a), or (IIF-1b) as defined in the Summary of the Invention or in any of the above embodiments where the disease is cancer where the cancer is leukemia, colon cancer, glioblastomas, lymphomas, melanomas, carcinomas of breast, ovarian cancer, pancreatic cancer, prostate cancer, tumors of the endometrium, lung cancer, hematological cancers, bowel tumors, cervical carcinomas, acute myelogenous leukemia, or multiple myeloma. In another embodiment the cancer is breast, ovarian, pancreatic or prostate cancer.

In another embodiment the method of treating cancer comprises administering to a subject in need of treatment a therapeutically effective amount of a compound of Formula (M), (I), (IA), (IA-1), (IA-1a), (IA-1b), (IB), (IB-1), (IB-1a), (IB-1b), (IC), (IC-1), (IC-1a), (IC-1b), (ID), (ID-1), (ID-1a), (ID-1b), (IE), (IE-1), (IE-1a), (IE-1b), (II), (IIF-1), (IIF-1a), or (IIF-1b) as defined in the Summary of the Invention or in any of the above embodiments in combination with ionizing radiation, one or more therapeutic agents, or a combination thereof. In one embodiment the compound of Formula (M), (I), (IA), (IA-1), (IA-1a), (IA-1b), (IB), (IB-1), (IB-1a), (IB-1b), (IC), (IC-1), (IC-1a), (IC-1b), (ID), (ID-1), (ID-1a), (ID-1b), (IE), (IE-1), (IE-1a), (IE-1b), (H), (IIF-1), (IIF-1a), or (IIF-1b) as defined in the Summary of the Invention or in any of the above embodiments is administered simultaneously with ionizing radiation, one or more therapeutic agents, or a combination thereof. In another embodiment the therapeutic agent is alemtuzumab, arsenic trioxide, asparaginase (pegylated or non-), bevacizumab, cetuximab, platinum-based compounds such as cisplatin, cladribine, daunorubicin/doxorubicin/idarubicin, irinotecan, fludarabine, 5-fluorouracil, gemtuzumab, methotrexate, Paclitaxel™, taxol, temozolomide, thioguanine, or classes of drugs including hormones (an antiestrogen, an antiandrogen, or gonadotropin releasing hormone analogues, interferons such as alpha interferon, nitrogen mustards such as busulfan or melphalan or mechlorethamine, retinoids such as tretinoin, topoisomerase inhibitors such as irinotecan or topotecan, tyrosine kinase inhibitors such as gefinitinib or imatinib, or agents to treat signs or symptoms induced by such therapy including allopurinol, filgrastim, granisetron/ondansetron/palonosetron, or dronabinol

In another embodiment the method of treating cancer comprises administering to a subject in need of treatment a therapeutically effective amount of a compound of Formula(M), (I), (IA), (IA-1), (IA-1a), (IA-1b), (IB), (IB-1), (IB-1a), (IB-1b), (IC), (IC-1), (IC-1a), (IC-1b), (ID), (ID-1), (ID-1a), (ID-1b), (IE), (IE-1), (IE-1a), (IE-1b), (II), (IIF-1), (IIF-1a), or (IIF-1b) as defined in the Summary of the Invention or in any of the above embodiments in combination with ionizing radiation, one or more therapeutic agents, or a combination thereof where the compound is administered sequentially with ionizing radiation, one or more therapeutic agents, or a combination thereof. In another embodiment the therapeutic agent is alemtuzumab, arsenic trioxide, asparaginase (pegylated or non-), bevacizumab, cetuximab, platinum-based compounds such as cisplatin, cladribine, daunonibicin/doxorubicin/idarubicin, irinotecan, fludarabine, 5-fluorouracil, gemtuzumab, methotrexate, Paclitaxel™, taxol, temozolomide, thioguanine, or classes of drugs including hormones (an antiestrogen, an antiandrogen, or gonadotropin releasing hormone analogues, interferons such as alpha interferon, nitrogen mustards such as busulfan or melphalan or mechlorethamine, retinoids such as tretinoin, topoisomerase inhibitors such as irinotecan or topotecan, tyrosine kinase inhibitors such as gefinitinib or imatinib, or agents to treat signs or symptoms induced by such therapy including allopurinol, filgrastim, granisetron/ondansetron/palonosetron, or dronabinol.

Certain embodiments provide a method of potentiation of cytotoxic cancer therapy in a subject in recognized need of such treatment comprising administering to the subject a therapeutically acceptable amount of a compound of Formula (M), (I), (IA), (IA-1), (IA-1a), (IA-1b), (IB), (IB-1), (IB-1a), (IB-1b), (IC), (IC-1), (IC-1a), (IC-1b), (ID), (ID-1), (ID-1a), (ID-1b), (IE), (IE-1), (IE-1a), (IE-1b), (II), (IIF-1), (IIF-1a), or (IIF-1b) as defined in the Summary of the Invention or in any of the above embodiments or a therapeutically acceptable salt thereof.

In another embodiment, disclosed herein is a method of treating a disease which comprises administering to a subject in need of treatment a therapeutically effective amount of a compound of Formula (M), (I), (IA), (IA-1), (IA-1a), (IA-1b), (IB), (IB-1), (IB-1a), (IB-1b), (IC), (IC-1), (IC-1a), (IC-1b), (ID), (ID-1), (ID-1a), (ID-1b), (IE), (IE-1), (IE-1a), (IE-1b), (II), (IIF-1), (IIF-1a), or (IIF-1b) as defined in the Summary of the Invention or in any of the above embodiments where the disease is vascular disease; septic shock; ischemic injury; reperfusion injury; neurotoxicity; degenerative disease; hemorrhagic shock; inflammatory diseases; multiple sclerosis; secondary effects of diabetes; myelodysplastic syndromes; or acute treatment of cytotoxicity following cardiovascular surgery. In another embodiment the disease is ameliorated by the inhibition of PARP.

In another embodiment, disclosed herein is a method of treating a disease which comprises administering to a subject in need of treatment a therapeutically effective amount of a compound of Formula (M), (I), (IA), (IA-1), (IA-1a), (IA-1b), (IB), (IB-1), (IB-1a), (IB-1b), (IC), (IC-1), (IC-1a), (IC-1b), (ID), (ID-1), (ID-1a), (ID-1b), (IE), (IE-1a), (IE-1b), (II), (IIF-1), (IIF-1a), or (IIF-1b) as defined in the Summary of the Invention or in any of the above embodiments where the disease is ischemia reperfusion injury associated with, but not limited to, myocardial infarction, stroke, other neural trauma, and organ transplantation. In another embodiment, disclosed herein is a method of treating reperfusion including, but not limited to, reperfusion of the eye, kidney, gut, and skeletal muscle.

In another embodiment, disclosed herein is a method of treating a disease which comprises administering to a subject in need of treatment a therapeutically effective amount of a compound of Formula (M), (I), (IA), (IA-1), (IA-1a), (IA-1b), (IB), (IB-1), (IB-1a), (IB-1b), (IC), (IC-1), (IC-1a), (IC-1b), (ID), (ID-1), (ID-1a), (ID-1b), (IE), (IE-1), (IE-1a), (IE-1b), (II), (IIF-1), (IIF-1a), or (IIF-1b) as defined in the Summary of the Invention or in any of the above embodiments where the disease is an inflammatory diseases including, but not limited to, arthritis, gout, inflammatory bowel disease, CNS inflammation, multiple sclerosis, allergic encephalitis, sepsis, septic shock, hemorrhagic shock, pulmonary fibrosis, and uveitis.

In another embodiment, disclosed herein is a method of treating a disease which comprises administering to a subject in need of treatment a therapeutically effective amount of a compound of Formula (M), (I), (IA), (IA-1), (IA-1a), (IA-1b), (IB), (IB-1), (IB-1a), (IB-1b), (IC), (IC-1), (IC-1a), (IC-1b), (ID), (ID-1), (ID-1a), (ID-1b), (IE), (IE-1), (IE-1a), (IE-1b), (II), (IIF-1), (IIF-1a), or (IIF-1b) as defined in the Summary of the Invention or in any of the above embodiments where the disease is an immunological disease or disorder such as rheumatoid arthritis and septic shock.

In another embodiment, disclosed herein is a method of treating a degenerative disease which comprises administering to a subject in need of treatment a therapeutically effective amount of a compound of Formula (M), (I), (IA), (IA-1), (IA-1a), (IA-1b), (IB), (IB-1), (IB-1a), (IB-1b), (IC), (IC-1), (IC-1a), (IC-1b), (ID), (ID-1), (ID-1a), (ID-1b), (IE), (IE-1), (IE-1a), (IE-1b), (II), (IIF-1), (IIF-1a), or (IIF-1b) as defined in the Summary of the Invention or in any of the above embodiments where the degenerative disease is Parkinson's disease.

In another embodiment, disclosed herein is a method of treating a disease which comprises administering to a subject in need of treatment a therapeutically effective amount of a compound of Formula (M), (I), (IA), (IA-1), (IA-1a), (IA-1b), (IB), (IB-1), (IB-1a), (IB-1b), (IC), (IC-1), (IC-1a), (IC-1b), (ID), (ID-1), (ID-1a), (ID-1b), (IE), (IE-1), (IE-1a), (IE-1b), (II), (IIF-1), (IIF-1a), or (IIF-1b) as defined in the Summary of the Invention or in any of the above embodiments where the disease is hypoglycemia.

In another embodiment, disclosed herein is a method of treating a disease which comprises administering to a subject in need of treatment a therapeutically effective amount of a compound of Formula (M), (I), (II), (IA), (IB), (IC), (ID), or (IE) as defined in the Summary of the Invention or in any of the above embodiments where the disease is retroviral infection.

In another embodiment, disclosed herein is a method of treating a disease which comprises administering to a subject in need of treatment a therapeutically effective amount of a compound of Formula (M), (I), (IA), (IA-1), (IA-1a), (IA-1b), (IB), (IB-1), (IB-1a), (IB-1b), (IC), (IC-1), (IC-1a), (IC-1b), (ID), (ID-1), (ID-1a), (ID-1b), (IE), (IE-1), (IE-1a), (IE-1b), (II), (IIF-1), (IIF-1a), or (IIF-1b) as defined in the Summary of the Invention or in any of the above embodiments where the disease is liver toxicity following acetaminophen overdose.

In another embodiment, disclosed herein is a method of treating a disease which comprises administering to a subject in need of treatment a therapeutically effective amount of a compound of Formula (M), (I), (IA), (IA-1), (IA-1a), (IA-1b), (IB), (IB-1), (IB-1a), (IB-1b), (IC), (IC-1), (IC-1a), (IC-1b), (ID), (ID-1), (ID-1a), (ID-1b), (IE), (IE-1), (IE-1a), (IE-1b), (II), (IIF-1), (IIF-1a), or (IIF-1b) as defined in the Summary of the Invention or in any of the above embodiments where the disease is cardiac and kidney toxicities from doxorubicin and platinum based antineoplastic agents.

In another embodiment, disclosed herein is a method of treating a disease which comprises administering to a subject in need of treatment a therapeutically effective amount of a compound of Formula (M), (I), (IA), (IA-1), (IA-1a), (IA-1b), (IB), (IB-1), (IB-1a), (IB-1b), (IC), (IC-1), (IC-1a), (IC-1b), (ID), (ID-1), (ID-1a), (ID-1b), (IE), (IE-1), (IE-1a), (IE-1b), (II), (IIF-1), (IIF-1a), or (IIF-1b) as defined in the Summary of the Invention or in any of the above embodiments where the disease is skin damage secondary to sulfur mustards.

In one aspect is the use of a compound of Formula (M), (I), (II), (IA), (IB), (IC), (ID), or (IE) as defined in the Summary of the Invention or in any of the above embodiments in the formulation of a medicament for the treatment of a disease or condition. In another embodiment, the disease is mediated by poly(ADP-ribose)polymerase.

In another aspect is an article of manufacture, comprising packaging material, a compound of Formula (M), (I), (IA), (IA-1), (IA-1a), (IA-1b), (IB), (IB-1), (IB-1a), (IB-1b), (IC), (IC-1), (IC-1a), (IC-1b), (ID), (ID-1), (ID-1a), (ID-1b), (IE), (IE-1), (IE-1a), (IE-1b), (II), (IIF-1), (IIF-1a), or (IIF-1b) as defined in the Summary of the Invention or in any of the above embodiments, and a label, where the compound is effective for modulating the activity of the enzyme poly(ADP-ribose)polymerase, or for treatment, prevention or amelioration of one or more symptoms of a poly(ADP-ribose)polymerase-dependent or poly(ADP-ribose)polymerase-mediated disease or condition, where the compound is packaged within the packaging material, and where the label indicates that the compound, or pharmaceutically acceptable salt, pharmaceutically acceptable N-oxide, pharmaceutically active metabolite, pharmaceutically acceptable prodrug, or pharmaceutically acceptable solvate thereof, or a pharmaceutical composition comprising such a compound is used for modulating the activity of poly(ADP-ribose)polymerase, or for treatment, prevention or amelioration of one or more symptoms of a poly(ADP-ribose)polymerase-dependent or poly(ADP-ribose)polymerase-mediated disease or condition.

Certain embodiments provide a use of a compound of Formula (M), (I), (IA), (IA-1), (IA-1a), (IA-1b), (IB), (IB-1), (IB-1a), (IB-1b), (IC), (IC-1), (IC-1a), (IC-1b), (ID), (ID-1), (ID-1a), (ID-1b), (IE), (IE-1), (IE-1a), (IE-1b), (II), (IIF-1), (IIF-1a), or (IIF-1b) as defined in the Summary of the Invention or in any of the above embodiments or a therapeutically acceptable salt thereof, to prepare a medicament for inhibiting the PARP enzyme in a subject in recognized need of such treatment.

Certain embodiments provide a use of a compound of Formula (M), (I), (IA), (IA-1), (IA-1a), (IA-1b), (IB), (IB-1), (IB-1a), (IB-1b), (IC), (IC-1), (IC-1a), (IC-1b), (ID), (ID-1), (ID-1a), (ID-1b), (IE), (IE-1), (IE-1a), (IE-1b), (II), (IIF-1), (IIF-1a), or (IIF-1b) as defined in the Summary of the Invention or in any of the above embodiments or a therapeutically acceptable salt thereof, to prepare a medicament for inhibiting tumor growth in a subject in recognized need of such treatment.

Certain embodiments provide a use of a compound of Formula (M), (I), (IA), (IA-1), (IA-1a), (IA-1b), (IB), (IB-1), (IB-1a), (IB-1b), (IC), (IC-1), (IC-1a), (IC-1b), (ID), (ID-1), (ID-1a), (ID-1b), (IE), (IE-1), (IE-1a), (IE-1b), (II), (IIF-1), (IIF-1a), or (IIF-1b) as defined in the Summary of the Invention or in any of the above embodiments or a therapeutically acceptable salt thereof, to prepare a medicament for treating cancer in a subject in recognized need of such treatment. In another embodiment the cancer is ameliorated by the inhibition of PARP.

Yet another embodiment provides a use of a compound of Formula (M), (I), (IA), (IA-1), (IA-1a), (IA-1b), (IB), (IB-1), (IB-1a), (IB-1b), (IC), (IC-1), (IC-1a), (IC-1b), (ID), (ID-1), (ID-1a), (ID-1b), (IE), (IE-1), (IE-1a), (IE-1b), (II), (IIF-1), (IIF-1a), or (IIF-1b) as defined in the Summary of the Invention or in any of the above embodiments or a therapeutically acceptable salt thereof, to prepare a medicament for treating a cancer deficient in Homologous Recombination (HR) dependent DNA double strand break (DSB) repair pathway, comprising administering to a subject in need of treatment a therapeutically effective amount of the compound where the cancer comprises one or more cancer cells having a reduced or abrogated ability to repair DNA DSB by HR relative to normal cells. In another embodiment the cancer cells have a BRCA1 or BRCA2 deficient phenotype. In yet another embodiment the cancer cells are deficient in BRCA1 or BRCA2. In a further embodiment the subject is heterozygous for a mutation in a gene encoding a component of the HR dependent DNA DSB repair pathway. In yet a further embodiment the subject is heterozygous for a mutation in BRCA1 and/or BRCA2. In one embodiment the cancer is breast, ovarian, pancreatic or prostate cancer.

Another embodiment provides a use of a compound of Formula (M), (I), (IA), (IA-1), (IA-1a), (IA-1b), (IB), (IB-1), (IB-1a), (IB-1b), (IC), (IC-1), (IC-1a), (IC-1b), (ID), (ID-1), (ID-1a), (ID-1b), (IE), (IE-1), (IE-1a), (IE-1b), (II), (IIF-1), (IIF-1a), or (IIF-1b) as defined in the Summary of the Invention or in any of the above embodiments or a therapeutically acceptable salt thereof, to prepare a medicament for treating a disease which comprises administering to a subject in need of treatment a therapeutically effective amount of the compound where the disease is cancer and the cancer is leukemia, colon cancer, glioblastomas, lymphomas, melanomas, carcinomas of breast, ovarian cancer, pancreatic cancer, prostate cancer, or cervical carcinomas. In another embodiment the cancer is breast, ovarian, pancreatic or prostate cancer.

Another embodiment provides a use of a compound of Formula (M), (I), (IA), (IA-1), (IA-1a), (IA-1b), (IB), (IB-1), (IB-1a), (IB-1b), (IC), (IC-1), (IC-1a), (IC-1b), (ID), (ID-1), (ID-1a), (ID-1b), (IE), (IE-1), (IE-1a), (IE-1b), (II), (IIF-1), (IIF-1a), or (IIF-1b) as defined in the Summary of the Invention or in any of the above embodiments or a therapeutically acceptable salt thereof, to prepare a medicament for treating a disease which comprises administering to a subject in need of treatment a therapeutically effective amount of the compound where the disease is vascular disease; septic shock; ischemic injury; reperfusion injury; neurotoxicity; hemorrhagic shock; inflammatory diseases; multiple sclerosis; secondary effects of diabetes; or acute treatment of cytotoxicity following cardiovascular surgery. In another embodiment the disease is ameliorated by the inhibition of PARP.

Another embodiment provides a use of a compound of Formula (M), (I), (IA), (IA-1), (IA-1a), (IA-1b), (IB), (IB-1), (IB-1a), (TB-1b), (IC), (IC-1), (IC-1a), (IC-1b), (ID), (ID-1), (ID-1a), (ID-1b), (IE), (IE-1), (IE-1a), (IE-1b), (II), (IIF-1), (IIF-1a), or (IIF-1b) as defined in the Summary of the

Invention or in any of the above embodiments or a therapeutically acceptable salt thereof, to prepare a medicament for treating a disease which comprises administering to a subject in need of treatment a therapeutically effective amount of the compound where the disease is ischemia reperfusion injury associated with, but not limited to, myocardial infarction, stroke, other neural trauma, and organ transplantation. In another embodiment, disclosed herein is a method of treating reperfusion including, but not limited to, reperfusion of the eye, kidney, gut, and skeletal muscle.

Another embodiment provides a use of a compound of Formula (M), (I), (IA), (IA-1), (IA-1a), (IA-1b), (IB), (IB-1), (IB-1a), (IB-1b), (IC), (IC-1), (IC-1a), (IC-1b), (ID), (ID-1), (ID-1a), (ID-1b), (IE), (IE-1), (IE-1a), (IE-1b), (II), (IIF-1), (IIF-1a), or (IIF-1b) as defined in the Summary of the Invention or in any of the above embodiments or a therapeutically acceptable salt thereof, to prepare a medicament for treating a disease which comprises administering to a subject in need of treatment a therapeutically effective amount of the compound where the disease is an inflammatory diseases including, but not limited to, arthritis, gout, inflammatory bowel disease, CNS inflammation, multiple sclerosis, allergic encephalitis, sepsis, septic shock, hemorrhagic shock, pulmonary fibrosis, and uveitis.

Another embodiment provides a use of a compound of Formula (M), (I), (IA), (IA-1), (IA-1a), (IA-1b), (IB), (IB-1), (IB-1a), (IB-1b), (IC), (IC-1), (IC-1a), (IC-1b), (ID), (ID-1), (ID-1a), (ID-1b), (IE), (IE-1), (IE-1a), (IE-1b), (II), (IIF-1), (IIF-1a), or (IIF-1b) as defined in the Summary of the Invention or in any of the above embodiments or a therapeutically acceptable salt thereof, to prepare a medicament for treating a disease which comprises administering to a subject in need of treatment a therapeutically effective amount of the compound where the disease is an immunological disease or disorder such as rheumatoid arthritis and septic shock.

Another embodiment provides a use of a compound of Formula (M), (I), (IA), (IA-1), (IA-1a), (IA-1b), (IB), (IB-1), (IB-1a), (IB-1b), (IC), (IC-1), (IC-1a), (IC-1b), (ID), (ID-1), (ID-1a), (ID-1b), (IE), (IE-1), (IE-1a), (IE-1b), (II), (IIF-1), (IIF-1a), or (IIF-1b) as defined in the Summary of the Invention or in any of the above embodiments or a therapeutically acceptable salt thereof, to prepare a medicament for treating a disease which comprises administering to a subject in need of treatment a therapeutically effective amount of the compound where the disease is Parkinson's disease.

Another embodiment provides a use of a compound of Formula (M), (I), (IA), (IA-1), (IA-1a), (IA-1b), (IB), (IB-1), (IB-1a), (IB-1b), (IC), (IC-1), (IC-1a), (IC-1b), (ID), (ID-1), (ID-1a), (ID-1b), (IE), (IE-1), (IE-1a), (IE-1b), (II), (IIF-1), (IIF-1a), or (IIF-1b) as defined in the Summary of the Invention or in any of the above embodiments or a therapeutically acceptable salt thereof, to prepare a medicament for treating a disease which comprises administering to a subject in need of treatment a therapeutically effective amount of a compound of Formula (M), (I), (IA), (IA-1), (IA-1a), (IA-1b), (IB), (IB-1), (IB-1a), (IB-1b), (IC), (IC-1), (IC-1a), (IC-1b), (ID), (ID-1), (ID-1a), (ID-1b), (IE), (IE-1), (IE-1a), (IE-1b), (II), (IIF-1), (IIF-1a), or (IIF-1b) as defined in the Summary of the Invention or in any of the above embodiments where the disease is hypoglycemia.

Another embodiment provides a use of a compound of Formula (M), (I), (IA), (IA-1), (IA-1a), (IA-1b), (IB), (IB-1), (IB-1a), (IB-1b), (IC), (IC-1), (IC-1a), (IC-1b), (ID), (ID-1), (ID-1a), (ID-1b), (IE), (IE-1), (IE-1a), (IE-1b), (II), (IIF-1), (IIF-1a), or (IIF-1b) as defined in the Summary of the Invention or in any of the above embodiments or a therapeutically acceptable salt thereof, to prepare a medicament for treating a disease which comprises administering to a subject in need of treatment a therapeutically effective amount of the compound where the disease is retroviral infection.

Another embodiment provides a use of a compound of Formula (M), (I), (IA), (IA-1), (IA-1a), (IA-1b), (IB), (IB-1), (IB-1a), (IB-1b), (IC), (IC-1), (IC-1a), (IC-1b), (ID), (ID-1), (ID-1a), (ID-1b), (IE), (IE-1), (IE-1a), (IE-1b), (II), (IIF-1), (IIF-1a), or (IIF-1b) as defined in the Summary of the Invention or in any of the above embodiments or a therapeutically acceptable salt thereof, to prepare a medicament for treating a disease which comprises administering to a subject in need of treatment a therapeutically effective amount of the compound where the disease is liver toxicity following acetaminophen overdose.

Another embodiment provides a use of a compound of Formula (M), (I), (IA), (IA-1), (IA-1a), (IA-1b), (IB), (IB-1), (IB-1a), (IB-1b), (IC), (IC-1), (IC-1a), (IC-1b), (ID), (ID-1), (ID-1a), (ID-1b), (IE), (IE-1), (IE-1a), (IE-1b), (II), (IIF-1), (IIF-1a), or (IIF-1b) as defined in the Summary of the Invention or in any of the above embodiments or a therapeutically acceptable salt thereof, to prepare a medicament for treating a disease which comprises administering to a subject in need of treatment a therapeutically effective amount of the compound where the disease is cardiac and kidney toxicities from doxorubicin and platinum based antineoplastic agents.

Another embodiment provides a use of a compound of Formula (M), (I), (IA), (IA-1), (IA-1a), (IA-1b), (IB), (IB-1), (IB-1a), (IB-1b), (IC), (IC-1), (IC-1a), (IC-1b), (ID), (ID-1), (ID-1a), (ID-1b), (IE), (IE-1), (IE-1a), (IE-1b), (II), (IIF-1), (IIF-1a), or (IIF-1b) as defined in the Summary of the Invention or in any of the above embodiments or a therapeutically acceptable salt thereof, to prepare a medicament for treating a disease which comprises administering to a subject in need of treatment a therapeutically effective amount of the compound where the disease is skin damage secondary to sulfur mustards.

In certain embodiments, PARP inhibitors, such as those of Formula (M), (I), (IA), (IA-1), (IA-1a), (IA-1b), (IB), (IB-1), (IB-1a), (IB-1b), (IC), (IC-1), (IC-1a), (IC-1b), (ID), (ID-1), (ID-1a), (ID-1b), (IE), (IE-1), (IE-1a), (IE-1b), (II), (IIF-1), (IIF-1a), or (IIF-1b), have utility in: (a) preventing or inhibiting poly(ADP-ribose) chain formation by, e.g., inhibiting the activity of cellular PARP (PARP-1 and/or PARP-2); (b) treating vascular disease; septic shock; ischemic injury, both cerebral and cardiovascular; reperfusion injury, both cerebral and cardiovascular; neurotoxicity, including acute and chronic treatments for stroke and Parkinson's disease; hemorrhagic shock; inflammatory diseases, such as arthritis, inflammatory bowel disease, ulcerative colitis and Crohn's disease; multiple sclerosis; secondary effects of diabetes; as well as the acute treatment of cytotoxicity following cardiovascular surgery or diseases ameliorated by the inhibition of the activity of PARP; (c) use as an adjunct in cancer therapy or for potentiating tumor cells for treatment with ionizing radiation and/or therapeutic agents.

In one embodiment, disclosed herein is a pharmaceutical composition comprising a compound, pharmaceutically acceptable salt, pharmaceutically acceptable N-oxide, pharmaceutically active metabolite, pharmaceutically acceptable prodrug, or pharmaceutically acceptable solvate of any of the compounds disclosed herein. In some embodiments, the pharmaceutical compositions further comprises a pharmaceutically acceptable diluent, excipient or binder. In certain embodiments, the pharmaceutical composition further comprises a second pharmaceutically active ingredient.

In one embodiment, the PARP mediated disease or condition in a patient, or the PARP dependent disease or condition in a patient is cancer or a non-cancerous disorder. In some embodiments, the disease or condition is iatrogenic.

In some embodiments are methods for reducing/inhibiting the activity of PARP in a subject that include administering to the subject at least once an effective amount of a compound described herein.

Certain embodiments provided herein are methods for modulating, including reducing and/or inhibiting the activity of PARP, directly or indirectly, in a subject comprising administering to the subject at least once an effective amount of at least one compound described herein.

In further embodiments are methods for treating PARP mediated conditions or diseases, comprising administering to the subject at least once an effective amount of at least one compound described herein.

Some embodiments include the use of a compound described herein in the manufacture of a medicament for treating a disease or condition in a subject in which the activity of at least one PARP protein contributes to the pathology and/or symptoms of the disease or condition.

In any of the aforementioned embodiments are further embodiments in which administration is enteral, parenteral, or both, and where:

(a) the effective amount of the compound is systemically administered to the subject;

(b) the effective amount of the compound is administered orally to the subject;

(c) the effective amount of the compound is intravenously administered to the subject;

(d) the effective amount of the compound administered by inhalation;

(e) the effective amount of the compound is administered by nasal administration;

(f) the effective amount of the compound is administered by injection to the subject;

(g) the effective amount of the compound is administered topically (dermal) to the subject;

(h) the effective amount of the compound is administered by ophthalmic administration; and/or

(i) the effective amount of the compound is administered rectally to the subject.

In any of the aforementioned embodiments are further embodiments that include single administrations of the effective amount of the compound, including further embodiments in which the compound is administered to the subject (i) once; (ii) multiple times over the span of one day; (iii) continually; or (iv) continuously.

In any of the aforementioned embodiments are further embodiments that include multiple administrations of the effective amount of the compound, including further embodiments where:

(i) the compound is administered in a single dose;

(ii) the time between multiple administrations is every 6 hours;

(iii) the compound is administered to the subject every 8 hours.

In further or alternative embodiments, the method includes a drug holiday, where the administration of the compound is temporarily suspended or the dose of the compound being administered is temporarily reduced; at the end of the drug holiday, dosing of the compound is resumed. In some embodiments, the length of the drug holiday varies from 2 days to 1 year.

In any of the aforementioned embodiments involving the treatment of proliferative disorders, including cancer, are further embodiments that include administering at least one additional therapeutic agent selected from among alemtuzumab, arsenic trioxide, asparaginase (pegylated or non-), bevacizumab, cetuximab, platinum-based compounds such as cisplatin, cladribine, daunorubicin/doxorubicin/idarubicin, irinotecan, fludarabine, 5-fluorouracil, gemtuzumab, methotrexate, paclitaxel, Taxol®, temozolomide, thioguanine, and classes of drugs including hormones (an antiestrogen, an antiandrogen, or gonadotropin releasing hormone analogues, interferons such as, for example, alpha interferon, nitrogen mustards such as, for example, busulfan, melphalan or mechlorethamine, retinoids such as, for example, tretinoin, topoisomerase inhibitors such as, for example, irinotecan or topotecan, tyrosine kinase inhibitors such as, for example, gefinitinib or imatinib, and agents to treat signs or symptoms induced by such therapy including allopurinol, filgrastim, granisetron/ondansetron/palonosetron, and dronabinol.

Other objects, features and advantages of the compounds, methods and compositions described herein will become apparent from the following description. It should be understood, however, that the description and the specific examples, while indicating specific embodiments, are given by way of illustration only, since various changes and modifications within the spirit and scope of the present description will become apparent from this detailed description.

Described herein are compounds, methods of making such compounds, pharmaceutical compositions and medicaments that include such compounds, and methods of using such compounds to treat or prevent diseases or conditions associated with PARP activity.

In specific embodiments, compounds provided herein, such as, for example, Formula (I) and (IA), are used in anti-cancer combination therapies (or as adjuncts) along with alkylating agents, such as methyl methanesulfonate (MMS), temozolomide and dacarbazine (DTIC), also with topoisomerase-1 inhibitors like Topotecan, Irinotecan, Rubitecan, Exatecan, Lurtotecan, Gimetecan, Diflomotecan (homocamptothecins); as well as 7-substituted non-silatecans; the 7-silyl camptothecins, BNP 1350; and non-camptothecin topoisomerase-I inhibitors such as indolocarbazoles also dual topoisomerase-I and II inhibitors like the benzophenazines, XR 11576/MLN 576 and benzopyridoindoles. In certain embodiments, such combinations are given, for example, as intravenous preparations or by oral administration as dependent on the method of administration for the particular agent.

In some embodiments, PARP inhibitors, such as, for example, compounds of Formula (I) and (IA), are used in the treatment of disease ameliorated by the inhibition of PARP, which includes administering to a subject in need of treatment a therapeutically-effective amount of a compound provided herein, and in one embodiment in the form of a pharmaceutical composition. In certain embodiments, PARP inhibitors, such as, for example, compounds of Formula (I) and (IA), are used in the treatment of cancer, which includes administering to a subject in need of treatment a therapeutically-effective amount of a compound provided herein in combination, and in one embodiment in the form of a pharmaceutical composition, simultaneously or sequentially with radiotherapy (ionizing radiation) or therapeutic agents.

In certain embodiments, PARP inhibitors, such as, for example, compounds of Formula (I) and (IA), are used in the preparation of a medicament for the treatment of cancer which is deficient in Homologous Recombination (HR) dependent DNA double strand break (DSB) repair activity, or in the treatment of a patient with a cancer which is deficient in HR dependent DNA DSB repair activity, which includes administering to said patient a therapeutically-effective amount of the compound.

In one embodiment is a method for treating a cancer deficient in mismatch DNA repair pathway. In another embodiment is a method for treating a cancer demonstrating microsatellite instability due to reduced or impaired DNA repair pathways. In another embodiment is a method for treating a cancer demonstrating genomic instability due to reduced or impaired DNA repair pathways.

In some embodiments, a cancer which is deficient in HR dependent DNA DSB repair includes one or more cancer cells which have a reduced or abrogated ability to repair DNA DSBs through that pathway, relative to normal cells, i.e. the activity of the HR dependent DNA DSB repair pathway are reduced or abolished in the one or more cancer cells.

In certain embodiments, the activity of one or more components of the HR dependent DNA DSB repair pathway is abolished in the one or more cancer cells of an individual having a cancer which is deficient in HR dependent DNA DSB repair. Components of the HR dependent DNA DSB repair pathway include the components listed above.

In some embodiments, the cancer cells have a BRCA1 and/or a BRCA2 deficient phenotype, i.e., BRCA1 and/or BRCA2 activity is reduced or abolished in the cancer cells. In certain embodiments, cancer cells with this phenotype are deficient in BRCA1 and/or BRCA2, i.e., expression and/or activity of BRCA1 and/or BRCA2 is reduced or abolished in the cancer cells, for example by means of mutation or polymorphism in the encoding nucleic acid, or by means of amplification, mutation or polymorphism in a gene encoding a regulatory factor, for example the EMSY gene which encodes a BRCA2 regulatory factor or by an epigenetic mechanism such as gene promoter methylation.

In certain instances, mutations and polymorphisms associated with cancer are detected at the nucleic acid level by detecting the presence of a variant nucleic acid sequence or at the protein level by detecting the presence of a variant (i.e. a mutant or allelic variant) polypeptide.

### DEFINITIONS

Unless defined otherwise, all technical and scientific terms used herein have the standard meaning pertaining to the claimed subject matter belongs. In the event that there are a plurality of definitions for terms herein, those in this section prevail.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. Furthermore, use of the term “including” as well as other forms, such as “include”, “includes,” and “included,” is not limiting.

Unless otherwise indicated, conventional methods of mass spectroscopy, NMR, HPLC, protein chemistry, biochemistry, recombinant DNA techniques and pharmacology are employed. Unless specific definitions are provided, the standard nomenclature employed in connection with, and the standard laboratory procedures and techniques of, analytical chemistry, synthetic organic chemistry, and medicinal and pharmaceutical chemistry are employed. In certain instances, standard techniques are used for chemical syntheses, chemical analyses, pharmaceutical preparation, formulation, and delivery, and treatment of patients. In certain embodiments, standard techniques are used for recombinant DNA, oligonucleotide synthesis, and tissue culture and transformation (e.g., electroporation, lipofection). In some embodiments, reactions and purification techniques are performed e.g., using kits of manufacturer's specifications or as commonly accomplished or as described herein.

As used throughout this application and the appended claims, the following terms have the following meanings:

“Alkenyl” means a linear monovalent hydrocarbon radical of two to ten carbons or a branched monovalent hydrocarbon radical of three to six carbon atoms which hydrocarbon contains at least one double bond, in another example one or two double bonds. Illustrative examples of alkenyl are ethenyl, 2-propenyl, 2-methyl-2-propenyl, 3-butenyl, 4-pentenyl, 5-hexenyl, 2-heptenyl, 2-methyl-1-heptenyl, and 3-cecenyl. “C₂-C₆alkenyl” as used herein means an alkenyl group comprising two to six carbons.

“Alkylene” means a linear, saturated, divalent hydrocarbon radical of one to ten carbons or a branched, saturated, divalent hydrocarbon radical of three to ten carbon atoms. For example, “C₁₋C₆alkylene” means an alkylene, as defined herein, of one to six carbons.

“Alkoxy” as used herein, means an —OR group where R is an alkyl group, as defined herein. Illustrative examples of alkoxy are methoxy, ethoxy, propoxy, 2-propoxy, butoxy, tert-butoxy, pentyloxy, and hexyloxy. “C₁-C₆alkoxy” as used herein means an alkoxy group where the alkyl portion is one to six carbons.

“Alkoxyalkyl,” as used herein, means an alkyl group substituted with at least one, for example one or two, alkoxy, as defined herein. Illustrative examples of alkoxyalkyl are 2-methoxyethyl, 2-ethoxyethyl, tert-butoxyethyl and methoxymethyl.

“Alkoxycarbonyl” as used herein, means a —C(O)OR group where R is alkyl, as defined herein. Illustrative examples of alkoxycarbonyl are methoxycarbonyl, ethoxycarbonyl, and tert-butoxycarbonyl. “C₁-C₆alkoxycarbonyl” as used herein means an alkoxycarbonyl group where the alkyl portion is one to six carbons.

“Alkoxycarbonylalkyl” means an alkyl group, as defined herein, substituted with one alkoxycarbonyl, as defined herein, e.g. tert-butoxycarbonylmethyl, and the like.

“Alkyl” as used herein means a linear saturated monovalent hydrocarbon radical of one to ten carbons or a branched saturated monovalent hydrocarbon radical of three to six carbon atoms. Illustrative examples of alkyl are methyl, ethyl, n-propyl, iso-propyl, n-butyl, sec-butyl, tert-butyl, n-pentyl, isopentyl, neopentyl, n-hexyl, 3-methylhexyl, 2,2-dimethylpentyl, 2,3-dimethylhexyl, n-heptyl, n-octyl, n-nonyl, and n-decyl. “C₁-C₆ alkyl” as used herein means a hydrocarbon of one to six carbons. “C₁-C₆-alkyl” as used herein means a hydrocarbon alkyl group comprising one to six carbons.

“Alkylcarbonyl” as used herein, means a —C(O)R group where R is alkyl, as defined herein. Illustrative examples of alkylcarbonyl are acetyl, 1-oxopropyl, 2,2-dimethyl-1-oxopropyl, 1-oxobutyl, and 1-oxopentyl. “C₁-C₆alkylcarbonyl” as used herein means an alkylcarbonyl group where the alkyl portion is one to six carbons.

“Alkynyl” as used herein, means a linear monovalent hydrocarbon radical of two to ten carbons or a branched monovalent hydrocarbon radical of three to six carbon atoms which hydrocarbon contains at least one triple bond, in another example one or two triple bonds. Illustrative examples of alkynyl are acetylenyl, 1-propynyl, 2-propynyl, 3-butynyl, 2-pentynyl, and 1-butynyl. “C₂-C₆alkynyl” as used herein means an alkynyl of two to six carbons.

“Alkylsulfonyl” means an —S(O)₂R group where R is alkyl, as defined herein. “C₁-C₆alkylsulfonyl” as used herein means an alkyl sulfonyl group where the alkyl portion is a hydrocarbon of one to six carbons.

“Aryl” means a monovalent six- to fourteen-membered, mono- or bi-carbocyclic ring, where the monocyclic ring is aromatic and at least one of the rings in the bicyclic ring is aromatic. Unless stated otherwise, the valency of the group may be located on any atom of any ring within the radical, valency rules permitting. Representative examples include phenyl, naphthyl, indanyl, and indanyl, and the like.

“Arylalkyl” as used herein, means an alkyl group substituted with one or two aryl group(s), as defined herein. Illustrative examples of arylalkyl are benzyl, 2-phenylethyl, 3-phenylpropyl, 1-methyl-3-phenylpropyl, and 2-naphth-2-ylethyl. For example, “aryl-C₁-C₆-alkyl” is a C₁-C₆-alkyl substituted with one or two, aryl groups.

“Arylcarbonyl” means a —C(O)R group where R is aryl, as defined herein.

“Arylsulfonyl” means an —S(O)₂R group where R is aryl, as defined herein.

“Cycloalkyl” means a monocyclic or fused or bridged bicyclic or tricyclic, saturated or partially unsaturated (but not aromatic), monovalent hydrocarbon radical of three to ten carbon ring atoms. Unless stated otherwise, the valency of the group may be located on any atom of any ring within the radical, valency rules permitting. One or two ring carbon atoms may be replaced with a —C(O)—, —C(S)—, or —C(=NH)— group. More specifically, the term cycloalkyl includes, but is not limited to, cyclopropyl, cyclobutyl, cyclopentyl, cyclohexyl, cyclohex-3-enyl and the like. For example, “C₃-C₈cycloalkyl” means a cycloalkyl comprising three to eight carbon atoms.

“Cycloalkylalkyl” means an alkyl substituted with a cycloalkyl group as defined herein.

“Ester” means a —C(O)OR group, where R is selected from among alkyl, cycloalkyl, aryl, heteroaryl (bonded through a ring carbon) and heterocycloalkyl (bonded through a ring carbon). In some embodiments, any hydroxy, or carboxyl side chain on the compounds described herein is esterified.

“Haloalkyl” means an alkyl group, as defined herein, substituted with at least one halo, for example 1, 2, 3, 4, or 5 halo. Where the haloalkyl has more than one halo, the halo can be the same or different. “C₁-C₆haloalkyl” means a haloalkyl where the alkyl portion comprises one to six carbon atoms.

“Haloalkoxy” means an —OR group where R is haloalkyl, as defined herein. “C₁-C₆haloalkoxy” means a haloalkoxy where the alkyl portion comprises one to six carbon atoms.

“Heteroaryl” means a monocyclic or fused or bridged bicyclic monovalent radical of 5 to 14 ring atoms containing one or more, specifically one, two, three, or four ring heteroatoms where each heteroatom is independently —O—, —S(O)ₙ— (n is 0, 1, or 2), —NH‘3, —N═, or N-oxide, with the remaining ring atoms being carbon, where the ring comprising a monocyclic radical is aromatic and where at least one of the fused and bridged rings comprising a bicyclic radical is aromatic. One or two ring carbon atoms of any nonaromatic rings comprising the bicyclic radical may be replaced by a —C(O)—, —C(S)—, or —C(=NH)— group. Unless stated otherwise, the valency of the group may be located on any atom of any ring within the radical, valency rules permitting. More specifically the term heteroaryl includes, but is not limited to, triazolyl, phthalimidyl, pyridinyl, pyrrolyl, imidazolyl, thienyl, furanyl, indolyl, 2,3-dihydro-1H-indolyl (including, for example, 2,3-dihydro-1H-indol-2-yl or 2,3-dihydro-1H-indol-5-yl, and the like), isoindolyl, indolinyl, isoindolinyl, benzimidazolyl, benzodioxol-4-yl, benzofuranyl, cinnolinyl, indazolyl, indolizinyl, naphthyridin-3-yl, phthalazin-3-yl, phthalazin-4-yl, pteridinyl, purinyl, quinazolinyl, quinoxalinyl, tetrazoyl, pyrazolyl, pyrazinyl, pyrimidinyl, pyridazinyl, oxazolyl, isooxazolyl, oxadiazolyl, benzoxazolyl, quinolinyl, isoquinolinyl, tetrahydroisoquinolinyl (including, for example, tetrahydroisoquinolin-4-yl or tetrahydroisoquinolin-6-yl, and the like), pyrrolo[3,2-c]pyridinyl (including, for example, pyrrolo[3,2-c]pyridin-2-yl or pyrrolo[3,2-c]pyridin-7-yl, and the like), benzopyranyl, thiazolyl, isothiazolyl, thiadiazolyl, benzothiazolyl, benzothienyl, thieno[3,2-b]thienyl, benzotriazolyl, 1H-[1,2,3]triazolo[4,5-d]pyrimidinyl, isobenzofuranyl, and the derivatives thereof, or N-oxide or a protected derivative thereof.

“Heteroarylalkyloxy” means an —OR group where R is heteroarylalkyl, as defined herein.

“Heteroarylalkyl” means an alkyl group, as defined herein, substituted with at least one, for example one or two, heteroaryl, as defined herein. “Heteroaryl-C₁-C₆-alkyl” means an C₁-C₆-alkyl, as defined herein, substituted with at least one, for example one or two, heteroaryl, as defined herein.

“Heteroarylcarbonyl” means a —C(O)R group where R is heteroaryl, as defined herein.

“Heteroaryloxy” means an —OR group where R is heteroaryl, as defined herein.

“Heteroarylsulfonyl” means an —S(O)₂R group where R is heteroaryl, as defined herein.

“Heteroarylthio” means an —SR group where R is heteroaryl, as defined herein.

“Heteroarylalkylthio” means an —SR group where R is heteroarylalkyl, as defined herein.

“Heterocyclyl” means a heteroaryl group, as defined herein, or a heterocycloalkyl group, as defined herein.

“Heterocycloalkylalkylthio” means an —SR group where R is heterocycloalkylalkyl, as defined herein.

“Non-aromatic heterocycle”, “non-aromatic heterocyclic”, “heterocycloalkyl” or “heteroalicyclic” means a saturated or partially unsaturated (but not aromatic) monovalent monocyclic group of 3 to 8 ring atoms or a saturated or partially unsaturated (but not aromatic) monovalent fused or bridged bicyclic or tricyclic group of 5 to 12 ring atoms in which one or more, specifically one, two, three, or four ring heteroatoms where each heteroatom is independently —O—, —S(O)ₙ— (n is 0, 1, or 2), —N═, or —NH—, the remaining ring atoms being carbon. One or two ring carbon atoms may be replaced by a —C(O)—, —C(S)—, or —C(═NH)— group. Unless otherwise stated, the valency of the group may be located on any atom of any ring within the radical, valency rules permitting. Illustrative examples include lactams, lactones, cyclic imides, cyclic thioimides, cyclic carbamates, More specifically the term heterocycloalkyl includes, but is not limited to, azetidinyl, pyrrolinyl, pyrrolidinyl, 2-oxopyrrolidinyl, 2,5-dioxo-1H-pyrrolyl, 2,5-dioxo-pyrrolidinyl, 2,5-dihydro-1H-pyrrolyl, piperidinyl, 4-piperidonyl, morpholinyl, piperazinyl, 2-oxopiperazinyl, dioxopiperazinyl, pyranyl, tetrahydropyranyl, tetrahydrothiopyranyl, 1,3-dioxinyl, 1,3-dioxanyl, 1,4-dioxinyl, 1,4-dioxanyl, 2-oxopiperidinyl, thiomorpholinyl, thiamorpholinyl, perhydroazepinyl, pyrazolidinyl, imidazolinyl, imidazolidinyl, 2,4-dioxo-imidazolidinyl, dihydropyridinyl, tetrahydropyridinyl, oxazolinyl, oxazolidinyl, isoxazolidinyl, thiazolinyl, thiazolidinyl, quinuclidinyl, isothiazolidinyl, octahydroindolyl, octahydroisoindolyl, decahydroisoquinolyl, 1,3-oxathianyl, 1,4-oxathiinyl, 1,4-oxathianyl, tetrahydro-1,4-thiazinyl, 2H-1,2-oxazinyl, tetrahydrofuryl, 2,4,6-trioxo-(1H,3H,5H)pyrimidinyl, 4,6-dioxo-2-(1H,5H)thioxodihydropyrimidinyl, 2,4(1H,3H)-dioxo-dihydropyrimidinyl, trioxanyl, hexahydro-1,3,5-triazinyl, tetrahydrothienyl, tetrahydrofuranyl, pyrazolinyl, 1,3-dioxolyl, 1,3-dioxolanyl, 1,3-dithiolyl, 1,3-dithiolanyl, isoxazolinyl, isoxazolidinyl, oxazolidinonyl, 1,3-oxathiolanyl, 2(3H)-oxo-dihydrothienyl, 2(3H)-oxo-dihydrofuranyl, 1,1-dioxo-tetrahydrothienyl, 2-oxo-1,3-dioxolanyl, 4,5-dihydrooxazolyl, oxiranyl, (1s,4s)-7-oxabicyclo[2.2.1]heptanyl, 2,3-dihydrobenzo[b][1,4]dioxinyl, 4H-1,4-thiazinyl, octahydro-1H-quinolizinyl, and tetrahydropyranyl, and the derivatives thereof and N-oxide or a protected derivative thereof. Additional examples include

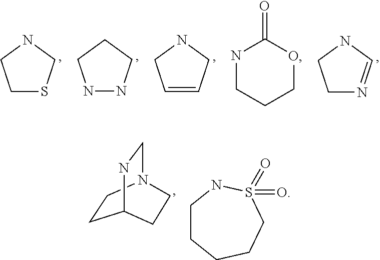

“Heterocycloalkylalkyl” means an alkyl substituted with at least one, in another example one or two heterocycloalkyl group(s), as defined herein.

“Heterocycloalkyloxy” means an —OR group where R is heterocycloalkyl as defined herein

“Heterocycloalkylalkyloxy means an —OR group where R is heterocycloalkylalkyl as defined herein.

“Hydroxyalkyl” means an alkyl group, as defined herein, substituted with at least one, in another example 1, 2, or 3, hydroxy groups. “C₁-C₆hydroxyalkyl” means a hydroxyalkyl group where the alkyl portion comprises one to six carbon atoms.

“Oxo” as used herein, means a ═O group.

As used herein, the term “moiety” refers to a specific segment or functional group of a molecule. Chemical moieties are often recognized chemical entities embedded in or appended to a molecule.

The term “protecting group” refers to a removable group which modifies the reactivity of a functional group, for example, a hydroxyl, ketone or amine, against undesirable reaction during synthetic procedures and to be later removed. Examples of hydroxy-protecting groups include, but not limited to, methylthiomethyl, tert-dimethylsilyl, tert-butyldiphenylsilyl, ethers such as methoxymethyl, and esters including acetyl, benzoyl, and the like. Examples of ketone protecting groups include, but not limited to, ketals, oximes, O-substituted oximes for example O-benzyl oxime, O-phenylthiomethyl oxime, 1-isopropoxycyclohexyl oxime, and the like. Examples of amine protecting groups include, but are not limited to, tert-butoxycarbonyl (Boc) and carbobenzyloxy (Cbz).

The term “optionally substituted” as defined herein, means the referenced group is substituted with zero, one or more substituents.

In some embodiments, compounds of the described herein exist as stereoisomers. The embodiments described herein specifically includes the various stereoisomers and mixtures thereof. Stereoisomers include (but are not limited to) geometric isomers, enantiomers, diastereomers, and mixtures of geometric isomers, enantiomers or diastereomers. In some embodiments, individual stereoisomers of compounds are prepared synthetically from commercially available starting materials which contain asymmetric or chiral centers or by preparation of racemic mixtures followed by resolution. These methods of resolution are exemplified by (1) attachment of a mixture of enantiomers to a chiral auxiliary, separation of the resulting mixture of diastereomers by recrystallization or chromatography and liberation of the optically pure product from the auxiliary or (2) direct separation of the mixture of optical enantiomers on chiral chromatographic column.

The methods and formulations described herein include the use of N-oxides, crystalline forms (also known as polymorphs), or pharmaceutically acceptable salts of compounds described herein, as well as active metabolites of these compounds having the same type of activity. In some situations, compounds exist as tautomers. All tautomers are included within the scope of the compounds presented herein. In some embodiments, the compounds described herein exist in unsolvated as well as solvated forms with pharmaceutically acceptable solvents such as water, ethanol, and the like. The solvated forms of the compounds presented herein are also considered to be disclosed herein.

Throughout the specification, groups and substituents thereof are chosen, in certain embodiments, to provide stable moieties and compounds.

### Certain Pharmaceutical Terminology

The term “acceptable” with respect to a formulation, composition or ingredient, as used herein, means having no persistent detrimental effect on the general health of the subject being treated.

As used herein, the term “selective binding compound” refers to a compound that selectively binds to any portion of one or more target proteins.

As used herein, the term “selectively binds” refers to the ability of a selective binding compound to bind to a target protein, such as, for example, PARP, with greater affinity than it binds to a non-target protein. In certain embodiments, specific binding refers to binding to a target with an affinity that is at least about 10, about 50, about 100, about 250, about 500, about 1000 or more times greater than the affinity for a non-target.

As used herein, the term “target protein” refers to a molecule or a portion of a protein capable of being bound by a selective binding compound. In certain embodiments, a target protein is the enzyme poly(ADP-ribose)polymerase (PARP).

As used herein, the terms “treating” or “treatment” encompass either or both responsive and prophylaxis measures, e.g., designed to inhibit, slow or delay the onset of a symptom of a disease or disorder, achieve a full or partial reduction of a symptom or disease state, and/or to alleviate, ameliorate, lessen, or cure a disease or disorder and/or its symptoms. The terms “treat,” “treating” or “treatment” include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

As used herein, “amelioration” of the symptoms of a particular disorder by administration of a particular compound or pharmaceutical composition refers to any lessening of severity, delay in onset, slowing of progression, or shortening of duration, whether permanent or temporary, lasting or transient that can be attributed to or associated with administration of the compound or composition.

As used herein, “potentiation” occurs when the effect of a combined therapy is more than the effect of the individual therapies added to together, i.e. one therapy intensifies the effect of the other.

As used herein, the term “modulator” refers to a compound that alters an activity of a molecule. For example, a modulator includes a compound that causes an increase or a decrease in the magnitude of a certain activity of a molecule compared to the magnitude of the activity in the absence of the modulator. In certain embodiments, a modulator is an inhibitor, which decreases the magnitude of one or more activities of a molecule. In certain embodiments, an inhibitor completely prevents one or more activities of a molecule. In certain embodiments, a modulator is an activator, which increases the magnitude of at least one activity of a molecule. In certain embodiments the presence of a modulator results in an activity that does not occur in the absence of the modulator.

As used herein, the term “selective modulator” refers to a compound that selectively modulates a target activity.

As used herein, the term “PARP ” refers to the family of the enzyme poly(ADP-ribose)polymerase which includes approximately 18 proteins, particularly poly(ADP-ribose)polymerase-1 (PARP-1) and poly(ADP-ribose)polymerase-2 (PARP-2).

As used herein, the term “selective PARP modulator” refers to a compound that selectively modulates at least one activity associated with the enzyme poly(ADP-ribose)polymerase (PARP). In various embodiments, the selective modulator selectively modulates the activity of PARP-1, PARP-2, both PARP-1 and PARP-2 or several members of the family of the enzyme poly(ADP-ribose)polymerase (PARP).

As used herein, the term “method of inhibiting PARP” refers to a method of inhibiting the activity of either one or more of the family of enzyme poly(ADP-ribose)polymerase (PARP). As used herein, the term “inhibition of PARP” refers to inhibition of the activity of either one or more of the family of enzyme poly(ADP-ribose)polymerase (PARP).

As used herein, the term “modulating the activity of the enzyme poly(ADP-ribose)polymerase” refers to a modulating the activity of either one or more of the family of enzyme poly(ADP-ribose)polymerase (PARP).

The term “modulate,” as used herein, means to interact with a target either directly or indirectly so as to alter the activity of the target, including, by way of example only, to enhance the activity of the target, to inhibit the activity of the target, to limit the activity of the target, or to extend the activity of the target.

As used herein, the term “selectively modulates” refers to the ability of a selective modulator to modulate a target activity to a greater extent than it modulates a non-target activity. In certain embodiments the target activity is selectively modulated by, for example about 2 fold up to more that about 500 fold, in some embodiments, about 2, 5, 10, 50, 100, 150, 200, 250, 300, 350, 400, 450 or more than 500 fold.

As used herein, the term “target activity” refers to a biological activity capable of being modulated by a selective modulator. Certain exemplary target activities include, but are not limited to, binding affinity, signal transduction, enzymatic activity, tumor growth, inflammation or inflammation-related processes, and amelioration of one or more symptoms associated with a disease or condition.

As used herein, the term “antagonist” or “inhibitor” refers to a compound, the presence of which results in a decrease in the magnitude of a biological activity of a protein. In certain embodiments, the presence of an antagonist results in complete inhibition of a biological activity of a protein, such as, for example, the enzyme poly(ADP-ribose)polymerase (PARP).

As used herein, the IC₅₀ refers to an amount, concentration or dosage of a particular test compound that achieves a 50% inhibition of a maximal response, such as modulation of PARP, in an assay that measures such response.

As used herein, EC₅₀ refers to a dosage, concentration or amount of a particular test compound that elicits a dose-dependent response at 50% of maximal expression of a particular response that is induced, provoked or potentiated by the particular test compound.

The term “cancer”, as used herein refers to an abnormal growth of cells which tend to proliferate in an uncontrolled way and, in some cases, to metastasize (spread). The types of cancer include, but are not limited to, solid tumors (such as those of the bladder, bowel, brain, breast, endometrium, heart, kidney, lung, lymphatic tissue (lymphoma), ovary, pancreas or other endocrine organ (thyroid), prostate, skin (melanoma) or hematological tumors (such as the leukemias).

The term “carrier,” as used herein, refers to relatively nontoxic chemical compounds or agents that facilitate the incorporation of a compound into cells or tissues.

The terms “co-administration” or the like, as used herein, are meant to encompass administration of the selected therapeutic agents to a single patient, and are intended to include treatment regimens in which the agents are administered by the same or different route of administration or at the same or different time.

The term “diluent” refers to chemical compounds that are used to dilute the compound of interest prior to delivery. Diluents include chemicals used to stabilize compounds because they provide a more stable environment. Salts dissolved in buffered solutions (which also can provide pH control or maintenance) are utilized as diluents in certain embodiments, including, but not limited to a phosphate buffered saline solution.

The terms “effective amount” or “therapeutically effective amount,” as used herein, refer to a sufficient amount of an agent or a compound being administered which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is determined using any suitable technique, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “inflammatory disorders” refers to those diseases or conditions that are characterized by one or more of the signs of pain (dolor, from the generation of noxious substances and the stimulation of nerves), heat (calor, from vasodilatation), redness (rubor, from vasodilatation and increased blood flow), swelling (tumor, from excessive inflow or restricted outflow of fluid), and loss of function (functio laesa, which may be partial or complete, temporary or permanent). Inflammation takes many forms and includes, but is not limited to, inflammation that is one or more of the following: acute, adhesive, atrophic, catarrhal., chronic, cirrhotic, diffuse, disseminated, exudative, fibrinous, fibrosing, focal., granulomatous, hyperplastic, hypertrophic, interstitial., metastatic, necrotic, obliterative, parenchymatous, plastic, productive, proliferous, pseudomembranous, purulent, sclerosing, seroplastic, serous, simple, specific, subacute, suppurative, toxic, traumatic, and/or ulcerative. Inflammatory disorders further include, without being limited to those affecting the blood vessels (polyarteritis, temporal arteritis); joints (arthritis: crystalline, osteo-, psoriatic, reactive, rheumatoid, Reiter's); gastrointestinal tract (Crohn's Disease, ulcerative colitis); skin (dermatitis); or multiple organs and tissues (systemic lupus erythematosus).

The term “PARP-mediated”, as used herein, refers to conditions or disorders that are ameliorated by the one or more of the family of enzyme poly(ADP-ribose)polymerase (PARP).

The terms “kit” and “article of manufacture” are used as synonyms.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, in certain instances, enzymes produce specific structural alterations to a compound. In some embodiments, metabolites of the compounds disclosed herein are identified either by administration of compounds to a host and analysis of tissue samples from the host, or by incubation of compounds with hepatic cells in vitro and analysis of the resulting compounds.

By “pharmaceutically acceptable” or “therapeutically acceptable”, as used herein, refers a material, such as a carrier or diluent, which does not abrogate the biological activity or properties of the compound, and is relatively nontoxic. In certain instances, nontoxic and non-abrogative materials includes materials that when administered to an individual do not cause substantial, undesirable biological effects and/or do not interact in a deleterious manner with any of the components of the composition in which it is contained.

The term “pharmaceutically acceptable salt” or “therapeutically acceptable salt”, refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound described herein and a co-agent, are both administered to a patient simultaneously in the form of a single entity or dosage. The term “non-fixed combination” means that the active ingredients, e.g. a compound described herein and a co-agent, are administered to a patient as separate entities either simultaneously, concurrently or sequentially with no specific intervening time limits, where such administration provides effective levels of the two compounds in the body of the patient. The latter also applies to cocktail therapy, e.g. the administration of three or more active ingredients.

The term “pharmaceutical composition” refers to a mixture of a compound described herein with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, pulmonary and topical administration.

A “prodrug” refers to an agent that is converted into the parent drug in vivo. Prodrugs are often useful because, in some situations, they are easier to administer than the parent drug. In certain instances, a prodrug is bioavailable by oral administration whereas the parent is not. In some instances, a prodrug has improved solubility in pharmaceutical compositions over the parent drug. An example, without limitation, of a prodrug is a compound described herein, which is administered as an ester (the “prodrug”) to facilitate transmittal across a cell membrane where water solubility is detrimental to mobility but which then is metabolically hydrolyzed to the carboxylic acid, the active entity, once inside the cell where water solubility is beneficial. A further example of a prodrug might be a short peptide (polyaminoacid) bonded to an acid or amino group where the peptide is metabolized to reveal the active moiety. In certain embodiments, upon in vivo administration, a prodrug is chemically converted to the biologically, pharmaceutically or therapeutically more active form of the compound. In certain embodiments, a prodrug is enzymatically metabolized by one or more steps or processes to the biologically, pharmaceutically or therapeutically active form of the compound. To produce a prodrug, a pharmaceutically active compound is modified such that the active compound will be regenerated upon in vivo administration. In some embodiments, the prodrug is designed to alter the metabolic stability or the transport characteristics of a drug, to mask side effects or toxicity, to improve the flavor of a drug or to alter other characteristics or properties of a drug.

The term “subject” or “patient” encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

### Abbreviations

Abbreviations as used herein have the following meanings.

| Abbreviation | Meaning |
|---|---|
| Boc | tert-butoxycarbonyl |
| BPO | benzoyl peroxide |
| cm | centimeter |
| conc. | concentrated |
| DBU | 1,8-diazabicyclo[5.4.0]undec-7-ene |
| DCM | dichloromethane |
| DMA | dimethylacetamide |
| DMF | dimethylformamide |
| DMSO | dimethyl sulfoxide |
| ESI | electron spray ionization |
| EtOAc | ethyl acetate |
| FBS | fetal bovine serum |
| g | aram |
| HPLC | high-performance liquid |
| | chromatography |
| HRP | horseradish peroxidase |
| ip | intraperitoneal |
| kg | kilogram |
| LC-MS | liquid chromatography-mass |
| | spectrometry |
| 2-Me-THF | 2-methyltetrahydrofuran |
| mg | milligram |
| mL | millimeter |
| μM | micromolar |
| mm | millimeter |
| mmol | millimoles |
| MS | mass spectrometry |
| NAD | nicotinamide adenine dinucleotide |
| NBS | N-bromosuccinimide |
| nM | nanomolar |
| NMR | nuclear magnetic resonance |
| PBS | phosphate buffered saline |
| PPh₃ | triphenylphosphine |
| TEA | triethylamine |
| THF | tetrahydrofuran |
| TLC | thin layer chromatography |

### Pharmaceutical Composition/Formulation

In certain embodiments, pharmaceutical compositions are formulated in any manner, including using one or more physiologically acceptable carriers comprising excipients and/or auxiliaries which facilitate processing of the active compounds into pharmaceutical preparations. In some embodiments, proper formulation is dependent upon the route of administration chosen. In various embodiments, any techniques, carriers, and excipients are used as suitable.

Provided herein are pharmaceutical compositions that include a compound described herein and a pharmaceutically acceptable diluent(s), excipient(s), and/or carrier(s). In addition, in some embodiments, the compounds described herein are administered as pharmaceutical compositions in which compounds described herein are mixed with other active ingredients, as in combination therapy.

A pharmaceutical composition, as used herein, refers to a mixture of a compound described herein with other chemical components, such as carriers, stabilizers, diluents, dispersing agents, suspending agents, thickening agents, and/or excipients. In certain embodiments, a pharmaceutical composition facilitates administration of the compound to an organism. In some embodiments, practicing the methods of treatment or use provided herein, includes administering or using a pharmaceutical composition comprising a therapeutically effective amount of a compound provided herein. In specific embodiments, the methods of treatment provided for herein include administering such a pharmaceutical composition to a mammal having a disease or condition to be treated. In one embodiment, the mammal is a human. In some embodiments, the therapeutically effective amount varies widely depending on the severity of the disease, the age and relative health of the subject, the potency of the compound used and other factors. In various embodiments, the compounds described herein are used singly or in combination with one or more therapeutic agents as components of mixtures.

In certain embodiments, the pharmaceutical compositions provided herein are formulated for intravenous injections. In certain aspects, the intravenous injection formulations provided herein are formulated as aqueous solutions, and, in some embodiments, in physiologically compatible buffers such as Hank's solution, Ringer's solution, or physiological saline buffer. In certain embodiments, the pharmaceutical compositions provided herein are formulated for transmucosal administration. In some aspects, transmucosal formulations include penetrants appropriate to the barrier to be permeated. In certain embodiments, the pharmaceutical compositions provided herein are formulated for other parenteral injections, appropriate formulations include aqueous or nonaqueous solutions, and in one embodiment, with physiologically compatible buffers or excipients.

In certain embodiments, the pharmaceutical compositions provided herein are formulated for oral administration. In certain aspects, the oral formulations provided herein comprise compounds described herein that are formulated with pharmaceutically acceptable carriers or excipients. Such carriers enable the compounds described herein to be formulated as tablets, powders, pills, dragees, capsules, liquids, gels, syrups, elixirs, slurries, suspensions and the like, for oral ingestion by a patient to be treated.

In some embodiments, pharmaceutical preparations for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are optionally added, such as the cross-linked croscarmellose sodium, polyvinylpyrrolidone, agar, or alginic acid or a salt thereof such as sodium alginate.

In certain embodiments, provided herein is a pharmaceutical composition formulated as dragee cores with suitable coatings. In certain embodiments, concentrated sugar solutions are used in forming the suitable coating, and optionally contain gum arabic, talc, polyvinylpyrrolidone, carbopol gel, polyethylene glycol, and/or titanium dioxide, lacquer solutions, and suitable organic solvents or solvent mixtures. In some embodiments, dyestuffs and/or pigments are added to tablets, dragees and/or the coatings thereof for, e.g., identification or to characterize different combinations of active compound doses.

In certain embodiments, pharmaceutical preparations which are used include orally include push-fit capsules made of gelatin, as well as soft, sealed capsules made of gelatin and a plasticizer, such as glycerol or sorbitol. In some embodiments, the push-fit capsules contain the active ingredients in admixture with filler such as lactose, binders such as starches, and/or lubricants such as talc or magnesium stearate and, optionally, stabilizers. In certain embodiments, in soft capsules, the active compounds are dissolved or suspended in suitable liquids, such as fatty oils, liquid paraffin, or liquid polyethylene glycols. In addition, stabilizers are optionally added. In certain embodiments, the formulations for oral administration are in dosages suitable for such administration.

In certain embodiments, the pharmaceutical compositions provided herein are formulated for buccal or sublingual administration. In certain embodiments, buccal or sublingual compositions take the form of tablets, lozenges, or gels formulated in a conventional manner. In certain embodiments, parenteral injections involve bolus injection or continuous infusion. In some embodiments, formulations for injection are presented in unit dosage form, e.g., in ampoules or in multi-dose containers, with an added preservative. In some embodiments, the pharmaceutical composition described herein is in a form suitable for parenteral injection as a sterile suspensions, solutions or emulsions in oily or aqueous vehicles, and optionally contains formulatory agents such as suspending, stabilizing and/or dispersing agents. Pharmaceutical formulations for parenteral administration include aqueous solutions of the active compounds in water-soluble form. In some embodiments, suspensions of the active compounds are prepared as appropriate oily injection suspensions. Suitable lipophilic solvents or vehicles include fatty oils such as sesame oil, or synthetic fatty acid esters, such as ethyl oleate or triglycerides, or liposomes. In certain embodiments, aqueous injection suspensions contain substances which increase the viscosity of the suspension, such as sodium carboxymethyl cellulose, sorbitol, or dextran. Optionally, the suspensions also contain suitable stabilizers or agents which increase the solubility of the compounds to allow for the preparation of highly concentrated solutions. In alternative embodiments, the active ingredient is in powder form for constitution with a suitable vehicle, e.g., sterile pyrogen-free water, before use.

In some embodiments, the compounds described herein are administered topically. In specific embodiments, the compounds described herein are formulated into a variety of topically administrable compositions, such as solutions, suspensions, lotions, gels, pastes, medicated sticks, balms, creams or ointments. Such pharmaceutical compounds optionally contain solubilizers, stabilizers, tonicity enhancing agents, buffers and/or preservatives.

In certain embodiments, the pharmaceutical compositions provided herein are formulated for transdermal administration of compounds described herein. In some embodiments, administration of such compositions employs transdermal delivery devices and transdermal delivery patches. In certain embodiments, the compositions are lipophilic emulsions or buffered, aqueous solutions, dissolved and/or dispersed in a polymer or an adhesive. Such patches include those constructed for continuous, pulsatile, or on demand delivery of pharmaceutical agents. In some embodiments, transdermal delivery of the compounds described herein is accomplished by use of iontophoretic patches and the like. In certain embodiments, transdermal patches provide controlled delivery of the compounds provided herein, such as, for example, compounds of Formula (I), (IA) or (II). In certain embodiments, the rate of absorption is slowed by using rate-controlling membranes or by trapping the compound within a polymer matrix or gel. Conversely, absorption enhancers are optionally used to increase absorption. Absorption enhancer and carrier include absorbable pharmaceutically acceptable solvents that assist in passage of the compound through the skin. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

In certain embodiments, the pharmaceutical compositions provided herein are formulated for administration by inhalation. In certain embodiments, in such pharmaceutical compositions formulated for inhalation, the compounds described herein are in a form as an aerosol, a mist or a powder. In some embodiments, pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In certain aspects of a pressurized aerosol, the dosage unit is determined by providing a valve to deliver a metered amount. In certain embodiments, capsules and cartridges of, such as, by way of example only, gelatin for use in an inhaler or insufflator is formulated containing a powder mix of the compound described herein and a suitable powder base such as lactose or starch.

In some embodiments, the compounds described herein are formulated in rectal compositions such as enemas, rectal gels, rectal foams, rectal aerosols, suppositories, jelly suppositories, or retention enemas. In certain embodiments, rectal compositions optionally contain conventional suppository bases such as cocoa butter or other glycerides, as well as synthetic polymers such as polyvinylpyrrolidone, PEG, and the like. In certain suppository forms of the compositions, a low-melting wax such as, but not limited to, a mixture of fatty acid glycerides, optionally in combination with cocoa butter is first melted.

In various embodiments provided herein, the pharmaceutical compositions are formulated in a conventional manner using one or more physiologically acceptable carriers comprising excipients and auxiliaries which facilitate processing of the active compounds into pharmaceutically acceptable preparations. In certain embodiments, proper formulation is dependent upon the route of administration chosen. In various embodiments, any of the techniques, carriers, and excipients is used as suitable. In some embodiments, pharmaceutical compositions comprising a compound described herein are manufactured in a conventional manner, such as, by way of example only, by means of conventional mixing, dissolving, granulating, dragee-making, levigating, emulsifying, encapsulating, entrapping or compression processes.

In certain embodiments, the pharmaceutical compositions include at least one pharmaceutically acceptable carrier, diluent or excipient and a compound described herein described herein as an active ingredient in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. In some situations, compounds described herein exist as tautomers. All tautomers are included within the scope of the compounds presented herein. Additionally, included herein are the solvated and unsolvated forms of the compounds described herein. Solvated compounds include those that are solvated with pharmaceutically acceptable solvents such as water, ethanol, and the like. The solvated forms of the compounds presented herein are also considered to be disclosed herein. In some embodiments, the pharmaceutical compositions described herein include other medicinal or pharmaceutical agents, carriers, adjuvants, such as preserving, stabilizing, wetting or emulsifying agents, solution promoters, salts for regulating the osmotic pressure, and/or buffers. In additional embodiments, the pharmaceutical compositions described herein also contain other therapeutically valuable substances.

Methods for the preparation of compositions containing the compounds described herein include formulating the compounds with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions and creams. In various embodiments, the compositions are in liquid solutions or suspensions, solid forms suitable for solution or suspension in a liquid prior to use, or as emulsions. These compositions optionally contain minor amounts of nontoxic, auxiliary substances, such as wetting or emulsifying agents, pH buffering agents, and so forth.

In some embodiments, a composition comprising a compound described herein takes the form of a liquid where the agents are present in solution, in suspension or both. In some embodiments, when the composition is administered as a solution or suspension a first portion of the agent is present in solution and a second portion of the agent is present in particulate form, in suspension in a liquid matrix. In some embodiments, a liquid composition includes a gel formulation. In other embodiments, the liquid composition is aqueous.

Useful aqueous suspension optionally contain one or more polymers as suspending agents. Useful polymers include water-soluble polymers such as cellulosic polymers, e.g., hydroxypropyl methylcellulose, and water-insoluble polymers such as cross-linked carboxyl-containing polymers. Useful compositions optionally comprise an mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

Useful compositions optionally include solubilizing agents to aid in the solubility of a compound described herein. The term “solubilizing agent” generally includes agents that result in formation of a micellar solution or a true solution of the agent. Solubilizing agents include certain acceptable nonionic surfactants, for example polysorbate 80, and ophthalmically acceptable glycols, polyglycols, e.g., polyethylene glycol 400, and glycol ethers.

Useful compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Useful compositions optionally include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Certain useful compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Some useful compositions optionally include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Certain useful compositions optionally one or more antioxidants to enhance chemical stability where required. Suitable antioxidants include, by way of example only, ascorbic acid and sodium metabisulfite.

In some embodiments, aqueous suspension compositions are packaged in single-dose non-reclosable containers. In alternative embodiments, multiple-dose reclosable containers are used, in which case it is typical to include a preservative in the composition.

In various embodiments, any delivery system for hydrophobic pharmaceutical compounds is employed. Liposomes and emulsions are examples of delivery vehicles or carriers for hydrophobic drugs. In certain embodiments, certain organic solvents such as N-methylpyrrolidone are employed. In some embodiments, the compounds are delivered using a sustained-release system, such as semipermeable matrices of solid hydrophobic polymers containing the therapeutic agent. Various sustained-release materials are utilized in the embodiments herein. In certain embodiments, sustained-release capsules release the compounds for a few weeks up to over 100 days. In some embodiments, depending on the chemical nature and the biological stability of the therapeutic reagent, additional strategies for protein stabilization are employed.

In certain embodiments, the formulations or compositions described herein benefit from and/or optionally comprise antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (1) pentosan polysulfate and other heparinoids, (m) divalent cations such as magnesium and zinc; or (n) combinations thereof.

### Methods of Dosing and Treatment Regimens

In certain embodiments, the compounds described herein are used in the preparation or manufacture of medicaments for the treatment of diseases or conditions that are mediated by the enzyme poly(ADP-ribose)polymerase (PARP) or in which inhibition of the enzyme poly(ADP-ribose)polymerase (PARP) ameliorates the disease or condition. In some embodiments, a method for treating any of the diseases or conditions described herein in a subject in need of such treatment, involves administration of pharmaceutical compositions containing at least one compound described herein, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said subject.

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest the symptoms of the disease or condition. In some embodiments, amounts effective for this use will depend on the severity and course of the disease or condition, previous therapy, the patients health status, weight, and response to the drugs, and the judgment of the treating physician. In certain instances, it is considered appropriate for the caregiver to determine such therapeutically effective amounts by routine experimentation (including, but not limited to, a dose escalation clinical trial).

In certain prophylactic applications, compositions containing the compounds described herein are administered to a patient susceptible to or otherwise at risk of a particular disease, disorder or condition. In some embodiments, the amount administered is defined to be a “prophylactically effective amount or dose.” In certain embodiments of this use, the precise amounts of compound administered depend on the patients state of health, weight, and the like. In some embodiments, it is considered appropriate for the caregiver to determine such prophylactically effective amounts by routine experimentation (e.g., a dose escalation clinical trial). In certain embodiments, when used in a patient, effective amounts for this use will depend on the severity and course of the disease, disorder or condition, previous therapy, the patients health status and response to the drugs, and the judgment of the treating physician.

In certain instances, a patient's condition does not improve or does not significantly improve following administration of a compound or composition described herein and, upon the doctor's discretion the administration of the compounds is optionally administered chronically, that is, for an extended period of time, including throughout the duration of the patient's life in order to ameliorate or otherwise control or limit the symptoms of the patient's disease or condition.

In certain cases where the patient's status does improve or does not substantially improve, upon the doctor's discretion the administration of the compounds are optionally given continuously; alternatively, the dose of drug being administered is optionally temporarily reduced or temporarily suspended for a certain length of time (i.e., a “drug holiday”). In certain embodiments, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes a reduction from about 10% to about 100%, including, by way of example only, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In certain embodiments, once improvement of the patient's conditions has occurred, a maintenance dose is administered if necessary. In some embodiments, the dosage, e.g., of the maintenance dose, or the frequency of administration, or both, are reduced, as a function of the symptoms, to a level at which the improved disease, disorder or condition is retained. In certain embodiments, however, patients are optionally given intermittent treatment on a long-term basis upon any recurrence of symptoms.

In certain embodiments, the amount of a given agent that corresponds to an effective amount varies depending upon factors such as the particular compound, disease or condition and its severity, the identity (e.g., weight) of the subject or host in need of treatment. In some embodiments, the effective amount is, nevertheless, determined according to the particular circumstances surrounding the case, including, e.g., the specific agent that is administered, the route of administration, the condition being treated, and the subject or host being treated. In certain embodiments, however, doses employed for adult human treatment is in the range of about 0.02 to about 5000 mg per day, in a specific embodiment about 1 to about 1500 mg per day. In various embodiments, the desired dose is conveniently presented in a single dose or as divided doses administered simultaneously (or over a short period of time) or at appropriate intervals, for example as two, three, four or more sub-doses per day.

In some embodiments, the pharmaceutical compositions described herein are in a unit dosage form suitable for single administration of precise dosages. In some instances, in unit dosage form, the formulation is divided into unit doses containing appropriate quantities of one or more compound. In certain embodiments, the unit dosage is in the form of a package containing discrete quantities of the formulation. Non-limiting examples are packaged tablets or capsules, and powders in vials or ampoules. In some embodiments, aqueous suspension compositions are packaged in single-dose non-reclosable containers. In alternative embodiments, multiple-dose reclosable containers are used, in which case it is typical to include a preservative in the composition. By way of example only, formulations for parenteral injection are, in some embodiments, presented in unit dosage form, which include, but are not limited to ampoules, or in multi-dose containers, with an added preservative.

In certain embodiments, the daily dosages appropriate for the compounds described herein described herein are from about 0.01 to about 2.5 mg/kg per body weight. In some embodiments, an indicated daily dosage in the larger subject, including, but not limited to, humans, is in the range from about 0.5 mg to about 100 mg, conveniently administered in divided doses, including, but not limited to, up to four times a day or in extended release form. In certain embodiments, suitable unit dosage forms for oral administration comprise from about 1 to about 50 mg active ingredient. The foregoing ranges are merely suggestive, as the number of variables in regard to an individual treatment regime is large, and considerable excursions from these recommended values are not uncommon. In certain embodiments, the dosages are altered depending on a number of variables, not limited to the activity of the compound used, the disease or condition to be treated, the mode of administration, the requirements of the individual subject, the severity of the disease or condition being treated, and the judgment of the practitioner.

In certain embodiments, toxicity and therapeutic efficacy of such therapeutic regimens are determined by standard pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. In certain embodiments, the compounds exhibit high therapeutic indices. In some embodiments, the data obtained from cell culture assays and animal studies is used in formulating a range of dosage for use in human. In specific embodiments, the dosage of such compounds lies within a range of circulating concentrations that include the ED₅₀ with minimal toxicity. In certain embodiments, the dosage varies within this range depending upon the dosage form employed and the route of administration utilized.

### Combination Treatments

In certain instances, it is appropriate to administer at least one compound described herein in combination with another therapeutic agent. By way of example only, if one of the side effects experienced by a patient upon receiving one of the compounds herein is inflammation, then, in some embodiments, it is appropriate to administer an anti-inflammatory agent in combination with the initial therapeutic agent. In some embodiments, the therapeutic effectiveness of one of the compounds described herein is enhanced by administration of an adjuvant (i.e., in some embodiments, by itself the adjuvant has minimal therapeutic benefit, but in combination with another therapeutic agent, the overall therapeutic benefit to the patient is enhanced). In certain embodiments, the benefit experienced by a patient is increased by administering one of the compounds described herein with another therapeutic agent (which also includes a therapeutic regimen) that also has therapeutic benefit. In some embodiments, regardless of the disease, disorder or condition being treated, the overall benefit experienced by the patient as a result of a combination treatment is additive or synergistic.

In certain embodiments, therapeutically-effective dosages vary when the drugs are used in treatment combinations. In some embodiments, therapeutically-effective dosages of drugs and other agents for use in combination treatment regimens is determined in any suitable manner, e.g., through the use of metronomic dosing, i.e., providing more frequent, lower doses in order to minimize toxic side effects. In some embodiments, combination treatment regimen described herein encompass treatment regimens in which administration of a PARP inhibitor described herein is initiated prior to, during, or after treatment with a second agent described above, and continues until any time during treatment with the second agent or after termination of treatment with the second agent. It also includes treatments in which a PARP inhibitor described herein and the second agent being used in combination are administered simultaneously or at different times and/or at decreasing or increasing intervals during the treatment period. Combination treatment further includes periodic treatments that start and stop at various times to assist with the clinical management of the patient. For example, in some embodiments, a PARP inhibitor described herein in the combination treatment is administered weekly at the onset of treatment, decreasing to biweekly, and decreasing further as appropriate.

In certain embodiments, compositions and methods for combination therapy are provided herein. In accordance with one aspect, the pharmaceutical compositions disclosed herein are used to in a method of treating a PARP mediated disease or condition or a disease or condition that is ameliorated by inhibition of PARP. In accordance with certain aspects, the pharmaceutical compositions disclosed herein are used to treat vascular disease; septic shock; ischemic injury; reperfusion injury; neurotoxicity; hemorrhagic shock; inflammatory diseases; multiple sclerosis; secondary effects of diabetes; and acute treatment of cytotoxicity following cardiovascular surgery. In a certain aspect, the pharmaceutical compositions disclosed herein are used in combination, either simultaneously or sequentially, with ionizing radiation or one or more therapeutic agents.

In certain embodiments, combination therapies described herein are used as part of a specific treatment regimen intended to provide a beneficial effect from the co-action of a PARP inhibitor described herein and a concurrent treatment. It is understood that the dosage regimen to treat, prevent, or ameliorate the condition(s) for which relief is sought, is optionally modified in accordance with a variety of factors.

In certain combination therapies described herein, dosages of the co-administered compounds vary depending on the type of co-drug employed, on the specific drug employed, on the disease or condition being treated and so forth. In some embodiments, when co-administered with one or more biologically active agents, the compound provided herein is administered either simultaneously with the biologically active agent(s), or sequentially. In certain aspects where the agents are administered sequentially, the attending physician will decide on the appropriate sequence of administering protein in combination with the biologically active agent(s).

In various embodiments, the multiple therapeutic agents (one of which is one of the compounds described herein) are administered in any order or even simultaneously. In certain instances, administration is simultaneous and the multiple therapeutic agents are, optionally, provided in a single, unified form, or in multiple forms (by way of example only, either as a single pill or as two separate pills). In some embodiments, one of the therapeutic agents is given in multiple doses, or both are given as multiple doses. In some instances, administration is not simultaneous and the timing between the multiple doses varies, by way of non-limiting example, from more than zero weeks to less than four weeks. In addition, the combination methods, compositions and formulations are not to be limited to the use of only two agents; the use of multiple therapeutic combinations are also envisioned.

In additional embodiments, the compounds described herein are used in combination with procedures that provide additional or synergistic benefit to the patient. By way of example only, patients are expected to find therapeutic and/or prophylactic benefit in the methods described herein, where pharmaceutical composition of a compound disclosed herein and/or combinations with other therapeutics are combined with genetic testing to determine whether that individual is a carrier of a mutant gene that is known to be correlated with certain diseases or conditions.

In certain embodiments, the compounds described herein and combination therapies are administered before, during or after the occurrence of a disease or condition. In certain embodiments, the timing of administering the composition containing a compound varies. Thus, for example, in some embodiments, the compounds are used as a prophylactic and are administered continuously to subjects with a propensity to develop conditions or diseases in order to prevent the occurrence of the disease or condition. In some embodiments, the compounds and compositions are administered to a subject during or as soon as possible after the onset of the symptoms. In certain embodiments, the administration of the compounds is initiated within the first 48 hours of the onset of the symptoms, within the first 6 hours of the onset of the symptoms, or within 3 hours of the onset of the symptoms. The initial administration is achieved via any route practical, such as, for example, an intravenous injection, a bolus injection, infusion over 5 minutes to about 5 hours, a pill, a capsule, transdermal patch, buccal delivery, and the like, or combination thereof. In some embodiments, a compound is administered as soon as is practicable after the onset of a disease or condition is detected or suspected, and for a length of time necessary for the treatment of the disease, such as, from about 1 month to about 3 months. In certain embodiments, the length of treatment varies for each subject, and the length is determined using any criteria. In exemplary embodiments, a compound or a formulation containing the compound is administered for at least 2 weeks, for about 1 month to about 5 years, or for about 1 month to about 3 years.

### Other Combination Therapies

In certain embodiments described herein, methods for treatment of PARP mediated conditions or diseases, such as proliferative disorders, including cancer, include administration to a patient compounds, pharmaceutical compositions, or medicaments described herein in combination with at least one additional therapeutic agent selected from the group consisting of alemtuzumab, arsenic trioxide, asparaginase (pegylated or non-), bevacizumab, cetuximab, platinum-based compounds such as cisplatin, cladribine, daunorubicin/doxorubicin/idarubicin, irinotecan, fludarabine, 5-fluorouracil, gemtuzumab, methotrexate, Paclitaxel™, taxol, temozolomide, thioguanine, or classes of drugs including hormones (an antiestrogen, an antiandrogen, or gonadotropin releasing hormone analogues, interferons such as alpha interferon, nitrogen mustards such as busulfan or melphalan or mechlorethamine, retinoids such as tretinoin, topoisomerase inhibitors such as irinotecan or topotecan, tyrosine kinase inhibitors such as gefinitinib or imatinib, or agents to treat signs or symptoms induced by such therapy including allopurinol, filgrastim, granisetron/ondansetron/palonosetron, and dronabinol.

### Kits/Articles of Manufacture

For use in the therapeutic applications described herein, kits and articles of manufacture are also described herein. In various embodiments, such kits comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In some embodiments, the containers are formed from a variety of materials such as glass or plastic.

In some embodiments, the articles of manufacture provided herein contain packaging materials. Packaging materials for use in packaging pharmaceutical products include, but are not limited to, blister packs, bottles, tubes, inhalers, pumps, bags, vials, containers, syringes, bottles, and any packaging material suitable for a selected formulation and intended mode of administration and treatment.

In some embodiments, the container(s) described herein comprise one or more compounds described herein, optionally in a composition or in combination with another agent as disclosed herein. The container(s) optionally have a sterile access port (for example in some embodiments the container is an intravenous solution bag or a vial having a stopper pierceable by a hypodermic injection needle). Such kits optionally comprise a compound with an identifying description or label or instructions relating to its use in the methods described herein.

In some embodiments, a kit will comprises one or more additional containers, each with one or more of various materials (such as reagents, optionally in concentrated form, and/or devices) desirable from a commercial and user standpoint for use of a compound described herein. Non-limiting examples of such materials include, but are not limited to, buffers, diluents, filters, needles, syringes; carrier, package, container, vial and/or tube labels listing contents and/or instructions for use, and package inserts with instructions for use. A set of instructions is optionally included.

In certain embodiments, a label is on or associated with the container. In some embodiments, a label is on a container when letters, numbers or other characters forming the label are attached, molded or etched into the container itself; a label is associated with a container when it is present within a receptacle or carrier that also holds the container, e.g., as a package insert. In certain embodiments, a label indicates that the contents are to be used for a specific therapeutic application. In some embodiments, the label indicates directions for use of the contents, such as in the methods described herein.

In certain embodiments, the pharmaceutical compositions are presented in a pack or dispenser device which contains one or more unit dosage forms containing a compound provided herein. In some embodiments, the pack contains a metal or plastic foil, such as a blister pack. The pack or dispenser device is optionally accompanied by instructions for administration. In some embodiments, the pack or dispenser is accompanied with a notice associated with the container in form prescribed by a governmental agency regulating the manufacture, use, or sale of pharmaceuticals, which notice is reflective of approval by the agency of the form of the drug for human or veterinary administration. In certain embodiments, such notice is, for example, the labeling approved by the U.S. Food and Drug Administration for prescription drugs, or the approved product insert. In some embodiments, compositions containing a compound provided herein are formulated in a compatible pharmaceutical carrier and are placed in an appropriate container labeled for treatment of an indicated condition.

### Preparation of Compounds Described Herein

In certain embodiments, the compounds described herein are synthesized using any synthetic techniques including standard synthetic techniques and the synthetic processes described herein. In specific embodiments, the following synthetic processes are utilized. In particular, the synthetic methods described in WO 2010/017055 can be used to prepare a compound described herein.

Formation of Covalent Linkages by Reaction of an Electrophile with a Nucleophile

Selected examples of covalent linkages and precursor functional groups which yield them are given in the Table entitled “Examples of Covalent Linkages and Precursors Thereof.” Precursor functional groups are shown as electrophilic groups and nucleophilic groups. In certain embodiments, a functional group on an organic substance is attached directly, or attached via any useful spacer or linker as defined below.

TABLE 1
Examples of Covalent Linkages and Precursors Thereof

| Covalent Linkage Product | Electrophile | Nucleophile |
|---|---|---|
| esters | acyl halides | alcohols/phenols |
| esters | acyl nitriles | alcohols/phenols |
| alkyl amines | alkyl halides | amines/anilines |
| esters | alkyl halides | carboxylic acids |
| esters | alkyl sulfonates | carboxylic acids |
| esters | anhydrides | alcohols/phenols |
| esters | carboxylic acids | alcohols |
| esters | diazoalkanes | carboxylic acids |
| alkyl amines | sulfonate esters | amines/anilines |
| esters | sulfonate esters | carboxylic acids |

In general, carbon electrophiles are susceptible to attack by complementary nucleophiles, including carbon nucleophiles, where an attacking nucleophile brings an electron pair to the carbon electrophile in order to form a new bond between the nucleophile and the carbon electrophile.

Suitable carbon nucleophiles include, but are not limited to alkyl, alkenyl, aryl and alkynyl Grignard, organolithium, organozinc, alkyl-, alkenyl, aryl- and alkynyl-tin reagents (organostannanes), alkyl-, alkenyl-, aryl- and alkynyl-borane reagents (organoboranes and organoboronates); these carbon nucleophiles have the advantage of being kinetically stable in water or polar organic solvents. Other carbon nucleophiles include phosphorus ylids, enol and enolate reagents; these carbon nucleophiles have the advantage of being relatively easy to generate from precursors. Carbon nucleophiles, when used in conjunction with carbon electrophiles, engender new carbon-carbon bonds between the carbon nucleophile and carbon electrophile.

Non-carbon nucleophiles suitable for coupling to carbon electrophiles include but are not limited to primary and secondary amines, thiols, thiolates, and thioethers, alcohols, alkoxides, azides, semicarbazides, and the like. These non-carbon nucleophiles, when used in conjunction with carbon electrophiles, typically generate heteroatom linkages (C—X—C), where X is a heteroatom, e. g, oxygen or nitrogen.

### Use of Protecting Groups

The term “protecting group” refers to chemical moieties that block some or all reactive moieties and prevent such groups from participating in chemical reactions until the protective group is removed. In specific embodiments, more than one protecting group is utilized. In more specific embodiments, each protective group is removable by a different process. Protective groups that are cleaved under totally disparate reaction conditions fulfill the requirement of differential removal. In various embodiments, protective groups are removed by acid, base, or hydrogenolysis. Groups such as trityl, dimethoxytrityl, acetal and t-butyldimethylsilyl are acid labile and are, in some embodiments, used to protect carboxy and hydroxy reactive moieties in the presence of amino groups protected with Cbz groups, which are removable by hydrogenolysis, and Fmoc groups, which are base labile. In some embodiments, carboxylic acid and hydroxy reactive moieties are blocked with base labile groups such as, without limitation, methyl, ethyl, and acetyl in the presence of amines blocked with acid labile groups such as t-butyl carbamate or with carbamates that are both acid and base stable but hydrolytically removable.

In certain embodiments, carboxylic acid and hydroxy reactive moieties are blocked with hydrolytically removable protective groups such as the benzyl group, while, in some embodiments, amine groups capable of hydrogen bonding with acids are blocked with base labile groups such as Fmoc. In various embodiments, carboxylic acid reactive moieties are protected by conversion to simple ester derivatives as exemplified herein, or they are blocked with oxidatively-removable protective groups such as 2,4-dimethoxybenzyl, while, in some embodiments, co-existing amino groups are blocked with fluoride labile silyl carbamates.

In certain instances, allyl blocking groups are useful in the presence of acid- and base-protecting groups since the former are stable. In some embodiments, such groups are subsequently removed by metal or pi-acid catalysts. For example, in some embodiments, an allyl-blocked carboxylic acid is deprotected with a Pd-catalyzed reaction in the presence of acid labile t-butyl carbamate or base-labile acetate amine protecting groups. In some embodiments, a protecting group is a resin to which a compound or intermediate is attached. As long as the residue is attached to the resin, that functional group is blocked and cannot react. Once released from the resin, the functional group is available to react.

In some embodiments, blocking/protecting groups are selected from, by way of non-limiting example:

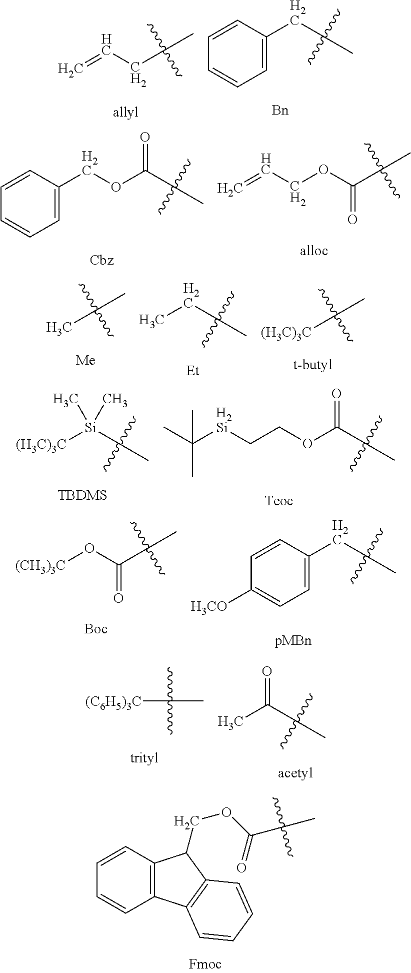

Other protecting groups are described in Greene and Wuts, Protective Groups in Organic Synthesis, 3rd Ed., John Wiley & Sons, New York, N.Y., 1999.

### EXAMPLES

The following Examples are intended as an illustration of the various embodiments disclosed herein. In certain embodiments, the compounds are prepared by a variety of synthetic routes. These examples are not intended, nor are they to be construed, as limiting the scope of the invention. All publications, patents, and patent applications cited herein are hereby incorporated by reference in their entireties for all purposes.

### Example A

### Parenteral Composition

To prepare a parenteral pharmaceutical composition suitable for administration by injection, 100 mg of a water-soluble salt of a compound described herein is dissolved in DMSO and then mixed with 10 mL of 0.9% sterile saline. The mixture is incorporated into a dosage unit form suitable for administration by injection.

### Example B

### Oral Composition

To prepare a pharmaceutical composition for oral delivery, 100 mg of a compound described herein is mixed with 750 mg of starch. The mixture is incorporated into an oral dosage unit for, such as a hard gelatin capsule, which is suitable for oral administration.

### Example C

### Sublingual (Hard Lozenge) Composition

To prepare a pharmaceutical composition for buccal delivery, such as a hard lozenge, mix 100 mg of a compound described herein, with 420 mg of powdered sugar mixed, with 1.6 mL of light corn syrup, 2.4 mL distilled water, and 0.42 mL mint extract. The mixture is gently blended and poured into a mold to form a lozenge suitable for buccal administration.

### Example D

### Inhalation Composition

To prepare a pharmaceutical composition for inhalation delivery, 20 mg of a compound described herein is mixed with 50 mg of anhydrous citric acid and 100 mL of 0.9% sodium chloride solution. The mixture is incorporated into an inhalation delivery unit, such as a nebulizer, which is suitable for inhalation administration.

### Example E

### Rectal Gel Composition

To prepare a pharmaceutical composition for rectal delivery, 100 mg of a compound described herein is mixed with 2.5 g of methylcelluose (1500 mPa), 100 mg of methylparapen, 5 g of glycerin and 100 mL of purified water. The resulting gel mixture is then incorporated into rectal delivery units, such as syringes, which are suitable for rectal administration.

### Example F

### Topical Gel Composition

To prepare a pharmaceutical topical gel composition, 100 mg of a compound described herein is mixed with 1.75 g of hydroxypropyl cellulose, 10 mL of propylene glycol, 10 mL of isopropyl myristate and 100 mL of purified alcohol USP. The resulting gel mixture is then incorporated into containers, such as tubes, which are suitable for topical administration.

### Example G

### Ophthalmic Solution Composition

To prepare a pharmaceutical ophthalmic solution composition, 100 mg of a compound described herein is mixed with 0.9 g of NaCl in 100 mL of purified water and filtered using a 0.2 micron filter. The resulting isotonic solution is then incorporated into ophthalmic delivery units, such as eye drop containers, which are suitable for ophthalmic administration.

### SYNTHETIC EXAMPLES

### Example 1

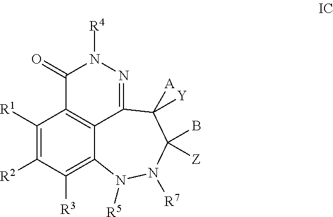

### Synthesis of tert-butyl 9-fluoro-3-(4-fluorophenyl)-4-(1-methyl-1H-1,2,4-triazol-5-yl)-7-oxo-3,4,6,7-tetrahydro-[1,2]diazepino[5,4,3-de]phthalazine-2(1H)-carboxylate (A)

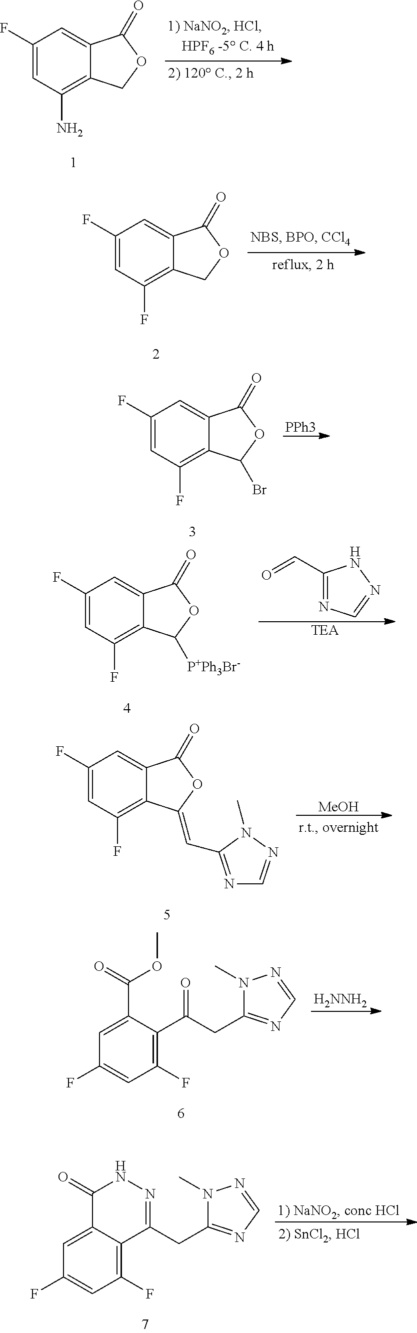

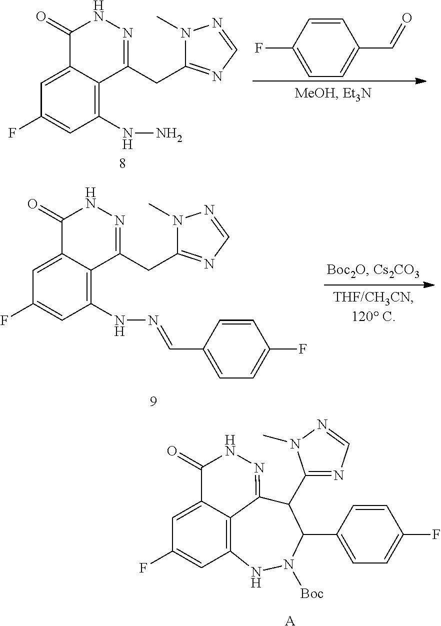

### Preparation of 4,6-difluoroisobenzofuran-1(3H)-one (2)

To a solution of 4-amino-6-fluoroisobenzofuran-1(3H)-one (1 g, 6 mmol) in concentrated HCl (12 mL) and water (30 mL) and cooled to −10 to 0° C., was added aqueous solution of NaNO₂ (480 mg, 6.64 mmol) dropwise. After the addition, 60% HPF₆ (60% solution in water) was added rapidly in one portion. The mixture was stirred for 4 hours and cooled to 0° C., the phosphorous hexafluoride salt was collected by filtration and washed with cold water followed by a mixture of diethyl ether and methanol (4:1). The solid was dried in vacuum over night to afford 800 mg of salt (yield 41%). Decomposition of the salt (3.4 g, 10.5 mmol) was carried out by portion-wise addition through the condenser fitted with a flask held at 120-150° C. After the decomposition was completed, the residue was extracted with ethyl acetate (20 mL×4) and the organic phase was washed with water and brine dried over sodium sulfate, concentrated to give crude product, which was purified by silica gel column (hexane:ethyl acetate 10:1) to give 4,6-difluoroisobenzofuran-1(3H)-one (2) (700 mg, yield 39%).MS (ESI) m/z 171(M+1).

### Preparation of 3-bromo-4,6-difluoroisobenzofuran-1(3H)-one (3)

4,6-Difluoroisobenzofuran-1(3H)-one (2) (170 g, 1 mmol), NBS (213 mg, 1.2 mmol) and benzoyl peroxide (BPO) (24 mg, 0.1 mmol) were dissolved in CCl₄ (10 mL) and refluxed for 2 h. Water was then added to the mixture and extracted with DCM (20 mL×3). The organic layer was washed with water and brine, dried over Na₂SO₄, concentrated to give 3-bromo-4,6-difluoroisobenzofuran-1(3H)-one (3), which was used in next step without further purification (250 mg, yield 100%).

### Preparation of (5,7-difluoro-3-oxo-1,3-dihydroisobenzofuran-1-yl)triphenylphosphonium bromide (4)

3-Bromo-4,6-difluoroisobenzofuran-1(3H)-one (3), (250 mg, 1 mmol) and PPh₃ (314 mg, 1.2 mmol) were dissolved in toluene (10 mL) and stirred for 3 hours under nitrogen at 100° C. Then the mixture was cooled to room temperature and filtered, washed with toluene (20 mL) and dried under vacuum to give (5,7-difluoro-3-oxo-1,3-dihydroisobenzofuran-1-yl)triphenylphosphonium bromide (4) (150 mg). MS (ESI) m/z 431(M+1).

### Preparation of (Z)-4,6-difluoro-3-((1-methyl-1H-1,2,4-triazol-5-yl)methylene)isobenzofuran-1(3H)-one (5)

To a solution of (5,7-difluoro-3-oxo-1,3-dihydroisobenzofuran-1-yl)triphenylphosphonium bromide (4) (260 mg, 0.5 mmol) and 2-methyl-2H-1,2,4-triazole-3-carbaldehyde (90 mg, 0.8 mmol) in DCM (6 mL) was added TEA (50 mg, 0.5 mmol) slowly at 0° C. After the addition the mixture was stirred at 10° C. for 3.5 hours. DCM was removed by concentration and the residue was washed with 2-Me-THF (10 mL×2) to obtain (Z)-4,6-difluoro-3-((1-methyl-1H-1,2,4-triazol-5-yl)methylene)isobenzofuran-1(3H)-one (5) as a yellow solid. LC-MS (ESI) m/z: 264 (M+1)⁺.

### Preparation of methyl 3,5-difluoro-2-(2-(1-methyl-1H-1,2,4-triazol-5-yl)acetyl)benzoate (6)

(Z)-4,6-difluoro-3-((1-methyl-1H-1,2,4-triazol-5-yl)methylene)isobenzofuran-1(3H)-one (5) (11.5 g, 43.7 mmol) was suspended in 250 mL of methanol and stirred at room temperature overnight. TLC (Petroleum ether:EtOAc=2:1) and LC-MS (acidic mobile phase) showed the starting material was consumed completely and the solution turned clear. Then reaction mixture was evaporated to dryness to obtain methyl 3,5-difluoro-2-(2-(1-methyl-1H-1,2,4-triazol-5-yl)acetyl)benzoate (6) as yellow solid (12.8 g, yield 99%). LC-MS (ESI) m/z: 297 (M+1)⁺.

### Preparation of 5,7-Difluoro-4-((1-methyl-1H-1,2,4-triazol-5-yl)methyl)phthalazin-1(2H)-one (7)

To a solution of (Z)-6-fluoro-3-((2-methyl-2H-1,2,4-triazol-3-yl)methylene)-4-nitroiso-benzofuran-1(3H)-one (4.0 g, 135 mmol) in THF (100 mL) was added hydrazine monohydrate (6 mL) at room temperature under nitrogen atmosphere. Two hours later, acetic acid (6 mL) was added and the mixture was heated to 60° C. overnight. The mixture was diluted with water (100 ml), and extracted with EtOAc (100 ml×3). The organic layer was dried over anhydrous Na₂SO₄ and concentrated to afford the crude product as a yellow solid. It was then purified by prep-HPLC to obtain 7 as a yellow solid (800 mg, yield: 21%). LC-MS (m/z) 275[M+H]⁺.

### Preparation of 7-fluoro-5-hydrazinyl-4-((1-methyl-1H-1,2,4-triazol-5-yl)methyl)phthalazin-1(2H)-one (8)

Compound 8 (300 mg, 1.09 mmol) was dissolved in conc. HCl (0.75 mL) in a microwave tube, then NaNO₂ (78 mg, 1.09 mmol) in 0.36 mL of H₂O was added dropwise in ice bath to control the temperature below 5° C. The reaction mixture was stirred at 0° C. for 1.5 hours and then SnCl₂ (624 mg, 3.28 mmol) in conc. HCl (0.18 mL) was added at 5° C. dropwise. The resulting mixture was stirred at 0° C. for 1 hour, and filtered to obtain yellow solid 8 (240 mg, yield: 77%).

### Preparation of (E)-7-fluoro-5-(2-(4-fluorobenzylidene)hydrazinyl)-4-((1-methyl-1H-1,2,4-triazol-5-yl)methyl)phthalazin-1(2H)-one (9)

To a solution of 4-fluorobenzaldehyde (210 mg, 1.66 mmol) and Et₃N (0.36 mL) in MeOH (8 mL) was added 8 (240 mg, 0.83 mmol) in MeOH (6 mL) dropwise at 29° C. under nitrogen atmosphere. Then the reaction was stirred at 29° C. for 12 hours. The mixture was filtered to obtain yellow solid 9 (320 mg, yield: 98%). LC-MS (m/z) 396[M+H]⁺. ¹H-NMR (400 MHz, DMSO-d₆) δ (ppm): 3.86 (s, 3H), 4.74 (s, 2H), 7.26-7.31 (t, J=8.4 Hz, 2H), 7.45 (d, J=8.4 Hz, 1H), 7.83-7.88 (m, 4H), 8.31 (s, 1H), 11.01 (s, 1H), 12.63 (s, 1H).

### Preparation of tert-butyl 9-fluoro-3-(4-fluorophenyl)-4-(1-methyl-1H-1,2,4-triazol-5-yl)-7-oxo-3,4,6,7-tetrahydro-[1,2]diazepino[5,4,3-de]phthalazine-2(1H)-carboxylate (A)

To a solution of 9 (56 mg, 0.14 mmol) and Cs₂CO₃ (136 mg, 0.42 mmol) in THF (1 mL) and CH₃CN (1 mL) was added Boc₂O (92 mg, 0.42 mmol) at 120° C. for 29 min. The mixture was diluted with water (10 ml), and extracted with EtOAc (10 ml×3). The organic layer was dried over anhydrous Na₂SO₄ and concentrated to afford the crude product, which was then purified by prep-TLC (hexane:ethyl acetate=1:1) to obtain A (4.8 mg, yield: 7%). LC-MS (m/z) 496[M+H]⁺. ¹H-NMR (400 MHz, MeOH-d₄) δ (ppm): 1.49 (s, 9H), 3.78 (s, 3H), 4.26-4.37 (q, 2H), 7.07-7.11 (m, 3H), 7.52 (s, 1H), 7.63-7.71 (m, 3H), 8.28-8.31 (m, 1H).

### Example 2

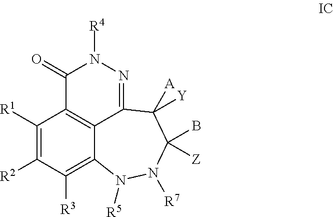

### Alternate Synthesis of 9-fluoro-3-(4-fluorophenyl)-4-(1-methyl-1H-1,2,4-triazol-5-yl)-1,2,3,4-tetrahydro-[1,2]diazepino[5,4,3-de]phthalazin-7(6H)-one (A)

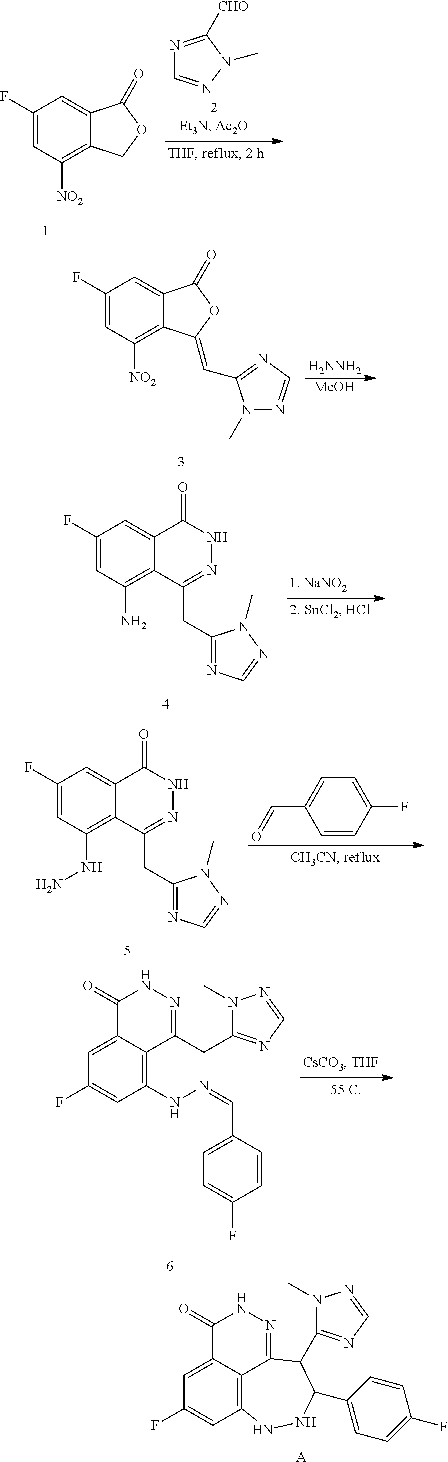

### Preparation of (Z)-6-fluoro-3-((1-methyl-1H-1,2,4-triazol-5-yl)methylene)-4-nitroisobenzofuran-1(3H)-one (3)

To a solution of 6-fluoro-4-nitroisobenzofuran-1(3H)-one (1) (10 g, 50.8 mmol), 2-methyl-2H-1,2,4-triazole-3-carbaldehyde (2) (11 g, 101.5 mmol) and Et₃N (20 mL, 152.4 mmol) in anhydrous THF (150 mL) was added acetic anhydride (35 mL) drop-wise with stirring at room temperature in 3 minutes. The mixture was then stirred at reflux for 45 minutes when a green solid was precipitated from solvent. The solvent was concentrated to 15 mL and the resulting mixture was cooled to 0° C. and filtered, washed with 15 mL of ethyl acetate to obtain (Z)-6-fluoro-3((1-methyl-1H-1,2,4-triazol-5-yl)methylene)-4-nitroisobenzofuran-1(3H)-one (3) as a green solid (11.5 g, yield: 78%). LC-MS (ESI) m/z: 291 (M+1)⁺. ¹H-NMR (400 MHz, DMSO-d₆) δ (ppm): 3.94 (s, 3H), 7.15 (s, 1H), 8.10 (s, 1H), 8.40-8.42 (dd, J₁=6.4 Hz, J₂=2.4 Hz, 1H), 8.58-8.61 (dd, J₁=8.8 Hz, J₂=2.4 Hz, 1H).

### Preparation of 5-amino-7-fluoro-4-((1-methyl-1H-1,2,4-triazol-5-yl)methyl)phthalazin-1(2H)-one (4)

To a solution of (Z)-6-fluoro-3-((1-methyl-1H-1,2,4-triazol-5-yl)methylene)-4-nitroisobenzofuran-1(3H)-one (3) (4.0 g, 13.8 mmol) in THF (100 mL) was added hydrazine monohydrate (6 mL) at room temperature under nitrogen atmosphere and the mixture was allowed to stir for 2 hours. Acetic acid (6 mL) was added and the mixture was heated to 60° C. overnight. The resulting mixture was diluted with water (100 mL) and extracted with EtOAc (100 mL×3). The organic layer was dried over anhydrous Na₂SO₄ and concentrated to afford 5-amino-7-fluoro-4-((1-methyl-1H-1,2,4-triazol-5-yl)methyl)phthalazin-1(2H)-one (4) as a yellow solid (1.6 g, yield 42%). LC-MS (ESI) m/z: 275(M+1)⁺.

The following steps were not, but could be, conducted as described below.

### Preparation of 7-fluoro-5-hydrazinyl-4-((1-methyl-1H-1,2,4-triazol-5-yl)methyl)phthalazin-1(2H)-one (5)

To a solution of 5-amino-7-fluoro-4-((1-methyl-1H-1,2,4-triazol-5-yl)methyl)phthalazin-1(2H)-one (4) (548 mg, 2.0 mmol) in dichloromethane (75 mL) is added NaNO₂ (550 mg, 8.0 mmol) and conc. HCl (0.5 mL) with stirring at 0° C. The mixture is warmed up to room temperature and stirred for 2 hours. It is transferred to a separatory funnel and washed with water (25 mL×3). The organic layer is then evaporated to dryness on a rotary evaporator. The residue is re-dissolved in THF (75 mL). To this solution SnCl, (1.5 g, 8.0 mmol) and conc. HCl (1 mL) are added with stirring at room temperature. After stirring for 4 hours, the mixture is concentrated to a volume of 15 mL and then diluted with ethyl acetate (100 mL). This solution is washed with dilute aqueous HCl (50 mL×3), saturated aqueous sodium bicarbonate (50 mL×3), and brine. Column chromatography purification on silica gel affords 7-fluoro-5-hydrazinyl-4-((1-methyl-1H-1,2,4-triazol-5-yl)methyl)phthalazin-1(2H)-one (5) as a solid. Expected LC-MS (ESI) m/z: 290 (M+H)⁺.

### Preparation of (E)-7-fluoro-5-(2-(4-fluorobenzylidene)hydrazinyl)-4-((1-methyl-1H-1,2,4-triazol-5-yl)methyl)phthalazin-1(2H)-one (6)

To a suspension of 7-fluoro-5-hydrazinyl-4-((1-methyl-1H-1,2,4-triazol-5-yl)methyl)phthalazin-1(2H)-one (5) (189 mg, 1.0 mmol) in acetonitrile (20 mL) is added 4-fluorobenzaldehyde (248 mg, 2.0 mmol) and stirred under reflux for 48 hours. The precipitate is filtered and washed with 10 mL of EtOAc/hexane (1:1) to afford (E)-7-fluoro-5-(2-(4-fluorobenzylidene)hydrazinyl)-4-((1-methyl-1H-1,2,4-triazol-5-yl)methyl)phthalazin-1(2H)-one (6) as a solid. Expected LC-MS (ESI) m/z: 396 (M+1)⁺.

### Preparation of 9-fluoro-3-(4-fluorophenyl)-4-(1-methyl-1H-1,2,4-triazol-5-yl)-1,2,3,4-tetrahydro-[1,2]diazepino[5,4,3-de]phthalazin-7(6H)-one (A)

To a suspension of (E)-7-fluoro-5-(2-(4-fluorobenzylidene)hydrazinyl)-4-((1-methyl-1H-1,2,4-triazol-5-yl)methyl)phthalazin-1(2H)-one (6) (158 mg, 0.4 mmol) in THF (20 mL) is added cesium carbonate (261 mg, 0.8 mmol) and stirred at 55° C. for 4 hours. The mixture is then diluted with water (20 mL) and extracted with EtOAc (30 mL×3). The organic layer is dried over anhydrous Na₂SO₄ and concentrated to afford 9-fluoro-3-(4-fluorophenyl)-4-(1-methyl-1H-1,2,4-triazol-5-yl)-1,2,3,4-tetrahydro-[1,2]diazepino[5,4,3-de]phthalazin-7(6H)-one (A) as a solid. Expected LC-MS (ESI) m/z: 396(M+1)⁺.

Using analogous methods and substituting with appropriate starting materials, the following compounds can be prepared using the procedures as described above in Examples 1 and 2.

9-fluoro-3-(4-fluorophenyl)-4-(1H-1,2,4-triazol-5-yl)-1,2,3,4-tetrahydro-[1,2]diazepino[5,4,3-de]phthalazin-7(6H)-one;

9-fluoro-3-(4-fluorophenyl)-4-(1-methyl-1H-1,2,4-triazol-5-yl)-1,2,3,4-tetrahydro-[1,2]diazepino[5,4,3-de]phthalazin-7(6H)-one;

4-(1-ethyl-1H-1,2,4-triazol-5-yl)-9-fluoro-3-(4-fluorophenyl)-1,2,3,4-tetrahydro-[1,2]diazepino[5,4,3-de]phthalazin-7(6H)-one;

9-fluoro-3-(4-fluorophenyl)-4-(1-propyl-1H-1,2,4-triazol-5-yl)-1,2,3,4-tetrahydro-[1,2]diazepino[5,4,3-de]phthalazin-7(6H)-one;

9-fluoro-3-(4-fluorophenyl)-4-(1-(2-hydroxyethyl)-1H-1,2,4-triazol-5-yl)-1,2,3,4-tetrahydro-[1,2]diazepino[5,4,3-de]phthalazin-7(6H)-one;

9-fluoro-3-(4-fluorophenyl)-4-(1-(3-hydroxypropyl)-1H-1,2,4-triazol-5-yl)-1,2,3,4-tetrahydro-[1,2]diazepino[5,4,3-de]phthalazin-7(6H)-one;

9-fluoro-3-phenyl-4-(1H-1,2,4-triazol-5-yl)-1,2,3,4-tetrahydro-[1,2]diazepino[5,4,3-de]phthalazin-7(6H)-one;

9-fluoro-4-(1-methyl-1H-1,2,4-triazol-5-yl)-3-phenyl-1,2,3,4-tetrahydro-[1,2]diazepino[5,4,3-de]phthalazin-7(6H)-one;

4-(1-ethyl-1H-1,2,4-triazol-5-yl)-9-fluoro-3-phenyl-1,2,3,4-tetrahydro-[1,2]diazepino[5,4,3-de]phthalazin-7(6H)-one;

9-fluoro-3-phenyl-4-(1-propyl-1H-1,2,4-triazol-5-yl)-1,2,3,4-tetrahydro-[1,2]diazepino[5,4,3-de]phthalazin-7(6H)-one;

9-fluoro-4-(1-(2-hydroxyethyl)-1H-1,2,4-triazol-5-yl)-3-phenyl-1,2,3,4-tetrahydro-[1,2]diazepino[5,4,3-de]phthalazin-7(6H)-one;

9-fluoro-4-(1-(3-hydroxypropyl)-1H-1,2,4-triazol-5-yl)-3-phenyl-1,2,3,4-tetrahydro-[1,2]diazepino[5,4,3-de]phthalazin-7(6H)-one;

9-fluoro-3-(4-((methylamino)methyl)phenyl)-4-(1H-1,2,4-triazol-5-yl)-1,2,3,4-tetrahydro-[1,2]diazepino[5,4,3-de]phthalazin-7(6H)-one;

9-fluoro-4-(1-methyl-1H -1,2,4-triazol-5-yl)-3-(4-((methylamino)methyl)phenyl)-1,2,3,4-tetrahydro-[1,2]diazepino[5,4,3-de]phthalazin-7(6H)-one;

4-(1-ethyl-1H-1,2,4-triazol-5-yl)-9-fluoro-3-(4-((methylamino)methyl)phenyl)-1,2,3,4-tetrahydro-[1,2]diazepino[5,4,3-de]phthalazin-7(6H)-one;

9-fluoro-4-(1-(2-hydroxyethyl)-1H-1,2,4-triazol-5-yl)-3-(4-((methylamino)methyl)phenyl)-1,2,3,4-tetrahydro-[1,2]diazepino[5,4,3-de]phthalazin-7(6H)-one;

9-fluoro-4-(1-(3-hydroxypropyl)-1H-1,2,4-triazol-5-yl)-3-(4-((methylamino)methyl)phenyl)-1,2,3,4-tetrahydro-[1,2]diazepino[5,4,3-de]phthalazin-7(6H)-one;

3-(4-((dimethylamino)methyl)phenyl)-9-fluoro-4-(1H-1,2,4-triazol-5-yl)-1,2,3,4-tetrahydro-[1,2]diazepino[5,4,3-de]phthalazin-7(6H)-one;

3-(4-((dimethylamino)methyl)phenyl)-9-fluoro-4-(1-methyl-1H-1,2,4-triazol-5-yl)-1,2,3,4-tetrahydro-[1,2]diazepino[5,4,3-de]phthalazin-7(6H)-one;

3-(4-((dimethylamino)methyl)phenyl)-4-(1-ethyl-1H-1,2,4-triazol-5-yl)-9-fluoro-1,2,3,4-tetrahydro-[1,2]diazepino[5,4,3-de]phthalazin-7(6H)-one;

3-(4-((dimethylamino)methyl)phenyl)-9-fluoro-4-(1-(2-hydroxyethyl)-1H-1,2,4-triazol-5-yl)-1,2,3,4-tetrahydro-[1,2]diazepino[5,4,3-de]phthalazin-7(6H)-one;

3-(4-((dimethylamino)methyl)phenyl)-9-fluoro-4-(1-(3-hydroxypropyl)-1H-1,2,4-triazol-5-yl)-1,2,3,4-tetrahydro-[1,2]diazepino[5,4,3-de]phthalazin-7(6H)-one;

3-(4-(azetidin-1-ylmethyl)phenyl)-9-fluoro-4-(1H-1,2,4-triazol-5-yl)-1,2,3,4-tetrahydro-[1,2]diazepino[5,4,3-de]phthalazin-7(6H)-one;

3-(4-(azetidin-1-ylmethyl)phenyl)-9-fluoro-4-(1-methyl-1H-1,2,4-triazol-5-yl)-1,2,3,4-tetrahydro-[1,2]diazepino[5,4,3-de]phthalazin-7(6H)-one;

3-(4-(azetidin-1-ylmethyl)phenyl)-4-(1-ethyl-1H-1,2,4-triazol-5-yl)-9-fluoro-1,2,3,4-tetrahydro-[1,2]diazepino[5,4,3-de]phthalazin-7(6H)-one;

3-(4-(azetidin-1-ylmethyl)phenyl)-9-fluoro-4-(1-propyl-1H-1,2,4-triazol-5-yl)-1,2,3,4-tetrahydro-[1,2]diazepino[5,4,3-de]phthalazin-7(6H)-one;

3-(4-(azetidin-1-ylmethyl)phenyl)-9-fluoro-4-(1-(2-hydroxyethyl)-1H-1,2,4-triazol-5-yl)-1,2,3,4-tetrahydro-[1,2]diazepino[5,4,3-de]phthalazin-7(6H)-one;

3-(4-(azetidin-1-ylmethyl)phenyl)-9-fluoro-4-(1-(3-hydroxypropyl)-1H-1,2,4-triazol-5-yl)-1,2,3,4-tetrahydro-[1,2]diazepino[5,4,3-de]phthalazin-7(6H)-one;

3-(4-fluorophenyl)-4-(1H-1,2,4-triazol -5-yl)-1,2,3,4-tetrahydro-[1,2]diazepino[5,4,3-de]phthalazin-7(6H)-one;

3-(4-fluorophenyl)-4-(1-methyl-1H-1,2,4-triazol-5-yl)-1,2,3,4-tetrahydro-[1,2]diazepino[5,4,3-de]phthalazin-7(6H)-one;

3-(4-fluorophenyl)-4-(1-(2-hydroxyethyl)-1H-1,2,4-triazol-5-yl)-1,2,3,4-tetrahydro-[1,2]diazepino[5,4,3-de]phthalazin-7(6H)-one;

3-(4-fluorophenyl)-4-(1-(3-hydroxypropyl)-1H-1,2,4-triazol-5-yl)-1,2,3,4-tetrahydro-[1,2]diazepino [5,4,3-de]phthalazin-7(6H)-one;

4-(1-(2-hydroxyethyl)-1H-1,2,4-triazol-5-yl)-3-(4-((methylamino)methyl)phenyl)-1,2,3,4-tetrahydro-[1,2]diazepino[5,4,3-de]phthalazin-7(6H)-one;

4-(1-methyl-1H-1,2,4-triazol-5-yl)-3-(4-((methylamino)methyl)phenyl)-1,2,3,4-tetrahydro-[1,2]diazepino[5,4,3-de]phthalazin-7(6H)-one;

3-(4-((methylamino)methyl)phenyl)-4-(1H-1,2,4-triazol-5-yl)-1,2,3,4-tetrahydro-[1,2]diazepino[5,4,3-de]phthalazin-7(6H)-one;

3-(4-(azetidin-1-ylmethyl)phenyl)-4-(1-(2-hydroxyethyl)-1H-1,2,4-triazol-5-yl)-1,2,3,4-tetrahydro-[1,2]diazepino[5,4,3-de]phthalazin-7(6H)-one;

3-(4-(azetidin-1-ylmethyl)phenyl)-4-(1-methyl-1H-1,2,4-triazol-5-yl)-1,2,3,4-tetrahydro-[1,2]diazepino[5,4,3-de]phthalazin-7(6H)-one; and

3-(4-(azetidin-1-ylmethyl)phenyl)-4-(1H-1,2,4-triazol-5-yl)-1,2,3,4-tetrahydro-[1,2]diazepino[5,4,3-de]phthalazin-7(6H)-one.

### Example 3

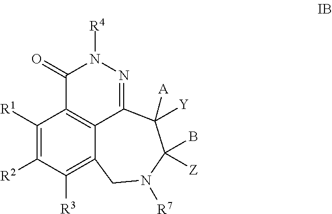

### Synthesis of 5-fluoro-9-(4-fluorophenyl)-10-(1-methyl-1H-1,2,4-triazol-5-yl)-7,8,9,10-tetrahydroazepino[5,4,3-de]phthalazin-3(2H)-one (B)

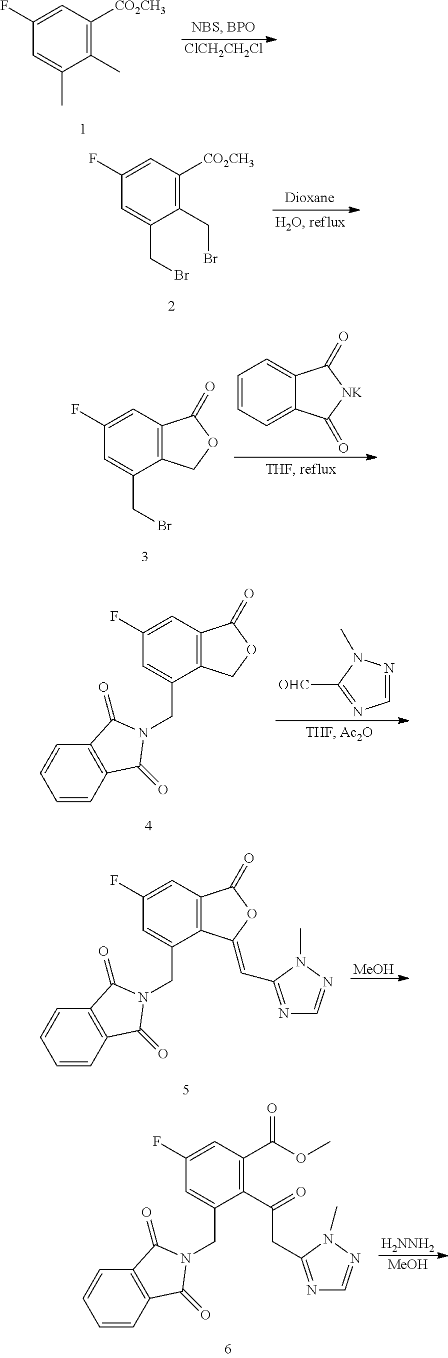

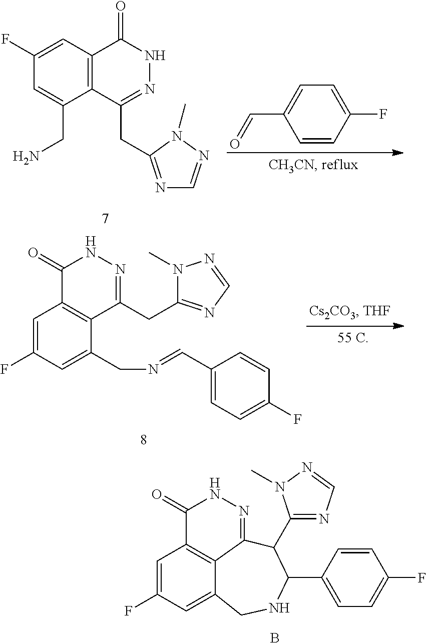

The following steps were not, but could be, conducted as described below.

### Preparation of methyl 2,3-bis(bromomethyl)-5-fluorobenzoate (2)

A mixture of methyl 5-fluoro-2,3-dimethylbenzoate (1) (24 g, 130 mmol), N-bromosuccinimide (NBS) (51 g, 286 mmol) and BPO (6.2 g, 26 mmol) in DCM (500 mL) is heated to reflux overnight. Water (200 mL) is added to the mixture. The organic layer is separated and the solvent is removed under reduced pressure. The residue is extracted with DCM (200 mL×3). The combined organic layers are washed with brine, dried over Na₂SO₄, concentrated to give crude product methyl 2,3-bis(bromomethyl)-5-fluorobenzoate (2) as a brown oil which is used in the next step without purification.

### Preparation of methyl 2,3-bis(bromomethyl)-5-fluorobenzoate (3)

A mixture of methyl 2,3-bis(bromomethyl)-5-fluorobenzoate (2) (36 g, 106 mmol) in 1,4-dioxane (250 mL) and water (62.5 mL) is heated to reflux for 4 days. Dioxane is removed under reduced pressure. The residue is extracted with EtOAc (300 mL×4). The combined organic layers are washed with brine, dried over Na₂SO₄, concentrated to give crude product. The crude product is purified by silica gel column chromatography (petroleum ether to petroleum ether/EtOAc=5:1) to give methyl 2,3-bis(bromomethyl)-5-fluorobenzoate (3) as a solid.

### Preparation of 2-((6-fluoro-1-oxo-1,3-dihydroisobenzofuran-4-yl)methyl)isoindoline-1,3-dione (4)

A solution of methyl 2,3-bis(bromomethyl)-5-fluorobenzoate (3) (15 g, 61 mmol) and potassium phthalimide (13.6 g, 73 mmol) in THF (150 mL) is heated to reflux under nitrogen for 6 hours. The mixture is then cooled to room temperature and washed with water (100 mL×3). The organic phase is dried over sodium sulfate and evaporated to dryness. The residue is purified with column chromatography on silica gel. This affords 2-((6-fluoro-1-oxo-1,3-dihydroisobenzofuran-4-yl)methyl)isoindoline-1,3-dione (4) as a solid. Expected LC-MS (ESI) m/z: 312 (M+H)⁺.

### Preparation of (Z)-2-((6-fluoro-3-((1-methyl-1H-1,2,4-triazol-5-yl)methylene)-1-oxo-1,3-dihydroisobenzofuran-4-yl)methyl)isoindoline-1,3-dione (5)

To a solution of 2-((6-fluoro-1-oxo-1,3-dihydroisobenzofuran-4-yl)methyl)isoindoline-1,3-dione (4) (13 g, 41.8 mmol), 2-methyl-2H-1,2,4-triazole-3-carbaldehyde (9.3 g, 83.6 mmol) and Et₃N (20 mL, 152.4 mmol) in anhydrous THF (150 mL) is added acetic anhydride (35 mL) drop-wise with stirring at room temperature for 5 minutes. Then the mixture is stirred under reflux for 1 hour, and a solid is precipitated out from the solvent. The solvent is concentrated to 15 mL, the resulting mixture is cooled to 0° C. and filtered, washed with 15 mL of ethyl acetate to obtain (Z)-2-((6-fluoro-3-((1-methyl-1H-1,2,4-triazol-5-yl)methylene)-1-oxo-1,3-dihydroisobenzofuran-4-yl)methyl)isoindoline-1,3-dione (5) as a solid. Expected LC-MS (ESI) m/z: 405 (M+1)⁺.

### Preparation of methyl 3-((1,3-dioxoisoindolin-2-yl)methyl)-5-fluoro-2-(2-(1-methyl-1H-1,2,4-triazol-5-yl)acetyl)benzoate (6)

(Z)-2-((6-Fluoro-3-((1-methyl-1H-1,2,4-triazol-5-yl)methylene)-1-oxo-1,3-dihydroisobenzofuran-4-yl)methyl)isoindoline-1,3-dione (5) (13 g, 32.2 mmol) is suspended in 250 mL of methanol and stirred at room temperature overnight. TLC (Petroleum ether:EtOAc=2:1) shows the starting material is consumed completely. The reaction mixture is evaporated to dryness to obtain methyl 3-((1,3-dioxoisoindolin-2-yl)methyl)-5-fluoro-2-(2-(1-methyl-1H-1,2,4-triazol-5-yl)acetyl)benzoate (6) as a solid. Expected LC-MS (ESI) m/z: 437 (M+1)⁺.

### Preparation of 5-(aminomethyl)-7-fluoro-4-((1-methyl-1H-1,2,4-triazol-5-yl)methyl)phthalazin-1(2H)-one (7)

To a solution of methyl 3-((1,3-dioxoisoindolin-2-yl)methyl)-5-fluoro-2-(2-(1-methyl-1H-1,2,4-triazol-5-yl)acetyl)benzoate (6) (4.4 g, 10.1 mmol) in THF (100 mL) is added hydrazine monohydrate (12 mL) at room temperature under nitrogen atmosphere and stirred for 2 hours, then acetic acid (12 mL) is added and the mixture was heated to 60° C. for 18 hours. The resulting mixture, after cooled to room temperature, is diluted with water (100 mL) and extracted with EtOAc (100 mL×3). The organic layer is dried over anhydrous Na₂SO₄ and concentrated to afford 5-(aminomethyl)-7-fluoro-4-((1-methyl-1H-1,2,4-triazol-5-yl)methyl)phthalazin-1(2H)-one (7) as a solid. Expected LC-MS (ESI) m/z: 289 (m+1)⁺.

### Preparation of (Z)-7-fluoro-5-(((4-fluorobenzylidene)amino)methyl)-4-((1-methyl-1H-1,2,4-triazol-5-yl)methyl)phthalazin-1(2H)-one (8)

To a suspension of 5-(aminomethyl)-7-fluoro-4-((1-methyl-1H-1,2,4-triazol-5-yl)methyl)phthalazin-1(2H)-one (7) (1.0 g, 3.5 mmol) in acetonitrile (50 mL) is added 4-fluorobenzaldehyde (1.3 g, 10.5 mmol). The mixture is stirred at reflux for 48 hours. The precipitate is filtered and washed with 10 mL of EtOAc/hexane (1:1) to afford (Z)-7-fluoro-5-(((4-fluorobenzylidene)amino)methyl)-4-((1-methyl-1H-1,2,4-triazol-5-yl)methyl)phthalazin-1(2H)-one (8) as a solid. Expected LC-MS (ESI) m/z: 395 (M+1)⁺.

### Preparation of 5-fluoro-9-(4-fluorophenyl)-10-(1-methyl-1H-1,2,4-triazol-5-yl)-7,8,9,10-tetrahydroazepino[5,4,3-de]phthalazin-3(2H)-one (B)

To a suspension of (Z)-7-fluoro-5-(((4-fluorobenzylidene)amino)methyl)-4-((1-methyl-1H-1,2,4-triazol-5-yl)methyl)phthalazin-1(2H)-one (8) (650 mg, 1.65 mmol) in THF (80 mL) is added cesium carbonate (1.07 g, 3.3 mmol) and stirred at 55° C. for 6 hours. Then the mixture, after cooled to room temperature, is diluted with water (50 mL) and extracted with EtOAc (50 mL×3). The organic layer is dried over anhydrous Na₂SO₄ and concentrated to afford 5-fluoro-9-(4-fluorophenyl)-10-(1-methyl-1H-1,2,4-triazol-5-yl)-7,8,9,10-tetrahydroazepino[5,4,3-de]phthalazin-3(2H)-one (B) as a solid. Expected LC-MS (ESI) m/z: 395 (M+1)⁺.

Using analogous methods and substituting with appropriate starting materials, the following compounds can be prepared using the procedures as described above.

5-fluoro-9-(4-fluorophenyl)-10-(1H-1,2,4-triazol-5-yl)-7,8,9,10-tetrahydroazepino[5,4,3-de]phthalazin-3(2H)-one;

5-fluoro-9-(4-fluorophenyl)-10-(1-methyl-1H-1,2,4-triazol-5-yl)-7,8,9,10-tetrahydroazepino[5,4,3-de]phthalazin-3(2H)-one;

10-(1-ethyl-1H-1,2,4-triazol-5-yl)-5-fluoro-9-(4-fluorophenyl)-7,8,9,10-tetrahydroazepino[5,4,3-de]phthalazin-3(2H)-one;

5-fluoro-9-(4-fluorophenyl)-10-(1-propyl-1H-1,2,4-triazol-5-yl)-7,8,9,10-tetrahydroazepino[5,4,3-de]phthalazin-3(2H)-one;

5-fluoro-9-(4-fluorophenyl)-10-(1-(2-hydroxyethyl)-1H-1,2,4-triazol-5-yl)-7,8,9,10-tetrahydroazepino[5,4,3-de]phthalazin-3(2H)-one;

5-fluoro-9-(4-fluorophenyl)-10-(1-(3-hydroxypropyl)-1H-1,2,4-triazol-5-yl)-7,8,9,10-tetrahydroazepino[5,4,3-de]phthalazin-3(2H)-one;

5-fluoro-9-phenyl-10-(1H-1,2,4-triazol-5-yl)-7,8,9,10-tetrahydroazepino[5,4,3-de]phthalazin-3(2H)-one;

5-fluoro-10-(1-methyl-1H-1,2,4-triazol-5-yl)-9-phenyl-7,8,9,10-tetrahydroazepino[5,4,3-de]phthalazin-3(2H)-one;

10-(1-ethyl-1H-1,2,4-triazol-5-yl)-5-fluoro-9-phenyl-7,8,9,10-tetrahydroazepino[5,4,3-de]phthalazin-3(2H)-one;

5-fluoro-9-phenyl-10-(1-propyl-1H-1,2,4-triazol-5-yl)-7,8,9,10-tetrahydroazepino[5,4,3-de]phthalazin-3(2H)-one;

5-fluoro-10-(1-(2-hydroxyethyl)-1H-1,2,4-triazol-5-yl)-9-phenyl-7,8,9,10-tetrahydroazepino[5,4,3-de]phthalazin-3(2H)-one;

5-fluoro-10-(1-(3-hydroxypropyl)-1H-1,2,4-triazol-5-yl)-9-phenyl-7,8,9,10-tetrahydroazepino[5,4,3-de]phthalazin-3(2H)-one;

5-fluoro-9-(4-((methylamino)methyl)phenyl)-10-(1H-1,2,4-triazol-5-yl)-7,8,9,10-tetrahydroazepino[5,4,3-de]phthalazin-3(2H)-one;

5-fluoro-10-(1-methyl-1H-1,2,4-triazol-5-yl)-9-(4-((methylamino)methyl)phenyl)-7,8,9,10-tetrahydroazepino[5,4,3-de]phthalazin-3(2H)-one;

10-(1-ethyl-1H-1,2,4-triazol-5-yl)-5-fluoro-9-(4-((methylamino)methyl)phenyl)-7,8,9,10-tetrahydroazepino[5,4,3-de]phthalazin-3(2H)-one;

5-fluoro-10-(1-(2-hydroxyethyl)-1H-1,2,4-triazol-5-yl)-9-(4-((methylamino)methyl)phenyl)-7,8,9,10-tetrahydroazepino[5,4,3-de]phthalazin-3(2H)-one;

5-fluoro-10-(1-(3-hydroxypropyl)-1H-1,2,4-triazol-5-yl)-9-(4-((methylamino)methyl)phenyl)-7,8,9,10-tetrahydroazepino[5,4,3-de]phthalazin-3(2H)-one;

9-(4-((dimethylamino)methyl)phenyl)-5-fluoro-10-(1H-1,2,4-triazol-5-yl)-7,8,9,10-tetrahydroazepino[5,4,3-de]phthalazin-3(2H)-one;

9-(4-((dimethylamino)methyl)phenyl)-5-fluoro-10-(1-methyl-1H-1,2,4-triazol-5-yl)-7,8,9,10-tetrahydroazepino[5,4,3-de]phthalazin-3(2H)-one;

9-(4-((dimethylamino)methyl)phenyl)-10-(1-ethyl-1H-1,2,4-triazol-5-yl)-5-fluoro-7,8,9,10-tetrahydroazepino[5,4,3-de]phthalazin-3(2H)-one;

9-(4-((dimethylamino)methyl)phenyl)-5-fluoro-10-(1-(2-hydroxyethyl)-1H-1,2,4-triazol-5-yl)-7,8,9,10-tetrahydroazepino[5,4,3-de]phthalazin-3(2H)-one;

9-(4-((dimethylamino)methyl)phenyl)-5-fluoro-10-(1-(3-hydroxypropyl)-1H-1,2,4-triazol-5-yl)-7,8,9,10-tetrahydroazepino[5,4,3-de]phthalazin-3(2H)-one;

9-(4-(azetidin-1-ylmethyl)phenyl)-5-fluoro-10-(1H-1,2,4-triazol-5-yl)-7,8,9,10-tetrahydroazepino[5,4,3-de]phthalazin-3(2H)-one;

9-(4-(azetidin-1-ylmethyl)phenyl)-5-fluoro-10-(1-methyl-1H-1,2,4-triazol-5-yl)-7,8,9,10-tetrahydroazepino[5,4,3-de]phthalazin-3(2H)-one;

9-(4-(azetidin-1-ylmethyl)phenyl)-10-(1-ethyl-1H-1,2,4-triazol-5-yl)-5-fluoro-7,8,9,10-tetrahydroazepino[5,4,3-de]phthalazin-3(2H)-one;

9-(4-(azetidin-1-ylmethyl)phenyl)-5-fluoro-10-(1-propyl-1H-1,2,4-triazol-5-yl)-7,8,9,10-tetrahydroazepino[5,4,3-de]phthalazin-3(2H)-one;

9-(4-(azetidin-1-ylmethyl)phenyl)-5-fluoro-10-(1-(2-hydroxyethyl)-1H-1,2,4-triazol-5-yl)-7,8,9,10-tetrahydroazepino[5,4,3-de]phthalazin-3(2H)-one;

9-(4-(azetidin-1-ylmethyl)phenyl)-5-fluoro-10-(1-(3-hydroxypropyl)-1H-1,2,4-triazol-5-yl)-7,8,9,10-tetrahydroazepino[5,4,3-de]phthalazin-3(2H)-one;

9-(4-fluorophenyl)-10-(1H-1,2,4-triazol-5-yl)-7,8,9,10-tetrahydroazepino[5,4,3-de]phthalazin-3(2H)-one;

9-(4-fluorophenyl)-10-(1-methyl-1H-1,2,4-triazol-5-yl)-7,8,9,10-tetrahydroazepino[5,4,3-de]phthalazin-3(2H)-one;

9-(4-fluorophenyl)-10-(1-(2-hydroxyethyl)-1H-1,2,4-triazol-5-yl)-7,8,9,10-tetrahydroazepino[5,4,3-de]phthalazin-3(2H)-one;

9-(4-fluorophenyl)-10-(1-(3-hydroxypropyl)-1H-1,2,4-triazol-5-yl)-7,8,9,10-tetrahydroazepino[5,4,3-de]phthalazin-3(2H)-one;

10-(1-(2-hydroxyethyl)-1H-1,2,4-triazol-5-yl)-9-(4-((methylamino)methyl)phenyl)-7,8,9,10-tetrahydroazepino[5,4,3-de]phthalazin-3(2H-one;

10-(1-methyl-1H-1,2,4-triazol-5-yl)-9-(4-((methylamino)methyl)phenyl)-7,8,9,10-tetrahydroazepino[5,4,3-de]phthalazin-3(2H)-one;

9-(4-((methylamino)methyl)phenyl)-10-(1H-1,2,4-triazol-5-yl)-7,8,9,10-tetrahydroazepino[5,4,3-de]phthalazin-3(2H)-one;

9-(4-(azetidin-1-ylmethyl)phenyl)-10-(1-(2-hydroxyethyl)-1H-1,2,4-triazol-5-yl)-7,8,9,10-tetrahydroazepino[5,4,3-de]phthalazin-3(2H)-one;

9-(4-(azetidin-1-ylmethyl)phenyl)-10-(1-methyl-1H-1,2,4-triazol-5-yl)-7,8,9,10-tetrahydroazepino[5,4,3-de]phthalazin-3(2H)-one; and

9-(4-(azetidin-1-ylmethyl)phenyl)-10-(1H-1,2,4-triazol-5-yl)-7,8,9,10-tetrahydroazepino[5,4,3-de]phthalazin-3(2H)-one.

### Example 4

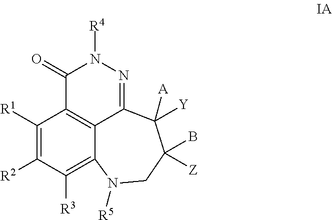

Synthesis of 5-fluoro-9-(4-fluorophenyl)-10-(1-methyl-1H-1,2,4-triazol-5-yl)-7,8,9,10-tetrahydroazepino[4,3,2-de]phthalazin-3(2H)-one (C)

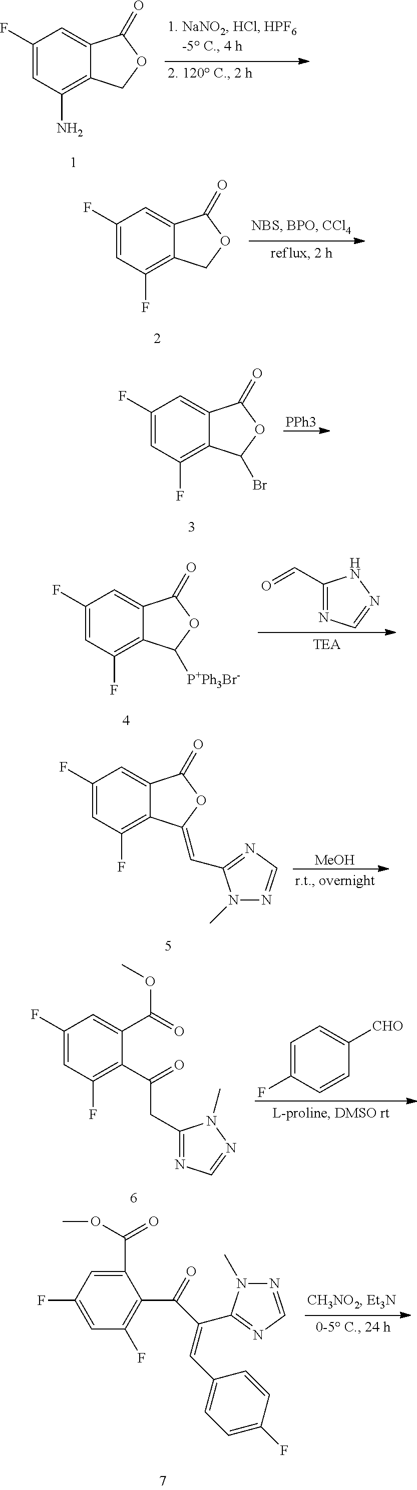

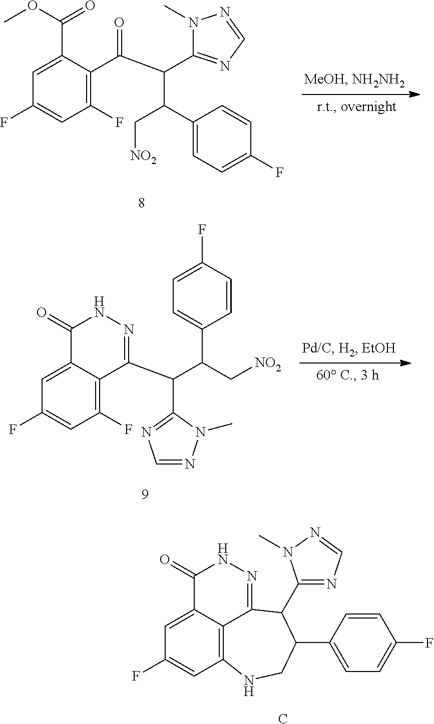

### Preparation of 4,6-difluoroisobenzofuran-1(3H)-one (2)

To a solution of 4-amino-6-fluoroisobenzofuran-1(3H)-one (1 g, 6 mmol) in concentrated HCl (12 mL) and water (30 mL) and cooled to −10 to 0° C., was added aqueous solution of NaNO₂ (480 mg, 6.64 mmol) dropwise. After the addition, 60% HPF₆ (60% solution in water) was added rapidly in one portion. The mixture was stirred for 4 hours and cooled to 0° C., the phosphorous hexafluoride salt was collected by filtration and washed with cold water followed by a mixture of diethyl ether and methanol (4:1). The solid was dried in vacuum over night to afford 800 mg of salt (yield 41%). Decomposition of the salt (3.4 g, 10.5 mmol) was carried out by portion-wise addition through the condenser fitted with a flask held at 120-150° C. After the decomposition was completed, the residue was extracted with ethyl acetate (20 mL×4) and the organic phase was washed with water and brine dried over sodium sulfate, concentrated to give crude product, which was purified by silica gel column (hexane:ethyl acetate 10:1) to give 4,6-difluoroisobenzofuran-1(3H)-one (2) (700 mg, yield 39%).MS (ESI) m/z 171(M+1).

### Preparation of 3-bromo-4,6-difluoroisobenzofuran-1(3H)-one (3)

4,6-Difluoroisobenzofuran-1(3H)-one (2) (170 g, 1 mmol), NBS (213 mg, 1.2 mmol) and BPO (24 mg, 0.1 mmol) were dissolved in CCl₄ (10 mL) and refluxed for 2 h. Water was then added to the mixture and extracted with DCM (20 mL×3). The organic layer was washed with water and brine, dried over Na₂SO₄, concentrated to give 3-bromo-4,6-difluoroisobenzofuran-1(3H)-one (3), which was used in next step without further purification (250 mg, yield 100%).

### Preparation of (5,7-difluoro-3-oxo-1,3-dihydroisobenzofuran-1-yl)triphenylphosphonium bromide (4)

3-Bromo-4,6-difluoroisobenzofuran-1(3H)-one (3), (250 mg, 1 mmol) and PPh₃ (314 mg, 1.2 mmol) were dissolved in toluene (10 mL) and stirred for 3 hours under nitrogen at 100° C. Then the mixture was cooled to room temperature and filtered, washed with toluene (20 mL) and dried under vacuum to give (5.7-difluoro-3-oxo-1,3-dihydroisobenzofuran-1-yl)triphenylphosphonium bromide (4) (150 mg). MS (ESI) m/z 431(M+1).

### Preparation of (Z)-4,6-difluoro-3-((1-methyl-1H-1,2,4-triazol-5-yl)methylene)isobenzofuran-1(3H)-one (5)

To a solution of (5,7-difluoro-3-oxo-1,3-dihydroisobenzofuran-1-yl)triphenylphosphonium bromide (4) (260 mg, 0.5 mmol) and 2-methyl-2H-1,2,4-triazole-3-carbaldehyde (90 mg, 0.8 mmol) in DCM (6 mL) was added TEA (50 mg, 0.5 mmol) slowly at 0° C. After the addition the mixture was stirred at 10° C. for 3.5 hours. DCM was removed by concentration and the residue was washed with 2-Me-THF (10 mL×2) to obtain (Z)-4,6-difluoro-3-((1-methyl-1H-1,2,4-triazol-5-yl)methylene)isobenzofuran-1(3H)-one (5) as a yellow solid. LC-MS (ESI) m/z: 264 (M+1)⁺.

### Preparation of methyl 3,5-difluoro-2-(2-(1-methyl-1H-1,2,4-triazol-5-yl)acetyl)benzoate (6)

(Z)-4,6-difluoro-3-((1-methyl-1H-1,2,4-triazol-5-yl)methylene)isobenzofuran-1(3H)-one (5) (11.5 g, 43.7 mmol) was suspended in 250 mL of methanol and stirred at room temperature overnight. TLC (Petroleum ether:EtOAc=2:1) and LC-MS (acid) showed the starting material was consumed completely and the solution turned clear. Then reaction mixture was evaporated to dryness to obtain methyl 3,5-difluoro-2-(2-(1-methyl-1H-1,2,4-triazol-5-yl)acetyl)benzoate (6) as yellow solid (12.8 g, yield 99%). LC-MS (ESI) m/z: 297 (M+1)⁺.

### Preparation of (E)-methyl 3,5-difluoro-2-(3-(4-fluorophenyl)-2-(1-methyl-1H-1,2,4-triazol-5-yl)acryloyl)benzoate (7)

To a stirred solution of methyl 3,5-difluoro-2-(2-(1-methyl-1H-1,2,4-triazol-5-yl)acetyl)benzoate (6) (590 mg, 2 mmol) and 4-fluorobenzaldehyde (488 mg, 4 mmol) in DMSO (2 mL) was added L-proline (230 mg, 2 mmol), the resulting mixture was kept with stirring at 45° C. for 48 hours. The reaction system was partitioned between ethyl acetate (50 mL) and water (30 mL), the organic phase was washed with water (20 mL×3), dried with Na₂SO₄, concentrated in vacuo, and purified by column chromatography (ethyl acetate:petroleum ether=1:3) to give (E)-methyl 3,5-difluoro-2-(3-(4-fluorophenyl)-2-(1-methyl-1H-1,2,4-triazol-5-yl)acryloyl)benzoate (7) as a pale yellow foam (321 mg, yield 40%). LC-MS (ESI) m/z: 402 (M+1)⁺.

### Preparation of methyl-3,5-difluoro-2-(3-(4-fluorophenyl)2-(1-methyl-1H-1,2,4-triazol-5-yl)-4-nitrobutanoyl)benzoate (8)

To a solution of (E)-methyl 3,5-difluoro-2-(3-(4-fluorophenyl)-2-(1-methyl-1H-1,2,4-triazol-5-yl)acryloyl)benzoate (7) (300 mg, 0.75 mmol) in nitromethane (5 mL) was added triethylamine (0.2 mL). The mixture was stirred at 0-5° C. for 24 hours, then neutralized with 1N HCl aqueous solution (pH=6˜7), and concentrated. Purification was carried out using prep-HPLC to obtain Compound 8 (60 mg, yield 17%). LC-MS (ESI) m/z: 463 (M+1)⁺. ¹H-NMR (400 MHz, CDCl₃) δ (ppm) 3.65 (s, 3H), 3.98 (s, 3H), 4.68-4.78 (m, 1H), 4.87-4.94 (t, J=11.2 Hz, 1H), 5.12-5.15 (d, J=11.6 Hz, 1H), 5.31-5.35 (dd, J₁=13.6 Hz, J₂=4 Hz, 1H), 6.86-6.96 (m, 3H), 7.13-7.16 (m, 2H), 7.52-7.54 (d, J=8 Hz, 1H), 7.60 (s, 1H)

### Preparation of 5,7-difluoro-4-(2-(4-fluorophenyl)-1-(1-methyl-1H-1,2,4-triazol-5-yl)-3-nitropropyl)phthalazin-1(2H)-one (9)

To a solution of 8 (70 mg, 0.15 mmol) in methanol (2 mL) was added hydrazine monohydrate (85%, 0.14 mL). The resulting mixture was stirred at room temperature for 3 hours and concentrated. The residues was dissolved in ethyl acetate (30 mL), washed with brine (20 mL×2), concentrated to obtain the desired Compound 9 (65 mg, yield 87%). LC-MS (ESI) m/z: 445 (M+1)⁺.

### Preparation of 5-fluoro-9-(4-fluorophenyl)-10-(1-methyl-1H-1,2,4-triazol-5-yl)-7,8,9,10-tetrahydroazepino[4,3,2-de]phthalazin-3(2H)-one (C)

To a solution of 9 (30 mg, 0.067 mmol) in EtOH (extensively purified to remove the residual aldehyde, 5 mL) was added methanamine in alcohol (30%, w/w, 0.001 mL) and Pd/C (10%, H₂O:71%, 50 mg). The resulting mixture was stirred under H₂ at 60° C. for 3 hours and then filtered. The filtrate was concentrated and purified by prep-HPLC to obtain Compound C (3 mg, yield 10%). LC-MS (ESI) m/z: 395 (M+1)⁺. ¹H-NMR (400 MHz, MeOH-d4) δ (ppm): 3.60 (s, 3H), 3.62-3.63 (d, J=3.2 Hz, 2H), 4.07-4.10 (m, 1H), 5.18-5.21 (d, J=11.2 Hz, 1H), 6.98-7.02 (t, J=8.8 Hz, 2H), 7.06-7.09 (dd, J₁=10.4 Hz, J₂=2.4 Hz, 1H), 7.35-7.38(dd, J₁=8.4 Hz, J₂=2.4 Hz, 1H), 7.52-7.56 (m, 2H), 7.80 (s, 1H).

### Example 4B

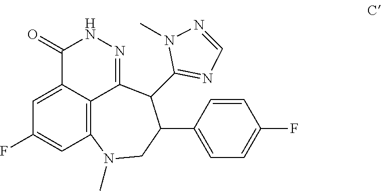 [SMILES: CN1CC(c2ccc(F)cc2)C(c2ncnn2C)c2n[nH]c(=O)c3cc(F)cc1c23]

### Synthesis of 5-Fluoro-9-(4-fluorophenyl)-7-methyl-10-(1-methyl-1H-1,2,4-triazol-5-yl)-7,8,9,10-tetrahydroazepino[4,3,2-de]phthalazin-3(2H)-one (C′)

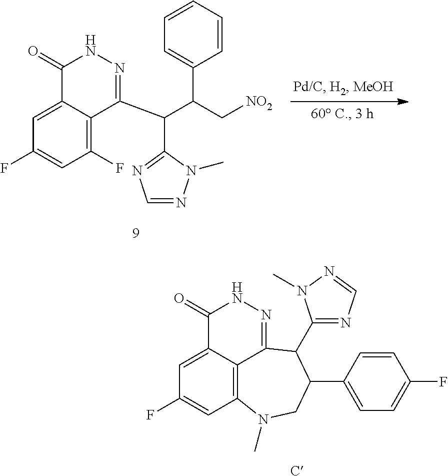

To a solution of 9 (30 mg, 0.067 mmol) in MeOH (5 mL) was added Pd/C (10%, 50 mg), then the resulting mixture was stirred under H₂ at 50° C. overnight. After filtration, the filtrate was heated to reflux for 5 hours, then concentrated and purified by prep-HPLC to obtain C′ (4.8 mg, yield 18%). LC-MS (ESI) m/z: 409 (M+1)⁺. ¹H-NMR (400 MHz, MeOH-d4) δ (ppm): 2.99 (s, 3H), 3.60 (s, 3H), 3.48-3.95 (d, J=14.8 Hz, 1H), 3.76-3.81 (dd, J₁=14.8 Hz, J₂=5.2 Hz, 1H), 4.16-4.20 (dd, J₁=11.2 Hz, J₂=5.2 Hz, 1H), 5.28-5.31 (d, J=10.8 Hz, 1H), 7.02-7.07 (t, J=8.8 Hz, 2H), 7.09-7.13 (dd, J_{b}=11.2 Hz, J₂=1.6 Hz, 1H), 7.39-7.41(dd, J₁=8.4 Hz, J₂=2.4 Hz, 1H), 7.51-7.55 (m, 2H), 7.76 (s, 1H).

### Example 5

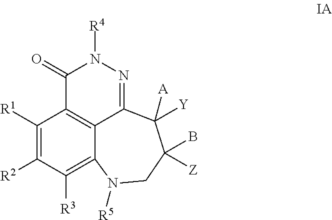

### Alternate Synthesis of 5-fluoro-9-(4-fluorophenyl)-10-(1-methyl-1H-1,2,4-triazol-5-yl)-7,8,9,10-tetrahydroazepino[4,3,2-de]phthalazin-3(2H)-one (C)

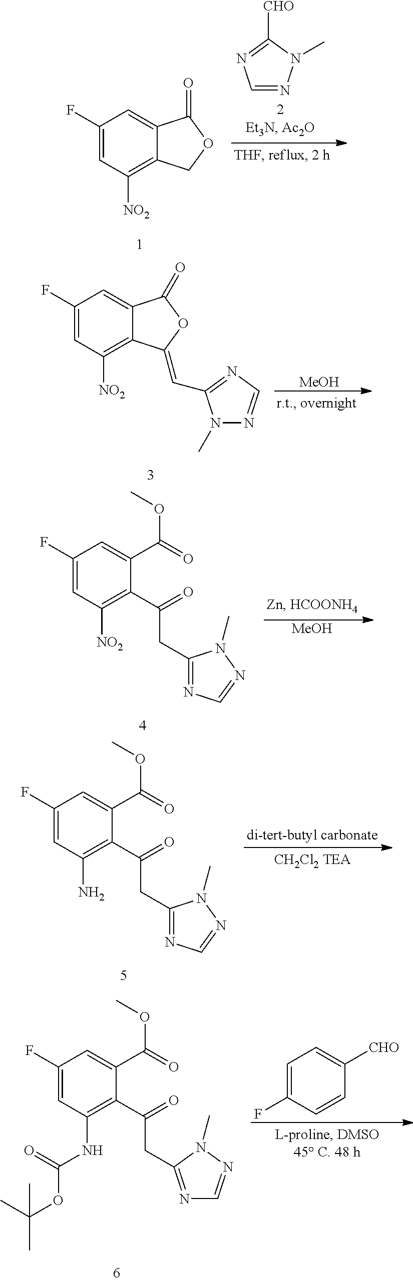

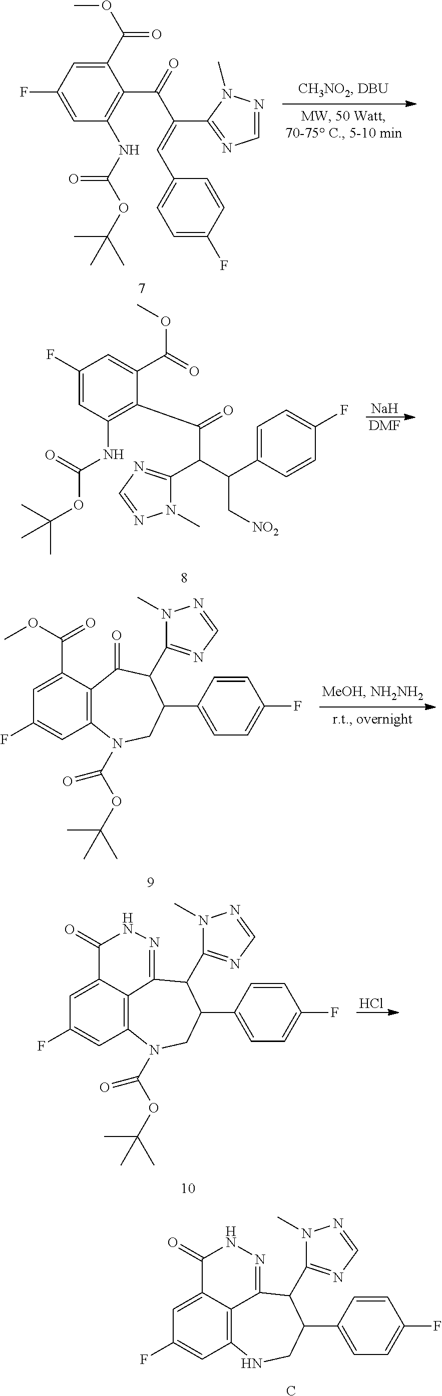

### Preparation of (Z)-6-fluoro-3-((1-methyl-1H-1,2,4-triazol-5-yl)methylene)-4-nitroisobenzofuran-1(3H)-one (3)

To a solution of 6-fluoro-4-nitroisobenzofuran-1(3H)-one (1) (10 g, 50.8 mmol), 2-methyl-2H-1,2,4-triazole-3-carbaldehyde (2) (11 g, 101.5 mmol) and Et₃N (20 mL, 152.4 mmol) in anhydrous THF (150 mL) was added acetic anhydride (35 mL) dropwise with stirring at room temperature in 3 minutes. The mixture was then stirred at reflux for 45 minutes when a green solid was precipitated from solvent. The solvent was concentrated to 15 mL, the resulting mixture was cooled to 0° C. and filtered, washed with 15 mL of ethyl acetate to obtain (Z)-6-fluoro-3-((1-methyl-1H-1,2,4-triazol-5-yl)methylene)-4-nitroisobenzofuran-1(3H)-one (3) as a green solid (11.5 g, yield: 78%). LC-MS (ESI) m/z: 291 (M+1)⁺. ¹H-NMR (400 MHz, DMSO-d₆) δ (ppm): 3.94 (s, 3H), 7.15 (s, 1H), 8.10 (s, 1H), 8.40-8.42 (dd, J₁=6.4 Hz, J₂=2.4 Hz, 1H), 8.58-8.61 (dd, J₁=8.8 Hz, J₂=2.4 Hz, 1H).

### Preparation of methyl 5-fluoro-2-(2-(1-methyl-1H-1,2,4-triazol-5-yl)acetyl)-3-nitrobenzoate (4)

(Z)-6-fluoro-3-((1-methyl-1H-1,2,4-triazol-5-yl)methylene)-4-nitroisobenzofuran-1(3H)-one (3) (177 g, 0.6 mol, 1.0 eq.), and HCl (2 N in methanol, 3 L, 6 mol, 10 eq.) were charged into a 5 L 3-neck flask equipped with mechanical stirrer, thermometer, nitrogen inlet/outlet. The reaction mixture was stirred at room temperature for 25 hours. The reaction mixture was monitored by HPLC, indicating 0.8% compound (3) remained. The reaction mixture was concentrated under vacuum at 40° C. to dryness, and methyl 5-fluoro-2-(2-(1-methyl-1H-1,2,4-triazol-5-yl)acetyl)-3-nitrobenzoate (4) was obtained as a yellow solid (201 g, yield: 93.4%). It was used for the next step without further purification. LC-MS (ESI) m/z: 323 (M+1)⁺. ¹H-NMR (400 MHz, DMSO-d₆) δ (ppm): 3.89 (s, 3H), 3.92 (s, 3H), 4.60 (s, 2H), 7.85 (s, 1H), 8.25-8.28 (dd, J₁=8.4 Hz, J₂=2.8 Hz, 2H), 8.52-8.54 (dd, J₁=8.4 Hz, J₂=2.8 Hz, 2H).

### Preparation of methyl 3-amino-5-fluoro-2-(2-(1-methyl-1H-1,2,4-triazol-5-yl)acetyl)benzoate (5)

To a suspension of methyl 5-fluoro-2-(2-(1-methyl-1H-1,2,4-triazol-5-yl)acetyl)-3-nitrobenzoate (4) (1.61 g, 5 mmol) and zinc dust (384 mg, 6 mmol) in 10 mL of methanol was stirred with ammonium formate (500 mg, 7.94 mmol) at room temperature overnight (with reaction completion monitored by TLC). The mixture was filtered. The organic solvent was evaporated and the residue was dissolved in DCM (100 mL). It was then washed with saturated brine water solution to remove ammonium formate. (50×2). The organic solvent was dried over magnesium sulfate and filtered. Upon evaporation to dryness, it gave methyl 3-amino-5-fluoro-2-(2-(1-methyl-1H-1,2,4-triazol-5-yl)acetyl)benzoate (5). LC-MS (ESI) m/z: 293 (M+1)⁺.

The following steps were not, but could be, conducted as described below.

### Preparation of methyl 3-((tert-butoxycarbonyl)amino)-5-fluoro-2-(2-(1-methyl-1H-1,2,4-triazol-5-yl)acetyl)benzoate (6)

To a solution of methyl 3-amino-5-fluoro-2-(2-(1-methyl-1H-1,2,4-triazol-5-yl)acetyl)benzoate (5) (584 mg, 2 mmol) in DCM (20 mL) is added 1 mL triethyl amine and di-tert-butyl carbonate (380 mg, 2.2 mmol) at room temperature under nitrogen atmosphere and the mixture is allowed to stir over night. DCM (100 mL) is added to the reaction and is washed with water (50 mL×2). The organic layer is dried over anhydrous Na₂SO₄ and concentrated to afford methyl 3-((tert-butoxycarbonyl)amino)-5-fluoro-2-(2-(1-methyl-1H-1,2,4-triazol-5-yl)acetyl)benzoate (6). Expected LC-MS (ESI) m/z: 393(M+1)⁺.

### Preparation of (E)-methyl 3-((tert-butoxycarbonyl)amino)-5-fluoro-2-(3-(4-fluorophenyl)-2-(1-methyl-1H-1,2,4-triazol-5-yl)acryloyl)benzoate (7)

To a stirred solution of methyl 3-((tert-butoxycarbonyl)amino)-5-fluoro-2-(2-(1-methyl-1H-1,2,4-triazol-5-yl)acetyl)benzoate (6). (590 mg, 1.5 mmol) and 4-fluorobenzaldehyde (366 mg, 3 mmol) in DMSO (2 mL) is added L-proline (172 mg, 1.5 mmol), the resulting mixture is kept with stirring at 45° C. for 48 hours. The reaction system is partitioned between ethyl acetate (50 mL) and water (30 mL), the organic phase is washed with water (20 mL×3), dried with Na₂SO₄, concentrated in vacuo, and purified by column chromatography (ethyl acetate:petroleum ether=1:3) to give (E)-methyl 3-((tert-butoxycarbonyl)amino)-5-fluoro-2-(3-(4-fluorophenyl)-2-(1-methyl-1H-1,2,4-triazol-5-yl)acryloyl)benzoate (7). Expected LC-MS (ESI) m/z: 499 (M+1)⁺.

### Preparation of methyl 3-((tert-butoxycarbonyl)amino)-5-fluoro-2-(3-(4-fluorophenyl)2-(1-methyl-1H-1,2,4-triazol-5-yl)-4-nitrobutanoyl)benzoate (8)

A solution of (E)-methyl 3-((tert-butoxycarbonyl)amino)-5-fluoro-2-(3-(4-fluorophenyl)-2-(1-methyl-1H-1,2,4-triazol-5-yl)acryloyl)benzoate (7) (4.98 g, 10 mmol), DBU (3.1 g, 20 mmol and nitromethane (2 mL, 37 mmol) in anhydrous THF (15 mL) is heated at 70-75° C. in a sealed vessel in a microware for 5 to 10 minutes. Then the mixture is added to water (100 mL). It is then extracted with DCM (50×2) and back washed with water (75 mL). The organic solvent was concentrated to dryness. The residue is then purified by column chromatography in silica gel column to obtain methyl 3-((tert-butoxycarbonyl)amino)-5-fluoro-2-(3-(4-fluorophenyl)2-(1-methyl-1H-1,2,4-triazol-5-yl)-4-nitrobutanoyl)benzoate (8). Expected LC-MS (ESI) m/z: 560 (M+1)⁺.

### Preparation of 1-tert-butyl 6-methyl 8-fluoro-3-(4-fluorophenyl)-4-(1-methyl-1H-1,2,4-triazol-5-yl)-5-oxo-2,3,4,5-tetrahydro-1H-benzo[b]azepine-1,6-dicarboxylate (9)

To a solution of methyl 3-((tert-butoxycarbonyl)amino)-5-fluoro-2-(3-(4-fluorophenyl)2-(1-methyl-1H-1,2,4-triazol-5-yl)-4-nitrobutanoyl)benzoate (8) (2.6 g, 5 mmol) in DMF (20 mL) is added 60% sodium hydride in oil dispersion (200 mg , 5 mmol) at ice temperature. It is then heated at 65° C. with stirring overnight. Water (2 ML) is added in and the organic solvent is removed under reduced pressure to dryness. The residue is dissolved in DCM (100 mL) washed with brine (50 mL×2) and the organic solution is dried over anhydrous sodium sulfate. The solvent is then removed under reduced pressure to afford 1-tert-butyl 6-methyl 8-fluoro-3-(4-fluorophenyl)-4-(1-methyl-1H-1,2,4-triazol-5-yl)-5-oxo-2,3,4,5-tetrahydro-1H-benzo[b]azepine-1,6-dicarboxylate (9) as a solid. Expected LC-MS (ESI) m/z: 513 (M+1)⁺.

### Preparation of tert-butyl 5-fluoro-9-(4-fluorophenyl)-10-(1-methyl-1H-1,2,4-triazol-5-yl)-3-oxo-2,3,9,10-tetrahydroazepino[4,3,2-de]phthalazine-7(8H)-carboxylate (10)

To a solution of 1-tert-butyl 6-methyl 8-fluoro-3-(4-fluorophenyl)-4-(1-methyl-1H-1,2,4-triazol-5-yl)-5-oxo-2,3,4,5-tetrahydro-1H-benzo[b]azepine-1,6-dicarboxylate (9) (1024 mg, 2 mmol) in THF (20 mL) is added hydrazine monohydrate (2.5 mL) at room temperature under nitrogen atmosphere and stirred for 2 hours, then acetic acid (2.5 mL) is added and the mixture was heated to 60° C. for 18 hours. The resulting mixture, after cooled to room temperature, is diluted with water (100 mL) and extracted with EtOAc (100 mL×3). The organic layer is dried over anhydrous Na₂SO₄ and concentrated to afford tert-butyl 5-fluoro-9-(4-fluorophenyl)-10-(1-methyl-1H-1,2,4-triazol-5-yl)-3-oxo-2,3,9,10-tetrahydroazepino[4,3,2-de]phthalazine-7(8H)-carboxylate (10). Expected LC-MS (ESI) m/z: 495 (M+1)⁺.

### Preparation of 5-fluoro-9-(4-fluorophenyl)-10-(1-methyl-1H-1,2,4-triazol-5-yl)-7,8,9,10-tetrahydroazepino[4,3,2-de]phthalazin-3(2H)-one (C)

To a solution of tert-butyl 5-fluoro-9-(4-fluorophenyl)-10-(1-methyl-1H-1,2,4-triazol-5-yl)-3-oxo-2,3,9,10-tetrahydroazepino[4,3,2-de]phthalazine-7(8H)-carboxylate (10) (494 mg, 1 mmol) in acetic acid (20 mL) is 5 mL of 5N hydrochloric acid (2.5 mL) at room temperature under nitrogen atmosphere and stirred for 5 hours. The solvent is removed under reduced pressure and the residue is dissolved in EtOAc (200 mL). It is then washed with brine (40 mL×2). The organic layer is dried over anhydrous Na₂SO₄ and concentrated to afford 5-fluoro-9-(4-fluorophenyl)-10-(1-methyl-1H-1,2,4-triazol-5-yl)-7,8,9,10-tetrahydroazepino[4,3,2-de]phthalazin-3(2H)-one (C). Expected LC-MS (ESI) m/z: 395 (M+1)⁺.

Using analogous methods and substituting with appropriate starting materials, the following compounds can be prepared using the procedures as described above in Examples 4 or 5.

5-fluoro-9-(4-fluorophenyl)-10-(1H-1,2,4-triazol-5-yl)-7,8,9,10-tetrahydroazepino[4,3,2-de]phthalazin-3(2H)-one;

5-fluoro-9-(4-fluorophenyl)-10-(1-methyl-1H-1,2,4-triazol-5-yl)-7,8,9,10-tetrahydroazepino[4,3,2-de]phthalazin-3(2H)-one;

10-(1-ethyl-1H-1,2,4-triazol-5-yl)-5-fluoro-9-(4-fluorophenyl)-7,8,9,10-tetrahydroazepino[4,3,2-de]phthalazin-3(2H)-one;

5-fluoro-9-(4-fluorophenyl)-10-(1-propyl-1H-1,2,4-triazol-5-yl)-7,8,9,10-tetrahydroazepino[4,3,2-de]phthalazin-3(2H)-one;

5-fluoro-9-(4-fluorophenyl)-10-(1-(2-hydroxyethyl)-1H-1,2,4-triazol-5-yl)-7,8,9,10-tetrahydroazepino[4,3,2-de]phthalazin-3(2H)-one;

5-fluoro-9-(4-fluorophenyl)-10-(1-(3-hydroxypropyl)-1H-1,2,4-triazol-5-yl)-7,8,9,10-tetrahydroazepino[4,3,2-de]phthalazin-3(2H)-one;

5-fluoro-9-phenyl-10-(1H-1,2,4-triazol-5-yl)-7,8,9,10-tetrahydroazepino[4,3,2-de]phthalazin-3(2H)-one;

5-fluoro-10-(1-methyl-1H-1,2,4-triazol-5-yl)-9-phenyl-7,8,9,10-tetrahydroazepino[4,3,2-de]phthalazin-3(2H)-one;

10-(1-ethyl-1H-1,2,4-triazol-5-yl)-5-fluoro-9-phenyl-7,8,9,10-tetrahydroazepino[4,3,2-de]phthalazin-3(2H)-one;

5-fluoro-9-phenyl-10-(1-propyl-1H-1,2,4-triazol-5-yl)-7,8,9,10-tetrahydroazepino[4,3,2-de]phthalazin-3(2H)-one;

5-fluoro-10-(1-(2-hydroxyethyl)-1H-1,2,4-triazol-5-yl)-9-phenyl-7,8,9,10-tetrahydroazepino[4,3,2-de]phthalazin-3(2H)-one;

5-fluoro-10-(1-(3-hydroxypropyl)-1H-1,2,4-triazol-5-yl)-9-phenyl-7,8,9,10-tetrahydroazepino[4,3,2-de]phthalazin-3(2H)-one;

5-fluoro-9-(4-((methylamino)methyl)phenyl)-10-(1H -1,2,4-triazol-5-yl)-7,8,9,10-tetrahydroazepino[4,3,2-de]phthalazin-3(2H)-one;

5-fluoro-10-(1-methyl-1H-1,2,4-triazol-5-yl)-9-(4-((methylamino)methyl)phenyl)-7,8,9,10-tetrahydroazepino[4,3,2-de]phthalazin-3(2H)-one;

10-(1-ethyl-1H-1,2,4-triazol-5-yl)-5-fluoro-9-(4-((methylamino)methyl)phenyl)-7,8,9,10-tetrahydroazepino[4,3,2-de]phthalazin-3(2H)-one;

5-fluoro-10-(1-(2-hydroxyethyl)-1H-1,2,4-triazol-5-yl)-9-(4-((methylamino)methyl)phenyl)-7,8,9,10-tetrahydroazepino[4,3,2-de]phthalazin-3(2H)-one;

5-fluoro-10-(1-(3-hydroxypropyl)-1H-1,2,4-triazol-5-yl)-9-(4-((methylamino)methyl)phenyl)-7,8,9,10-tetrahydroazepino[4,3,2-de]phthalazin-3(2H)-one;

9-(4-((dimethylamino)methyl)phenyl)-5-fluoro-10-(1H-1,2,4-triazol-5-yl)-7,8,9,10-tetrahydroazepino[4,3,2-de]phthalazin-3(2H)-one;

9-(4-((dimethylamino)methyl)phenyl)-5-fluoro-10-(1-methyl-1H-1,2,4-triazol-5-yl)-7,8,9,10-tetrahydroazepino[4,3,2-de]phthalazin-3(2H)-one;

9-(4-((dimethylamino)methyl)phenyl)-10-(1-ethyl-1H-1,2,4-triazol-5-yl)-5-fluoro-7,8,9,10-tetrahydroazepino[4,3,2-de]phthalazin-3(2H)-one;

9-(4-((dimethylamino)methyl)phenyl)-5-fluoro-10-(1-(2-hydroxyethyl)-1H-1,2,4-triazol-5-yl)-7,8,9,10-tetrahydroazepino[4,3,2-de]phthalazin-3(2H)-one;

9-(4-((dimethylamino)methyl)phenyl)-5-fluoro-10-(1-(3-hydroxypropyl)-1H-1,2,4-triazol-5-yl)-7,8,9,10-tetrahydroazepino[4,3,2-de]phthalazin-3(2H)-one;

9-(4-(azetidin-1-ylmethyl)phenyl)-5-fluoro-10-(1H-1,2,4-triazol-5-yl)-7,8,9,10-tetrahydroazepino[4,3,2-de]phthalazin-3(2H)-one;

9-(4-(azetidin-1-ylmethyl)phenyl)-5-fluoro-10-(1-methyl-1H-1,2,4-triazol-5-yl)-7,8,9,10-tetrahydroazepino[4,3,2-de]phthalazin-3(2H)-one;

9-(4-(azetidin-1-ylmethyl)phenyl)-10-(1-ethyl-1H-1,2,4-triazol-5-yl)-5-fluoro-7,8,9,10-tetrahydroazepino[4,3,2-de]phthalazin-3(2H)-one;

9-(4-(azetidin-1-ylmethyl)phenyl)-5-fluoro-10-(1-propyl-1H-1,2,4-triazol-5-yl)-7,8,9,10-tetrahydroazepino[4,3,2-de]phthalazin-3(2H)-one;

9-(4-(azetidin-1-ylmethyl)phenyl)-5-fluoro-10-(1-(2-hydroxyethyl)-1H-1,2,4-triazol-5-yl)-7,8,9,10-tetrahydroazepino[4,3,2-de]phthalazin-3(2H)-one;

9-(4-(azetidin-1-ylmethyl)phenyl)-5-fluoro-10-(1-(3-hydroxypropyl)-1H-1,2,4-triazol-5-yl)-7,8,9,10-tetrahydroazepino[4,3,2-de]phthalazin-3(2H)-one;

9-(4-fluorophenyl)-10-(1H-1,2,4-triazol-5-yl)-7,8,9,10-tetrahydroazepino[4,3,2-de]phthalazin-3(2H)-one;

9-(4-fluorophenyl)-10-(1-methyl-1H-1,2,4-triazol-5-yl)-7,8,9,10-tetrahydroazepino[4,3,2-de]phthalazin-3(2H)-one;

9-(4-fluorophenyl)-10-(1-(2-hydroxyethyl)-1H-1,2,4-triazol-5-yl)-7,8,9,10-tetrahydroazepino[4,3,2-de]phthalazin-3(2H)-one;

9-(4-fluorophenyl)-10-(1-(3-hydroxypropyl)-1H-1,2,4-triazol-5-yl)-7,8,9,10-tetrahydroazepino[4,3,2-de]phthalazin-3(2H)-one;

10-(1-(2-hydroxyethyl)-1H-1,2,4-triazol-5-yl)-9-(4-((methylamino)methyl)phenyl)-7,8,9,10-tetrahydroazepino[4,3,2-de]phthalazin-3(2H)-one;

10-(1-methyl-1H-1,2,4-triazol-5-yl)-9-(4-((methylamino)methyl)phenyl)-7,8,9,10-tetrahydroazepino[4,3,2-de]phthalazin-3(2H)-one;

9-(4-((methylamino)methyl)phenyl)-10-(1H-1,2,4-triazol-5-yl)-7,8,9,10-tetrahydroazepino[4,3,2-de]phthalazin-3(2H)-one;

9-(4-(azetidin-1-ylmethyl)phenyl)-10-(1-(2-hydroxyethyl)-1H-1,2,4-triazol-5-yl)-7,8,9,10-tetrahydroazepino[4,3,2-de]phthalazin-3(2H)-one;

9-(4-(azetidin-1-ylmethyl)phenyl)-10-(1-methyl-1H-1,2,4-triazol-5-yl)-7,8,9,10-tetrahydroazepino[4,3,2-de]phthalazin-3(2H)-one; and

9-(4-(azetidin-1-ylmethyl)phenyl)-10-(1H-1,2,4-triazol-5-yl)-7,8,9,10-tetrahydroazepino[4,3,2-de]phthalazin-3(2H)-one.

### Example 6

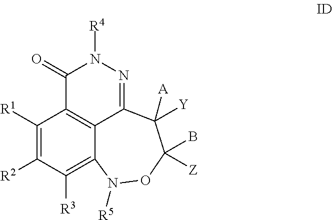

### Synthesis of 9-fluoro-3-(4-fluorophenyl)-4-(1-methyl-1H-1,2,4-triazol-5-yl)-3,4-dihydro-1H-[1,2]oxazepino[5,4,3-de]phthalazin-7(6H)-one (D)

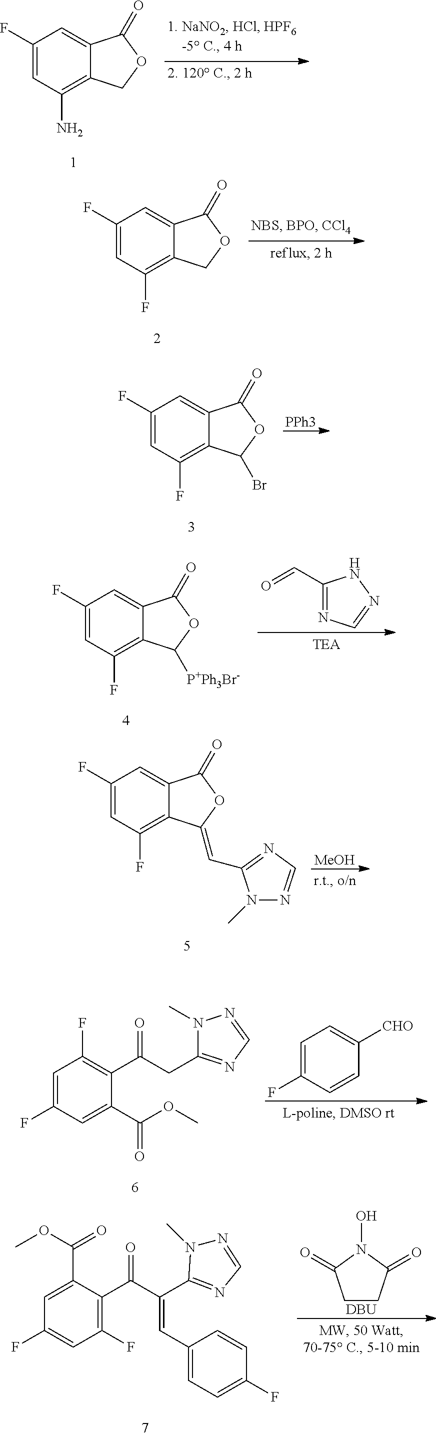

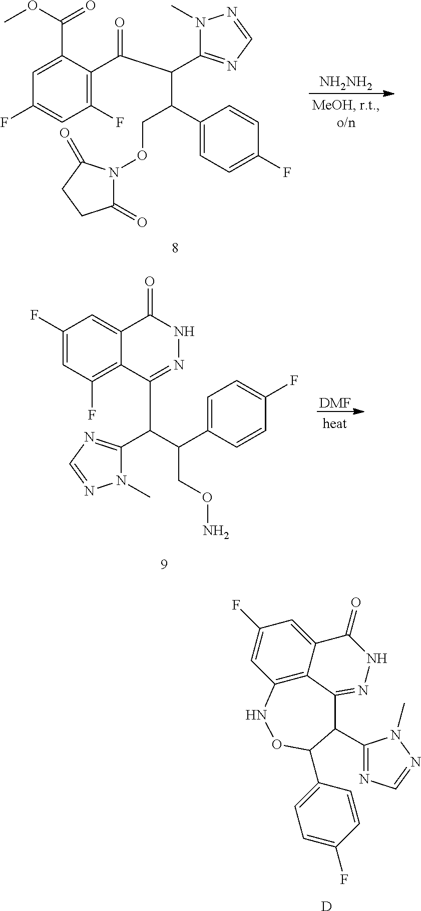

### Preparation of 4,6-difluoroisobenzofuran-1(3H)-one (2)

To a solution of 4-amino-6-fluoroisobenzofuran-1(3H)-one (1 g, 6 mmol) in concentrated HCl (12 mL) and water (30 mL) and cooled to −10 to 0° C., was added aqueous solution of NaNO₂ (480 mg, 6.64 mmol) dropwise. After the addition, 60% HPF₆ (60% solution in water) was added rapidly in one portion. The mixture was stirred for 4 hours and cooled to 0° C., the phosphorous hexafluoride salt was collected by filtration and washed with cold water followed by a mixture of diethyl ether and methanol (4:1). The solid was dried in vacuum over night to afford 800 mg of salt (yield 41%). Decomposition of the salt (3.4 g, 10.5 mmol) was carried out by portion-wise addition through the condenser fitted with a flask held at 120-150° C. After the decomposition was completed, the residue was extracted with ethyl acetate (20 mL×4) and the organic phase was washed with water and brine dried over sodium sulfate, concentrated to give crude product, which was purified by silica gel column (hexane:ethyl acetate 10:1) to give 4,6-difluoroisobenzofuran-1(3H)-one (2) (700 mg, yield 39%). MS (ESI) m/z 171(M+1).

### Preparation of 3-bromo-4,6-difluoroisobenzofuran-1(3H)-one (3)

4,6-Difluoroisobenzofuran-1(3H)-one (2) (170 g, 1 mmol), NBS (213 mg, 1.2 mmol) and BPO (24 mg, 0.1 mmol) were dissolved in CCl₄ (10 mL) and refluxed for 2 h. Water was then added to the mixture and extracted with DCM (20 mL×3). The organic layer was washed with water and brine, dried over Na₂SO₄, concentrated to give 3-bromo-4,6-difluoroisobenzofuran-1(3H)-one (3), which was used in next step without further purification (250 mg, yield 100%).

### Preparation of (5,7-difluoro-3-oxo-1,3-dihydroisobenzofuran-1-yl)triphenylphosphonium bromide (4)

3-Bromo-4,6-difluoroisobenzofuran-1(3H)-one (3), (250 mg, 1 mmol) and PPh₃ (314 mg, 1.2 mmol) were dissolved in toluene (10 mL) and stirred for 3 hours under nitrogen at 100° C. Then the mixture was cooled to room temperature and filtered, washed with toluene (20 mL) and dried under vacuum to give (5,7-difluoro-3-oxo-1,3-dihydroisobenzofuran-1-yl)triphenylphosphonium bromide (4) (150 mg). MS (ESI) m/z 431(M+1).

### Preparation of (Z)-4,6-difluoro-3-((1-methyl-1H-1,2,4-triazol-5-yl)methylene)isobenzofuran-1(3H)-one (5)

To a solution of (5,7-difluoro-3-oxo-1,3-dihydroisobenzofuran-1-yl)triphenylphosphonium bromide (4) (260 mg, 0.5 mmol) and 2-methyl-2H-1,2,4-triazole-3-carbaldehyde (90 mg, 0.8 mmol) in DCM (6 mL) was added TEA (50 mg, 0.5 mmol) slowly at 0° C. After the addition the mixture was stirred at 10° C. for 3.5 hours. DCM was removed by concentration and the residue was washed with 2-Me-THF (10 mL×2) to obtain (Z)-4,6-difluoro-3-((1-methyl-1H-1,2,4-triazol-5-yl)methylene)isobenzofuran-1(3H)-one (3) as a yellow solid. LC-MS (ESI) m/z: 264 (M+1)⁺.

### Preparation of methyl 3,5-difluoro-2-(2-(1-methyl-1H-1,2,4-triazol-5-yl)acetyl)benzoate (6)

(Z)-4,6-difluoro-3-((1-methyl-1H-1,2,4-triazol-5-yl)methylene)isobenzofuran-1(3H)-one (5) (11.5 g, 43.7 mmol) was suspended in 250 mL of methanol and stirred at room temperature overnight. TLC (Petroleum ether:EtOAc=2:1) and LC-MS (acid) showed the starting material was consumed completely and the solution turned clear. Then reaction mixture was evaporated to dryness to obtain methyl 3,5-difluoro-2-(2-(1-methyl-1H-1,2,4-triazol-5-yl)acetyl)benzoate (6) as yellow solid (12.8 g, yield 99%). LC-MS (ESI) m/z: 297 (M+1)⁺.

### Preparation of (E)-methyl 3,5-difluoro-2-(3-(4-fluorophenyl)-2-(1-methyl-1H-1,2,4-triazol-5-yl)acryloyl)benzoate (7)

To a stirred solution of methyl 3,5-difluoro-2-(2-(1-methyl-1H-1,2,4-triazol-5-yl)acetyl)benzoate (6) (590 mg, 2 mmol) and 4-fluorobenzaldehyde (488 mg, 4 mmol) in DMSO (2 mL) was added L-proline (230 mg, 2 mmol), the resulting mixture was kept with stirring at 45° C. for 48 hours. The reaction system was partitioned between ethyl acetate (50 mL) and water (30 mL), the organic phase was washed with water (20 mL×3), dried with Na₂SO₄, concentrated in vacuo, and purified by column chromatography (ethyl acetate:petroleum ether=1:3) to give (E)-methyl 3,5-difluoro-2-(3-(4-fluorophenyl)-2-(1-methyl-1H-1,2,4-triazol-5-yl)acryloyl)benzoate (7) as a pale yellow foam (321 mg, yield 40%). LC-MS (ESI) m/z: 402 (M+1)⁺.

The following steps were not, but could be conducted, as described below.

### Preparation of methyl-2-(4-((2,5-dioxopyrrolidin-1-yl)oxy)-3(4-fluorophenyl)-2-(1-methyl-1H-1,2,4-triazol-5-yl)butanoyl)-3,5-difluorobenzoate (8)

A solution of (E)-methyl 3,5-difluoro-2-(3-(4-fluorophenyl)-2-(1-methyl-1H-1,2,4-triazol-5-yl)acryloyl)benzoate (7) (2 g, 5 mmol), DBU (1.55 g, 10 mmol and N hydroxysuccinimide (1.15 g, 10 mmol) in anhydrous THF (15 mL) is heated at 70-75° C. over night. Then the mixture is added to water (100 mL). It is then extracted with DCM (50×2) and back washed with water (75 mL). The organic solvent was concentrated to dryness. The residue is then purified by column chromatography in silica gel column to obtain methyl-2-(4-((2,5-dioxopyrrolidin-1-yl)oxy)-3(4-fluorophenyl)-2-(1-methyl-1H-1,2,4-triazol-5-yl)butanoyl)-3,5-difluorobenzoate (8). Expected LC-MS (ESI) m/z: 531 (M+1)⁺.

### Synthesis of 4-(3-(aminooxy)-2-(4-fluorophenyl)-1-(1-methyl-1H-1,2,4-triazol-5-yl)propyl)-5,7-difluorophthalazin-1(2H)-one (9)

To a solution of methyl-2-(4-((2,5-dioxopyrrolidin-1-yl)oxy)-3(4-fluorophenyl)-2-(1-methyl-1H-1,2,4-triazol-5-yl)butanoyl)-3,5-difluorobenzoate (8). (1.06 g, 2 mmol) in THF (50 mL) is added hydrazine monohydrate (3 mL) at room temperature under nitrogen atmosphere and stirred for 6 hours. Acetic acid (2 mL) is added and the mixture is heated to 60° C. overnight. The resulting mixture is diluted with water (50 mL) and extracted with EtOAc (50 mL×3). The organic layer is dried over anhydrous Na₂SO₄ and concentrated to afford 4-(3-(aminooxy)-2-(4-fluorophenyl)-1-(1-methyl-1H-1,2,4-triazol-5-yl)propyl)-5,7-difluorophthalazin-1(2H)-one (9) as a solid. Expected LC-MS (ESI) m/z: 431(M+1)⁺.

### Preparation of 9-fluoro-3-(4-fluorophenyl)-4-(1-methyl-1H-1,2,4-triazol-5-yl)-3,4-dihydro-1H-[1,2]oxazepino[5,4,3-de]phthalazin-7(6H)-one (D)

A stirred solution of 4-(3-(aminooxy)-2-(4-fluorophenyl)-1-(1-methyl-1H-1,2,4-triazol-5-yl)propyl)-5,7-difluorophthalazin-1(2H)-one (9) 430 mg, 1 mmol) in dimethylformamide (DMF) (15 mL) is heated at 90° C. for 24 hours. The solvent is evaporated under reduced pressure to dryness. The residue is dissolved in DCM (100 mL) and is washed with water (50 mL×2). The organic layer is separated and dried over anhydrous sodium sulfate to give 9-fluoro-3-(4-fluorophenyl)-4-(1-methyl-1H-1,2,4-triazol-5-yl)-3,4-dihydro-1H-[1,2]oxazepino[5,4,3-de]phthalazin-7(6H)-one (D). Expected LC-MS (ESI) m/z: 397 (M+1)⁺.

Using analogous methods and substituting with appropriate starting materials, the following compounds can be prepared using the procedures as described above.

9-fluoro-3-(4-fluorophenyl)-4-(1H-1,2,4-triazol-5-yl)-3,4-dihydro-1H -[1,2]oxazepino[5,4-de]phthalazin-7(6H)-one;

9-fluoro-3-(4-fluorophenyl)-4-(1-methyl-1H-1,2,4-triazol-5-yl)-3,4-dihydro-1H-[1,2]oxazepino[5,4,3-de]phthalazin-7(6H)-one;

4-(1-ethyl-1H-1,2,4-triazol-5-yl)-9-fluoro-3-(4-fluorophenyl)-3,4-dihydro-1H-[1,2]oxazepino[5,4,3-de]phthalazin-7(6H)-one;

9-fluoro-3-(4-fluorophenyl)-4-(1-propyl-1H-1,2,4-triazol-5-yl)-3,4-dihydro-1H-[1,2]oxazepino[5,4,3-de]phthalazin-7(6H)-one;

9-fluoro-3-(4-fluorophenyl)-4-(1-(2-hydroxyethyl)-1H-1,2,4-triazol-5-yl)-3,4-dihydro-1H-[1,2]oxazepino[5,4,3-de]phthalazin-7(6H)-one;

9-fluoro-3-(4-fluorophenyl)-4-(1-(3-hydroxypropyl)-1H-1,2,4-triazol-5-yl)-3,4-dihydro-1H-[1,2]oxazepino[5,4,3-de]phthalazin-7(6H)-one;

9-fluoro-3-phenyl-4-(1H-1,2,4-triazol-5-yl)-3,4-dihydro-1H-[1,2]oxazepino[5,4,3-de]phthalazin-7(6H)-one;

9-fluoro-4-(1-methyl-1H-1,2,4-triazol-5-yl)-3-phenyl-3,4-dihydro-1H-[1,2]oxazepino[5,4,3-de]phthalazin-7(6H)-one;

4-(1-ethyl-1H-1,2,4-triazol-5-yl)-9-fluoro-3-phenyl-3,4-dihydro-1H-[1,2]oxazepino[5,4,3-de]phthalazin-7(6H)-one;

9-fluoro-3-phenyl-4-(1-propyl-1H-1,2,4-triazol-5-yl)-3,4-dihydro-1H-[1,2]oxazepino[5,4,3-de]phthalazin-7(6H)-one;

9-fluoro-4-(1-(2-hydroxyethyl)-1H-1,2,4-triazol-5-yl)-3-phenyl-3,4-dihydro-1H-[1,2]oxazepino[5,4,3-de]phthalazin-7(6H)-one;

9-fluoro-4-(1-(3-hydroxypropyl)-1H-1,2,4-triazol-5-yl)-3-phenyl-3,4-dihydro-1H-[1,2]oxazepino[5,4,3-de]phthalazin-7(6H)-one;

9-fluoro-3-(4-((methylamino)methyl)phenyl)-4-(1,2,4-triazol-5-yl)-3,4-dihydro-1H-[1,2]oxazepino[5,4,3-de]phthalazin-7(6H)-one;

9-fluoro-4-(1-methyl-1H-1,2,4-triazol-5-yl)-3-(4-((methylamino)methyl)phenyl)-3,4-dihydro-1H-[1,2]oxazepino[5,4,3-de]phthalazin-7(6H)-one;

4-(1-ethyl-1H-1,2,4-triazol-5-yl)-9-fluoro-3-(4-((methylamino)methyl)phenyl)-3,4-dihydro-1H-[1,2]oxazepino[5,4,3-de]phthalazin-7(6H)-one;

9-fluoro-4-(1-(2-hydroxyethyl)-1H-1,2,4-triazol-5-yl)-3-(4-((methylamino)methyl)phenyl)-3,4-dihydro-1H-[1,2]oxazepino[5,4,3-de]phthalazin-7(6H)-one;

9-fluoro-4-(1-(3-hydroxypropyl)-1H-1,2,4-triazol-5-yl)-3-(4-((methylamino)methyl)phenyl)-3,4-dihydro-1H-[1,2]oxazepino[5,4,3-de]phthalazin-7(6H)-one;

3-(4-((dimethylamino)methyl)phenyl)-9-fluoro-4-(1H-1,2,4-triazol-5-yl)-3,4-dihydro-1H-[1,2]oxazepino[5,4,3-de]phthalazin-7(6H)-one;

3-(4-((dimethylamino)methyl)phenyl)-9-fluoro-4-(1-methyl-1H-1,2,4-triazol-5-yl)-3,4-dihydro-1H-[1,2]oxazepino[5,4,3-de]phthalazin-7(6H)-one;

3-(4-((dimethylamino)methyl)phenyl)-4-(1-ethyl-1H-1,2,4-triazol-5-yl)-9-fluoro-3,4-dihydro-1H-[1,2]oxazepino[5,4,3-de]phthalazin-7(6H)-one;

3-(4-((dimethylamino)methyl)phenyl)-9-fluoro-4-(1-(2-hydroxyethyl)-1H-1,2,4-triazol-5-yl)-3,4-dihydro-1H-[1,2]oxazepino[5,4,3-de]phthalazin-7(6H)-one;

3-(4-((dimethylamino)methyl)phenyl)-9-fluoro-4-(1-(3-hydroxypropyl)-1H-1,2,4-triazol-5-yl)-3,4-dihydro-1H-[1,2]oxazepino [5,4,3-de]phthalazin-7(6H)-one;

3-(4-(azetidin-1-ylmethyl)phenyl)-9-fluoro-4-(1H-1,2,4-triazol-5-yl)-3,4-dihydro-1H-[1,2]oxazepino[5,4,3-de]phthalazin-7(6H)-one;

3-(4-(azetidin-1-ylmethyl)phenyl)-9-fluoro-4-(1-methyl-1H-1,2,4-triazol-5-yl)-3,4-dihydro-1H-[1,2]oxazepino[5,4,3-de]phthalazin-7(6H)-one;

3-(4-(azetidin-1-ylmethyl)phenyl)-4-(1-ethyl-1H-1,2,4-triazol-5-yl)-9-fluoro-3,4-dihydro-1H-[1,2]oxazepino[5,4,3-de]phthalazin-7(6H)-one;

3-(4-(azetidin-1-ylmethyl)phenyl)-9-fluoro-4-(1-propyl-1H-1,2,4-triazol-5-yl)-3,4-dihydro-1H-[1,2]oxazepino[5,4,3-de]phthalazin-7(6H)-one;

3-(4-(azetidin-1-ylmethyl)phenyl)-9-fluoro-4-(1-(2-hydroxyethyl)-1H-1,2,4-triazol-5-yl)-3,4-dihydro-1H-[1,2]oxazepino[5,4,3-de]phthalazin-7(6H)-one;

3-(4-(azetidin-1-ylmethyl)phenyl)-9-fluoro-4-(1-(3-hydroxypropyl)-1H-1,2,4-triazol-5-yl)-3,4-dihydro-1H-[1,2]oxazepino[5,4,3-de]phthalazin-7(6H)-one;

3-(4-fluorophenyl)-4-(1H-1,2,4-triazol-5-yl)-3,4-dihydro-1H-[1,2]oxazepino[5,4,3-de]phthalazin-7(6H)-one;

3-(4-fluorophenyl)-4-(1-methyl-1H-1,2,4-triazol-5-yl)-3,4-dihydro-1H-[1,2]oxazepino[5,4,3-de]phthalazin-7(6H)-one;

3-(4-fluorophenyl)-4-(1-(2-hydroxyethyl)-1H-1,2,4-triazol-5-yl)-3,4-dihydro-1H-[1,2]oxazepino[5,4,3-de]phthalazin-7(6H)-one;

3-(4-fluorophenyl)-4-(1-(3-hydroxypropyl)-1H-1,2,4-triazol-5-yl)-3,4-dihydro-1H-[1,2]oxazepino[5,4,3-de]phthalazin-7(6H)-one;

4-(1-(2-hydroxyethyl)-1H-1,2,4-triazol-5-yl)-3-(4-((methylamino)methyl)phenyl)-3,4-dihydro-1H-[1,2]oxazepino[5,4,3-de]phthalazin-7(6H)-one;

4-(1-methyl-1H-1,2,4-triazol-5-yl)-3-(4-((methylamino)methyl)phenyl)-3,4-dihydro-1H-[1,2]oxazepino[5,4,3-de]phthalazin-7(6H)-one;

3-(4-((methylamino)methyl)phenyl)-4-(1H-1,2,4-triazol-5-yl)-3,4-dihydro-1H-[1,2]oxazepino[5,4,3-de]phthalazin-7(6H)-one;

3-(4-(azetidin-1-ylmethyl)phenyl)-4-(1-(2-hydroxyethyl)-1H-1,2,4-triazol-5-y l)-3,4-dihydro-1H-[1,2]oxazepino[5,4,3-de]phthalazin-7(6H)-one;

3-(4-(azetidin-1-ylmethyl)phenyl)-4-(1-methyl-1H-1,2,4-triazol-5-yl)-3,4-dihydro-1H-[1,2]oxazepino[5,4,3-de]phthalazin-7(6H)-one; and

3-(4-(azetidin-1-ylmethyl)phenyl)-4-(1H-1,2,4-triazol-5-yl)-3,4-dihydro-1H-[1,2]oxazepino[5,4,3-de]phthalazin-7(6H)-one.

### Example 7

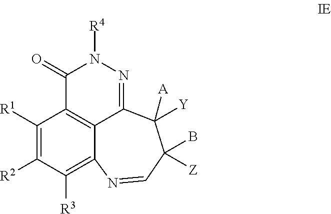

### Synthesis of 5-fluoro-9-(4-fluorophenyl)-10-(1-methyl-1H-1,2,4-triazol-5-yl)-9,10-dihydroazepino[4,3,2-de]phthalazin-3(2H)-one (E)

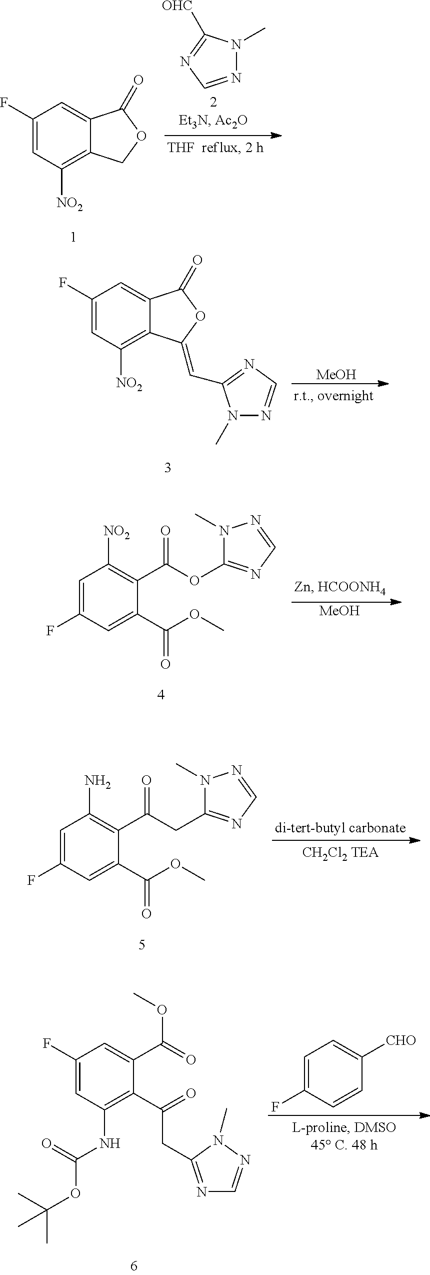

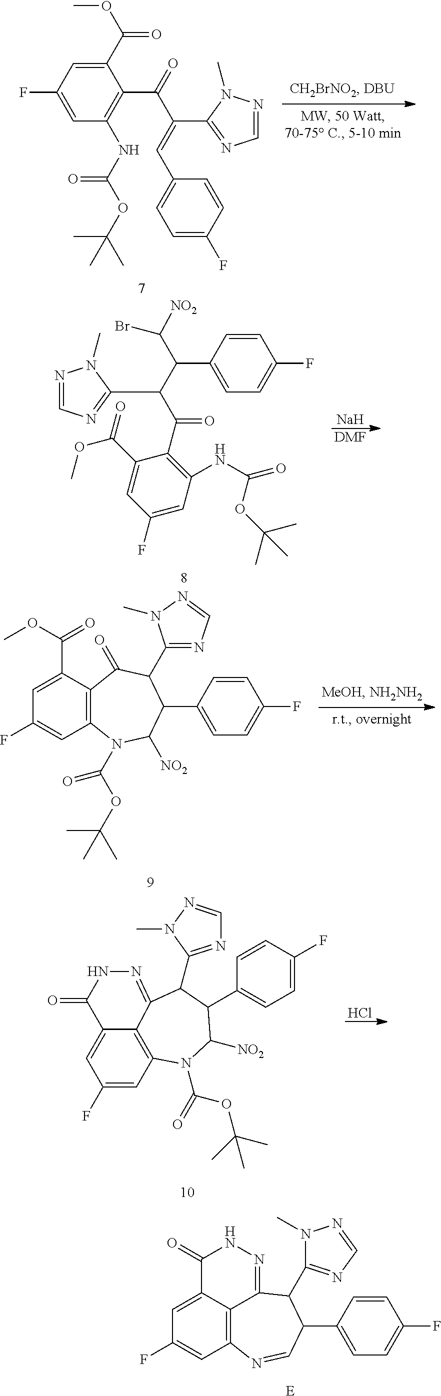

### Preparation of (Z)-6-fluoro-3-((1-methyl-1H-1,2,4-triazol-5-yl)methylene)-4-nitroisobenzofuran-1(3H)-one (3)

To a solution of 6-fluoro-4-nitroisobenzofuran-1(3H)-one (1) (10 g, 50.8 mmol), 2-methyl-2H-1,2,4-triazole-3-carbaldehyde (2) (11 g, 101.5 mmol) and Et₃N (20 mL, 152.4 mmol) in anhydrous THF (150 mL) was added acetic anhydride (35 mL) dropwise with stirring at room temperature in 3 minutes. The mixture was then stirred at reflux for 45 minutes when a green solid was precipitated from solvent. The solvent was concentrated to 15 mL, the resulting mixture was cooled to 0° C. and filtered, washed with 15 mL of ethyl acetate to obtain (Z)-6-fluoro-3-((1-methyl-1H-1,2,4-triazol-5-yl)methylene)-4-nitroisobenzofuran-1(3H)-one (3) as a green solid (11.5 g, yield: 78%). LC-MS (ESI) m/z: 291 (M+1)⁺. ¹H-NMR (400 MHz, DMSO-d₆) δ (ppm): 3.94 (s, 3H), 7.15 (s, 1H), 8.10 (s, 1H), 8.40-8.42 (dd, J₁=6.4 Hz, J₂=2.4 Hz, 1H), 8.58-8.61 (dd, J₁=8.8 Hz, J₂=2.4 Hz, 1H).

### Preparation of methyl 5-fluoro-2-(2-(1-methyl-1H-1,2,4-triazol-5-yl)acetyl)-3-nitrobenzoate (4)

(Z)-6-fluoro-3-((1-methyl-1H-1,2,4-triazol-5-yl)methylene)-4-nitroisobenzofuran-1(3H)-one (3) (177 g, 0.6 mol, 1.0 eq.), and HCl (2 N in methanol, 3 L, 6 mol, 10 eq.) were charged into a 5 L 3-neck flask equipped with mechanical stirrer, thermometer, nitrogen inlet/outlet. The reaction mixture was stirred at room temperature for 25 hours. The reaction mixture was monitored by HPLC, indicating 0.8% compound (3) remained. The reaction mixture was concentrated under vacuum at 40° C. to dryness, and methyl 5-fluoro-2-(2-(1-methyl-1H-1,2,4-triazol-5-yl)acetyl)-3-nitrobenzoate (4) was obtained as a yellow solid (201 g, yield: 93.4%). It was used for the next step without further purification. LC-MS (ESI) m/z: 323 (M+1)⁺. ¹H-NMR (400 MHz, DMSO-d₆) δ (ppm): 3.89 (s, 3H), 3.92 (s, 3H), 4.60 (s, 2H), 7.85 (s, 1H), 8.25-8.28 (dd, J₁=8.4 Hz, J₂=2.8 Hz, 2H), 8.52-8.54 (dd, J₁=8.4 Hz, J₂=2.8 Hz, 2H).

### Preparation of methyl 3-amino-5-fluoro-2-(2-(1-methyl-1H-1,2,4-triazol-5-yl)acetyl)benzoate (5)

To a suspension of methyl 5-fluoro-2-(2-(1-methyl-1H-1,2,4-triazol-5-yl)acetyl)-3-nitrobenzoate (4) (1.61 g, 5 mmol) and zinc dust (384 mg, 6 mmol) in 10 mL of methanol was stirred with ammonium formate (500 mg, 7.94 mmol) at room temperature overnight (with reaction completion monitored by TLC). The mixture was filtered. The organic solvent was evaporated and the residue was dissolved in DCM (100 mL). It was then washed with saturated brine water solution to remove ammonium formate. (50×2). The organic solvent was dried over magnesium sulfate and filtered. Upon evaporation to dryness, it gave methyl 3-amino-5-fluoro-2-(2-(1-methyl-1H-1,2,4-triazol-5-yl)acetyl)benzoate (5). LC-MS (ESI) m/z: 293 (M+1)⁺.

The following steps were not, but could be conducted, as described below.

### Preparation of methyl 3-((tert-butoxycarbonyl)amino)-5-fluoro-2-(2-(1-methyl-1H-1,2,4-triazol-5-yl)acetyl)benzoate (6)

To a solution of methyl 3-amino-5-fluoro-2-(2-(1-methyl-1H-1,2,4-triazol-5-yl)acetyl)benzoate (5) (584 m g, 2 mmol) in DCM (20 mL) is added 1 mL triethyl amine and di-tert-butyl carbonate (380 mg, 2.2 mmol) at room temperature under nitrogen atmosphere and the mixture is allowed to stir over night. DCM (100 mL) is added to the reaction and is washed with water (50 mL×2). The organic layer is dried over anhydrous Na₂SO₄ and concentrated to afford methyl 3-((tert-butoxycarbonyl)amino)-5-fluoro-2-(2-(1-methyl-1H-1,2,4-triazol-5-yl)acetyl)benzoate (6). Expected LC-MS (ESI) m/z: 393(M+1)⁺.

### Preparation of (E)-methyl 3-((tert-butoxycarbonyl)amino)-5-fluoro-2-(3-(4-fluorophenyl)-2-(1-methyl-1H-1,2,4-triazol-5-yl)acryloyl)benzoate (7)

To a stirred solution of methyl 3-((tert-butoxycarbonyl)amino)-5-fluoro-2-(2-(1-methyl-1H-1,2,4-triazol-5-yl)acetyl)benzoate (6). (590 mg, 1.5 mmol) and 4-fluorobenzaldehyde (366 mg, 3 mmol) in DMSO (2 mL) is added L-proline (172 mg, 1.5 mmol), the resulting mixture is kept with stirring at 45° C. for 48 hours. The reaction system is partitioned between ethyl acetate (50 mL) and water (30 mL), the organic phase is washed with water (20 mL×3), dried with Na₂SO₄, concentrated in vacuo, and purified by column chromatography (ethyl acetate:petroleum ether=1:3) to give (E)-methyl 3-((tert-butoxycarbonyl)amino)-5-fluoro-2-(3-(4-fluorophenyl)-2-(1-methyl-1H-1,2,4-triazol-5-yl)acryloyl)benzoate (7). Expected LC-MS (ESI) m/z: 499 (M+1)⁺.

### Preparation of methyl 2-(4-bromo-3-(4-fluorophenyl)-2-(1-methyl-1H-1,2,4-triazol-5-yl)-4-nitrobutanoyl)-3-((tert-butoxycarbonyl)amino)-5-fluorobenzoate (8)

A solution of (E)-methyl 3-((tert-butoxycarbonyl)amino)-5-fluoro-2-(3-(4-fluorophenyl)-2-(1-methyl-1H-1,2,4-triazol-5-yl)acryloyl)benzoate (7) (4.98 g, 10 mmol), DBU (3.1 g, 20 mmol and 1-bromonitromethane (5.1 g, 37 mmol) in anhydrous THF (25 mL) is heated at 70-75° C. in a sealed vessel in a microware CEM Discover apparatus for 5 to 10 minutes. Then the mixture is added to water (100 mL). It is then extracted with DCM (50×3) and back washed with water (75 mL). The organic solvent is concentrated to dryness. The residue is then purified by column chromatography in silica gel column to obtain methyl 2-(4-bromo-3-(4-fluorophenyl)-2-(1-methyl-1H-1,2,4-triazol-5-yl)-4-nitrobutanoyl)-3-((tert-butoxycarbonyl)amino)-5-fluorobenzoate (8). Expected LC-MS (ESI) m/z: 638 (M+1)⁺.

### Preparation of 1-tert-butyl 6-methyl 8-fluoro-3-(4-fluorophenyl)-4-(1-methyl-1H-1,2,4-triazol-5-yl)-2-nitro-5-oxo-2,3,4,5-tetrahydro-1H-benzo[b]azepine-1,6-dicarboxylate (9)

To a solution of methyl 2-(4-bromo-3-(4-fluorophenyl)-2-(1-methyl-1H-1,2,4-triazol-5-yl)-4-nitrobutanoyl)-3-((tert-butoxycarbonyl)amino)-5-fluorobenzoate (8) (3.19 g, 5 mmol) in DMF (20 mL) is added 60% sodium hydride in oil dispersion (200 mg , 5 mmol) at ice temperature. It is then heated at 65° C. with stirring overnight. Water (2 ML) is added and the mixture is evaporated to dryness under reduced pressure to dryness. DCM (100 mL) is added and the mixture is washed with brine (50 mL×2) and the organic solution is dried over anhydrous sodium sulfate. The solvent is then removed under reduced pressure to afford 1-tert-butyl 6-methyl 8-fluoro-3-(4-fluorophenyl)-4-(1-methyl-1H-1,2,4-triazol-5-yl)-2-nitro-5-oxo-2,3,4,5-tetrahydro-1H-benzo[b]azepine-1,6-dicarboxylate (9) as a solid. Expected LC-MS (ESI) m/z: 558 (M+1)⁺.

### Preparation of tert-butyl 5-fluoro-9-(4-fluorophenyl)-10-(1-methyl-1H-1,2,4-triazol-5-yl)-8-nitro-3-oxo-2,3,9,10-tetrahydroazepino[4,3,2-de]phthalazine-7(8H)-carboxylate (10)

To a solution of 1-tert-butyl 6-methyl 8-fluoro-3-(4-fluorophenyl)-4-(1-methyl-1H-1,2,4-triazol-5-yl)-2-nitro-5-oxo-2,3,4,5-tetrahydro-1H-benzo[b]azepine-1,6-dicarboxylate (9) (1,116 mg, 2 mmol) in THF (20 mL) is added hydrazine monohydrate (2.5 mL) at room temperature under nitrogen atmosphere and stirred for 2 hours, then acetic acid (2.5 mL) is added and the mixture was heated to 60° C. for 18 hours. The resulting mixture, after cooled to room temperature, is diluted with water (100 mL) and extracted with EtOAc (100 mL×3). The organic layer is dried over anhydrous Na₂SO₄ and concentrated to afford tert-butyl 5-fluoro-9-(4-fluorophenyl)-10-(1-methyl-1H-1,2,4-triazol-5-yl)-8-nitro-3-oxo-2,3,9,10-tetrahydroazepino[4,3,2-de]phthalazine-7(8H)-carboxylate (10). Expected LC-MS (ESI) m/z: 540 (M+1)⁺

### Preparation of 5-fluoro-9-(4-fluorophenyl)-10-(1-methyl-1H-1,2,4-triazol-5-yl)-9,10-dihydroazepino[4,3,2-de]phthalazin-3(2H)-one (E)

To a solution of tert-butyl 5-fluoro-9-(4-fluorophenyl)-10-(1-methyl-1H-1,2,4-triazol-5-yl)-8-nitro-3-oxo-2,3,9,10-tetrahydroazepino[4,3,2-de]phthalazine-7(8H)-carboxylate (10) (539 mg, 1 mmol) in acetic acid (20 mL) is 5 mL of 5 N hydrochloric acid (2.5 mL) at room temperature under nitrogen atmosphere and stirred for 5 hours. The solvent is removed under reduced pressure and the residue is dissolved in EtOAc (200 mL). It is then washed with brine (40 mL×2). The organic layer is dried over anhydrous Na₂SO₄ and concentrated to afford 5-fluoro-9-(4-fluorophenyl)-10-(1-methyl-1H-1,2,4-triazol-5-yl)-9,10-dihydroazepino[4,3,2-de]phthalazin-3(2H)-one (E). Expected LC-MS (ESI) m/z: 393 (M+1)⁺.

Using analogous methods and substituting with appropriate starting materials, the following compounds can be prepared using the procedures as described above.

5-fluoro-9-(4-fluorophenyl)-10-(1H-1,2,4-triazol-5-yl)-9,10-dihydroazepino[4,3,2-de]phthalazin-3(2H)-one;

5-fluoro-9-(4-fluorophenyl)-10-(1-methyl-1H-1,2,4-triazol-5-yl)-9,10-dihydroazepino[4,3,2-de]phthalazin-3(2H)-one;

10-(1-ethyl-1H-1,2,4-triazol-5-yl)-5-fluoro-9-(4-fluorophenyl)-9,10-dihydroazepino[4,3,2-de]phthalazin-3(2B)-one;

5-fluoro-9-(4-fluorophenyl)-10-(1-propyl-1H-1,2,4-triazol-5-yl)-9,10-dihydroazepino[4,3,2-de]phthalazin-3(2H)-one;

5-fluoro-9-(4-fluorophenyl)-10-(1-(2-hydroxyethyl)-1H-1,2,4-triazol-5-yl)-9,10-dihydroazepino[4,3,2-de]phthalazin-3(2H)-one;

5-fluoro-9-(4-fluorophenyl)-10-(1-(3-hydroxypropyl)-1H-1,2,4-triazol-5-yl)-9,10-dihydroazepino[4,3,2-de]phthalazin-3(2H)-one;

5-fluoro-9-phenyl-10-(1H-1,2,4-triazol-5-yl)-9,10-dihydroazepino [4,3,2-de]phthalazin-3(2H)-one;

5-fluoro-10-(1-methyl-1H-1,2,4-triazol-5-yl)-9-phenyl-9,10-dihydroazepino[4,3,2-de]phthalazin-3(2H)-one;

10-(1-ethyl-1H-1,2,4-triazol-5-yl)-5-fluoro-9-phenyl-9,10-dihydroazepino[4,3,2-de]phthalazin-3(2H)-one;

5-fluoro-9-phenyl-10-(1-propyl-1H-1,2,4-triazol-5-yl)-9,10-dihydroazepino[4,3,2-de]phthalazin-3(2H)-one;

5-fluoro-10-(1-(2-hydroxyethyl)-1H-1,2,4-triazol-5-yl)-9-phenyl-9,10-dihydroazepino[4,3,2-de]phthalazin-3(2H)-one;

5-fluoro-10-(1-(3-hydroxypropyl)-1H-1,2,4-triazol-5-yl)-9-phenyl-9,10-dihydroazepino[4,3,2-de]phthalazin-3(2H)-one;

5-fluoro-9-(4-((methylamino)methyl)phenyl)-10-(1H-1,2,4-triazol-5-yl)-9,10-dihydroazepino[4,3,2-de]phthalazin-3(2H)-one;

5-fluoro-10-(1-methyl-1H-1,2,4-triazol-5-yl)-9-(4-((methylamino)methyl)phenyl)-9,10-dihydroazepino[4,3,2-de]phthalazin-3(2H)-one;

10-(1-ethyl-1H-1,2,4-triazol-5-yl)-5-fluoro-9-(4-((methylamino)methyl)phenyl)-9,10-dihydroazepino[4,3,2-de]phthalazin-3(2H)-one;

5-fluoro-10-(1-(2-hydroxyethyl)-1H-1,2,4-triazol-5-yl)-9-(4-((methylamino)methyl)phenyl)-9,10-dihydroazepino[4,3,2-de]phthalazin-3(2H)-one;

5-fluoro-10-(1-(3-hydroxypropyl)-1H-1,2,4-triazol-5-yl)-9-(4-((methylamino)methyl)phenyl)-9,10-dihydroazepino[4,3,2-de]phthalazin-3(2H)-one;

9-(4-((dimethylamino)methyl)phenyl)-5-fluoro-10-(1H-1,2,4-triazol-5-yl)-9,10-dihydroazepino[4,3,2-de]phthalazin-3(2H)-one;

9-(4-((dimethylamino)methyl)phenyl)-5-fluoro-10-(1-methyl-1H-1,2,4-triazol-5-yl)-9,10-dihydroazepino[4,3,2-de]phthalazin-3(2H)-one;

9-(4-((dimethylamino)methyl)phenyl)-10-(1-ethyl-1H-1,2,4-triazol-5-yl)-5-fluoro-9,10-dihydroazepino[4,3,2-de]phthalazin-3(2H)-one;

9-(4-((dimethylamino)methyl)phenyl)-5-fluoro-10-(1-(2-hydroxyethyl)-1H-1,2,4-triazol-5-yl)-9,10-dihydroazepino[4,3,2-de]phthalazin-3(2H)-one;

9-(4-((dimethylamino)methyl)phenyl)-5-fluoro-10-(1-(3-hydroxypropyl)-1H-1,2,4-triazol-5-yl)-9,10-dihydroazepino[4,3,2-de]phthalazin-3(2H)-one;

9-(4-(azetidin-1-ylmethyl)phenyl)-5-fluoro-10-(1H-1,2,4-triazol-5-yl)-9,10-dihydroazepino[4,3,2-de]phthalazin-3(2H)-one;

9-(4-(azetidin-1-ylmethyl)phenyl)-5-fluoro-10-(1-methyl-1H-1,2,4-triazol-5-yl)-9,10-dihydroazepino[4,3,2-de]phthalazin-3(2H)-one;

9-(4-(azetidin-1-ylmethyl)phenyl)-10-(1-ethyl-1H-1,2,4-triazol-5-yl)-5-fluoro-9,10-dihydroazepino[4,3,2-de]phthalazin-3(2H)-one;

9-(4-(azetidin-1-ylmethyl)phenyl)-5-fluoro-10-(1-propyl-1H-1,2,4-triazol-5-yl)-9,10-dihydroazepino[4,3,2-de]phthalazin-3(2H)-one;

9-(4-(azetidin-1-ylmethyl)phenyl)-5-fluoro-10-(1-(2-hydroxyethyl)-1H-1,2,4-triazol-5-yl)-9,10-dihydroazepino[4,3,2-de]phthalazin-3(2H)-one;

9-(4-(azetidin-1-ylmethyl)phenyl)-5-fluoro-10-(1-(3-hydroxypropyl)-1H-1,2,4-triazol-5-yl)-9,10-dihydroazepino[4,3,2-de]phthalazin-3(2H)-one;

9-(4-fluorophenyl)-10-(1H-1,2,4-triazol-5-yl)-9,10-dihydroazepino[4,3,2-de]phthalazin-3(2H)-one;

9-(4-fluorophenyl)-10-(1-methyl-1H-1,2,4-triazol-5-yl)-9,10-dihydroazepino[4,3,2-de]phthalazin-3(2H)-one;

9-(4-fluorophenyl)-10-(1-(2-hydroxyethyl)-1H-1,2,4-triazol-5-yl)-9,10-dihydroazepino[4,3,2-de]phthalazin-3(2H)-one;

9-(4-fluorophenyl)-10-(1-(3-hydroxypropyl)-1H-1,2,4-triazol-5-yl)-9,10-dihydroazepino[4,3,2-de]phthalazin-3(2H)-one;

10-(1-(2-hydroxyethyl)-1H-1,2,4-triazol-5-yl)-9-(4-((methylamino)methyl)phenyl)-9,10-dihydroazepino[4,3,2-de]phthalazin-3(2H)-one;

10-(1-methyl-1H-1,2,4-triazol-5-yl)-9-(4-((methylamino)methyl)phenyl)-9,10-dihydroazepino[4,3,2-de]phthalazin-3(2H)-one;

9-(4-((methylamino)methyl)phenyl)-10-(1H-1,2,4-triazol-5-yl)-9,10-dihydroazepino[4,3,2-de]phthalazin-3(2H)-one;

9-(4-(azetidin-1-ylmethyl)phenyl)-10-(1-(2-hydroxyethyl)-1H-1,2,4-triazol-5-yl)-9,10-dihydroazepino[4,3,2-de]phthalazin-3(2H)-one;

9-(4-(azetidin-1-ylmethyl)phenyl)-10-(1-methyl-1H-1,2,4-triazol-5-yl)-9,10-dihydroazepino[4,3,2-de]phthalazin-3(2H)-one; and

9-(4-(azetidin-1-ylmethyl)phenyl)-10-(1H-1,2,4-triazol-5-yl)-9,10-dihydroazepino[4,3,2-de]phthalazin-3(2H)-one.

### Example 8

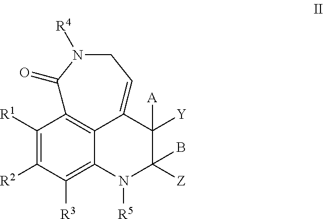

### Synthesis of 9-fluoro-2-(4-fluorophenyl)-3-(1-methyl-1H-1,2,4-triazol-5-yl)-2,3,5,6-tetrahydroazepino[5,4,3-de]quinolin-7(1H)-one (F)

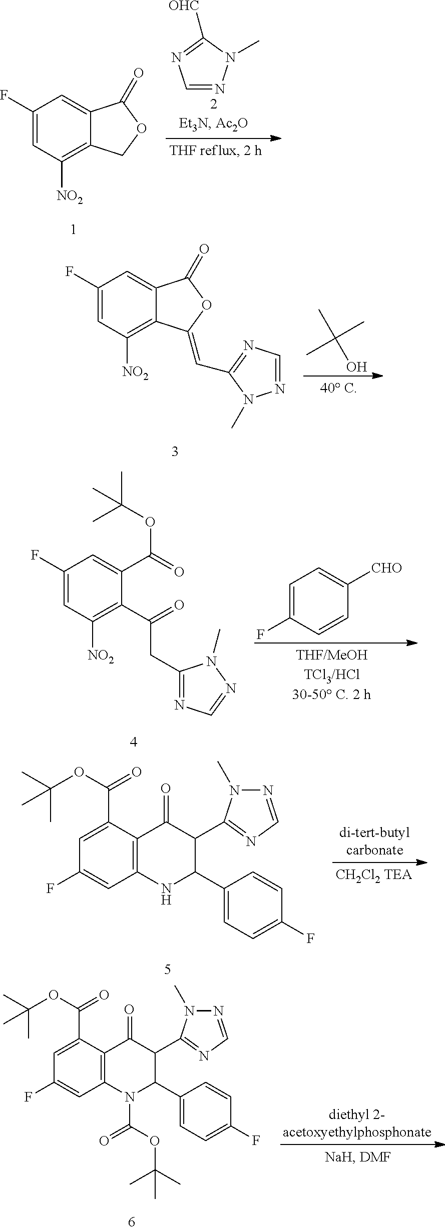

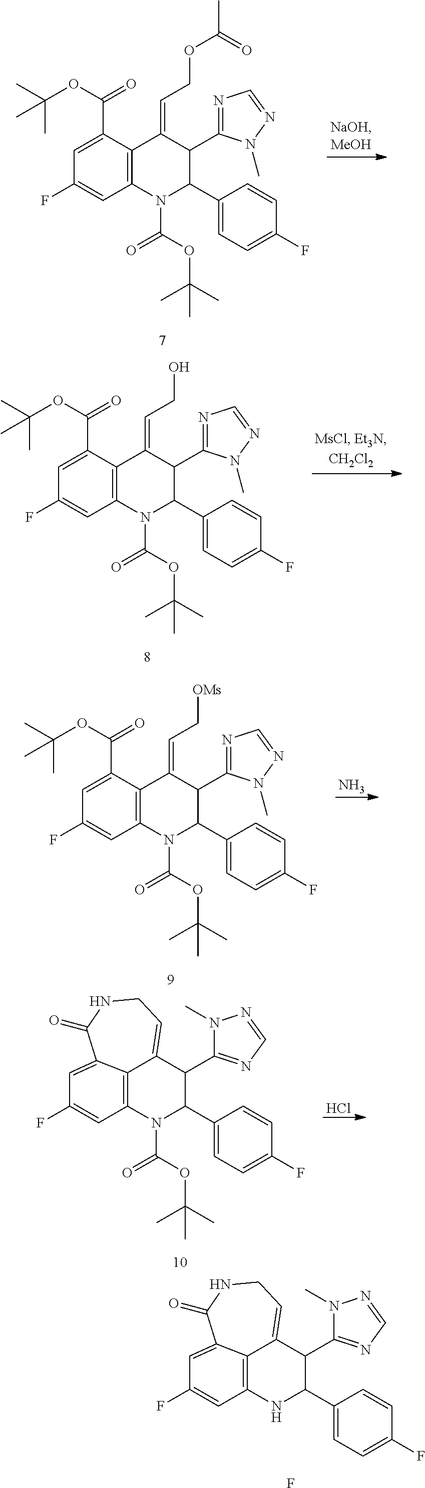

### Preparation of (Z)-6-fluoro-3-((1-methyl-1H-1,2,4-triazol-5-yl)methylene)-4-nitroisobenzofuran-1(3H)-one (3)

To a solution of 6-fluoro-4-nitroisobenzofuran-1(3H)-one (1) (10 g, 50.8 mmol), 2-methyl-2H-1,2,4-triazole-3-carbaldehyde (2) (11 g, 101.5 mmol) and Et₃N (20 mL, 152.4 mmol) in anhydrous THF (150 mL) was added acetic anhydride (35 mL) dropwise with stirring at room temperature in 3 minutes. The mixture was then stirred at reflux for 45 minutes when a green solid was precipitated from solvent. The solvent was concentrated to 15 mL, the resulting mixture was cooled to 0° C. and filtered, washed with 15 mL of ethyl acetate to obtain (Z)-6-fluoro-3-(0-methyl-1H-1,2,4-triazol-5-yl)methylene)-4-nitroisobenzofuran-1(3H)-one (3) as a green solid (11.5 g, yield: 78%). LC-MS (ESI) m/z: 291 (M+1)⁺. ¹H-NMR (400 MHz, DMSO-d₆) δ (ppm): 3.94 (s, 3H), 7.15 (s, 1H), 8.10 (s, 1H), 8.40-8.42 (dd, J₁=6.4 Hz, J₂=2.4 Hz, 1H), 8.58-8.61 (dd, J₁=8.8 Hz, J₂=2.4 Hz, 1H).

The following steps were not, but could be conducted, as described below.

### Preparation of tert-butyl 5-fluoro-2-(2-(1-methyl-1H-1,2,4-triazol-5-yl)acetyl)-3-nitrobenzoate (4)

(Z)-6-fluoro-3-((1-methyl-1H-1,2,4-triazol-5-yl)methylene)-4-nitroisobenzofuran-1(3H)-one (3) (29.5 g, 0.1 mol), and HCl (2 N in tert-butanol, 500 mL, 1 mol) is charged into a 1 L 3-neck flask equipped with mechanical stirrer, thermometer, nitrogen inlet/outlet. The reaction mixture is stirred at room temperature for 48 hours. The reaction mixture is monitored by HPLC. The reaction mixture is concentrated under vacuum at 40° C. to dryness, and tert-butyl 5-fluoro-2-(2-(1-methyl-1H-1,2,4-triazol-5-yl)acetyl)-3-nitrobenzoate (4) is obtained as a solid. It is used for the next step without further purification. Expected LC-MS (ESI) m/z: 365 (M+1)⁺.

### Preparation of tert-butyl 7-fluoro-2-(4-fluorophenyl)-3-(1-methyl-1H-1,2,4-triazol-5-yl)-4-oxo-1,2,3,4-tetrahydroquinoline-5-carboxylate (5)

To a suspension of tert-butyl 5-fluoro-2-(2-(1-methyl-1H-1,2,4-triazol-5-yl)acetyl)-3-nitrobenzoate (4) (5 g, 15.5 mmol, 1 eq) and 4-fluorobenzaldehyde (9.7 g, 29 mmol) in a mixture of solvents tetrahydrofuran (60 mL) and MeOH (10 mL) is added titanium(III) chloride (20% w/w solution in 2 N hydrochloric acid) (80 mL) dropwise with stirring at room temperature. The reaction mixture is allowed to stir at 30-50° C. for 2 hours. Then the mixture is diluted with water (160 mL), the resulting solution is extracted with ethyl acetate (150 mL×4). The combined organic layers are washed with saturated NaHCO₃ (75 mL×3) and aqueous NaHSO₃ (100 mL×3), dried by Na₂SO₄, and concentrated to dryness. This affords a crude solid, which is washed with petroleum ether (200 mL) to obtain tert-butyl 7-fluoro-2-(4-fluorophenyl)-3-(1-methyl-1H-1,2,4-triazol-5-yl)-4-oxo-1,2,3,4-tetrahydroquinoline-5-carboxylate (5) as a solid (5.9 g, yield: 95%, purity: 97%). Expected LC-MS (ESI) m/z: 441 (M+1)⁺.

### Preparation of di-tert-butyl 7-fluoro-2-(4-fluorophenyl)-3-(1-methyl-1H-1,2,4-triazol-5-yl)-4-oxo-3,4-dihydroquinoline-1,5(2H)-dicarboxylate (6)

To a solution of tert-butyl 7-fluoro-2-(4-fluorophenyl)-3-(1-methyl-1H-1,2,4-triazol-5-yl)-4-oxo-1,2,3,4-tetrahydroquinoline-5-carboxylate (5) (882 mg, 2 mmol) in DCM (20 mL) is added 1 mL triethyl amine and di-tert-butyl carbonate (380 mg, 2.2 mmol) at room temperature under nitrogen atmosphere and the mixture is allowed to stir overnight. DCM (100 mL) is added to the reaction and is washed with water (50 mL×2). The organic layer was dried over anhydrous Na₂SO₄ and concentrated to afford di-tert-butyl 7-fluoro-2-(4-fluorophenyl)-3-(1-methyl-1H-1,2,4-triazol-5-yl)-4-oxo-3,4-dihydroquinoline-1,5(2H)-dicarboxylate (6)). LC-MS (ESI) m/z: 541(M+1)⁺.

### Preparation of (E)-di-tert-butyl 4-(2-acetoxyethylidene)-7-fluoro-2-(4-fluorophenyl)-3-(1-methyl-1H-1,2,4-triazol-5-yl)-3,4-dihydroquinoline-1,5(2H)-dicarboxylate (7)

To a solution of di-tert-butyl 7-fluoro-2-(4-fluorophenyl)-3-(1-methyl-1H-1,2,4-triazol-5-yl)-4-oxo-3,4-dihydroquinoline-1,5(2H)-dicarboxylate (6) (541 mg, 1 mmol) and diethyl 2-acetoxyethylphosphonate (246 mg, 1.1 mmol) in DMF (15 mL) is added 60% sodium hydride in oil dispersion (45 mg, 1.1 mmol) at ice temperature. It is then warm to room temperature and is then heated at 65° C. with stirring overnight. It is cooled and at ice temperature water (2 mL) is added and solution is adjusted to ph 7 with acetic acid. The solvent is removed under reduced pressure to dryness. The residue is dissolved in DCM (100 mL) washed with brine (50 mL×2) and the organic solution is dried over anhydrous sodium sulfate. The solvent is then removed under reduced pressure to afford (E)-di-tert-butyl 4-(2-acetoxyethylidene)-7-fluoro-2-(4-fluorophenyl)-3-(1-methyl-1H-1,2,4-triazol-5-yl)-3,4-dihydroquinoline-1,5(2H)-dicarboxylate (7). LC-MS (ESI) m/z: 611 (M+1)⁺.

### Preparation of (E)-di-tert-butyl 7-fluoro-2-(4-fluorophenyl)-4-(2-hydroxyethylidene)-3-(1-methyl-1H-1,2,4-triazol-5-yl)-3,4-dihydroquinoline-1,5(2H)-dicarboxylate (8)

To a solution of di-tert-butyl 4-(2-acetoxyethylidene)-7-fluoro-2-(4-fluorophenyl)-3-(1-methyl-1H-1,2,4-triazol-5-yl)-3,4-dihydroquinoline-1,5(2H)-dicarboxylate (7) (611 mg, 1 mmol) in methanol (5 mL) is added IN sodium hydroxide (2 mL) (2 mmol) and stirred at room temperature for 4 hours. The mixture is then diluted with water (20 mL) and extracted with EtOAc (30 mL×4). The organic layer is dried over anhydrous Na₂SO₄ and concentrated to afford (E)-di-tert-butyl 7-fluoro-2-(4-fluorophenyl)-4-(2-hydroxyethylidene)-3-(1-methyl-1H-1,2,4-triazol-5-yl)-3,4-dihydroquinoline-1,5(2H)-dicarboxylate (8). Expected LC-MS (ESI) m/z: 569(M+1)⁺.

### Preparation of (E)-di-tert-butyl 7-fluoro-2-(4-fluorophenyl)-3-(1-methyl-1H-1,2,4-triazol-5-yl)-4-(2-((methylsulfonyl)oxy)ethylidene)-3,4-dihydroquinoline-1,5(2H)-dicarboxylate (9)

To a solution of (E)-di-tert-butyl 7-fluoro-2-(4-fluorophenyl)-4-(2-hydroxyethylidene)-3-(1-methyl-1H-1,2,4-triazol-5-yl)-3,4-dihydroquinoline-1,5(2H)-dicarboxylate (8) (568 mg, 1 mmol) in DCM (10 mL) and 0.5 mL triethyl amine, is added methanesulfonyl chloride (150 mg, 1.1 mmol) at ice temperature under nitrogen atmosphere. The mixture is allowed to warm up to room temperature and stir overnight. DCM (100 mL) is added to the reaction and is washed with water (50 mL×2). The organic layer is dried over anhydrous Na₂SO₄ and concentrated to afford (E)-di-tert-butyl 7-fluoro-2-(4-fluorophenyl)-3-(1-methyl-1H-1,2,4-triazol-5-yl)-4-(2-((methylsulfonyl)oxy)ethylidene)-3,4-dihydroquinoline-1,5(2H)-dicarboxylate (9). Expected LC-MS (ESI) m/z: 647(M+1)⁺.

### Synthesis of tert-butyl 9-fluoro-2-(4-fluorophenyl)-3-(1-methyl-1H-1,2,4-triazol-5-yl)-7-oxo-2,3,6,7-tetrahydroazepino[5,4,3-de]quinolin-1(5H)-carboxylate (10)

To a solution of (E)-di-tert-butyl 7-fluoro-2-(4-fluorophenyl)-3-(1-methyl-1H-1,2,4-triazol-5-yl)-4-(2-((methylsulfonyl)oxy)ethylidene)-3,4-dihydroquinoline-1,5(2H)-dicarboxylate (9) (323 mg, 0.5 mmol), in anhydrous THF (15 mL) is added liquid ammonia (0.5 mL) at dry ice temperature. The mixture is heated at 70-75° C. in a sealed vessel for 4 hours. Then the solvent is removed under reduced pressure and the residue is dissolved in DCM (50 mL). It is then washed with brine (20 mL×2). The organic solvent is concentrated to dryness. The residue is then purified by column chromatography in silica gel column to obtain tert-butyl 9-fluoro-2-(4-fluorophenyl)-3-(1-methyl-1H-1,2,4-triazol-5-yl)-7-oxo-2,3,6,7-tetrahydroazepino[5,4,3-de]quinolin-1(5H)-carboxylate (10). Expected LC-MS (ESI) m/z: 494 (M+1)⁺.

### Synthesis of 9-fluoro-2-(4-fluorophenyl)-3-(1-methyl-1H-1,2,4-triazol-5-yl)-2,3,5,6-tetrahydroazepino[5,4,3-de]quinolin-7(1H)-one (F)

To a solution of tert-butyl 9-fluoro-2-(4-fluorophenyl)-3-(1-methyl-1H-1,2,4-triazol-5-yl)-7-oxo-2,3,6,7-tetrahydroazepino[5,4,3-de]quinolin-1(5H)-carboxylate (10) (197 mg, 0.4 mmol) in acetic acid (10 mL) is 2.6 mL of 5N hydrochloric acid (1.3 mL) at room temperature under nitrogen atmosphere and stirred for 5 hours. The solvent is removed under reduced pressure and the residue is dissolved in EtOAc (200 mL). It is then washed with brine (40 mL×2). The organic layer is dried over anhydrous Na₂SO₄ and concentrated to afford 9-fluoro-2-(4-fluorophenyl)-3-(1-methyl-1H-1,2,4-triazol-5-yl)-2,3,5,6-tetrahydroazepino[5,4,3-de]quinolin-7(1H)-one (F). Expected LC-MS (ESI) m/z: 394 (M+1)⁺.

Using analogous methods and substituting with appropriate starting materials, the following compounds can be prepared using the procedures as described above.

9-fluoro-2-(4-fluorophenyl)-3-(1H-1,2,4-triazol-5-yl)-2,3,5,6-tetrahydroazepino[5,4,3-de]quinolin-7(1H)-one;

9-fluoro-2-(4-fluorophenyl)-3-(1-methyl-1H-1,2,4-triazol-5-yl)-2,3,5,6-tetrahydroazepino[5,4,3-de]quinolin-7(1H)-one;

3-(1-ethyl-1H-1,2,4-triazol-5-yl)-9-fluoro-2-(4-fluorophenyl)-2,3,5,6-tetrahydroazepino[5,4,3-de]quinolin-7(1H)-one;

9-fluoro-2-(4-fluorophenyl)-3-(1-propyl-1H-1,2,4-triazol-5-yl)-2,3,5,6-tetrahydroazepino[5,4,3-de]quinolin-7(1H)-one;

9-fluoro-2-(4-fluorophenyl)-3-(1-(2-hydroxyethyl)-1H-1,2,4-triazol-5-yl)-2,3,5,6-tetrahydroazepino[5,4,3-de]quinolin-7(1H)-one;

9-fluoro-2-(4-fluorophenyl)-3-(1-(3-hydroxypropyl)-1H-1,2,4-triazol-5-yl)-2,3,5,6-tetrahydroazepino[5,4,3-de]quinolin-7(1H)-one;

9-fluoro-2-phenyl-3-(1H-1,2,4-triazol-5-yl)-2,3,5,6-tetrahydroazepino[5,4,3-de]quinolin-7(1H)-one;

9-fluoro-3-(1-methyl-1H-1,2,4-triazol-5-yl)-2-phenyl-2,3,5,6-tetrahydroazepino[5,4,3-de]quinolin-7(1H)-one;

3-(1-ethyl-1H-1,2,4-triazol-5-yl)-9-fluoro-2-phenyl-2,3,5,6-tetrahydroazepino[5,4,3-de]quinolin-7(1H)-one:

9-fluoro-2-phenyl-3-(1-propyl-1H-1,2,4-triazol-5-yl)-2,3,5,6-tetrahydroazepino[5,4,3-de]quinolin-7(1H)-one;

9-fluoro-3-(1-(2-hydroxyethyl)-1H-1,2,4-triazol-5-yl)-2-phenyl-2,3,5,6-tetrahydroazepino[5,4,3-de]quinolin-7(1H)-one;

9-fluoro-3-(1-(3-hydroxypropyl)-1H-1,2,4-triazol-5-yl)-2-phenyl-2,3,5,6-tetrahydroazepino[5,4,3-de]quinolin-7(1H)-one;

9-fluoro-2-(4-((methylamino)methyl)phenyl)-3-(1H-1,2,4-triazol-5-yl)-2,3,5,6-tetrahydroazepino[5,4,3-de]quinolin-7(1H)-one;

9-fluoro-3-(1-methyl-1H-1,2,4-triazol-5-yl)-2-(4-((methylamino)methyl)phenyl)-2,3,5,6-tetrahydroazepino[5,4,3-de]quinolin-7(1H)-one;

3-(1-ethyl-1H-1,2,4-triazol-5-yl)-9-fluoro-2-(4-((methylamino)methyl)phenyl)-2,3,5,6-tetrahydroazepino[5,4,3-de]quinolin-7(1H)-one;

9-fluoro-3-(1-(2-hydroxyethyl)-1H-1,2,4-triazol-5-yl)-2-(4-((methylamino)methyl)phenyl)-2,3,5,6-tetrahydroazepino[5,4,3-de]quinolin-7(1H)-one;

9-fluoro-3-(1-(3-hydroxypropyl)-1H-1,2,4-triazol-5-yl)-2-(4-((methylamino)methyl)phenyl)-2,3,5,6-tetrahydroazepino[5,4,3-de]quinolin-7(1H)-one;

2-(4-((dimethylamino)methyl)phenyl)-9-fluoro-3-(1H-1,2,4-triazol-5-yl)-2,3,5,6-tetrahydroazepino[5,4,3-de]quinolin-7(1H)-one;

2-(4-((dimethylamino)methyl)phenyl)-9-fluoro-3-(1-methyl-1H-1,2,4-triazol-5-yl)-2,3,5,6-tetrahydroazepino[5,4,3-de]quinolin-7(1H)-one;

2-(4-((dimethylamino)methyl)phenyl)-3-(1-ethyl-1H-1,2,4-triazol-5-yl)-9-fluoro-2,3,5,6-tetrahydroazepino[5,4,3-de]quinolin-7(1H)-one;

2-(4-((dimethylamino)methyl)phenyl)-9-fluoro-3-(1-(2-hydroxyethyl)-1H-1,2,4-triazol-5-yl)-2,3,5,6-tetrahydroazepino[5,4,3-de]quinolin-7(1H)-one;

2-(4-((dimethylamino)methyl)phenyl)-9-fluoro-3-(1-(3-hydroxypropyl)-1H -1,2,4-triazol-5-yl)-2,3,5,6-tetrahydroazepino[5,4,3-de]quinolin-7(1H)-one;

2-(4-(azetidin-1-ylmethyl)phenyl)-9-fluoro-3-(1H-1,2,4-triazol-5-yl)-2,3,5,6-tetrahydroazepino[5,4,3-de]quinolin-7(1H)-one;

2-(4-(azetidin-1-ylmethyl)phenyl)-9-fluoro-3-(1-methyl-1H-1,2,4-triazol-5-yl)-2,3,5,6-tetrahydroazepino[5,4,3-de]quinolin-7(1H)-one;

2-(4-(azetidin-1-ylmethyl)phenyl)-3-(1-ethyl-1H-1,2,4-triazol-5-yl)-9-fluoro-2,3,5,6-tetrahydroazepino[5,4,3-de]quinolin-7(1H)-one;

2-(4-(azetidin-1-ylmethyl)phenyl)-9-fluoro-3-(1-propyl-1H-1,2,4-triazol-5-yl)-2,3,5,6-tetrahydroazepino[5,4,3-de]quinolin-7(1H)-one;

2-(4-(azetidin-1-ylmethyl)phenyl)-9-fluoro-3-(1-(2-hydroxyethyl)-1H-1,2,4-triazol-5-yl)-2,3,5,6-tetrahydroazepino[5,4,3-de]quinolin-7(1H)-one;

2-(4-(azetidin-1-ylmethyl)phenyl)-9-fluoro-3-(1-(3-hydroxypropyl)-1H-1,2,4-triazol-5-yl)-2,3,5,6-tetrahydroazepino[5,4,3-de]quinolin-7(1H)-one;

2-(4-fluorophenyl)-3-(1H-1,2,4-triazol-5-yl)-2,3,5,6-tetrahydroazepino[5,4,3-de]quinolin-7(1H)-one;

2-(4-fluorophenyl)-3-(1-methyl-1H-1,2,4-triazol-5-yl)-2,3,5,6-tetrahydroazepino [5,4,3-de]quinolin-7(1H)-one;

2-(4-fluorophenyl)-3-(1-(2-hydroxyethyl)-1H-1,2,4-triazol-5-yl)-2,3,5,6-tetrahydroazepino[5,4,3-de]quinolin-7(1H)-one;

2-(4-fluorophenyl)-3-(1-(3-hydroxypropyl)-1H-1,2,4-triazol-5-yl)-2,3,5,6-tetrahydroazepino[5,4,3-de]quinolin-7(1H)-one;

3-(1-(2-hydroxyethyl)-1H-1,2,4-triazol-5-yl)-2-(4-((methylamino)methyl)phenyl)-2,3,5,6-tetrahydroazepino[5,4,3-de]quinolin-7(1H)-one;

3-(1-methyl-1H-1,2,4-triazol-5-yl)-2-(4-((methylamino)methyl)phenyl)-2,3,5,6-tetrahydroazepino[5,4,3-de]quinolin-7(1H)-one;

2-(4-((methylamino)methyl)phenyl)-3-(1H-1,2,4-triazol-5-yl)-2,3,5,6-tetrahydroazepino[5,4,3-de]quinolin-7(1H)-one;

2-(4-(azetidin-1-ylmethyl)phenyl)-3-(1-(2-hydroxyethyl)-1H-1,2,4-triazol-5-yl)-2,3,5,6-tetrahydroazepino[5,4,3-de]quinolin-7(1H)-one;

2-(4-(azetidin-1-ylmethyl)phenyl)-3-(1-methyl-1H-1,2,4-triazol-5-yl)-2,3,5,6-tetrahydroazepino[5,4,3-de]quinolin-7(1H)-one; and

2-(4-(azetidin-1-ylmethyl)phenyl)-3-(1H-1,2,4-triazol-5-yl)-2,3,5,6-tetrahydroazepino [5,4,3-de]quinolin-7(1H)-one.

### Biological Studies

Inhibitory effects of test compounds against human PARP 1 enzyme are assessed using Trevigen's Universal Chemiluminescent PARP Assay Kit (Trevigen CAT #4676-096-K) following the manufacturer's recommended protocol.

Immediately prior to performing the assay, the following reagents are prepared: A) 20× PARP Assay Buffer is diluted to 1× with dH₂O; B) 10× PARP Cocktail, which contains a mixture of NAD and biotinylated NAD, is diluted by the addition of 10× Activated DNA and 1× PARP Assay Buffer. Both the PARP Cocktail and Activated DNA are 1× after the dilution; C) all test compounds are initially dissolved in DMSO, and subsequently serial diluted with 1× PARP Assay Buffer; D) recombinant human PARP 1 enzyme is diluted with 1× PARP Assay Buffer to generate 0.5 unit/15 μL; E) 10× Strep-Diluent is diluted to 1× with 1× PBS/0.1% Triton X-100; F) Just before use, Strep-HRP is diluted 500-fold with 1× Strep-Diluent.

The chemiluminescent assays for PARP activity is performed in white 96-well plates that are pre-coated with histones. Briefly, strip wells are removed from the wrapper, 50 μL/well of 1× PARP Buffer is added to rehydrate the histones and incubation is allowed for 30 minutes at room temperature. Removal of the 1× PARP Buffer from the wells is accomplished by tapping the strip wells on paper towel. Serial dilutions of the test compounds are added to duplicate wells in 10 μL/well volume. Final assay concentrations of test compounds are typically between 1 and 0.0001 μM. Subsequently, recombinant human PARP 1 enzyme is added to 0.5 unit of PARP 1 enzyme/well in 15 μL/well volume. Combined volume of enzyme and inhibitor is 25 μL. Incubate the enzyme/inhibitor mixtures for 10 minutes at room temperature. To start the reaction, 25 μL/well of the 1× PARP Cocktail is added to all the wells. Controls included background wells with 1× Assay Buffer alone (no PARP) and wells with no inhibitor for determining the maximum or 100% PARP activity value. In all cases the final reaction volume is 50 μL.

The reactions are allowed to proceed for 1 hour at room temperature. The plate is then washed 4 times with 200 μL/well 1× PBS/0.1% Triton X-100, using ELx50 Automated Strip Washer (BIO-TEK). After washing, all wells are incubated for 60 minutes with 50 μL/well Strep-HRP, diluted 1:500 with 1× Strep-Diluent. The plate is washed 4 times with 200 μL/well 1× PBS/0.1% Triton X-100 using ELx50 Automated Strip Washer (BIO-TEK). After washing, the wells are dried by tapping plate onto paper towels. Equal volumes of PeroxyGlow™ A and B together are mixed and 100 μL per well is added. The light output is immediately determined in a plate reader (EnVision, by Perkin Elmer) set up for measuring chemiluminescence.

The % enzyme activity for each compound is then calculated using the following equation:

$$\% Inhibition = \frac{Activity Ctrl - X}{Activity Ctrl - Negative Ctrl} \times 100 \%$$

IC₅₀ values (the concentration at which 50% of the enzyme activity is inhibited) of each test compound is calculated using GraphPad Prism5 software.

All of the compounds are expected to have enzymatic PARP inhibitory activity. Compound 116 with a 2-butoxycarbonyl group at the 2-position of the diazepino ring and Compound 40 have PARP-inhibitory activity of less than 800 nM. Compound 40 has a PARP-inhibitory activity of about 250-300 nM.

Chemosensitization assay determines the extent by which a PARP inhibitor enhances the tumor cell-killing effect of cytotoxic drugs expressed as PF₅₀ (potentiation factor at GI₅₀)]. 8000 LoVo cells are seeded into each well of a flat-bottomed 96-well microtiter plate in a volume of 50 μL and incubated in F12K containing 10% (v/v) FBS (medium) overnight at 37° C. Cells are added with 50 μL medium alone, medium containing 2 μM PARP inhibitor, medium containing increasing concentration of Temozolomide (0-2000 μM), and medium containing 2 μM PARP inhibitor and increasing concentration of Temozolomide (0-2000 μM). Final concentration range for Temozolomide is 0-1000 μM where applicable, final concentration of PARP inhibitor is 1 μM where applicable. Final concentration of DMSO is 1% in each well. Cells are allowed to grow for 5 days before cell survival is determined by CellTiter Glo staining (Promega, Madison, Wis., USA). Cell growth, determined after subtraction of time 0 values, is expressed as a percentage of the control well that contained medium with 1% DMSO. GI₅₀ (concentration of drug that inhibited growth by 50%) values are calculated from the computer generated curves (GraphPad Software, Inc. San Diego Calif.). The potentiation factor [PE₅₀ (potentiation factor at GI₅₀)] is calculated as GI₅₀ of Temozolomide alone/GI50 of Temozolomide+PARP inhibitor. Reference: Thomas H. D. et al. (2007). Preclinical selection of a novel poly(ADP-ribose) polymerase inhibitor for clinical trial. Molecular Cancer Therapy 6, 945-956.

### Xenograft Studies

### Antitumor In Vivo Animal Efficacy Study

Female athymic nu/nu mice (8-10 weeks old) are used for all in vivo xenograft studies. Mice are quarantined for at least 1 week before experimental manipulation. Exponentially growing cells or in vivo passaged tumor fragments are implanted subcutaneously at the right flank of nude mice. Tumor-bearing mice are randomized according to tumor size into 6-8 mice/group in each study (average tumor size ˜150 mm³). Mice are observed daily for survival and tumors are measured twice weekly by caliper in two dimensions and converted to tumor mass using the formula for a prolate ellipsoid (V=0.5 a×b²), where a and b are the long and short diameters of the tumor, respectively, and assuming unit density (1 mm³=1 mg).

Single agent activity: PARP inhibitors are evaluated in Capan-1 and MX-1 xenografts for single agent activities. Compounds are dosed orally (p.o.), once daily for 28 days in vehicle 10% DMA/6% Solutal/84% PBS, and the same vehicle is used as control. Mice are continuously monitored for 10 more days after the last day of dosing.

Combination study: PARP inhibitory compounds described herein in vehicle 10% DMA/6% Solutal/84% PBS, are either administered orally, once daily for 5 days with Temozolomide (17mg/kg or 34 mg/kg, p.o., qdx5, 30 min after each dose of compounds) in SW620 xenograft model or dosed orally, once daily for 8 days with 6 mg/kg Cisplatin (once by ip on day 3, 30 min after first dose of PARP inhibitors) in MX-1 xenograft model. Mice are observed and individual tumor is measured for 30 more days after the last dosing of PARP inhibitory compounds.

### BRCA2-Deficient V-C8 or BRCA2-Complimented V-C8+B2 Cells

BRCA2-deficient V-C8 or BRCA2-complimented V-C8+B2 cells are implanted intramuscularly into the thigh of 40 CD-1 nude mice. Treatments are initiated when tumors are of measurable size (approximate leg diameter of 11 mm). Animals receive either a compound of Formula (I) or (IA) (two doses of 25 or 50 mg/kg in saline) or saline (10 mg/mL) intraperitoneally administered on days 1-5, and are monitored on a daily basis during treatment (tumor measurements, body weights and clinical evidence are recorded); and as required after the last treatment.

### ES-Cell-Derived Tumors

ES-cell-derived tumors (teratomas) are produced by subcutaneous injection of 2×10⁶ ES cells into 6-8 week athymic BALB/c-nude (nu/nu) mice. 40 mice are injected with BRCA2-deficient ES cells or isogenic wild-type cells. Two days after cell injection, treatment with a compound of Formula (I) or (IA) is initiated. For three consecutive days, two intraperitoneal doses of a compound of Formula (I), (IA) or vehicle is administered, 6 h apart, each at a dosage of 15 mg/kg per animal. This treatment is stopped for 5 days and then re-initiated for another three consecutive days. Growth of tumors is monitored from a minimum volume of 0.2 cm³.

The in vitro assays disclosed herein, along with other known in vitro assays (Farmer et al., Nature 2005; 434:913-7: clonogenic survival assay finding that a BRCA2-deficient cell line V-C8, compared with the BRCA2 wild type control exhibited sensitivity to AG14361, a PARP-1 inhibitor, (Ki=5 nm) and NU1025, a moderately potent PARP-1 inhibitor (Ki=50 nM); & Mcabe et al., Cancer Biology & Therapy 2005; 4:9, 934-36; clonogenic survival assay using CAPAN-1 cells maintained in DMEM supplemented with FCS (20% v/v), glutamine and antibiotics showing sensitivity to PARP inhibition using AZD2281) demonstrates the activity of PARP-inhibitors in a static test situation. Additionally, animal models have been used to analyze the relationship between in vitro tests and parameters of in vivo efficacy. By way of example only, Farmer et al., has shown in vivo efficacy in blocking the growth of BRCA2-deficient tumors using AZD2281, a PARP-1 inhibitor. This indicates that PARP-1 inhibition is a viable cancer treatment for BRCA1/2 mutation carriers. Furthermore, AZD2281, a PARP-1 inhibitor is currently in Phase I clinical trials for patients with advanced solid tumors. Thus, compounds disclosed herein which have in vitro inhibitory activity are likely to show analogous in vivo (mouse and human) activity.

What is claimed is:

1. A compound of Formula (M): 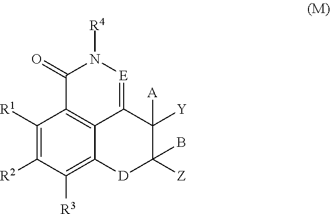
where Y and Z are each independently
a) aryl optionally substituted with 1, 2, or 3 R⁶;
b) heteroaryl optionally substituted with 1, 2, or 3 R⁶; or
c) a substituent independently selected from the group consisting of hydrogen, C₂-C₆alkenyl, C₁-C₆alkoxy, alkoxyalkyl, C₁-C₆alkoxycarbonyl, alkoxycarbonylalkyl, C₁-C₆alkyl, C₂-C₆alkynyl, arylalkyl, C₃-C₈cycloalkyl, cycloalkylalkyl, C₁-C₆haloalkyl, C₁-C₆hydroxyalkyl, oxo, C₃-C₈heterocycloalkyl, heterocycloalkylalkyl, C₁-C₆alkylcarbonyl, arylcarbonyl, heteroarylcarbonyl, C₁-C₆alkylsulfonyl, arylsulfonyl, heteroarylsulfonyl, —C₁₋C₆alkylene-(NR^{A}R^{B}), —C(O)(NR^{A}R^{B}), —C₁₋C₆alkylene-C(O)(NR^{A}R^{B}), —S(O)₂(NR^{A}R^{B}), and —C₁₋C₆alkylene-S(O)₂(NR^{A}R^{B});
E is —CH₂—CH═ and D is —N(R⁵)—; or
E is —N═ and D is —N(R⁵)—CH₂—, —CH₂—N(R⁷)—, —N(R³)—N(R⁷)—, —N(R⁵)—O—, —N═CH—, or —CH═N—;
R¹, R², and R³ are each independently selected from the group consisting of hydrogen, halogen, C₂-C₆alkenyl, alkoxy, alkoxycarbonyl, C₁-C₆alkyl, C₃-C₈cycloalkyl, C₂-C₆alkynyl, cyano, C₁-C₆haloalkoxy, C₁-C₆haloalkyl, hydroxyl, C₁-C₆hydroxyalkyl, nitro, —NR^{A}R^{B}, —C₁₋C₆alkylene-(NR^{A}R^{B}), and —C(O)(NR^{A}R^{B});
A and B are each independently selected from hydrogen, Br, Cl, F, I, OH, C₁-C₆alkyl, C₃-C₈cycloalkyl, C₁-C₆alkoxy, and alkoxyalkyl, where the C₁-C₆alkyl, C₃-C₈cycloalkyl, C₁-C₆alkoxy, and alkoxyalkyl are optionally substituted with 1, 2, 3, or 4 groups independently selected from OH, NO₂, CN, Br, Cl, F, I, C₁-C₆alkyl, and C₃-C₈cycloalkyl, where B is not OH;
R^{A} and R^{B} are independently selected from the group consisting of hydrogen, C₁-C₆alkyl, C₃-C₈cycloalkyl, and C₁-C₆alkylcarbonyl; or
R^{A} and R^{B} taken together with the atom to which they are attached form a 3-10 membered heterocyclyl optionally having one to three heteroatoms or hetero functionalities selected from the group consisting of —O—, —S— and —S(O)_{q}—, where q is 1 or 2 and R^{C} is H, C₁-C₆-alkyl, —C(O)(C₁-C₆-alkyl), aryl, aryl-C₁-C₆-alkyl, heteroaryl, or heteroaryl-C₁-C₆-alkyl;
R⁴, R⁵, and R⁷ are each independently selected from the group consisting of hydrogen, C₁-C₆alkyl, C₃-C₈cycloalkyl, alkoxyalkyl, C₁-C₆haloalkyl, C₁-C₆hydroxyalkyl, alkoxycarbonyl, and —C₁₋C₆alkylene-(NR^{A}R^{B}); and
R⁶ is selected from OH, NO₂, CN, Br, Cl, F, I, C₁-C₆alkyl, C₃-C₈cycloalkyl, C₃-C₈heterocycloalkyl;
C₂-C₆alkenyl, alkoxy, alkoxyalkyl, alkoxycarbonyl, alkoxycarbonylalkyl, C₂-C₆alkynyl, aryl, arylalkyl, C₃-C₈cycloalkylalkyl, C₁-C₆haloalkoxy, C₁-C₆haloalkyl, C₁-C₆hydroxyalkyl, oxo, heteroaryl, heteroarylalkyloxy, heteroaryloxy, heteroarylthio, heteroarylalkylthio, heterocycloalkylalkyloxy, C₃-C₈heterocycloalkylthio, C₃-C₈heterocycloalkyloxy, heterocycloalkylalkylthio, —NR^{A}R^{B}, —C₁₋C₆alkylene-(NR^{A}R^{B}), —C(O)(NR^{A}R^{B}), —C₁₋C₆alkylene-C(O)(NR^{A}R^{B}), —S(O)₂(NR^{A}R^{B}), and —C₁₋C₆alkylene-S(O)₂(NR^{A}R^{B});
optionally as a tautomer, a single stereoisomer or mixture of stereoisomers thereof and additionally optionally as a pharmaceutically acceptable salt thereof.

2. The Compound of claim 1 where the Compound of Formula (M) is according to Formula (I)

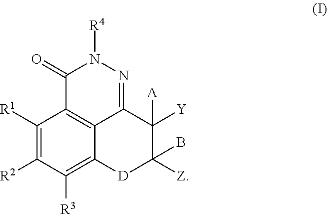

3. The Compound of claim 1 where the Compound of Formula (M) or (I) is according to Formula (IA)

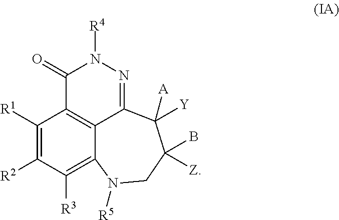

4. The Compound of claim 1 where the Compound of Formula (M) or (I) is according to Formula (IB)

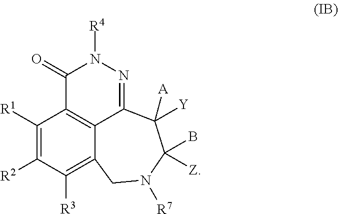

5. The Compound of claim 1 where the Compound of Formula (M) or (I) is according to Formula (IC)

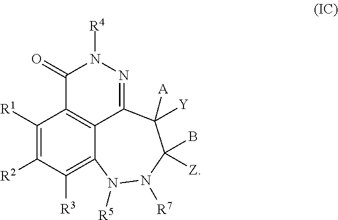

6. The Compound of claim 1 where the Compound of Formula (M) or (I) is according to Formula (ID)

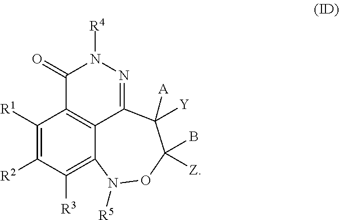

7. The Compound of claim 1 where the Compound of Formula (M) or (I) is according to Formula (IE)

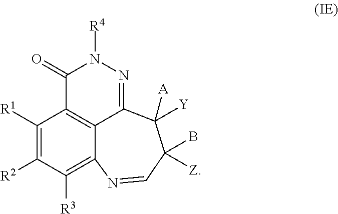

8. The Compound of claim 1 where the Compound of Formula (M) is according to Formula (II)

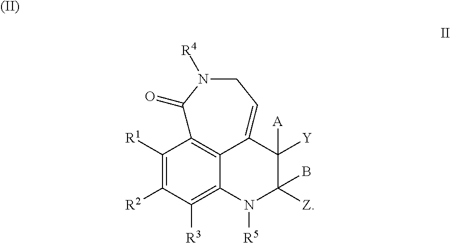

9. The Compound of claim 3 where R⁴ is hydrogen.

10. The Compound of claim 3 where A and B are hydrogen.

11. The Compound of claim 3 where R⁵ is hydrogen.

12. The Compound of claim 5 where R⁷ is hydrogen or alkoxycarbonyl.

13. The Compound of claim 3 where R¹ and R³ are hydrogen.

14. The Compound of claim 3 where R² is hydrogen or halo.

15. The Compound of claim 3 where Y is imidazolyl or triazolyl, each of which is optionally substituted with 1 or 2 R⁶.

16. The Compound of claim 3 where Z is phenyl optionally substituted with 1 or 2 R⁶.

17. The Compound of claim 3 where the Y group is unsubstituted or each R⁶ on the Y group is independently C₁-C₆alkyl or C₁-C₆hydroxyalkyl.

18. The compound of claim 1, optionally as a single stereoisomer or mixture of stereoisomers thereof and additionally optionally as a pharmaceutically acceptable salt thereof, selected from
9-fluoro-2-(4-fluorophenyl)-3-(1H-1,2,4-triazol-5-yl)-2,3,5,6-tetrahydroazepino[5,4,3-de]quinolin-
7(1H)-one;
9-fluoro-2-(4-fluorophenyl)-3-(1-methyl-1H-1,2,4-triazol-5-yl)-2,3,5,6-tetrahydroazepino[5,4,3-
de]quinolin-7(1H)-one;
3-(1-ethyl-1H-1,2,4-triazol-5-yl)-9-fluoro-2-(4-fluorophenyl)-2,3,5,6-tetrahydroazepino[5,4,3-
de]quinolin-7(1H)-one;
9-fluoro-2-(4-fluorophenyl)-3-(1-propyl-1H-1,2,4-triazol-5-yl)-2,3,5,6-tetrahydroazepino[5,4,3-
de]quinolin-7(1H)-one;
9-fluoro-2-(4-fluorophenyl)-3-(1-(2-hydroxyethyl)-1H-1,2,4-triazol-5-yl)-2,3,5,6-
tetrahydroazepino[5,4,3-de]quinolin-7(1H)-one;
9-fluoro-2-(4-fluorophenyl)-3-(1-(3-hydroxypropyl)-1H-1,2,4-triazol-5-yl)-2,3,5,6-
tetrahydroazepino[5,4,3-de]quinolin-7(1H)-one;
9-fluoro-2-phenyl-3-(1H-1,2,4-triazol-5-yl)-2,3,5,6-tetrahydroazepino[5,4,3-de]quinolin-7(1H)-one;
9-fluoro-3-(1-methyl-1H-1,2,4-triazol-5-yl)-2-phenyl-2,3,5,6-tetrahydroazepino[5,4,3-de]quinolin-
7(1H)-one;
3-(1-ethyl-1H-1,2,4-triazol-5-yl)-9-fluoro-2-phenyl-2,3,5,6-tetrahydroazepino[5,4,3-de]quinolin-
7(1H)-one;
9-fluoro-2-phenyl-3-(1-propyl-1H-1,2,4-triazol-5-yl)-2,3,5,6-tetrahydroazepino[5,4,3-de]quinolin-
7(1H)-one;
9-fluoro-3-(1-(2-hydroxyethyl)-1H-1,2,4-triazol-5-yl)-2-phenyl-2,3,5,6-tetrahydroazepino[5,4,3-
de]quinolin-7(1H)-one;
9-fluoro-3-(1-(3-hydroxypropyl)-1H-1,2,4-triazol-5-yl)-2-phenyl-2,3,5,6-tetrahydroazepino[5,4,3-
de]quinolin-7(1H)-one;
9-fluoro-2-(4-((methylamino)methyl)phenyl)-3-(1H-1,2,4-triazol-5-yl)-2,3,5,6-
tetrahydroazepino[5,4,3-de]quinolin-7(1H)-one;
9-fluoro-3-(1-methyl-1H-1,2,4-triazol-5-yl)-2-(4-((methylamino)methyl)phenyl)-2,3,5,6-
tetrahydroazepino[5,4,3-de]quinolin-7(1H)-one;
3-(1-ethyl-1H-1,2,4-triazol-5-yl)-9-fluoro-2-(4-((methylamino)methyl)phenyl)-2,3,5,6-
tetrahydroazepino[5,4,3-de]quinolin-7(1H)-one;
9-fluoro-3-(1-(2-hydroxyethyl)-1H-1,2,4-triazol-5-yl)-2-(4-((methylamino)methyl)phenyl)-2,3,5,6-
tetrahydroazepino[5,4,3-de]quinolin-7(1H)-one;
9-fluoro-3-(1-(3-hydroxypropyl)-1H-1,2,4-triazol-5-yl)-2-(4-((methylamino)methyl)phenyl)-2,3,5,6-
tetrahydroazepino[5,4,3-de]quinolin-7(1H)-one;
2-(4-((dimethylamino)methyl)phenyl)-9-fluoro-3-(1H-1,2,4-triazol-5-yl)-2,3,5,6-
tetrahydroazepino[5,4,3-de]quinolin-7(1H)-one;
2-(4-((dimethylamino)methyl)phenyl)-9-fluoro-3-(1-methyl-1H-1,2,4-triazol-5-yl)-2,3,5,6-
tetrahydroazepino[5,4,3-de]quinolin-7(1H)-one;
2-(4-((dimethylamino)methyl)phenyl)-3-(1-ethyl-1H-1,2,4-triazol-5-yl)-9-fluoro-2,3,5,6-
tetrahydroazepino[5,4,3-de]quinolin-7(1H)-one;
2-(4-((dimethylamino)methyl)phenyl)-9-fluoro-3-(1-(3-hydroxyethyl)-1H-1,2,4-triazol-5-yl)-
2,3,5,6-tetrahydroazepino[5,4,3-de]quinolin-7(1H)-one;
2-(4-((dimethylamino)methyl)phenyl)-9-fluoro-3-(1-(3-hydroxypropyl)-1H-1,2,4-triazol-5-yl)-
2,3,5,6-tetrahydroazepino[5,4,3-de]quinolin-7(1H)-one;
2-(4-(azetidin-1-ylmethyl)phenyl)-9-fluoro-3-(1H-1,2,4-triazol-5-yl)-2,3,5,6-tetrahydroazepino[5,4,3-
de]quinolin-7(1H)-one;
2-(4-(azetidin-1-ylmethyl)phenyl)-9-fluoro-3-(1-methyl-1H-1,2,4-triazol-5-yl)-2,3,5,6-
tetrahydroazepino[5,4,3-de]quinolin-7(1H)-one;
2-(4-(azetidin-1-ylmethyl)phenyl)-3-(1-ethyl-1H-1,2,4-triazol-5-yl)-9-fluoro-2,3,5,6-
tetrahydroazepino[5,4,3-de]quinolin-7(1H)-one;
2-(4-(azetidin-1-ylmethyl)phenyl)-9-fluoro-3-(1-propyl-1H-1,2,4-triazol-5-yl)-2,3,5,6-
tetrahydroazepino[5,4,3-de]quinolin-7(1H)-one;
2-(4-(azetidin-1-ylmethyl)phenyl)-9-fluoro-3-(1-(2-hydroxyethyl)-1H-1,2,4-triazol-5-yl)-2,3,5,6-
tetrahydroazepino[5,4,3-de]quinolin-7(1H)-one;
2-(4-(azetidin-1-ylmethyl)phenyl)-9-fluoro-3-(1-(3-hydroxypropyl)-1H-1,2,4-triazol-5-yl)-2,3,5,6-
tetrahydroazepino[5,4,3-de]quinolin-7(1H)-one;
2-(4-fluorophenyl)-3-(1H-1,2,4-triazol-5-yl)-2,3,5,6-tetrahydroazepino[5,4,3-de]quinolin-7(1H)-one;
2-(4-fluorophenyl)-3-(1-methyl-1H-1,2,4-triazol-5-yl)-2,3,5,6-tetrahydroazepino[5,4,3-de]quinolin-
7(1H)-one;
2-(4-fluorophenyl)-3-(1-(2-hydroxyethyl)-1H-1,2,4-triazol-5-yl)-2,3,5,6-tetrahydroazepino[5,4,3-
de]quinolin-7(1H)-one;
2-(4-fluorophenyl)-3-(1-(3-hydroxypropyl)-1H-1,2,4-triazol-5-yl)-2,3,5,6-tetrahydroazepino[5,4,3-
de]quinolin-7(1H)-one;
3-(1-(2-hydroxyethyl)-1H-1,2,4-triazol-5-yl)-2-(4-((methylamino)methyl)phenyl)-2,3,5,6-
tetrahydroazepino[5,4,3-de]quinolin-7(1H)-one;
3-(1-methyl-1H-1,2,4-triazol-5-yl)-2-(4-((methylamino)methyl)phenyl)-2,3,5,6-
tetrahydroazepino[5,4,3-de]quinolin-7(1H)-one;
2-(4-((methylamino)methyl)phenyl)-3-(1H-1,2,4-triazol-5-yl)-2,3,5,6-tetrahydroazepino[5,4,3-
de]quinolin-7(1H)-one;
2-(4-(azetidin-1-ylmethyl)phenyl)-3-(1-(2-hydroxyethyl)-1H-1,2,4-triazol-5-yl)-2,3,5,6-
tetrahydroazepino[5,4,3-de]quinolin-7(1H)-one;
2-(4-(azetidin-1-ylmethyl)phenyl)-3-(1-methyl-1H-1,2,4-triazol-5-yl)-2,3,5,6-
tetrahydroazepino[5,4,3-de]quinolin-7(1H)-one;
2-(4-(azetidin-1-ylmethyl)phenyl)-3-(1H-1,2,4-triazol-5-yl)-2,3,5,6-tetrahydroazepino[5,4,3-
de]quinolin-7(1H)-one;
5-fluoro-9-(4-fluorophenyl)-10-(1H-1,2,4-triazol-5-yl)-7,8,9,10-tetrahydroazepino[4,3,2-
de]phthalazin-3(2H)-one;
5-fluoro-9-(4-fluorophenyl)-10-(1-methyl-1H-1,2,4-triazol-5-yl)-7,8,9,10-tetrahydroazepino[4,3,2-
de]phthalazin-3(2H)-one;
10-(1-ethyl-1H-1,2,4-triazol-5-yl)-5-fluoro-9-(4-fluorophenyl)-7,8,9,10-tetrahydroazepino[4,3,2-
de]phthalazin-3(2H)-one;
5-fluoro-9-(4-fluorophenyl)-10-(1-propyl-1H-1,2,4-triazol-5-yl)-7,8,9,10-tetrahydroazepino[4,3,2-
de]phthalazin-3(2H)-one;
5-fluoro-9-(4-fluorophenyl)-10-(1-(2-hydroxyethyl)-1H-1,2,4-triazol-5-yl)-7,8,9,10-
tetrahydroazepino[4,3,2-de]phthalazin-3(2H)-one;
5-fluoro-9-(4-fluorophenyl)-10-(1-(3-hydroxypropyl)-1H-1,2,4-triazol-5-yl)-7,8,9,10-
tetrahydroazepino[4,3,2-de]phthalazin-3(2H)-one;
5-fluoro-9-phenyl-10-(1H-1,2,4-triazol-5-yl)-7,8,9,10-tetrahydroazepino[4,3,2-de]phthalazin-3(2H)-
one;
5-fluoro-10-(1-methyl-1H-1,2,4-triazol-5-yl)-9-phenyl-7,8,9,10-tetrahydroazepino[4,3,2-
de]phthalazin-3(2H)-one;
10-(1-ethyl-1H-1,2,4-triazol-5-yl)-5-fluoro-9-phenyl-7,8,9,10-tetrahydroazepino[4,3,2-de]phthalazin-
3(2H)-one;
5-fluoro-9-phenyl-10-(1-propyl-1H-1,2,4-triazol-5-yl)-7,8,9,10-tetrahydroazepino[4,3,2-
de]phthalazin-3(2H)-one;
5-fluoro-10-(1-(2-hydroxyethyl)-1H-1,2,4-triazol-5-yl)-9-phenyl-7,8,9,10-tetrahydroazepino[4,3,2-
de]phthalazin-3(2H)-one;
5-fluoro-10-(1-(3-hydroxypropyl)-1H-1,2,4-triazol-5-yl)-9-phenyl-7,8,9,10-tetrahydroazepino[4,3,2-
de]phthalazin-3(2H)-one;
5-fluoro-9-(4-((methylamino)methyl)phenyl)-10-(1H-1,2,4-triazol-5-yl)-7,8,9,10-
tetrahydroazepino[4,3,2-de]phthalazin-3(2H)-one;
5-fluoro-10-(1-methyl-1H-1,2,4-triazol-5-yl)-9-(4-((methylamino)methyl)phenyl)-7,8,9,10-
tetrahydroazepino[4,3,2-de]phthalazin-3(2H)-one;
10-(1-ethyl-1H-1,2,4-triazol-5-yl)-5-fluoro-9-(4-((methylamino)methyl)phenyl)-7,8,9,10-
tetrahydroazepino[4,3,2-de]phthalazin-3(2H)-one;
5-fluoro-10-(1-(2-hydroxyethyl)-1H-1,2,4-triazol-5-yl)-9-(4-((methylamino)methyl)phenyl)-7,8,9,10-
tetrahydroazepino[4,3,2-de]phthalazin-3(2H)-one;
5-fluoro-10-(1-(3-hydroxypropyl)-1H-1,2,4-triazol-5-yl)-9-(4-((methylamino)methyl)phenyl)-
7,8,9,10-tetrahydroazepino[4,3,2-de]phthalazin-3(2H)-one;
9-(4-((dimethylamino)methyl)phenyl)-5-fluoro-10-(1H-1,2,4-triazol-5-yl)-7,8,9,10-
tetrahydroazepino[4,3,2-de]phthalazin-3(2H)-one;
9-(4-((dimethylamino)methyl)phenyl)-5-fluoro-10-(1-methyl-1H-1,2,4-triazol-5-yl)-7,8,9,10-
tetrahydroazepino[4,3,2-de]phthalazin-3(2H)-one;
9-(4-((dimethylamino)methyl)phenyl)-10-(1-ethyl-1H-1,2,4-triazol-5-yl)-5-fluoro-7,8,9,10-
tetrahydroazepino[4,3,2-de]phthalazin-3(2H)-one;
9-(4-((dimethylamino)methyl)phenyl)-5-fluoro-10-(1-(2-hydroxyethyl)-1H-1,2,4-triazol-5-yl)-
7,8,9,10-tetrahydroazepino[4,3,2-de]phthalazin-3(2H)-one;
9-(4-((dimethylamino)methyl)phenyl)-5-fluoro-10-(1-(3-hydroxypropyl)-1H-1,2,4-triazol-5-yl)-
7,8,9,10-tetrahydroazepino[4,3,2-de]phthalazin-3(2H)-one;
9-(4-(azetidin-1-ylmethyl)phenyl)-5-fluoro-10-(1H-1,2,4-triazol-5-yl)-7,8,9,10-
tetrahydroazepino[4,3,2-de]phthalazin-3(2H)-one;
9-(4-(azetidin-1-ylmethyl)phenyl)-5-fluoro-10-(1-methyl-1H-1,2,4-triazol-5-yl)-7,8,9,10-
tetrahydroazepino[4,3,2-de]phthalazin-3(2H)-one;
9-(4-(azetidin-1-ylmethyl)phenyl)-10-(1-ethyl-1H-1,2,4-triazol-5-yl)-5-fluoro-7,8,9,10-
tetrahydroazepino[4,3,2-de]phthalazin-3(2H)-one;
9-(4-(azetidin-1-ylmethyl)phenyl)-5-fluoro-10-(1-propyl-1H-1,2,4-triazol-5-yl)-7,8,9,10-
tetrahydroazepino[4,3,2-de]phthalazin-3(2H)-one;
9-(4-(azetidin-1-ylmethyl)phenyl)-5-fluoro-10-(1-(2-hydroxyethyl)-1H-1,2,4-triazol-5-yl)-7,8,9,10-
tetrahydroazepino[4,3,2-de]phthalazin-3(2H)-one;
9-(4-(azetidin-1-ylmethyl)phenyl)-5-fluoro-10-(1-(3-hydroxypropyl)-1H-1,2,4-triazol-5-yl)-7,8,9,10-
tetrahydroazepino[4,3,2-de]phthalazin-3(2H)-one;
9-(4-fluorophenyl)-10-(1H-1,2,4-triazol-5-yl)-7,8,9,10-tetrahydroazepino[4,3.2-de]phthalazin-3(2H)-
one;
9-(4-fluorophenyl)-10-(1-methyl-1H-1,2,4-triazol-5-yl)-7,8,9,10-tetrahydroazepino[4,3,2-
de]phthalazin-3(2H)-one;
9-(4-fluorophenyl)-10-(1-(2-hydroxyethyl)-1H-1,2,4-triazol-5-yl)-7,8,9,10-tetrahydroazepino[4,3,2-
de]phthalazin-3(2H)-one;
9-(4-fluorophenyl)-10-(1-(3-hydroxypropyl)-1H-1,2,4-triazol-5-yl)-7,8,9,10-tetrahydroazepino[4,3,2-
de]phthalazin-3(2H)-one;
10-(1-(2-hydroxyethyl)-1H-1,2,4-triazol-5-yl)-9-(4-((methylamino)methyl)phenyl)-7,8,9,10-
tetrahydroazepino[4,3,2-de]phthalazin-3(2H)-one;
10-(1-methyl-1H-1,2,4-triazol-5-yl)-9-(4-((methylamino)methyl)phenyl)-7,8,9,10-
tetrahydroazepino[4,3,2-de]phthalazin-3(2H)-one;
9-(4-((methylamino)methyl)phenyl)-10-(1H-1,2,4-triazol-5-yl)-7,8,9,10-tetrahydroazepino[4,3,2-
de]phthalazin-3(2H)-one;
9-(4-(azetidin-1-ylmethyl)phenyl)-10-(1-(2-hydroxyethyl)-1H-1,2,4-triazol-5-yl)-7,8,9,10-
tetrahydroazepino[4,3,2-de]phthalazin-3(2H)-one;
9-(4-(azetidin-1-ylmethyl)phenyl)-10-(1-methyl-1H-1,2,4-triazol-5-yl)-7,8,9,10-
tetrahydroazepino[4,3,2-de]phthalazin-3(2H)-one;
9-(4-(azetidin-1-ylmethyl)phenyl)-10-(1H-1,2,4-triazol-5-yl)-7,8,9,10-tetrahydroazepino[4,3,2-
de]phthalazin-3(2H)-one;
5-fluoro-9-(4-fluorophenyl)-10-(1H-1,2,4-triazol-5-yl)-7,8,9,10-tetrahydroazepino[5,4,3-
de]phthalazin-3(2H)-one;
5-fluoro-9-(4-fluorophenyl)-10-(1-methyl-1H-1,2,4-triazol-5-yl)-7,8,9,10-tetrahydroazepino[5,4,3-
de]phthalazin-3(2H)-one;
10-(1-ethyl-1H-1,2,4-triazol-5-yl)-5-fluoro-9-(4-fluorophenyl)-7,8,9,10-tetrahydroazepino[5,4,3-
de]phthalazin-3(2H)-one;
5-fluoro-9-(4-fluorophenyl)-10-(1-propyl-1H-1,2,4-triazol-5-yl)-7,8,9,10-tetrahydroazepino[5,4,3-
de]phthalazin-3(2H)-one;
5-fluoro-9-(4-fluorophenyl)-10-(1-(2-hydroxyethyl)-1H-1,2,4-triazol-5-yl)-7,8,9,10-
tetrahydroazepino[5,4,3-de]phthalazin-3(2H)-one;
5-fluoro-9-(4-fluorophenyl)-10-(1-(3-hydroxypropyl)-1H-1,2,4-triazol-5-yl)-7,8,9,10-
tetrahydroazepino[5,4,3-de]phthalazin-3(2H)-one;
5-fluoro-9-phenyl-10-(1H-1,2,4-triazol-5-yl)-7,8,9,10-tetrahydroazepino[5,4,3-de]phthalazin-3(2H)-
one;
5-fluoro-10-(1-methyl-1H-1,2,4-triazol-5-yl)-9-phenyl-7,8,9,10-tetrahydroazepino[5,4,3-
de]phthalazin-3(2H)-one;
10-(1-ethyl-1H-1,2,4-triazol-5-yl)-5-fluoro-9-phenyl-7,8,9,10-tetrahydroazepino[5,4,3-de]phthalazin-
3(2H)-one;
5-fluoro-9-phenyl-10-(1-propyl-1H-1,2,4-triazol-5-yl)-7,8,9,10-tetrahydroazepino[5,4,3-
de]phthalazin-3(2H)-one;
5-fluoro-10-(1-(2-hydroxyethyl)-1H-1,2,4-triazol-5-yl)-9-phenyl-7,8,9,10-tetrahydroazepino[5,4,3-
de]phthalazin-3(2H)-one;
5-fluoro-10-(1-(3-hydroxypropyl)-1H-1,2,4-triazol-5-yl)-9-phenyl-7,8,9,10-tetrahydroazepino[5,4,3-
de]phthalazin-3(2H)-one;
5-fluoro-9-(4-((methylamino)methyl)phenyl)-10-(1H-1,2,4-triazol-5-yl)-7,8,9,10-
tetrahydroazepino[5,4,3-de]phthalazin-3(2H)-one;
5-fluoro-10-(1-methyl-1H-1,2,4-triazol-5-yl)-9-(4-((methylamino)methyl)phenyl)-7,8,9,10-
tetrahydroazepino[5,4,3-de]phthalazin-3(2H)-one;
10-(1-ethyl-1H-1,2,4-triazol-5-yl)-5-fluoro-9-(4-((methylamino)methyl)phenyl)-7,8,9,10-
tetrahydroazepino[5,4,3-de]phthalazin-3(2H)-one;
5-fluoro-10-(1-(2-hydroxyethyl)-1H-1,2,4-triazol-5-yl)-9-(4-((methylamino)methyl)phenyl)-7,8,9,10-
tetrahydroazepino[5,4,3-de]phthalazin-3(2H)-one;
5-fluoro-10-(1-(3-hydroxypropyl)-1H-1,2,4-triazol-5-yl)-9-(4-((methylamino)methyl)phenyl)-
7,8,9,10-tetrahydroazepino[5,4,3-de]phthalazin-3(2H)-one;
9-(4-((dimethylamino)methyl)phenyl)-5-fluoro-10-(1H-1,2,4-triazol-5-yl)-7,8,9,10-
tetrahydroazepino[5,4,3-de]phthalazin-3(2H)-one;
9-(4-((dimethylamino)methyl)phenyl)-5-fluoro-10-(1-methyl-1H-1,2,4-triazol-5-yl)-7,8,9,10-
tetrahydroazepino[5,4,3-de]phthalazin-3(2H)-one;
9-(4-((dimethylamino)methyl)phenyl)-10-(1-ethyl-1H-1,2,4-triazol-5-yl)-5-fluoro-7,8,9,10-
tetrahydroazepino[5,4,3-de]phthalazin-3(2H)-one;
9-(4-((dimethylamino)methyl)phenyl)-5-fluoro-10-(1-(2-hydroxyethyl)-1H-1,2,4-triazol-5-yl)-
7,8,9,10-tetrahydroazepino[5,4,3-de]phthalazin-3(2H)-one;
9-(4-((dimethylamino)methyl)phenyl)-5-fluoro-10-(1-(3-hydroxypropyl)-1H-1,2,4-triazol-5-yl)-
7,8,9,10-tetrahydroazepino[5,4,3-de]phthalazin-3(2H)-one;
9-(4-(azetidin-1-ylmethyl)phenyl)-5-fluoro-10-(1H-1,2,4-triazol-5-yl)-7,8,9,10-
tetrahydroazepino[5,4,3-de]phthalazin-3(2H)-one;
9-(4-(azetidin-1-ylmethyl)phenyl)-5-fluoro-10-(1-methyl-1H-1,2,4-triazol-5-yl)-7,8,9,10-
tetrahydroazepino[5,4,3-de]phthalazin-3(2H)-one;
9-(4-(azetidin-1-ylmethyl)phenyl)-10-(1-ethyl-1H-1,2,4-triazol-5-yl)-5-fluoro-7,8,9,10-
tetrahydroazepino[5,4,3-de]phthalazin-3(2H)-one;
9-(4-(azetidin-1-ylmethyl)phenyl)-5-fluoro-10-(1-propyl-1H-1,2,4-triazol-5-yl)-7,8,9,10-
tetrahydroazepino[5,4,3-de]phthalazin-3(2H)-one;
9-(4-(azetidin-1-ylmethyl)phenyl)-5-fluoro-10-(1-(2-hydroxyethyl)-1H-1,2,4-triazol-5-yl)-7,8,9,10-
tetrahydroazepino[5,4,3-de]phthalazin-3(2H)-one;
9-(4-(azetidin-1-ylmethyl)phenyl)-5-fluoro-10-(1-(3-hydroxypropyl)-1H-1,2,4-triazol-5-yl)-7,8,9,10-
tetrahydroazepino[5,4,3-de]phthalazin-3(2H)-one;
9-(4-fluorophenyl)-10-(1H-1,2,4-triazol-5-yl)-7,8,9,10-tetrahydroazepino[5,4,3-de]phthalazin-3(2H)-
one;
9-(4-fluorophenyl)-10-(1-methyl-1H-1,2,4-triazol-5-yl)-7,8,9,10-tetrahydroazepino[5,4,3-
de]phthalazin-3(2H)-one;
9-(4-fluorophenyl)-10-(1-(2-hydroxyethyl)-1H-1,2,4-triazol-5-yl)-7,8,9,10-tetrahydroazepino[5,4,3-
de]phthalazin-3(2H)-one;
9-(4-fluorophenyl)-10-(1-(3-hydroxypropyl)-1H-1,2,4-triazol-5-yl)-7,8,9,10-tetrahydroazepino[5,4,3-
de]phthalazin-3(2H)-one;
10-(1-(2-hydroxyethyl)-1H-1,2,4-triazol-5-yl)-9-(4-((methylamino)methyl)phenyl)-7,8,9,10-
tetrahydroazepino[5,4,3-de]phthalazin-3(2H)-one;
10-(1-methyl-1H-1,2,4-triazol-5-yl)-9-(4-((methylamino)methyl)phenyl)-7,8,9,10-
tetrahydroazepino[5,4,3-de]phthalazin-3(2H)-one;
9-(4-((methylamino)methyl)phenyl)-10-(1H-1,2,4-triazol-5-yl)-7,8,9,10-tetrahydroazepino[5,4,3-
de]phthalazin-3(2H)-one;
9-(4-(azetidin-1-ylmethyl)phenyl)-10-(1-(2-hydroxyethyl)-1H-1,2,4-triazol-5-yl)-7,8,9,10-
tetrahydroazepino[5,4,3-de]phthalazin-3(2H)-one;
9-(4-(azetidin-1-ylmethyl)phenyl)-10-(1-methyl-1H-1,2,4-triazol-5-yl)-7,8,9,10-
tetrahydroazepino[5,4,3-de]phthalazin-3(2H)-one;
9-(4-(azetidin-1-ylmethyl)phenyl)-10-(1H-1,2,4-triazol-5-yl)-7,8,9,10-tetrahydroazepino[5,4,3-
de]phthalazin-3(2H)-one;
9-fluoro-3-(4-fluorophenyl)-4-(1H-1,2,4-triazol-5-yl)-1,2,3,4-tetrahydro-[1,2]diazepino[5,4,3-
de]phthalazin-7(6H)-one;
9-fluoro-3-(4-fluorophenyl)-4-(1-methyl-1H-1,2,4-triazol-5-yl)-1,2,3,4-tetrahydro-
[1,2]diazepino[5,4,3-de]phthalazin-7(6H)-one;
4-(1-ethyl-1H-1,2,4-triazol-5-yl)-9-fluoro-3-(4-fluorophenyl)-1,2,3,4-tetrahydro-[1,2]diazepino[5,4,3-
de]phthalazin-7(6H)-one;
9-fluoro-3-(4-fluorophenyl)-4-(1-propyl-1H-1,2,4-triazol-5-yl)-1,2,3,4-tetrahydro-
[1,2]diazepino[5,4,3-de]phthalazin-7(6H)-one;
9-fluoro-3-(4-fluorophenyl)-4-(1-(2-hydroxyethyl)-1H-1,2,4-triazol-5-yl)-1,2,3,4-tetrahydro-
[1,2]diazepino[5,4,3-de]phthalazin-7(6H)-one;
9-fluoro-3-(4-fluorophenyl)-4-(1-(3-hydroxypropyl)-1H-1,2,4-triazol-5-yl)-1,2,3,4-tetrahydro-
[1,2]diazepino[5,4,3-de]phthalazin-7(6H)-one;
9-fluoro-3-phenyl-4-(1H-1,2,4-triazol-5-yl)-1,2,3,4-tetrahydro-[1,2]diazepino[5,4,3-de]phthalazin-
7(6H)-one;
9-fluoro-4-(1-methyl-1H-1,2,4-triazol-5-yl)-3-phenyl-1,2,3,4-tetrahydro-[1,2]diazepino[5,4,3-
de]phthalazin-7(6H)-one;
4-(1-ethyl-1H-1,2,4-triazol-5-yl)-9-fluoro-3-phenyl-1,2,3,4-tetrahydro-[1,2]diazepino[5,4,3-
de]phthalazin-7(6H)-one;
9-fluoro-3-phenyl-4-(1-propyl-1H-1,2,4-triazol-5-yl)-1,2,3,4-tetrahydro-[1,2]diazepino[5,4,3-
de]phthalazin-7(6H)-one;
9-fluoro-4-(1-(2-hydroxyethyl)-1H-1,2,4-triazol-5-yl)-3-phenyl-1,2,3,4-tetrahydro-
[1,2]diazepino[5,4,3-de]phthalazin-7(6H)-one;
9-fluoro-4-(1-(3-hydroxypropyl)-1H-1,2,4-triazol-5-yl)-3-phenyl-1,2,3,4-tetrahydro-
[1,2]diazepino[5,4,3-de]phthalazin-7(6H)-one;
9-fluoro-3-(4-((methylamino)methyl)phenyl)-4-(1H-1,2,4-triazol-5-yl)-1,2,3,4-tetrahydro-
[1,2]diazepino[5,4,3-de]phthalazin-7(6H)-one;
9-fluoro-4-(1-methyl-1H-1,2,4-triazol-5-yl)-3-(4-((methylamino)methyl)phenyl)-1,2,3,4-tetrahydro-
[1,2]diazepino[5,4,3-de]phthalazin-7(6H)-one;
4-(1-ethyl-1H-1,2,4-triazol-5-yl)-9-fluoro-3-(4-((methylamino)methyl)phenyl)-1,2,3,4-tetrahydro-
[1,2]diazepino[5,4,3-de]phthalazin-7(6H)-one;
9-fluoro-4-(1-(2-hydroxyethyl)-1H-1,2,4-triazol-5-yl)-3-(4-((methylamino)methyl)phenyl)-1,2,3,4-
tetrahydro-[1,2]diazepino[5,4,3-de]phthalazin-7(6H)-one;
9-fluoro-4-(1-(3-hydroxypropyl)-1H-1,2,4-triazol-5-yl)-3-(4-((methylamino)methyl)phenyl)-1,2,3,4-
tetrahydro-[1,2]diazepino[5,4,3-de]phthalazin-7(6H)-one;
3-(4-((dimethylamino)methyl)phenyl)-9-fluoro-4-(1H-1,2,4-triazol-5-yl)-1,2,3,4-tetrahydro-
[1,2]diazepino[5,4,3-de]phthalazin-7(6H)-one;
3-(4-((dimethylamino)methyl)phenyl)-9-fluoro-4-(1-methyl-1H-1,2,4-triazol-5-yl)-1,2,3,4-tetrahydro-
[1,2]diazepino[5,4,3-de]phthalazin-7(6H)-one;
3-(4-((dimethylamino)methyl)phenyl)-4-(1-ethyl-1H-1,2,4-triazol-5-yl)-9-fluoro-1,2,3,4-tetrahydro-
[1,2]diazepino[5,4,3-de]phthalazin-7(6H)-one;
3-(4-((dimethylamino)methyl)phenyl)-9-fluoro-4-(1-(2-hydroxyethyl)-1H-1,2,4-triazol-5-yl)-1,2,3,4-
tetrahydro-[1,2]diazepino[5,4,3-de]phthalazin-7(6H)-one;
3-(4-((dimethylamino)methyl)phenyl)-9-fluoro-4-(1-(3-hydroxypropyl)-1H-1,2,4-triazol-5-yl)-
1,2,3,4-tetrahydro-[1,2]diazepino[5,4,3-de]phthalazin-7(6H)-one;
3-(4-(azetidin-1-ylmethyl)phenyl)-9-fluoro-4-(1H-1,2,4-triazol-5-yl)-1,2,3,4-tetrahydro-
[1,2]diazepino[5,4,3-de]phthalazin-7(6H)-one;
3-(4-(azetidin-1-ylmethyl)phenyl)-9-fluoro-4-(1-methyl-1H-1,2,4-triazol-5-yl)-1,2,3,4-tetrahydro-
[1,2]diazepino[5,4,3-de]phthalazin-7(6H)-one;
3-(4-(azetidin-1-ylmethyl)phenyl)-4-(1-ethyl-1H-1,2,4-triazol-5-yl)-9-fluoro-1,2,3,4-tetrahydro-
[1,2]diazepino[5,4,3-de]phthalazin-7(6H)-one;
3-(4-(azetidin-1-ylmethyl)phenyl)-9-fluoro-4-(1-propyl-1H-1,2,4-triazol-5-yl)-1,2,3,4-tetrahydro-
[1,2]diazepino[5,4,3-de]phthalazin-7(6H)-one;
3-(4-(azetidin-1-ylmethyl)phenyl)-9-fluoro-4-(1-(2-hydroxyethyl)-1H-1,2,4-triazol-5-yl)-1,2,3,4-
tetrahydro-[1,2]diazepino[5,4,3-de]phthalazin-7(6H)-one;
3-(4-(azetidin-1-ylmethyl)phenyl)-9-fluoro-4-(1-(3-hydroxypropyl)-1H-1,2,4-triazol-5-yl)-1,2,3,4-
tetrahydro-[1,2]diazepino[5,4,3-de]phthalazin-7(6H)-one;
3-(4-fluorophenyl)-4-(1H-1,2,4-triazol-5-yl)-1,2,3,4-tetrahydro-[1,2]diazepino[5,4,3-de]phthalazin-
7(6H)-one;
3-(4-fluorophenyl)-4-(1-methyl-1H-1,2,4-triazol-5-yl)-1,2,3,4-tetrahydro-[1,2]diazepino[5,4,3-
de]phthalazin-7(6H)-one;
3-(4-fluorophenyl)-4-(1-(2-hydroxyethyl)-1H-1,2,4-triazol-5-yl)-1,2,3,4-tetrahydro-
[1,2]diazepino[5,4,3-de]phthalazin-7(6H)-one;
3-(4-fluorophenyl)-4-(1-(3-hydroxypropyl)-1H-1,2,4-triazol-5-yl)-1,2,3,4-tetrahydro-
[1,2]diazepino[5,4,3-de]phthalazin-7(6H)-one;
4-(1-(2-hydroxyethyl)-1H-1,2,4-triazol-5-yl)-3-(4-((methylamino)methyl)phenyl)-1,2,3,4-tetrahydro-
[1,2]diazepino[5,4,3-de]phthalazin-7(6H)-one;
4-(1-methyl-1H-1,2,4-triazol-5-yl)-3-(4-((methylamino)methyl)phenyl)-1,2,3,4-tetrahydro-
[1,2]diazepino[5,4,3-de]phthalazin-7(6H)-one;
3-(4-((methylamino)methyl)phenyl)-4-(1H-1,2,4-triazol-5-yl)-1,2,3,4-tetrahydro-[1,2]diazepino[5,4,3-
de]phthalazin-7(6H)-one;
3-(4-(azetidin-1-ylmethyl)phenyl)-4-(1-(2-hydroxyethyl)-1H-1,2,4-triazol-5-yl)-1,2,3,4-tetrahydro-
[1,2]diazepino[5,4,3-de]phthalazin-7(6H)-one;
3-(4-(azetidin-1-ylmethyl)phenyl)-4-(1-methyl-1H-1,2,4-triazol-5-yl)-1,2,3,4-tetrahydro-
[1,2]diazepino[5,4,3-de]phthalazin-7(6H)-one;
3-(4-(azetidin-1-ylmethyl)phenyl)-4-(1H-1,2,4-triazol-5-yl)-1,2,3,4-tetrahydro-[1,2]diazepino[5,4,3-
de]phthalazin-7(6H)-one;
9-fluoro-3-(4-fluorophenyl)-4-(1H-1,2,4-triazol-5-yl)-3,4-dihydro-1H-[1,2]oxazepino[5,4,3-
de]phthalazin-7(6H)-one;
9-fluoro-3-(4-fluorophenyl)-4-(1-methyl-1H-1,2,4-triazol-5-yl)-3,4-dihydro-1H-[1,2]oxazepino[5,4,3-
de]phthalazin-7(6H)-one;
4-(1-ethyl-1H-1,2,4-triazol-5-yl)-9-fluoro-3-(4-fluorophenyl)-3,4-dihydro-1H-[1,2]oxazepino[5,4,3-
de]phthalazin-7(6H)-one;
9-fluoro-3-(4-fluorophenyl)-4-(1-propyl-1H-1,2,4-triazol-5-yl)-3,4-dihydro-1H-[1,2]oxazepino[5,4,3-
de]phthalazin-7(6H)-one;
9-fluoro-3-(4-fluorophenyl)-4-(1-(2-hydroxyethyl)-1H-1,2,4-triazol-5-yl)-3,4-dihydro-1H-
[1,2]oxazepino[5,4,3-de]phthalazin-7(6H)-one;
9-fluoro-3-(4-fluorophenyl)-4-(1-(3-hydroxypropyl)-1H-1,2,4-triazol-5-yl)-3,4-dihydro-1H-
[1,2]oxazepino[5,4,3-de]phthalazin-7(6H)-one;
9-fluoro-3-phenyl-4-(1H-1,2,4-triazol-5-yl)-3,4-dihydro-1H-[1,2]oxazepino[5,4,3-de]phthalazin-
7(6H)-one;
9-fluoro-4-(1-methyl-1H-1,2,4-triazol-5-yl)-3-phenyl-3,4-dihydro-1H-[1,2]oxazepino[5,4,3-
de]phthalazin-7(6H)-one;
4-(1-ethyl-1H-1,2,4-triazol-5-yl)-9-fluoro-3-phenyl-3,4-dihydro-1H-[1,2]oxazepino[5,4,3-
de]phthalazin-7(6H)-one;
9-fluoro-3-phenyl-4-(1-propyl-1H-1,2,4-triazol-5-yl)-3,4-dihydro-1H-[1,2]oxazepino[5,4,3-
de]phthalazin-7(6H)-one;
9-fluoro-4-(1-(2-hydroxyethyl)-1H-1,2,4-triazol-5-yl)-3-phenyl-3,4-dihydro-1H-[1,2]oxazepino[5,4,3-
de]phthalazin-7(6H)-one;
9-fluoro-4-(1-(3-hydroxypropyl)-1H-1,2,4-triazol-5-yl)-3-phenyl-3,4-dihydro-1H-
[1,2]oxazepino[5,4,3-de]phthalazin-7(6H)-one;
9-fluoro-3-(4-((methylamino)methyl)phenyl)-4-(1H-1,2,4-triazol-5-yl)-3,4-dihydro-1H-
[1,2]oxazepino[5,4,3-de]phthalazin-7(6H)-one;
9-fluoro-4-(1-methyl-1H-1,2,4-triazol-5-yl)-3-(4-((methylamino)methyl)phenyl)-3,4-dihydro-1H-
[1,2]oxazepino[5,4,3-de]phthalazin-7(6H)-one;
4-(1-ethyl-1H-1,2,4-triazol-5-yl)-9-fluoro-3-(4-((methylamino)methyl)phenyl)-3,4-dihydro-1H-
[1,2]oxazepino[5,4,3-de]phthalazin-7(6H)-one;
9-fluoro-4-(1-(2-hydroxyethyl)-1H-1,2,4-triazol-5-yl)-3-(4-((methylamino)methyl)phenyl)-3,4-
dihydro-1H-[1,2]oxazepino[5,4,3-de]phthalazin-7(6H)-one;
9-fluoro-4-(1-(3-hydroxypropyl)-1H-1,2,4-triazol-5-yl)-3-(4-((methylamino)methyl)phenyl)-3,4-
dihydro-1H-[1,2]oxazepino[5,4,3-de]phthalazin-7(6H)-one;
3-(4-((dimethylamino)methyl)phenyl)-9-fluoro-4-(1H-1,2,4-triazol-5-yl)-3,4-dihydro-1H-
[1,2]oxazepino[5,4,3-de]phthalazin-7(6H)-one;
3-(4-((dimethylamino)methyl)phenyl)-9-fluoro-4-(1-methyl-1H-1,2,4-triazol-5-yl)-3,4-dihydro-1H-
[1,2]oxazepino[5,4,3-de]phthalazin-7(6H)-one;
3-(4-((dimethylamino)methyl)phenyl)-4-(1-ethyl-1H-1,2,4-triazol-5-yl)-9-fluoro-3,4-dihydro-1H-
[1,2]oxazepino[5,4,3-de]phthalazin-7(6H)-one;
3-(4-((dimethylamino)methyl)phenyl)-9-fluoro-4-(1-(2-hydroxyethyl)-1H-1,2,4-triazol-5-yl)-3,4-
dihydro-1H-[1,2]oxazepino[5,4,3-de]phthalazin-7(6H)-one;
3-(4-((dimethylamino)methyl)phenyl)-9-fluoro-4-(1-(3-hydroxypropyl)-1H-1,2,4-triazol-5-yl)-3,4-
dihydro-1H-[1,2]oxazepino[5,4,3-de]phthalazin-7(6H)-one;
3-(4-(azetidin-1-ylmethyl)phenyl)-9-fluoro-4-(1H-1,2,4-triazol-5-yl)-3,4-dihydro-1H-
[1,2]oxazepino[5,4,3-de]phthalazin-7(6H)-one;
3-(4-(azetidin-1-ylmethyl)phenyl)-9-fluoro-4-(1-methyl-1H-1,2,4-triazol-5-yl)-3,4-dihydro-1H-
[1,2]oxazepino[5,4,3-de]phthalazin-7(6H)-one;
3-(4-(azetidin-1-ylmethyl)phenyl)-4-(1-ethyl-1H-1,2,4-triazol-5-yl)-9-fluoro-3,4-dihydro-1H-
[1,2]oxazepino[5,4,3-de]phthalazin-7(6H)-one;
3-(4-(azetidin-1-ylmethyl)phenyl)-9-fluoro-4-(1-propyl-1H-1,2,4-triazol-5-yl)-3,4-dihydro-1H-
[1,2]oxazepino[5,4,3-de]phthalazin-7(6H)-one;
3-(4-(azetidin-1-ylmethyl)phenyl)-9-fluoro-4-(1-(2-hydroxyethyl)-1H-1,2,4-triazol-5-yl)-3,4-dihydro-
1H-[1,2]oxazepino[5,4,3-de]phthalazin-7(6H)-one;
3-(4-(azetidin-1-ylmethyl)phenyl)-9-fluoro-4-(1-(3-hydroxypropyl)-1H-1,2,4-triazol-5-yl)-3,4-
dihydro-1H-[1,2]oxazepino[5,4,3-de]phthalazin-7(6H)-one;
3-(4-fluorophenyl)-4-(1H-1,2,4-triazol-5-yl)-3,4-dihydro-1H-[1,2]oxazepino[5,4,3-de]phthalazin-
7(6H)-one;
3-(4-fluorophenyl)-4-(1-methyl-1H-1,2,4-triazol-5-yl)-3,4-dihydro-1H-[1,2]oxazepino[5,4,3-
de]phthalazin-7(6H)-one;
3-(4-fluorophenyl)-4-(1-(2-hydroxyethyl)-1H-1,2,4-triazol-5-yl)-3,4-dihydro-1H-
[1,2]oxazepino[5,4,3-de]phthalazin-7(6H)-one;
3-(4-fluorophenyl)-4-(1-(3-hydroxypropyl)-1H-1,2,4-triazol-5-yl)-3,4-dihydro-1H-
[1,2]oxazepino[5,4,3-de]phthalazin-7(6H)-one;
4-(1-(2-hydroxyethyl)-1H-1,2,4-triazol-5-yl)-3-(4-((methylamino)methyl)phenyl)-3,4-dihydro-1H-
[1,2]oxazepino[5,4,3-de]phthalazin-7(6H)-one;
4-(1-methyl-1H-1,2,4-triazol-5-yl)-3-(4-((methylamino)methyl)phenyl)-3,4-dihydro-1H-
[1,2]oxazepino[5,4,3-de]phthalazin-7(6H)-one;
3-(4-((methylamino)methyl)phenyl)-4-(1H-1,2,4-triazol-5-yl)-3,4-dihydro-1H-[1,2]oxazepino[5,4,3-
de]phthalazin-7(6H)-one;
3-(4-(azetidin-1-ylmethyl)phenyl)-4-(1-(2-hydroxyethyl)-1H-1,2,4-triazol-5-yl)-3,4-dihydro-1H-
[1,2]oxazepino[5,4,3-de]phthalazin-7(6H)-one;
3-(4-(azetidin-1-ylmethyl)phenyl)-4-(1-methyl-1H-1,2,4-triazol-5-yl)-3,4-dihydro-1H-
[1,2]oxazepino[5,4,3-de]phthalazin-7(6H)-one;
3-(4-(azetidin-1-ylmethyl)phenyl)-4-(1H-1,2,4-triazol-5-yl)-3,4-dihydro-1H-[1,2]oxazepino[5,4,3-
de]phthalazin-7(6H)-one;
5-fluoro-9-(4-fluorophenyl)-10-(1H-1,2,4-triazol-5-yl)-9,10-dihydroazepino[4,3,2-de]phthalazin-
3(2H)-one;
5-fluoro-9-(4-fluorophenyl)-10-(1-methyl-1H-1,2,4-triazol-5-yl)-9,10-dihydroazepino[4,3,2-
de]phthalazin-3(2H)-one;
10-(1-ethyl-1H-1,2,4-triazol-5-yl)-5-fluoro-9-(4-fluorophenyl)-9,10-dihydroazepino[4,3,2-
de]phthalazin-3(2H)-one;
5-fluoro-9-(4-fluorophenyl)-10-(1-propyl-1H-1,2,4-triazol-5-yl)-9,10-dihydroazepino[4,3,2-
de]phthalazin-3(2H)-one;
5-fluoro-9-(4-fluorophenyl)-10-(1-(2-hydroxyethyl)-1H-1,2,4-triazol-5-yl)-9,10-
dihydroazepino[4,3,2-de]phthalazin-3(2H)-one;
5-fluoro-9-(4-fluorophenyl)-10-(1-(3-hydroxypropyl)-1H-1,2,4-triazol-5-yl)-9,10-
dihydroazepino[4,3,2-de]phthalazin-3(2H)-one;
5-fluoro-9-phenyl-10-(1H-1,2,4-triazol-5-yl)-9,10-dihydroazepino[4,3,2-de]phthalazin-3(2H)-one;
5-fluoro-10-(1-methyl-1H-1,2,4-triazol-5-yl)-9-phenyl-9,10-dihydroazepino[4,3,2-de]phthalazin-
3(2H)-one;
10-(1-ethyl-1H-1,2,4-triazol-5-yl)-5-fluoro-9-phenyl-9,10-dihydroazepino[4,3,2-de]phthalazin-3(2H)-
one;
5-fluoro-9-phenyl-10-(1-propyl-1H-1,2,4-triazol-5-yl)-9,10-dihydroazepino[4,3,2-de]phthalazin-
3(2H)-one;
5-fluoro-10-(1-(2-hydroxyethyl)-1H-1,2,4-triazol-5-yl)-9-phenyl-9,10-dihydroazepino[4,3,2-
de]phthalazin-3(2H)-one;
5-fluoro-10-(1-(3-hydroxypropyl)-1H-1,2,4-triazol-5-yl)-9-phenyl-9,10-dihydroazepino[4,3,2-
de]phthalazin-3(2H)-one;
5-fluoro-9-(4-((methylamino)methyl)phenyl)-10-(1H-1,2,4-triazol-5-yl)-9,10-dihydroazepino[4,3,2-
de]phthalazin-3(2H)-one;
5-fluoro-10-(1-methyl-1H-1,2,4-triazol-5-yl)-9-(4-((methylamino)methyl)phenyl)-9,10-
dihydroazepino[4,3,2-de]phthalazin-3(2H)-one;
10-(1-ethyl-1H-1,2,4-triazol-5-yl)-5-fluoro-9-(4-((methylamino)methyl)phenyl)-9,10-
dihydroazepino[4,3,2-de]phthalazin-3(2H)-one;
5-fluoro-10-(1-(2-hydroxyethyl)-1H-1,2,4-triazol-5-yl)-9-(4-((methylamino)methyl)phenyl)-9,10-
dihydroazepino[4,3,2-de]phthalazin-3(2H)-one;
5-fluoro-10-(1-(3-hydroxypropyl)-1H-1,2,4-triazol-5-yl)-9-(4-((methylamino)methyl)phenyl)-9,10-
dihydroazepino[4,3,2-de]phthalazin-3(2H)-one;
9-(4-((dimethylamino)methyl)phenyl)-5-fluoro-10-(1H-1,2,4-triazol-5-yl)-9,10-dihydroazepino[4,3,2-
de]phthalazin-3(2H)-one;
9-(4-((dimethylamino)methyl)phenyl)-5-fluoro-10-(1-methyl-1H-1,2,4-triazol-5-yl)-9,10-
dihydroazepino[4,3,2-de]phthalazin-3(2H)-one;
9-(4-((dimethylamino)methyl)phenyl)-10-(1-ethyl-1H-1,2,4-triazol-5-yl)-5-fluoro-9,10-
dihydroazepino[4,3,2-de]phthalazin-3(2H)-one;
9-(4-((dimethylamino)methyl)phenyl)-5-fluoro-10-(1-(2-hydroxyethyl)-1H-1,2,4-triazol-5-yl)-9,10-
dihydroazepino[4,3,2-de]phthalazin-3(2H)-one;
9-(4-((dimethylamino)methyl)phenyl)-5-fluoro-10-(1-(3-hydroxypropyl)-1H-1,2,4-triazol-5-yl)-9,10-
dihydroazepino[4,3,2-de]phthalazin-3(2H)-one;
9-(4-(azetidin-1-ylmethyl)phenyl)-5-fluoro-10-(1H-1,2,4-triazol-5-yl)-9,10-dihydroazepino[4,3,2-
de]phthalazin-3(2H)-one;
9-(4-(azetidin-1-ylmethyl)phenyl)-5-fluoro-10-(1-methyl-1H-1,2,4-triazol-5-yl)-9,10-
dihydroazepino[4,3,2-de]phthalazin-3(2H)-one;
9-(4-(azetidin-1-ylmethyl)phenyl)-10-(1-ethyl-1H-1,2,4-triazol-5-yl)-5-fluoro-9,10-
dihydroazepino[4,3,2-de]phthalazin-3(2H)-one;
9-(4-(azetidin-1-ylmethyl)phenyl)-5-fluoro-10-(1-propyl-1H-1,2,4-triazol-5-yl)-9,10-
dihydroazepino[4,3,2-de]phthalazin-3(2H)-one;
9-(4-(azetidin-1-ylmethyl)phenyl)-5-fluoro-10-(1-(2-hydroxyethyl)-1H-1,2,4-triazol-5-yl)-9,10-
dihydroazepino[4,3,2-de]phthalazin-3(2H)-one;
9-(4-(azetidin-1-ylmethyl)phenyl)-5-fluoro-10-(1-(3-hydroxypropyl)-1H-1,2,4-triazol-5-yl)-9,10-
dihydroazepino[4,3,2-de]phthalazin-3(2H)-one;
9-(4-fluorophenyl)-10-(1H-1,2,4-triazol-5-yl)-9,10-dihydroazepino[4,3,2-de]phthalazin-3(2H)-one;
9-(4-fluorophenyl)-10-(1-methyl-1H-1,2,4-triazol-5-yl)-9,10-dihydroazepino[4,3,2-de]phthalazin-
3(2H)-one;
9-(4-fluorophenyl)-10-(1-(2-hydroxyethyl)-1H-1,2,4-triazol-5-yl)-9,10-dihydroazepino[4,3,2-
de]phthalazin-3(2H)-one;
9-(4-fluorophenyl)-10-(1-(3-hydroxypropyl)-1H-1,2,4-triazol-5-yl)-9,10-dihydroazepino[4,3,2-
de]phthalazin-3(2H)-one;
10-(1-(2-hydroxyethyl)-1H-1,2,4-triazol-5-yl)-9-(4-((methylamino)methyl)phenyl)-9,10-
dihydroazepino[4,3,2-de]phthalazin-3(2H)-one;
10-(1-methyl-1H-1,2,4-triazol-5-yl)-9-(4-((methylamino)methyl)phenyl)-9,10-dihydroazepino[4,3,2-
de]phthalazin-3(2H)-one;
9-(4-((methylamino)methyl)phenyl)-10-(1H-1,2,4-triazol-5-yl)-9,10-dihydroazepino[4,3,2-
de]phthalazin-3(2H)-one;
9-(4-(azetidin-1-ylmethyl)phenyl)-10-(1-(2-hydroxyethyl)-1H-1,2,4-triazol-5-yl)-9,10-
dihydroazepino[4,3,2-de]phthalazin-3(2H)-one;
5-fluoro-9-(4-fluorophenyl)-7-methyl-10-(1-methyl-1H-1,2,4-triazol-5-yl)-7,8,9,10-
tetrahydroazepino[4,3,2-de]phthalazin-3(2H)-one;
tert-butyl 9-fluoro-3-(4-fluorophenyl)-4-(1-methyl-1H-1,2,4-triazol-5-yl)-7-oxo-3,4,6,7-tetrahydro-
[1,2]diazepino[5,4,3-de]phthalazine-2(1H)-carboxylate;
9-(4-(azetidin-1-ylmethyl)phenyl)-10-(1-methyl-1H-1,2,4-triazol-5-yl)-9,10-dihydroazepino[4,3,2-
de]phthalazin-3(2H)-one; and
9-(4-(azetidin-1-ylmethyl)phenyl)-10-(1H-1,2,4-triazol-5-yl)-9,10-dihydroazepino[4,3,2-
de]phthalazin-3(2H)-one.

19. A pharmaceutical composition comprising a therapeutically effective amount of a compound of any of claims 1, optionally as a pharmaceutically acceptable salt, and one or more pharmaceutically acceptable carrier(s), excipient(s), binder(s) or diluent(s) thereof.

20. A method of treating a disease comprising administering to a subject in need of treatment a therapeutically effective amount of a compound of any of claims 1 optionally as a pharmaceutically acceptable salt, or a pharmaceutical composition comprising the compound of claim 1 and one or more pharmaceutically acceptable carrier(s), excipient(s), binder(s) or diluent(s) thereof where the disease is cancer; vascular disease; septic shock; ischemic injury; reperfusion injury; neurotoxicity; hemorrhagic shock; inflammatory diseases; multiple sclerosis; secondary effects of diabetes; myelodysplastic syndromes; or acute treatment of cytotoxicity following cardiovascular surgery.

21. The method of claim 20 where the cancer is breast cancer, ovarian cancer, tumors of the endometrium, cervical carcinoma, lung cancer, prostate cancer, pancreatic cancer, hematological cancers, leukemias, colon cancer, bowel tumors, glioblastomas, lymphomas, melanomas, acute myelogenous leukemia, or multiple myeloma.
